(19) United States
(12) Patent Application Publication
(10) Pub. No.: US 2008/0188528 A1
(43) Pub. Date: Aug. 7, 2008

(54) Modulators of C3a receptor and methods of use thereof

(76) Inventors: Ronald J. Biediger, Houston, TX (US); Huong M. Bui, Pearland, TX (US); Jie Chen, Houston, TX (US); Kevin M. Henry, Pearland, TX (US); Thomas Thrash, Houston, TX (US)

Correspondence Address: JONES DAY, 222 EAST 41ST ST, NEW YORK, NY, 10017, US

(21) Appl. No.: 12/004,956

(22) Filed: Dec. 21, 2007

Related U.S. Application Data
(60) Provisional application No. 60/876,906, filed on Dec. 22, 2006.

(51) Int. Cl. A61K 31/4439; C07D 213/02; A61K 31/44; A61K 31/381; A61K 31/34; A61P 25/00; A61P 31/00; A61P 15/08; A61P 9/00; C07D 307/34; C07D 333/02; A61K 31/19

(52) U.S. Cl. 514/341; 546/298; 514/350; 514/557; 562/400; 546/274.7; 549/29; 514/438; 549/487; 514/461

(57) ABSTRACT

Provided are compounds that are modulators of C3a receptor activity, compositions containing the compounds and methods of use of the compounds and compositions. In certain embodiments, the compounds are pyridones. In certain embodiments, provided are methods for treatment or amelioration of diseases associated with modulation of C3a receptor activity.

### 1. RELATED APPLICATION DATA

This application claims priority to U.S. provisional application Ser. No. 60/876,906, filed Dec. 22, 2006, entitled “MODULATORS OF C3A RECEPTOR AND METHODS OF USE THEREOF” to Biediger et al. The disclosure of the above referenced application is incorporated by reference herein in its entirety.

### 2. FIELD

Provided herein are compounds, compositions and methods for treating, preventing or ameliorating conditions associated with C3a receptor activity.

### 3. BACKGROUND

C3a receptor activation causes leukocyte activation, smooth muscle contraction and vascular permeability increase. Inhibition of this response is believed to retard inflammation. The diseases that involve C3a-induced inflammation are asthma, rheumatoid arthritis, psoriasis, septic shock and myocardial ischemic injury. The following studies directly or indirectly indicate that blocking of C3a receptor may be beneficial in several diseases. For example, C3a receptor-deficient mice have decreased airway eosinophilia and lung IL-4-producing cells and therefore diminished bronchoalveolar lavage levels of the Th2 cytokines, IL-5 and IL-13 (Drouin et al., J Immunol. 2002; 169(10):5926-33). C3a and C5a are increased in human bronchoalveolar lavage fluid after segmental allergen provocation in asthmatic subjects (Krug et al., Am J Respir Crit. Care Med. 2001; 164(10 Pt 1): 1841-3). In a murine model, administration of the antibody of Crry (complement receptor-related gene y), which is related to C3a and C5a, significantly suppressed development of airway hyperresponsiveness and decreased levels of inflammatory markers in bronchoalveolar lavage fluid (Taube et al., Am J Respir Crit Care Med. 2003; 168(11):1333-41). Gliatech Inc.'s antiproperdin monoclonal antibody, which causes near complete inhibition of C3a and C5b-9 formation, reduces inflammation in a rabbit immune complex deposition rheumatoid arthritis model (Gupta-Bansal et. Al., Mol Immunol. 2000 April; 37(5):191-201). Small molecule C3a inhibitor SB-290157 displays in vivo activity, including decreased paw edema, inhibition of neutrophil recruitment and reduction of airway inflammation in an asthma model (Ames et al., J Immunol. 2001; 166(10):6341-8).

Expression of C3a receptor in airway smooth muscle cells and on cells associated with allergic responses, suggested that C3a receptor may be involved in the pathophysiology of asthma and allergy. Asthma is a chronic inflammatory disease of the airways and lung mucosa with a strong correlation to atopy and acquired (IgE) immunity., However, many features of bronchial asthma, such as smooth muscle contraction, mucus secretion and recruitment of inflammatory cells, are consistent with the actions of complement anaphylatoxins, in particular C3a and C5a. The anaphylatoxins C3a and C5a are liberated as activation byproducts and are potent pro-inflammatory mediators that bind to specific cell surface receptors and cause leukocyte activation, smooth muscle contraction and vascular permeability. Genetic deletion of the C3a receptor protects against the changes in lung physiology seen after allergen challenge. Furthermore, human asthmatics develop significant levels of C3a following intra-pulmonary deposition of allergen, but not saline. Humbles et al., (2000) Nature 406:998-1001).

Because of the involvement of the C3a receptor in a variety of diseases, there is a continuing need for compounds that modulate the expression of C3a receptor and/or modulate the biological activity of C3a receptor.

### SUMMARY

Provided herein are compounds that are C3a receptor modulators, pharmaceutical compositions containing the compounds and methods of use thereof. In certain embodiments, the compounds for use in the compositions and methods provided herein have formula selected from:

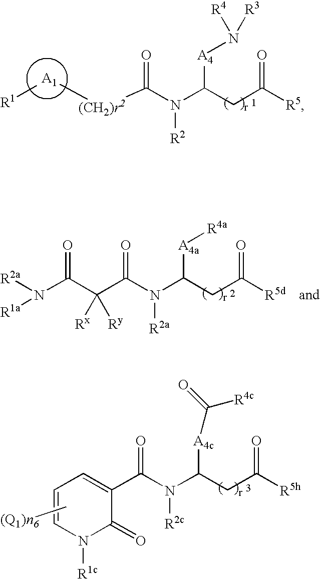

or a pharmaceutically acceptable derivative thereof, wherein the variables are chosen such that the resulting compound shows activity as a C3a modulator. In one embodiment, the compounds are C3a receptor antagonists. In other embodiments, the compounds are C3a receptor agonists.

Pharmaceutical compositions containing a compound of Formula I and a pharmaceutically acceptable carrier are provided herein. Also provided are methods for treating, preventing, or ameliorating one or more symptoms of C3a receptor mediated diseases by administering the compounds and compositions provided herein.

In certain embodiments, provided herein are methods for modulating an action of C3a receptor by contacting the receptor with a compound or composition provided herein. In one embodiment, provided herein are methods for antagonizing an action of C3a receptor by contacting the receptor with a compound or composition provided herein. In another embodiment, provided herein are methods for agonizing an action of C3a receptor by contacting the receptor with a compound or composition provided herein. In other embodiments, provided herein are methods for treatment, prevention, or amelioration of one or more symptoms of diseases or conditions associated with C3a receptor activity, including, but not limited to acute inflammatory disease, atherosclerosis, chronic polyarthritis, systemic vasculitis, multiple sclerosis, Alzheimer's Disease, CNS inflammatory disease, Crohn's Disease, food allergies, non-bronchial allergies, osteoarthritis, osteoporosis, thyroid disease, and coronary heart disease. In one embodiment, the diseases that involve C3a-induced inflammation are asthma, rheumatoid arthritis, psoriasis, septic shock and myocardial ischemic injury.

### 5. DETAILED DESCRIPTION

### 5.1 Definitions

Unless defined otherwise, all technical and scientific terms used herein have the same meaning as is commonly understood by one of ordinary skill in the art. All patents, applications, published applications and other publications are incorporated by reference in their entirety. In the event that there are a plurality of definitions for a term herein, those in this section prevail unless stated otherwise.

As used herein “subject” is an animal, such as a mammal, including human, such as a patient.

The terms “C3a receptor mediated disease, or “C3a receptor mediated condition”, as used herein, mean any disease or other deleterious condition or state in which C3a receptor is known to play a role. Such diseases or conditions include, without limitation, acute inflammatory disease, atherosclerosis, chronic polyarthritis, systemic vasculitis, multiple sclerosis, Alzheimer's Disease, CNS inflammatory disease, Crohn's Disease, food allergies, non-bronchial allergies, osteoarthritis, osteoporosis, thyroid disease, and coronary heart disease. Also included are diseases that involve C3a-induced inflammation, including asthma, rheumatoid arthritis, psoriasis, septic shock and myocardial ischemic injury.

As used herein, biological activity refers to the in vivo activities of a compound or physiological responses that result upon in vivo administration of a compound, composition or other mixture. Biological activity, thus, encompasses therapeutic effects and pharmacokinetic behaviour of such compounds, compositions and mixtures. Biological activities can be observed in in vitro systems designed to test for such activities.

As used herein, pharmaceutically acceptable derivatives of a compound include salts, esters, enol ethers, enol esters, acetals, ketals, orthoesters, hemiacetals, hemiketals, acids, bases, solvates, hydrates or prodrugs thereof. Such derivatives may be readily prepared by those of skill in this art using known methods for such derivatization.

The compounds produced may be administered to animals or humans without substantial toxic effects and either are pharmaceutically active or are prodrugs. Pharmaceutically acceptable salts include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and inorganic salts, such as but not limited to, sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates, mesylates, and fumarates. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfonic acids, sulfinic acids and boronic acids. Pharmaceutically acceptable enol ethers include, but are not limited to, derivatives of formula C-C(OR) where R is hydrogen, alkyl, alkenyl, alkynyl, aryl, aralkyl and cycloalkyl. Pharmaceutically acceptable enol esters include, but are not limited to, derivatives of formula C═C(OC(O)R) where R is hydrogen, alkyl, alkenyl, alkynyl, aryl, aralkyl and cycloalkyl. Pharmaceutically acceptable solvates and hydrates are complexes of a compound with one or more solvent or water molecules, or 1 to about 100, or 1 to about 10, or one to about 2, 3 or 4, solvent or water molecules.

As used herein, treatment means any manner in which one or more of the symptoms of a disease or disorder are ameliorated or otherwise beneficially altered. Treatment also encompasses any pharmaceutical use of the compositions herein, such as use for treating inflammation.

As used herein, amelioration of the symptoms of a particular disorder by administration of a particular compound or pharmaceutical composition refers to any lessening, whether permanent or temporary, lasting or transient that can be attributed to or associated with administration of the composition.

As used herein, and unless otherwise indicated, the terms “manage,” “managing” and “management” encompass preventing the recurrence of the specified disease or disorder in a patient who has already suffered from the disease or disorder, and/or lengthening the time that a patient who has suffered from the disease or disorder remains in remission. The terms encompass modulating the threshold, development and/or duration of the disease or disorder, or changing the way that a patient responds to the disease or disorder.

As used herein, the IC₅₀ refers to an amount, concentration or dosage of a particular test compound that achieves a 50% inhibition of a maximal response in an assay that measures such response.

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound. The instant disclosure is meant to include all such possible isomers, as well as, their racemic and optically pure forms. Optically active (+) and (−), (R)- and (S)-, or (D)- and (L)-isomers may be prepared using chiral synthons or chiral reagents, or resolved using conventional techniques, such as reverse phase HPLC. When the compounds described herein contain olefinic double bonds or other centers of geometric asymmetry, and unless specified otherwise, it is intended that the compounds include both E and Z geometric isomers. Likewise, all tautomeric forms are also intended to be included.

As used herein, the nomenclature alkyl, alkoxy, carbonyl, etc. is used as is generally understood by those of skill in this art.

As used herein, alkyl, alkenyl and alkynyl carbon chains, if not specified, contain from 1 to 20 carbons, or 1 to 16 carbons, and are straight or branched. Alkenyl carbon chains of from 2 to 20 carbons, in certain embodiments, contain 1 to 8 double bonds, and the alkenyl carbon chains of 2 to 16 carbons, in certain embodiments, contain 1 to 5 double bonds. Alkynyl carbon chains of from 2 to 20 carbons, in certain embodiments, contain 1 to 8 triple bonds, and the alkynyl carbon chains of 2 to 16 carbons, in certain embodiments, contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-pentyl, isohexyl, ethenyl, propenyl, butenyl, pentenyl, acetylenyl and hexynyl. As used herein, lower alkyl, lower alkenyl, and lower alkynyl refer to carbon chains having from about 1 or about 2 carbons up to about 6 carbons. As used herein, “alkten)(yn)yl” refers to an alkyl group containing at least one double bond and at least one triple bond.

As used herein, “heteroalkyl” refers to a straight, branched or cyclic, in certain embodiments straight or branched, aliphatic hydrocarbon group having, inserted in the hydrocarbon chain one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl, S(O)₂R′ or COR′, where R′ is alkyl, aryl, aralkyl, heteroaryl, heteroaralkyl, OY or —NYY′, where Y and Y′ are each independently hydrogen, alkyl, aryl, heteroaryl, cycloalkyl or heterocyclyl, in one embodiment having from 1 to about 20 atoms, in another embodiment having from 1 to 12 atoms in the chain.

As used herein, “cycloalkyl” refers to a saturated mono- or multicyclic ring system, in certain embodiments of 3 to 10 carbon atoms, in other embodiments of 3 to 6 carbon atoms; cycloalkenyl and cycloalkynyl refer to mono- or multicyclic ring systems that respectively include at least one double bond and at least one triple bond. Cycloalkenyl and cycloalkynyl groups may, in certain embodiments, contain 3 to 10 carbon atoms, with cycloalkenyl groups, in further embodiments, containing 4 to 7 carbon atoms and cycloalkynyl groups, in further embodiments, containing 8 to 10 carbon atoms. The ring systems of the cycloalkyl, cycloalkenyl and cycloalkynyl groups may be composed of one ring or two or more rings which may be joined together in a fused, bridged or spiro-connected fashion. “Cycloalk(en)(yn)yl” refers to a cycloalkyl group containing at least one double bond and at least one triple bond.

As used herein, “substituted alkyl,” “substituted alkenyl,” “substituted alkynyl,” “substituted cycloalkyl,” “substituted cycloalkenyl,” and “substituted cycloalkynyl” refer to alkyl, alkenyl, alkynyl, cycloalkyl, cycloalkenyl and cycloalkynyl groups, respectively, that are substituted with one or more substituents, in certain embodiments one to three or four substituents, where the substituents are as defined herein, generally selected from Q1.

As used herein, “aryl” refers to aromatic monocyclic or multicyclic groups containing from 6 to 19 carbon atoms. Aryl groups include, but are not limited to groups such as fluorenyl, substituted fluorenyl, phenyl, substituted phenyl, naphthyl and substituted naphthyl.

As used herein, “heteroaryl” refers to a monocyclic or multicyclic aromatic ring system, in certain embodiments, of about 5 to about 15 members where one or more, in one embodiment 1 to 3, of the atoms in the ring system is a heteroatom, that is, an element other than carbon, including but not limited to, nitrogen, oxygen or sulfur. The heteroaryl group may be optionally fused to a benzene ring. Heteroaryl groups include, but are not limited to, furyl, imidazolyl, pyrrolidinyl, pyrimidinyl, tetrazolyl, thienyl, pyridyl, pyrrolyl, N-methylpyrrolyl, quinolinyl and isoquinolinyl.

As used herein, a “heteroarylium” group is a heteroaryl group that is positively charged on one or more of the heteroatoms.

As used herein, “heterocyclyl” refers to a monocyclic or multicyclic non-aromatic ring system, in one embodiment of 3 to 10 members, in another embodiment of 4 to 7 members, in a further embodiment of 5 to 6 members, where one or more, in certain embodiments, 1 to 3, of the atoms in the ring system is a heteroatom, that is, an element other than carbon, including but not limited to, nitrogen, oxygen or sulfur. In embodiments where the heteroatom(s) is(are) nitrogen, the nitrogen is optionally substituted with alkyl, alkenyl, alkynyl, aryl, heteroaryl, aralkyl, heteroaralkyl, cycloalkyl, heterocyclyl, cycloalkylalkyl, heterocyclylalkyl, acyl, guanidino, amidino or the nitrogen may be quaternized to form an ammonium group where the substituents are selected as above.

As used herein, “substituted aryl,” “substituted heteroaryl” and “substituted heterocyclyl” refer to aryl, heteroaryl and heterocyclyl groups, respectively, that are substituted with one or more substituents, in certain embodiments one to three or four substituents, where the substituents are as defined herein, generally selected from Q1.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “alkylene” refers to a straight, branched or cyclic, in certain embodiments straight or branched, divalent aliphatic hydrocarbon group, in one embodiment having from 1 to about 20 carbon atoms, in another embodiment having from 1 to 12 carbons. In a further embodiment alkylene includes lower alkylene. There may be optionally inserted along the alkylene group one or more oxygen, sulfur, including S(═O) and S(═O)₂ groups, or substituted or unsubstituted nitrogen atoms, including —NR— and —N⁺RR— groups, where the nitrogen substituent(s) is(are) alkyl, aryl, aralkyl, heteroaryl, heteroaralkyl, S(═O)₂R′ or COR′, where R′ is alkyl, aryl, aralkyl, heteroaryl, heteroaralkyl, —OY or —NYY′, where Y and Y′ are each independently hydrogen, alkyl, aryl, heteroaryl, cycloalkyl or heterocyclyl. Alkylene groups include, but are not limited to, methylene (—CH₂—), ethylene (—CH₂CH₂—), propylene (—(CH₂)₃—), methylenedioxy (—O—CH₂—O—) and ethylenedioxy (—O—(CH₂)₂—O—). The term “lower alkylene” refers to alkylene groups having 1 to 6 carbons. In certain embodiments, alkylene groups are lower alkylene, including alkylene of 1 to 3 carbon atoms.

As used herein, “alkenylene” refers to a straight, branched or cyclic, in one embodiment straight or branched, divalent aliphatic hydrocarbon group, in certain embodiments having from 2 to about 20 carbon atoms and at least one double bond, in other embodiments 1 to 12 carbons. In further embodiments, alkenylene groups include lower alkenylene. There may be optionally inserted along the alkenylene group one or more oxygen, sulfur or substituted or unsubstituted nitrogen atoms, where the nitrogen substituent is alkyl. Alkenylene groups include, but are not limited to, —CH═CH—CH═CH— and —CH═CH—CH₂—. The term “lower alkenylene” refers to alkenylene groups having 2 to 6 carbons. In certain embodiments, alkenylene groups are lower alkenylene, including alkenylene of 3 to 4 carbon atoms.

As used herein, “alkynylene” refers to a straight, branched or cyclic, in certain embodiments straight or branched, divalent aliphatic hydrocarbon group, in one embodiment having from 2 to about 20 carbon atoms and at least one triple bond, in another embodiment 1 to 12 carbons. In a further embodiment, alkynylene includes lower alkynylene. There may be optionally inserted along the alkynylene group one or more oxygen, sulfur or substituted or unsubstituted nitrogen atoms, where the nitrogen substituent is alkyl. Alkynylene groups include, but are not limited to, —C≡C—C═C—, —C≡C— and —C≡C—CH₂—. The term “lower alkynylene” refers to alkynylene groups having 2 to 6 carbons. In certain embodiments, alkynylene groups are lower alkynylene, including alkynylene of 3 to 4 carbon atoms.

As used herein, “alk(en)(yn)ylene” refers to a straight, branched or cyclic, in certain embodiments straight or branched, divalent aliphatic hydrocarbon group, in one embodiment having from 2 to about 20 carbon atoms and at least one triple bond, and at least one double bond; in another embodiment 1 to 12 carbons. In further embodiments, alk(en)(yn)ylene includes lower alk(en)(yn)ylene. There may be optionally inserted along the alkynylene group one or more oxygen, sulfur or substituted or unsubstituted nitrogen atoms, where the nitrogen substituent is alkyl. Alk(en)(yn)ylene groups include, but are not limited to, —C═C—(CH₂)ₙ—C≡C—, where n is 1 or 2. The term “lower alk(en)(yn)ylene” refers to alk(en)(yn)ylene groups having up to 6 carbons. In certain embodiments, alk(en)(yn)ylene groups have about 4 carbon atoms.

As used herein, “cycloalkylene” refers to a divalent saturated mono- or multicyclic ring system, in certain embodiments of 3 to 10 carbon atoms, in other embodiments 3 to 6 carbon atoms; cycloalkenylene and cycloalkynylene refer to divalent mono- or multicyclic ring systems that respectively include at least one double bond and at least one triple bond. Cycloalkenylene and cycloalkynylene groups may, in certain embodiments, contain 3 to 10 carbon atoms, with cycloalkenylene groups in certain embodiments containing 4 to 7 carbon atoms and cycloalkynylene groups in certain embodiments containing 8 to 10 carbon atoms. The ring systems of the cycloalkylene, cycloalkenylene and cycloalkynylene groups may be composed of one ring or two or more rings which may be joined together in a fused, bridged or spiro-connected fashion. “Cycloalk(en)(yn)ylene” refers to a cycloalkylene group containing at least one double bond and at least one triple bond.

As used herein, “substituted alkylene,” “substituted alkenylene,” “substituted alkynylene,” “substituted cycloalkylene,” “substituted cycloalkenylene,” and “substituted cycloalkynylene” refer to alkylene, alkenylene, alkynylene, cycloalkylene, cycloalkenylene and cycloalkynylene groups, respectively, that are substituted with one or more substituents, in certain embodiments one to three or four substituents, where the substituents are as defined herein, generally selected from Q¹.

As used herein, “arylene” refers to a monocyclic or polycyclic, in certain embodiments monocyclic, divalent aromatic group, in one embodiment having from 5 to about 20 carbon atoms and at least one aromatic ring, in another embodiment 5 to 12 carbons. In further embodiments, arylene includes lower arylene. Arylene groups include, but are not limited to, 1,2-, 1,3- and 1,4-phenylene. The term “lower arylene” refers to arylene groups having 5 or 6 carbons.

As used herein, “heteroarylene” refers to a divalent monocyclic or multicyclic aromatic ring system, in one embodiment of about 5 to about 15 members where one or more, in certain embodiments 1 to 3, of the atoms in the ring system is a heteroatom, that is, an element other than carbon, including but not limited to, nitrogen, oxygen or sulfur.

As used herein, “heterocyclylene” refers to a divalent monocyclic or multicyclic non-aromatic ring system, in certain embodiments of 3 to 10 members, in one embodiment 4 to 7 members, in another embodiment 5 to 6 members, where one or more, including 1 to 3, of the atoms in the ring system is a heteroatom, that is, an element other than carbon, including but not limited to, nitrogen, oxygen or sulfur.

As used herein, “substituted arylene,” “substituted heteroarylene” and “substituted heterocyclylene” refer to arylene, heteroarylene and heterocyclylene groups, respectively, that are substituted with one or more substituents, in certain embodiments one to three or four substituents, where the substituents are as defined herein, generally selected from Q¹.

As used herein, “halo”, “halogen” or “halide” refers to F, Cl, Br or I.

As used herein, pseudohalides or pseudohalo groups are groups that behave substantially similar to halides. Such compounds can be used in the same manner and treated in the same manner as halides. Pseudohalides include, but are not limited to, cyano, thiocyanate, selenocyanate, trifluoromethoxy, and azide.

As used herein, “haloalkyl” refers to an alkyl group in which one or more of the hydrogen atoms are replaced by halogen. Such groups include, but are not limited to, chloromethyl, trifluoromethyl and 1 chloro 2 fluoroethyl.

As used herein, “haloalkoxy” refers to RO in which R is a haloalkyl group.

As used herein, “carboxy” refers to a divalent radical, —C(O)O—.

As used herein, “aminocarbonyl” refers to C(O)NH₂.

As used herein, “alkylaminocarbonyl” refers to C(O)NHR in which R is alkyl, including lower alkyl. As used herein, “dialkylaminocarbonyl” refers to C(O)NR′R in which R′ and R are independently alkyl, including lower alkyl; “carboxamide” refers to groups of formula —NR′COR in which R′ and R are independently alkyl, including lower alkyl.

As used herein, “arylalkylaminocarbonyl” refers to —C(O)NRR′ in which one of R and R′ is aryl, including lower aryl, such as phenyl, and the other of R and R′ is alkyl, including lower alkyl.

As used herein, “arylaminocarbonyl” refers to —C(O)NHR in which R′ is aryl, including lower aryl, such as phenyl.

As used herein, “hydroxycarbonyl” refers to —COOH.

As used herein, “alkoxycarbonyl” refers to —C(O)OR in which R is alkyl, including lower alkyl.

As used herein, “aryloxycarbonyl” refers to —C(O)OR in which R is aryl, including lower aryl, such as phenyl.

As used herein, “alkoxy” and “alkylthio” refer to RO— and RS—, in which R is alkyl, including lower alkyl.

As used herein, “aryloxy” and “arylthio” refer to RO— and RS—, in which R is aryl, including lower aryl, such as phenyl.

As used herein, “oxo” refers to ═O.

As used herein, “thioxo” refers to ═S.

Where the number of any given substituent is not specified (e.g., “haloalkyl”), there may be one or more substituents present. For example, “haloalkyl” may include one or more of the same or different halogens. As another example,

“C₁₋₃alkoxyphenyl” may include one or more of the same or different alkoxy groups containing one, two or three carbons.

As used herein, the abbreviations for any protective groups, amino acids and other compounds, are, unless indicated otherwise, in accord with their common usage, recognized abbreviations, or the IUPAC-IUB Commission on Biochemical Nomenclature (see, (1972) Biochem. 11:942-944).

5.2 Compounds

In certain embodiments, the compounds for use in the compositions and methods provided herein have Formula I:

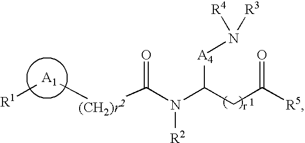

or pharmaceutically acceptable derivatives thereof,

wherein A₁ is arylene, heteroarylene or heterocyclylene;

R¹ is alkyl, alkenyl, alkynyl, aryl, aralkyl, alkylaryl, cycloalkyl, cycloalkylalkyl, heterocyclyl, heterocyclylalkyl, heteroaryl or heteroarylalkyl;

R² is hydrogen, alkyl, alkenyl, alkynyl, cycloalkyl, aryl, heteroaryl or heterocyclyl;

R⁵ is OR or NR^{5a}R^{5b};

R^{5a} and R^{5b} are selected as follows:

i) R^{5a} and R^{5b} are each independently hydrogen, alkyl, alkenyl, alkynyl, cycloalkyl, aryl, heteroaryl or heterocyclyl; or

ii) R^{5a} and R^{5b} together with the nitrogen atom on which they are substituted form a 3-7 membered heterocyclic or heteroaryl ring;

A₄ is alkylene, alkenylene, alkynylene, alk(en)(yn)ylene, cycloalkylene, arylene, aralkylene, alkylarylene, heteroarylene or heterocyclylene;

R³ and R⁴ are selected as follow:

i) R³ is hydrogen, alkyl, alkenyl, alkynyl, cycloalkyl, aryl, heteroaryl or heterocyclyl; and R⁴ is R³, —C(═R⁶)NR⁷R⁸, —C(═NR)R, C(O)R⁹, —S(O)ₙR⁹, —S(O)₂NHR^{9a}, —C(O)NHR^{9a} or —(CH₂)ₓOH;

ii) R³ and R⁴ together form ═CRNR^{5a}R^{5b}; or

iii) R³ is absent, hydrogen or lower alkyl, and R⁴ forms a 5-7 membered heteroaromatic or heterocyclic ring along with A₄ and the nitrogen atom on which it is substituted;

each R is independently hydrogen, hydroxy, alkoxy, alkyl, alkenyl, alkynyl, aryl, alkylaryl, heteroaryl, heterocyclyl, cycloalkyl or aralkyl;

R⁶ is NR^{6x} or O;

R^{6x} is hydrogen, hydroxy, alkyl, alkenyl, alkynyl, aryl, alkoxy, C(O)R⁹ or S(O)ₙR⁹;

R⁷ and R⁸ are selected as follows:

i) R⁷ is hydrogen, alkyl, alkenyl, alkynyl, cycloalkyl, aryl, heteroaryl or heterocyclyl; and R⁸ is selected from R⁷, nitro, C(O)R⁹ and S(O)ₙR⁹; or

ii) R⁷ and R⁸ together with the nitrogen atom on which they are substituted form a 3-7 membered heterocyclic or heteroaryl ring;

R⁹ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy or —C(O)R;

R^{9a} is hydrogen, alkyl, alkenyl, alkynyl, cycloalkyl, aryl, heteroaryl, heterocyclyl or —C(O)R;

n is 0-2; r¹ is 0-3, r² is 0-3 and x is 1-6.

In certain embodiments, the compounds for use in the compositions and methods provided herein are of Formula I or pharmaceutically acceptable derivatives thereof,

wherein A₁ is arylene, heteroarylene or heterocyclylene;

R¹ is alkyl, alkenyl, alkynyl, aryl, aralkyl, alkylaryl, cycloalkyl, cycloalkylalkyl, heterocyclyl, heterocyclylalkyl, heteroaryl or heteroarylalkyl;

R² is hydrogen, alkyl, alkenyl, alkynyl, cycloalkyl, aryl, heteroaryl or heterocyclyl;

R⁵ is OR or NR^{5a}R^{5b};

R^{5a} and R^{5b} are selected as follows:

i) R^{5a} and R^{5b} are each independently hydrogen, alkyl, alkenyl, alkynyl, cycloalkyl, aryl, heteroaryl or heterocyclyl; or

ii) R^{5a} and R^{5b} together with the nitrogen atom on which they are substituted form a 3-7 membered heterocyclic or heteroaryl ring;

A₄ is alkylene, alkenylene, alkynylene, alk(en)(yn)ylene, cycloalkylene, arylene, aralkylene, alkylarylene, heteroarylene or heterocyclylene;

R³ and R⁴ are selected as follow:

i) R³ is hydrogen, alkyl, alkenyl, alkynyl, cycloalkyl, aryl, heteroaryl or heterocyclyl; and R⁴ is R³, C(═R⁶)NR⁷R⁸, —C(NR)R, —C(O)R⁹, S(O)ₙR⁹, —S(O)₂NHR^{9a}, —C(O)NHR^{9a} or —(CH₂)ₓOH;

ii) R³ and R⁴ together form ═CRNR^{5a}R^{5b}; or

iii) R³ is absent, hydrogen or lower alkyl, and R⁴ forms a 5-7 membered heteroaromatic or heterocyclic ring along with A₄ and the nitrogen atom on which it is substituted;

each R is independently hydrogen, alkyl, alkenyl, alkynyl, aryl, alkylaryl, heteroaryl, heterocyclyl, cycloalkyl or aralkyl;

R⁶ is NR^{6x} or O;

R^{6x} is hydrogen, alkyl, alkenyl, alkynyl, aryl, C(O)R⁹ or S(O)ₙR⁹;

R⁷ and R⁸ are selected as follows:

i) R⁷ is hydrogen, alkyl, alkenyl, alkynyl, cycloalkyl, aryl, heteroaryl or heterocyclyl; and R⁸ is selected from R⁷, nitro, C(O)R⁹ and S(O)ₙR⁹; or

ii) R⁷ and R⁸ together with the nitrogen atom on which they are substituted form a 3-7 membered heterocyclic or heteroaryl ring;

R⁹ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy, aralkoxy or —C(O)R;

R^{9a} is hydrogen, alkyl, alkenyl, alkynyl, cycloalkyl, aryl, heteroaryl, heterocyclyl or —C(O)R;

n is 0-2; r¹ is 0-3, r² is 0-3 and x is 1-6.

In certain embodiments, R, R¹—R⁹, A₁, R^{5a}, R^{5b}, R^{6x} and R^{9a} are optionally substituted with one or more, in certain embodiments, 1, 2, 3 or 4 substituents, each independently selected from Q¹, where Q¹ is halo, pseudohalo, hydroxy, oxo, thioxo, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q¹ groups, which substitute atoms in a 1, 2 or 1,3 arrangement, together form alkylene, alkyleneoxy (i.e., —O—(CH₂)_{y}—), alkylenethioxy (i.e., —S—(CH₂)_{y}—), alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form alkylene; and

each Q¹ is independently unsubstituted or substituted with one, two or three substituents, each independently selected from Q²;

each Q² is independently halo, pseudohalo, hydroxy, oxo, thioxo, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylene, alkyleneoxy (i.e, —O—(CH₂)_{y}—), alkylenethioxy (i.e., —S—(CH₂—)y), alkylenedioxy (i.e., —O—(CH₂)_{y}—O—), thioalkylenoxy (i.e., —S—(CH₂)—O—) or alkylenedithioxy (i.e., —S—(CH₂)_{y}—S—) where y is 1 or 2; or two Q² groups, which substitute the same atom, together form alkylene;

R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;

R⁵¹, R⁵² and R¹³ are each independently hydrogen, alkyl, aryl, aralkyl, heteroaryl, heteroaralkyl, heterocyclyl or heterocyclylalkyl;

R⁶⁰ is hydrogen, alkyl, aryl, aralkyl, heteroaryl, heteroaralkyl, heterocyclyl or heterocyclylalkyl; and

R⁶³ is alkoxy, aralkoxy, alkyl, heteroaryl, heterocyclyl, aryl or —NR⁷⁰R⁷¹.

In certain embodiments, A₁ is arylene or heteroarylene. In certain embodiments, A₁ is a 5 to 7 membered heteroarylene containing one, two, three or more heteroatoms selected from N, S and O. In one embodiment, A₁ is a five or six membered heteroarylene ring, for example heteroarylene ring containing one or more oxygen, sulfur and/or nitrogen atoms. In one embodiment, A₁ is a 5 to 7 membered heterocyclylene containing one, two, three or more heteroatoms selected from N, S and O. In one embodiment, A₁ is a five or six membered heterocyclylene ring, for example heterocyclylene ring containing one or more oxygen, sulfur and/or nitrogen atoms. In one embodiment, A₁ is pyridinyl, optionally substituted with an oxo group. In one embodiment, A₁ is phenylene or 2-oxo-1,2-dihydropyridinyl. In one embodiment, A₁ is a furanyl. In one embodiment, A₁ is a thienyl.

In certain embodiments, the compounds provided herein are such that when A₁ is furanyl, R⁴ is other than —C(═NH)NH₂. In certain embodiments, the compounds provided herein are such that when A₁ is furanyl, R⁴ is other than —C(═R⁶)NR⁷R⁸. In certain embodiments, the compounds provided herein are such that when A₁ is 5-membered heteroarylene, R⁴ is other than —C(═R⁶)NR⁷R⁸. In certain embodiments, the compounds provided herein are such that when A₁ is other than furanyl. In certain embodiments, the compounds provided herein are such that A₁ is other than a 5-membered heteroarylene.

In one embodimen, r² is 1. In one embodimen, r² is 0.

In one embodiment, the compound has formula II:

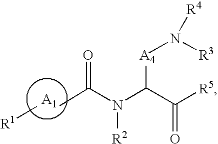

or a pharmaceutically acceptable derivative thereof, wherein the variables are as described herein.

In one embodiment, the compound has formula III:

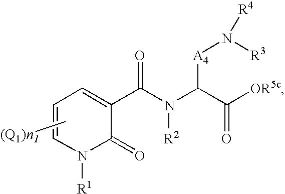

or a pharmaceutically acceptable derivative thereof, wherein

R^{5c} is hydrogen or lower alkyl;

n₁ is 0 to 3 and the other variables are as described elsewhere herein.

In certain embodiments, R¹ is substituted or unsubstituted alkyl, aryl, aralkyl, alkylaryl, cycloalkyl, cycloalkylalkyl, heterocyclylalkyl or heteroarylalkyl. In one embodiment, R¹ is methyl, benzyl, phenyl, 2,2-diphenylethyl, 3,3-diphenylpropyl, naphthylmethyl, biphenylmethyl, dithiophen-2-yl-methyl or naphthyl. In one embodiment, R¹ is methyl, benzyl, phenyl, 2,2-diphenylethyl, 3,3-diphenylpropyl, naphthylmethyl, biphenylmethyl or naphthyl.

In one embodiment, the substituents on R¹ are selected from one or more groups, in one embodiment, one, two, three or four groups, selected from alkyl, halo, haloalkyl, aryl, aralkyl, alkylaryl, haloaryl, alkoxy, haloaryl and haloalkylaryl. In one embodiment, the substituents on R¹ are selected from one, two, three or four groups, selected from methyl, fluoro, trifluoromethyl, bromo, isopropyl, phenyl, benzyl, naphthyl, isopropylphenyl, fluorophenyl, methoxy, o-tolyl, m-tolyl, p-tolyl, fluorophenyl, dimethylphenyl and trifluoromethylphenyl.

In one embodiment, R¹ has formula:

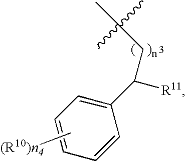

wherein n₃ is 0 to 3; n₄ is 0 to 5;

R¹¹ is hydrogen, alkyl, aryl, alkylaryl, haloaryl or haloalkylaryl; and

R¹⁰ is hydrogen, alkyl, halo, haloalkyl, aryl, aralkyl, alkylaryl, haloaryl, alkoxy, haloaryl or haloalkylaryl.

In one embodiment, R¹¹ is hydrogen, methyl, phenyl, o-tolyl, m-tolyl, p-tolyl, 4-fluorophenyl, 3-fluorophenyl, 2-fluorophenyl, 3,5-dimethylphenyl, 3-trifluoromethylphenyl or 4-trifluoromethylphenyl. In one embodiment, R¹⁰ is hydrogen, methyl, fluoro, bromo, isopropyl, phenyl, benzyl, naphthyl, isopropylphenyl, fluorophenyl or methoxy. In one embodiment, n₃ is 0, 1, 2 or 3. In one embodiment, n₃ is 1 or 2. In one embodiment, n₄ is 0, 1, 2 or 3. In one embodiment, n4 is 1 or 2.

In one embodiment, R¹ has formula:

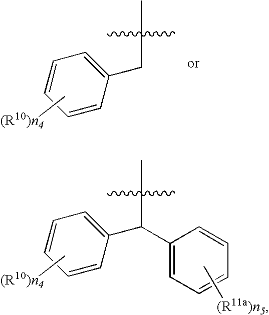

wherein R¹⁰ is hydrogen, methyl, fluoro, chloro, iodo, bromo, isopropyl, trifluoromethyl, phenyl, benzyl, naphthyl, isopropylphenyl, fluorophenyl or methoxy; R^{11a} is hydrogen, methyl, fluoro or trifluoromethyl; n₅ is 1, 2 or 3; and other variables are as described elsewhere herein.

In one embodiment, R¹ has formula:

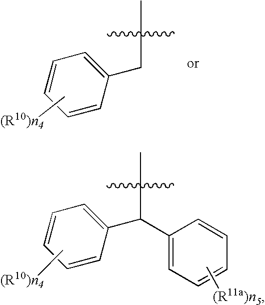

wherein R¹⁰ is hydrogen, methyl, fluoro, bromo, isopropyl, phenyl, benzyl, naphthyl, isopropylphenyl, fluorophenyl or methoxy; R^{11a} is hydrogen, methyl, fluoro or trifluoromethyl; n₅ is 1, 2 or 3; and other variables are as described elsewhere herein.

In one embodiment, R¹ has formula:

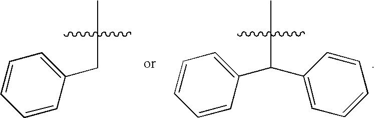

In one embodiment, R² is hydrogen or lower alkyl. In one embodiment, R² is hydrogen or methyl. In one embodiment, R² is hydrogen.

In one embodiment, R³ is hydrogen, lower alkyl or aryl. In one embodiment, R³ is hydrogen, methyl, ethyl or phenyl. In one embodiment, R³ is hydrogen, methyl or phenyl. In one embodiment, R³ is hydrogen.

In one embodiment, R⁵ is hydroxy, alkoxy, alkenyloxy, alkynyloxy, aryloxy, alkylaryloxy, heteroaryloxy, heterocyclyloxy, cycloalkyloxy, aralkoxy. In one embodiment, R⁵ is hydroxy or alkoxy. In one embodiment, R⁵ is hydroxy or lower alkoxy. In one embodiment, R⁵ is hydroxy or methoxy.

In one embodiment, R⁴ is alkyl, C(═NR)R, C(═R⁶)NR⁷R⁸, C(O)R⁹ or S(O)ₙR⁹. In one embodiment, R⁴ is C(═R⁶)NR⁷R⁸, C(O)R⁹ or S(O)ₙR⁹. In one embodiment, R⁴ is aryloxycarbonyl, alkylarylsulfonyl, heterocyclylsulfonyl, alkylsulfonyl, haloalkylsulfonyl, heterocyclyl, heteroaryl or C(═R⁶)NR⁷R⁸. In one embodiment, R⁴ is ethyl, benzyloxycarbonyl, p-tolylsulfonyl, methylsulfonyl, trifluoromethylsulfonyl, 4,5-dihydro-1H-imidazol-2-yl, pyrimidin-2-yl or C(═R⁶)NR⁷R⁸. In one embodiment, R⁴ is benzyloxycarbonyl, p-tolylsulfonyl, methylsulfonyl, trifluoromethylsulfonyl, 4,5-dihydro-1H-imidazol-2-yl, pyrimidin-2-yl or C(═R⁶)NR⁷R⁸.

In one embodiment, R⁷ is hydrogen, alkyl, alkenyl, alkynyl, cycloalkyl, aryl, heteroaryl or heterocyclyl; and R⁸ is selected from hydrogen, nitro, C(O)R⁹ and S(O)ₙR⁹. In one embodiment, R⁷ and R⁸ together with the nitrogen atom on which they are substituted form a 3-7 membered heterocyclic or heteroaryl ring.

In one embodiment, R⁹ is alkyl, alkoxy or aryl. In one embodiment, R⁹ is alkoxy or aryl.

In one embodiment, R⁴ is —C(═NR)R or —C(═R⁶)NR⁷R⁸, wherein

R⁶ is NR^{6x} or O;

R^{6x} is hydrogen, hydroxy, alkyl, —C(O)R⁹ or —S(O)ₙR⁹;

R⁷ is hydrogen or alkyl; and

R⁸ is hydrogen, alkyl, nitro, C(O)R⁹ or S(O)ₙR⁹; and

each R is independently selected from hydrogen, hydroxy, alkyl, carboxyalkyl, cycloalkyl, alkoxycarbonyl, aryl and heteroaryl.

In one embodiment, R⁴ is —C(═R⁶)NR⁷R⁸, wherein

R⁶ is NR^{6x} or O;

R^{6x} is hydrogen, alkyl, —C(O)R⁹ or —S(O)ₙR⁹;

R⁷ is hydrogen or alkyl; and

R⁸ is hydrogen, alkyl, nitro, C(O)R⁹ or S(O)ₙR⁹.

In one embodiment, R⁴ is —C(═R⁶)NR⁷R⁸, wherein

R⁶ is NR^{6x} or O;

R^{6x} is hydrogen, hydroxy, methyl, isopropyl, or ethoxycarbonyl;

R⁷ is hydrogen; and

R⁸ is hydrogen, nitro, isopropyl, ethoxycarbonyl or p-tolylsulfonyl.

In one embodiment, R⁴ is —C(═R⁶)NR⁷R⁸, wherein

R⁶ is NR^{6x} or O;

R^{6x} is hydrogen, methyl or ethoxycarbonyl;

R⁷ is hydrogen; and

R⁸ is hydrogen, nitro, ethoxycarbonyl or p-tolylsulfonyl.

In one embodiment, R⁴ has formula:

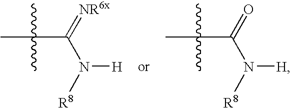

wherein R^{6x} is hydrogen, methyl or ethoxycarbonyl; and R⁸ is hydrogen, nitro, ethoxycarbonyl or p-tolysulfonyl.

In one embodiment, R⁴ has formula:

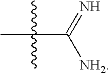

In one embodiment, R⁴ has formula:

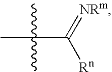

wherein R^{m} is hydrogen, hydroxy or alkyl; and Rⁿ is hydrogen, alkyl, cycloalkyl, aryl, alkoxycarbonylalyl or carboxyalkyl. In one embodiment, R^{m} is hydrogen, hydroxy or isopropyl; and Rⁿ is hydrogen, methyl, cyclopropyl, phenyl, pyridinyl, ethoxycarbonylmethyl or carboxymethyl.

In certain embodiments, A₄ is alkylene, arylene, aralkylene or alkylarylene.

In one embodiment, is —(CH₂)n₂— or arylene, where n₂ is 1-5. In one embodiment, A₄ is phenylene. In one embodiment, n₂ is 1, 2, 3, 4 or 5. In one embodiment, n₂ is 2, 3 or 4. In one embodiment, n₂ is 3.

In one embodiment, the compounds provided herein have formula IV:

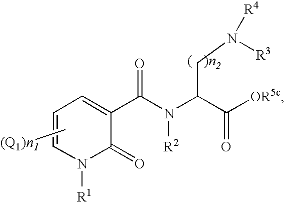

or pharmaceutically acceptable derivatives thereof, wherein n₂ is 1, 2, 3, 4 or 5. In one embodiment, the compounds have formula III, wherein R⁴ is —C(═NH)NH₂; n₂ is 3; n₁ is 0; R²R³ and R⁵ are each hydrogen and R¹ is selected from

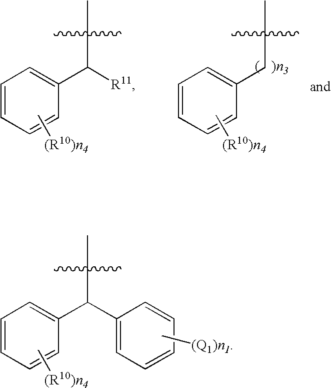

In one embodiment, the compounds have formula IV, wherein n₁ and n₄ are each independently 0, 1 or 2; R¹⁰ is halo, lower alkyl, halolower alkyl or lower alkoxy. In one embodiment, the compounds have formula IV, wherein R¹⁰ is chloro, bromo, fluoro, methyl, isopropyl or methoxy.

In one embodiment, the compound has formula V:

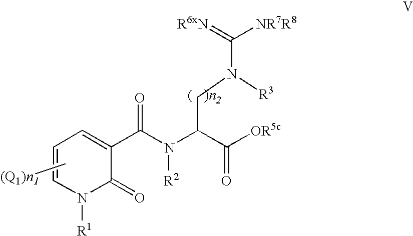

or a pharmaceutically acceptable derivative thereof, wherein the variables are as described elsewhere herein. In one embodiment, R^{6x} is hydrogen, hydroxy, alkyl, —C(O)R⁹ or —S(O)ₙR⁹; R⁷ is hydrogen or alkyl; and R⁸ is hydrogen or alkyl and the other variables are as described elsewhere herein.

In one embodiment, the compound has formula VA or VB:

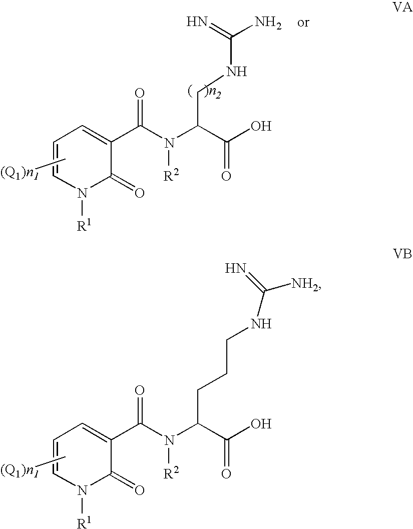

or a pharmaceutically acceptable derivative thereof, wherein the variables are as described elsewhere herein.

In one embodiment, the compound has formula VI or VII:

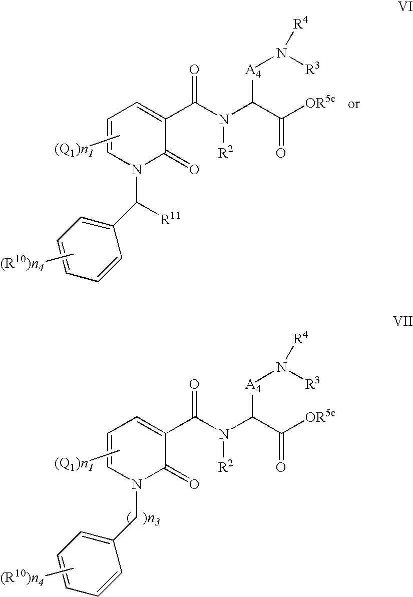

or a pharmaceutically acceptable derivative thereof, wherein the variables are as described elsewhere herein.

In one embodiment, the compound has formula VIA:

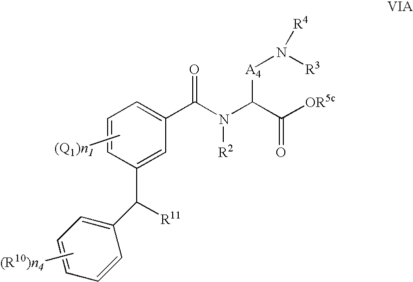

or a pharmaceutically acceptable derivative thereof, wherein the variables are as described elsewhere herein.

In one embodiment, the compound has formula:

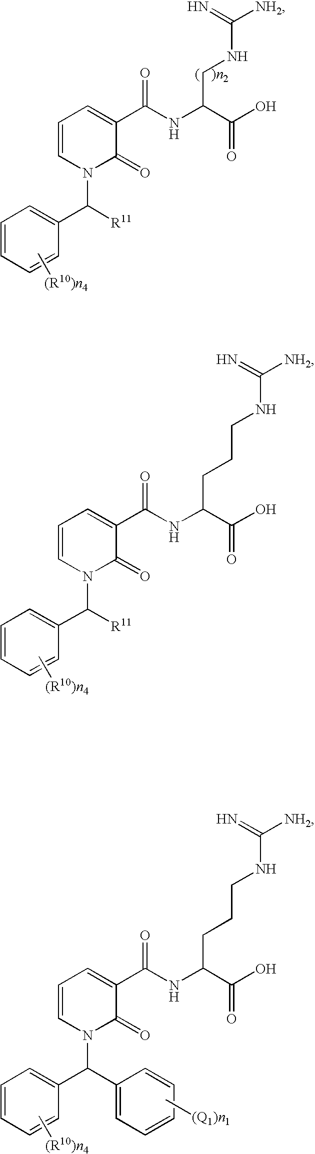

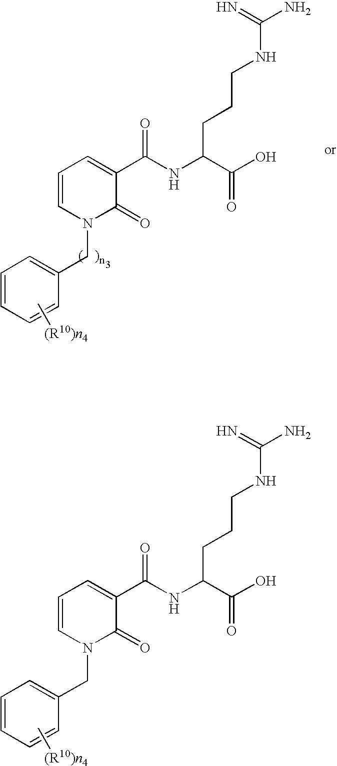

or a pharmaceutically acceptable derivative thereof, wherein the variables are as described elsewhere herein.

In one embodiment, the compound has formula:

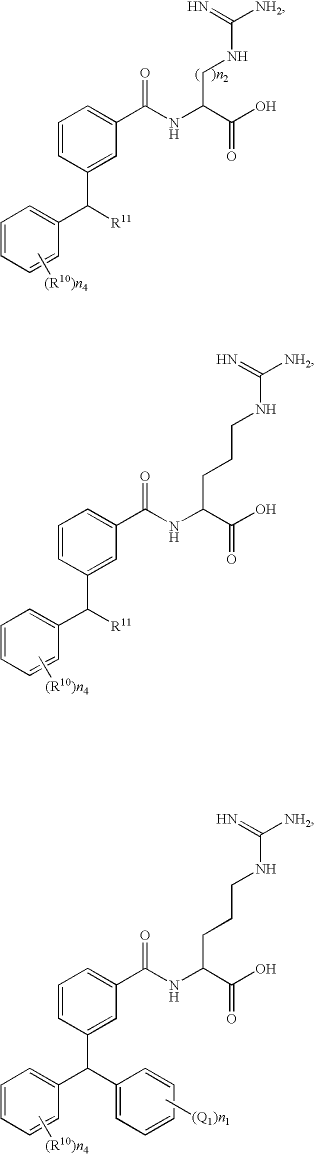

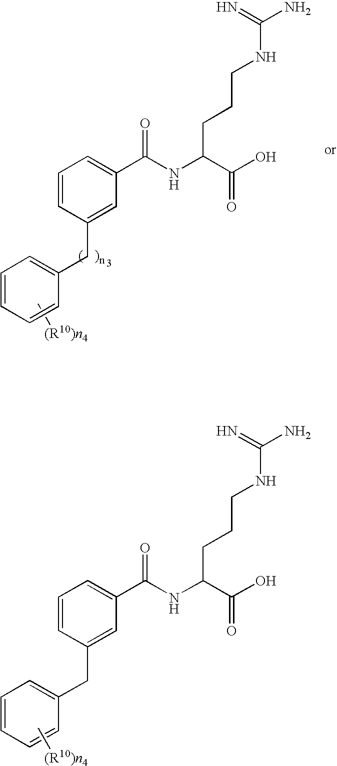

or a pharmaceutically acceptable derivative thereof, wherein the variables are as described elsewhere herein.

In one embodiment, the compound has formula:

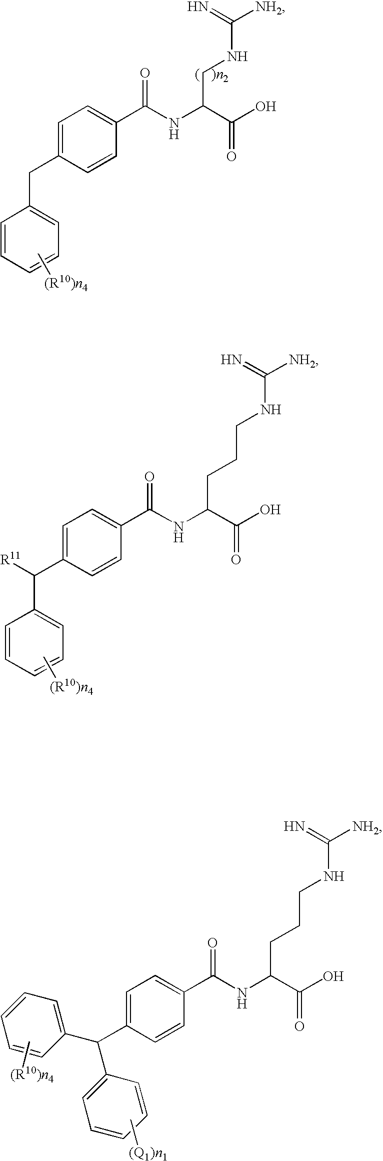

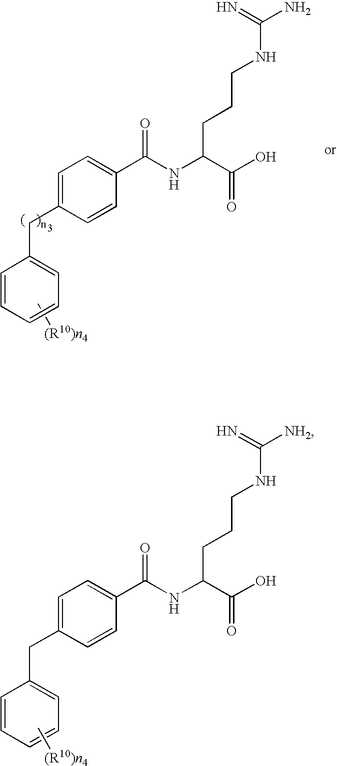

or a pharmaceutically acceptable derivative thereof, wherein the variables are as described elsewhere herein.

In one embodiment, the compound has formula:

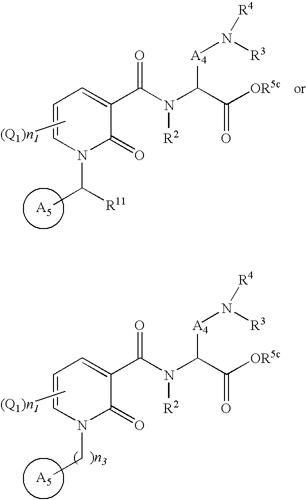

or a pharmaceutically acceptable derivative thereof, where A₅ is

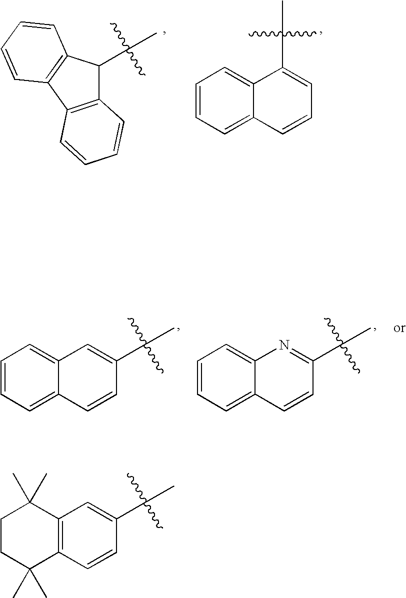

and where A₅ is optionally substituted with one or more, in one embodiment, one, two, three, four or five groups selected from halo, alkyl, and alkoxy and the other variables are as described elsewhere herein. In one embodiment, A₅ is substituted with fluoro, methyl or methoxy.

In one embodiment, the compound has formula:

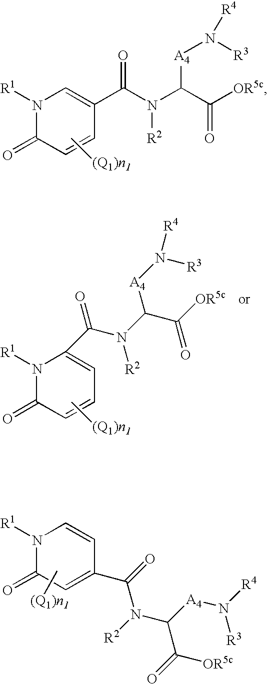

or a pharmaceutically acceptable derivative thereof, wherein the variables are as described elsewhere herein.

In one embodiment, the compound has formula:

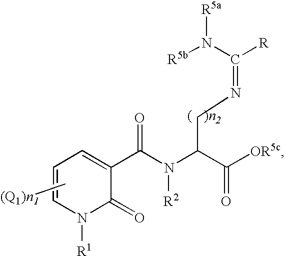

or a pharmaceutically acceptable derivative thereof, wherein the variables are as described elsewhere herein.

In one embodiment, the compound has formula:

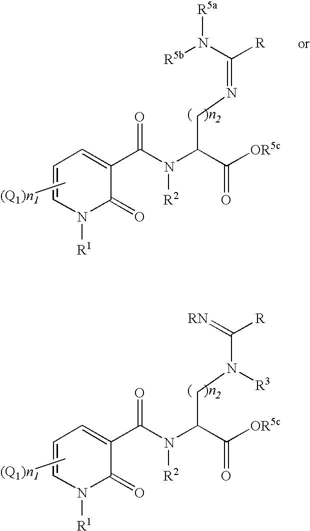

or a pharmaceutically acceptable derivative thereof, wherein the variables are as described elsewhere herein.

In one embodiment, the compound has formula:

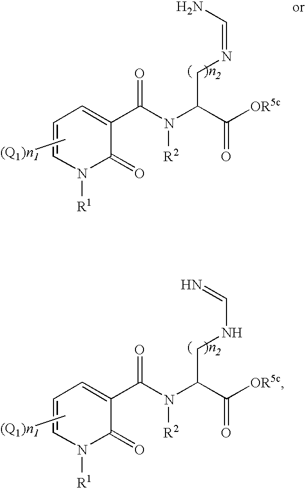

or a pharmaceutically acceptable derivative thereof, wherein the variables are as described elsewhere herein.

In one embodiment, the compound has formula:

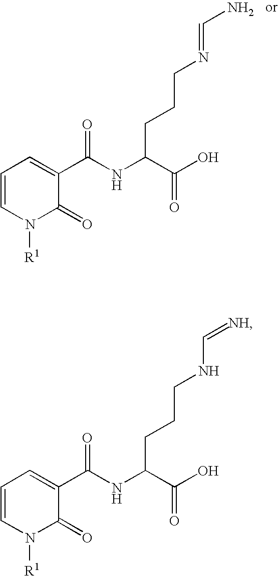

or a pharmaceutically acceptable derivative thereof, wherein the variables are as described elsewhere herein.

In one embodiment, the compound has formula:

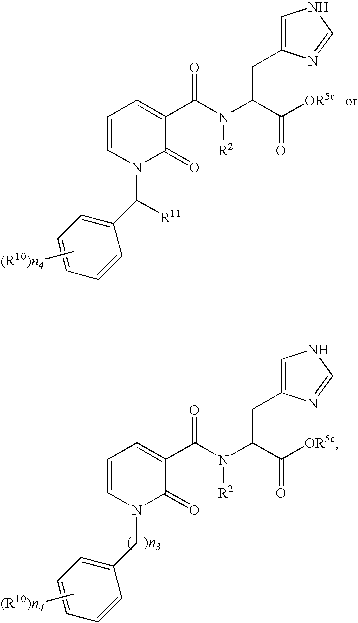

or a pharmaceutically acceptable derivative thereof, wherein the variables are as described elsewhere herein.

In one embodiment, the compound has formula:

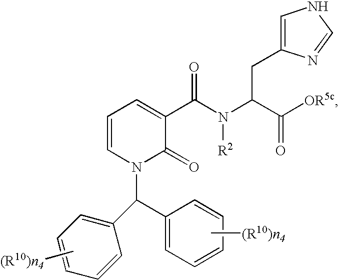

or a pharmaceutically acceptable derivative thereof, wherein the variables are as described elsewhere herein.

In one embodiment, the compound has formula VIII:

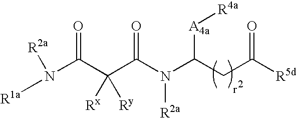

or a pharmaceutically acceptable derivative thereof, wherein

R^{1a} is alkyl, alkenyl, alkynyl, aryl, aralkyl, alkylaryl, cycloalkyl, cycloalkylalkyl, heterocyclyl, heterocyclylalkyl, heteroaryl or heteroarylalkyl;

R^{2a} and R^{3a} are each independently hydrogen, alkyl, alkenyl, alkynyl, cycloalkyl, aryl, heteroaryl or heterocyclyl;

R^{5d} is OR^{a} or NR^{5e}R^{5f};

R^{a} is hydrogen, alkyl, alkenyl, alkynyl, aryl, alkylaryl, heteroaryl, heterocyclyl, cycloalkyl or aralkyl;

R^{5e} and R^{5f} are selected as follows:

i) R^{5e} and R^{5f} are each independently hydrogen, alkyl, alkenyl, alkynyl, cycloalkyl, aryl, heteroaryl or heterocyclyl; or

ii) R^{5e} and R^{5f} together with the nitrogen atom on which they are substituted form a 3-7 membered heterocyclic or heteroaryl ring;

A₄ₐ is alkylene, alkenylene, alkynylene, alk(en)(yn)ylene, cycloalkylene, arylene, aralkylene, alkylarylene, heteroarylene or heterocyclylene;

R^{4a} is

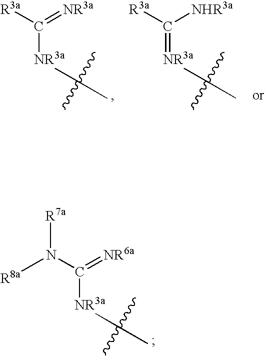

R^{6a} is hydrogen, alkyl, alkenyl, alkynyl, aryl, C(O)R^{9a} or S(O)ₚR^{9a};

R^{7a} and R^{8a} are selected as follows:

i) R^{7a} is hydrogen, alkyl, alkenyl, alkynyl, cycloalkyl, aryl, heteroaryl or heterocyclyl; and R^{8a} is selected from R^{7a}, nitro, C(O)R^{9a} and S(O)ₚR^{9a}; or

ii) R^{7a} and R^{8a} together with the nitrogen atom on which they are substituted form a 3-7 membered heterocyclic or heteroaryl ring;

R^{9a} is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy or aralkoxy;

R^{x} and R^{y} are selected as follows:

i) R^{x} and R^{y} are each independently hydrogen, alkyl, alkenyl, alkynyl, cycloalkyl, aryl, heteroaryl or heterocyclyl; or

ii) R^{x} and R^{y} together with the carbon on which they are substituted form a 3-7 membered ring;

r² iso-3 and p is 0-2.

In one embodiment, the compound has formula VIII, wherein

R^{a} is alkyl, alkenyl, alkynyl, aryl, aralkyl, alkylaryl, cycloalkyl, cycloalkylalkyl, heterocyclyl, heterocyclylalkyl, heteroaryl or heteroarylalkyl;

R^{2a} and R^{3a} are each independently hydrogen, alkyl, alkenyl, alkynyl, cycloalkyl, aryl, heteroaryl or heterocyclyl;

R^{5d} is OR^{a} or NR^{5e}R^{5f};

R^{a} is hydrogen, alkyl, alkenyl, alkynyl, aryl, alkylaryl, heteroaryl, heterocyclyl, cycloalkyl or aralkyl;

R^{5e} and R^{5f} are selected as follows:

i) R^{5e} and R^{5f} are each independently hydrogen, alkyl, alkenyl, alkynyl, cycloalkyl, aryl, heteroaryl or heterocyclyl; or

ii) R^{5e} and R^{5f} together with the nitrogen atom on which they are substituted form a 3-7 membered heterocyclic or heteroaryl ring;

A₄ₐ is alkylene, alkenylene, alkynylene, alk(en)(yn)ylene, cycloalkylene, arylene, aralkylene, alkylarylene, heteroarylene or heterocyclylene;

R^{4a} is

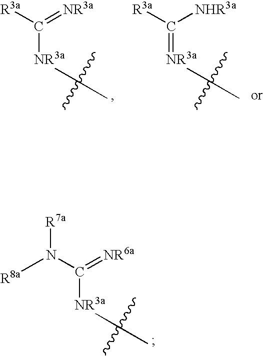

R^{6a} is hydrogen, alkyl, alkenyl, alkynyl, aryl, C(O)R^{9a} or S(O)ₚR^{9a};

R^{7a} and R^{8a} are selected as follows:

i) R^{7a} is hydrogen, alkyl, alkenyl, alkynyl, cycloalkyl, aryl, heteroaryl or heterocyclyl; and R^{8a} is selected from R^{7a}, nitro, C(O)R^{9a} and S(O)ₚR^{9a}; or

ii) R^{7a} and R^{8a} together with the nitrogen atom on which they are substituted form a 3-7 membered heterocyclic or heteroaryl ring;

R^{9a} is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy or aralkoxy;

R^{x} and R^{y} are selected as follows:

i) R^{x} and R^{y} are each independently alkyl, alkenyl, alkynyl, cycloalkyl, aryl, heteroaryl or heterocyclyl; or

ii) R^{x} and R^{y} together with the carbon on which they are substituted form a 3-7 membered ring;

r² is 0-3 and p is 0-2.

In certain embodiments, R^{1a}, R^{2a}, R^{3a}, R^{6a}, R^{7a}, R^{8a}, R^{5d}, R^{x}, R^{y}, A₄ₐ, R^{5e} and R^{5f} are optionally substituted with one or more, in certain embodiments, 1, 2, 3 or 4 substituents, each independently selected from Q¹, where Q¹ is as defined elsewhere herein.

In one embodiment, R^{1a} aralkyl. In one embodiment, R^{1a} is benzhydryl. In one embodiment, R^{2a} and R^{3a} are each independently hydrogen or lower alkyl. In one embodiment, R^{2a} and R^{3a} are hydrogen. In one embodiment, R^{5d} is OR^{a}, where R^{a} is hydrogen or lower alkyl. In one embodiment, R^{5d} is OH. In one embodiment, A₄ₐ is alkylene. In one embodiment, R^{4a} is

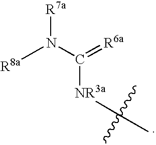

In one embodiment, R^{x} and R^{y} are lower alkyl. In one embodiment, R^{x} and R^{y} are methyl. In one embodiment, r² is 0 or 1. In one embodiment, p is 0, 1 or 2.

In one embodiment, R^{6a} is alkyl, alkenyl, alkynyl, aryl, C(O)R^{9a} or S(O)ₚR^{9a}.

In one embodiment, the compound has formula IX:

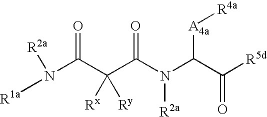

or a pharmaceutically acceptable derivative thereof, wherein the variables are as described elsewhere herein.

In one embodiment, the compound has formula X:

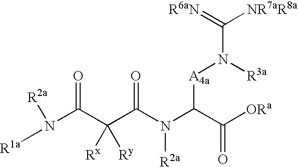

or a pharmaceutically acceptable derivative thereof, wherein the variables are as described elsewhere herein.

In one embodiment, the compound has formula XI:

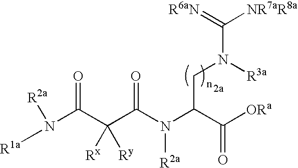

or a pharmaceutically acceptable derivative thereof, wherein n₂ₐ is 1-6 and the other variables are as described elsewhere herein.

In one embodiment, the compound has formula XII:

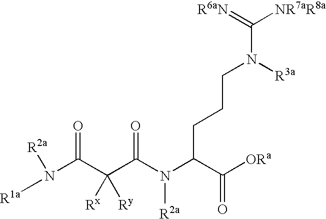

or a pharmaceutically acceptable derivative thereof, wherein the variables are as described elsewhere herein.

In one embodiment, the compound has formula XIII:

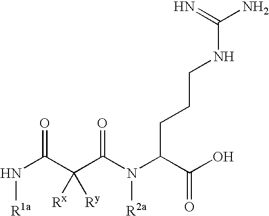

or a pharmaceutically acceptable derivative thereof, wherein the variables are as described elsewhere herein.

In one embodiment, the compound has formula:

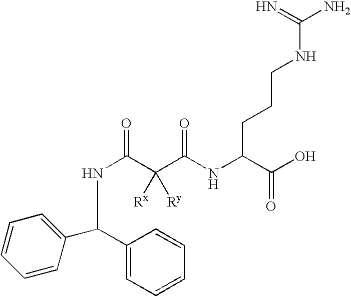

or a pharmaceutically acceptable derivative thereof, wherein R^{x} and R^{y} are each lower alkyl.

In one embodiment, R^{x} and R^{y} are both methyl.

In one embodiment, the compound has formula XIV:

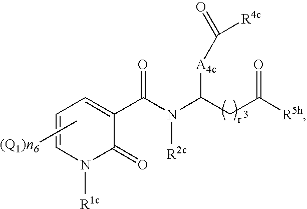

or a pharmaceutically acceptable derivative thereof, wherein

R^{1c} is aralkyl;

R^{2c} is hydrogen, alkyl, alkenyl, alkynyl, cycloalkyl, aryl, heteroaryl or heterocyclyl;

R^{5h} is OR^{c} or NR⁵ⁱR^{5j};

R^{c} is hydrogen, alkyl, alkenyl, alkynyl, aryl, alkylaryl, heteroaryl, heterocyclyl, cycloalkyl or aralkyl;

R⁵ⁱ and R^{5j} are selected as follows:

i) R⁵ⁱ and R^{5j} are each independently hydrogen, alkyl, alkenyl, alkynyl, cycloalkyl, aryl, heteroaryl or heterocyclyl; or

ii) R⁵ⁱ and R^{5j} together with the nitrogen atom on which they are substituted form a 3-7 membered heterocyclic or heteroaryl ring;

A_{4c} is alkylene, alkenylene, alkynylene, alk(en)(yn)ylene, cycloalkylene, arylene, aralkelene, alkylarylene, heteroarylene or heterocyclylene;

R^{4c} is R^{5h},

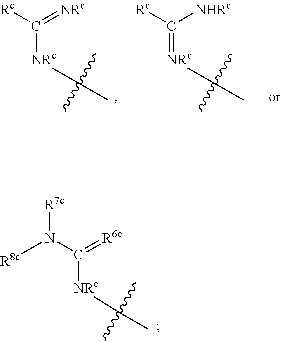

R^{6c} is NR^{6y} or O;

R^{6y} is hydrogen, alkyl, alkenyl, alkynyl, aryl, C(O)R^{9c} or S(O)ₚR^{9c};

R^{7c} and R^{8c} are selected as follows:

i) R^{7c} is hydrogen, alkyl, alkenyl, alkynyl, cycloalkyl, aryl, heteroaryl or heterocyclyl; and R^{8c} is selected from R^{7c}, nitro, C(O)R^{9c} and S(O)ₚR^{9c}; or

ii) R^{7c} and R^{8c} together with the nitrogen atom on which they are substituted form a 3-7 membered heterocyclic or heteroaryl ring;

R^{9c} is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy or aralkoxy;

r³ is 0-3; p is 0-2 and n₆ is 0-3.

In certain embodiments, R^{1c}, R^{2c}, R^{4c}, R^{5h}, R^{c}, R⁵ⁱ, R^{5k} and A_{4c} are optionally substituted with one or more, in certain embodiments, 1, 2, 3 or 4 substituents, each independently selected from Q¹, where is as defined elsewhere herein.

In one embodiment, R^{1c} is benzhydryl. In one embodiment, R^{2c} is hydrogen or lower alkyl. In one embodiment, R^{2c} is hydrogen. In one embodiment, R^{5h} is OR^{c}, where R^{c} is hydrogen or lower alkyl. In one embodiment, R^{5h} is OH. In one embodiment, A_{4c} is alkylene. In one embodiment, R^{4c} is OH. In one embodiment, R^{4c} is

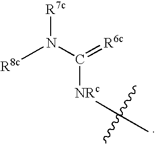

In one embodiment, r³ is 0 or 1.

In one embodiment, the compound has formula XV:

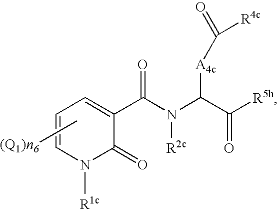

or a pharmaceutically acceptable derivative thereof, wherein the variables are as described elsewhere herein.

In one embodiment, the compound has formula XVI:

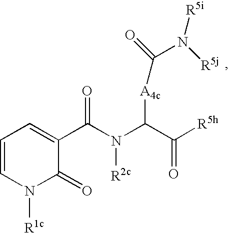

or a pharmaceutically acceptable derivative thereof, wherein the variables are as described elsewhere herein.

In one embodiment, the compound has formula XVIIA or XVIIB:

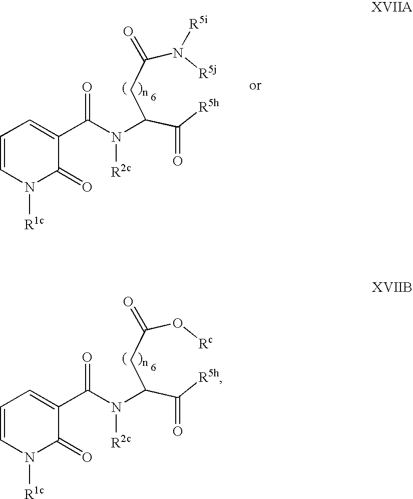

or a pharmaceutically acceptable derivative thereof, wherein n6 is 1-5 and the other variables are as described elsewhere herein.

In one embodiment, the compound has formula XVIIIA, XVIIIB, XVIIIC or XVIIID:

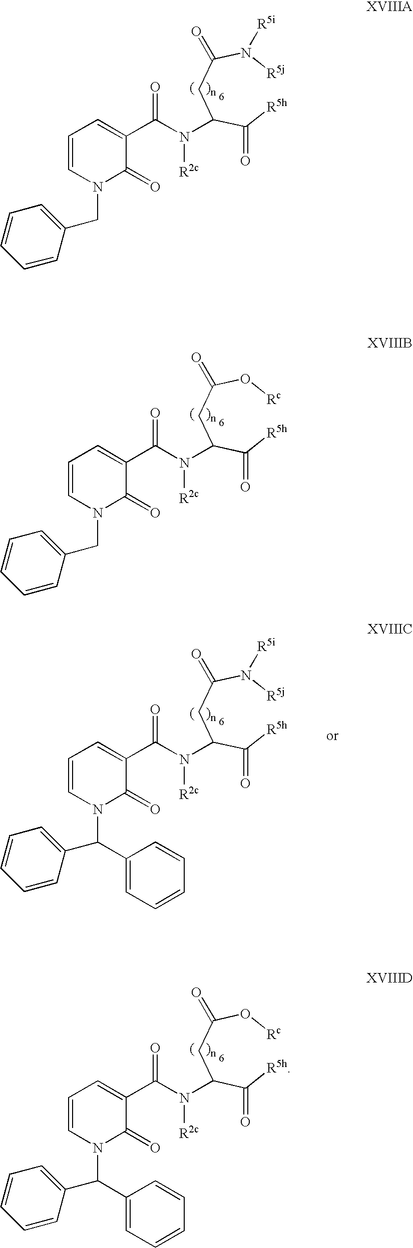

or a pharmaceutically acceptable derivative thereof, wherein the variables are as described elsewhere herein.

In one embodiment, the compound has formula XIX:

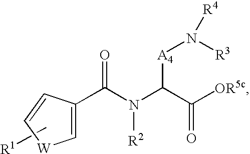

or a pharmaceutically acceptable derivative thereof, wherein

W is O or S;

R is aralkyl;

A₄ is alkylene;

R¹ is alkyl or aralkyl, optionally substituted with one or two alkyl or halo;

R² is hydrogen or alkyl;

R^{5c} is hydrogen or alkyl;

R^{5a} and R^{5b} are selected as follows:

R³ is hydrogen or alkyl;

R⁴ is alkyl, —C(═R⁶)NR⁷R⁸ or —C(═NR^{m})Rⁿ;

R^{m} is hydrogen or hydroxy;

Rⁿ is hydrogen, alkyl, aryl, heteroaryl, cycloalkyl, alkoxycarbonylalkyl or hydroxy;

R⁶ is NR^{6x};

R^{6x} is hydrogen, OH or alkyl;

R⁷ is hydrogen or alkyl; and

R⁸ is hydrogen or alkyl.

In one embodiment, the compound has formula XIX, or a pharmaceutically acceptable derivative thereof, wherein

W is O or S;

R is aralkyl;

A₄ is alkylene;

R¹ is alkyl or aralkyl, optionally substituted with one or two alkyl or halo;

R² is hydrogen or alkyl;

R^{5c} is hydrogen or alkyl;

R^{5a} and R^{5b} are selected as follows:

R³ is hydrogen or alkyl;

R⁴ is (═NR^{m})Rⁿ;

R^{m} is hydrogen or hydroxy;

Rⁿ is hydrogen, alkyl, aryl, heteroaryl, cycloalkyl, alkoxycarbonylalkyl or hydroxy;

R⁶ is NR^{6x};

R^{6x} is hydrogen, OH or alkyl;

R⁷ is hydrogen or alkyl; and

R⁸ is hydrogen or alkyl.

In one embodiment, the compound has formula:

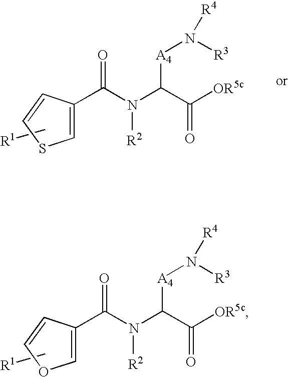

or a pharmaceutically acceptable derivative thereof, where the variables are as described elsewhere herein.

In one embodiment, the compound has formula:

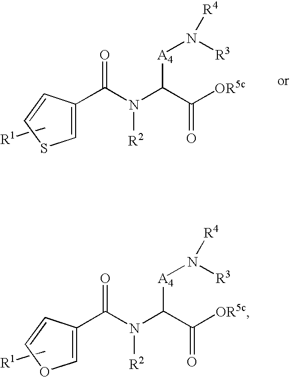

or a pharmaceutically acceptable derivative thereof, where the variables are as described elsewhere herein.

In one embodiment, the compound has formula:

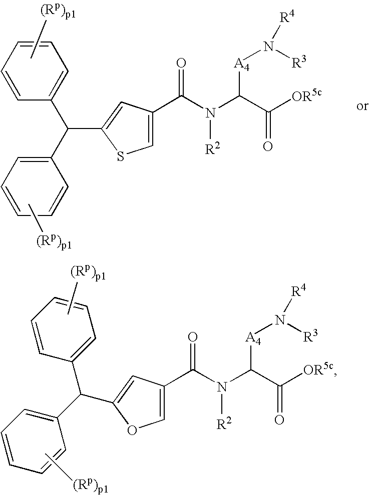

or a pharmaceutically acceptable derivative thereof, where each R^{p} is independently hydrogen, halo or alkyl; p₁ is 1 or 2; and the other variables are as described elsewhere herein.

In one embodiment, the compound is:

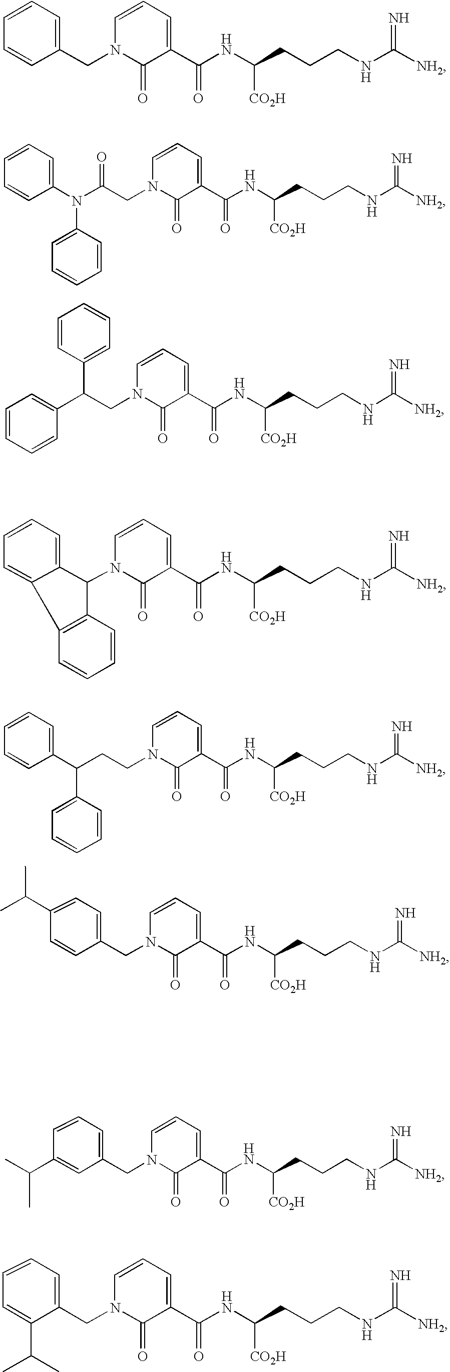

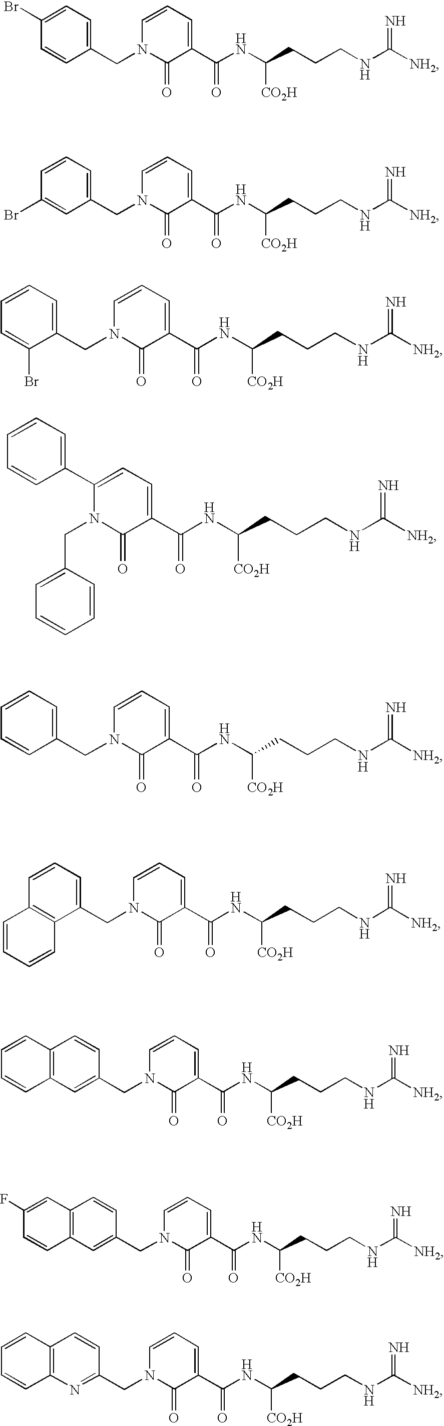

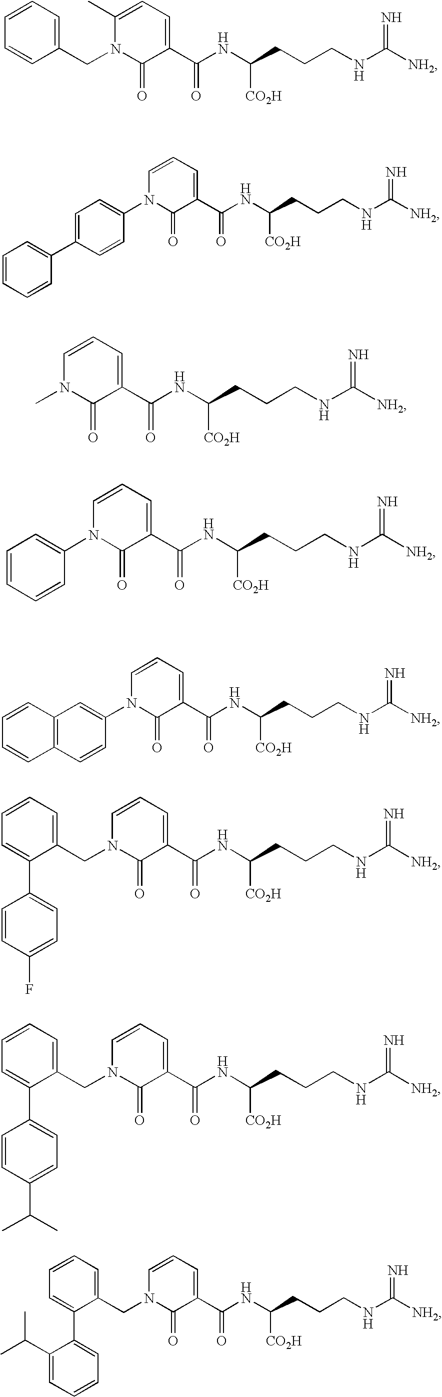

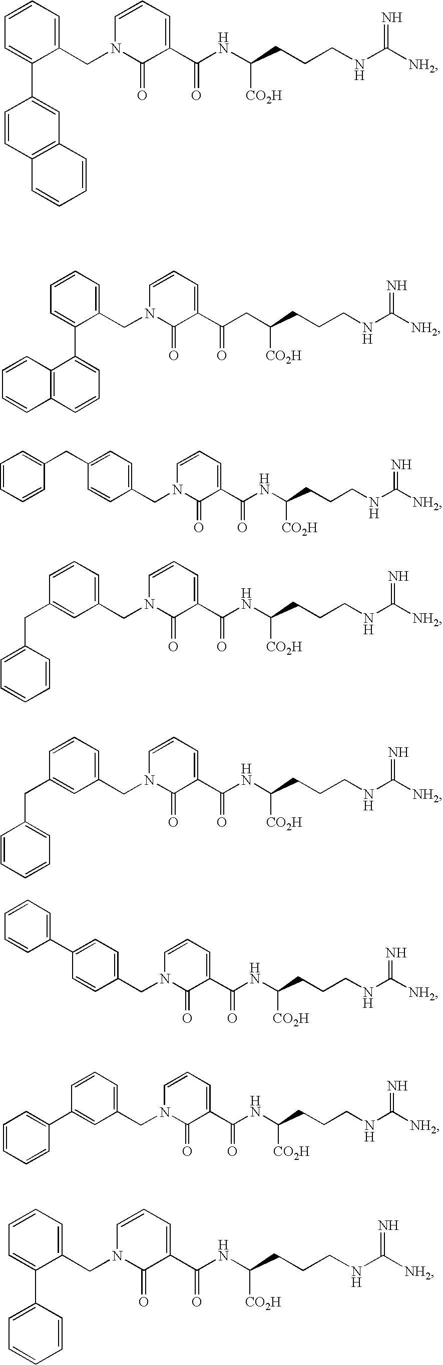

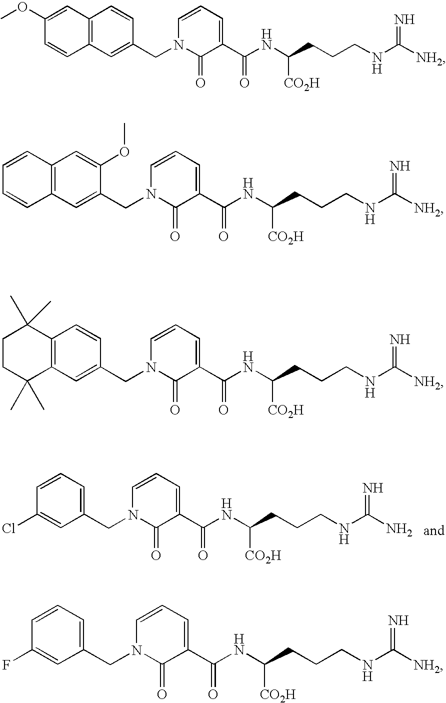

and pharmaceutically acceptable derivatives thereof.

In one embodiment, the compound is selected from:

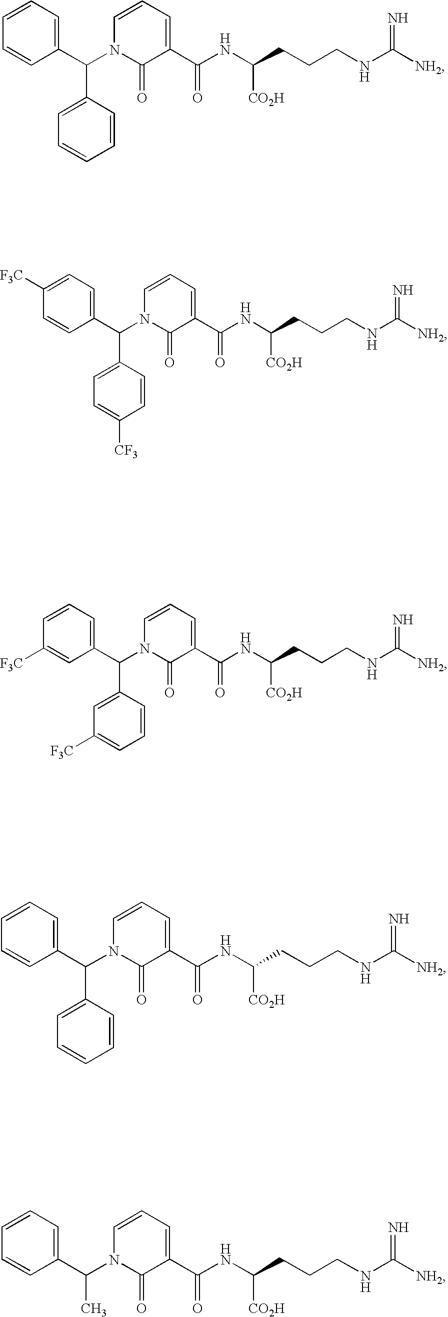

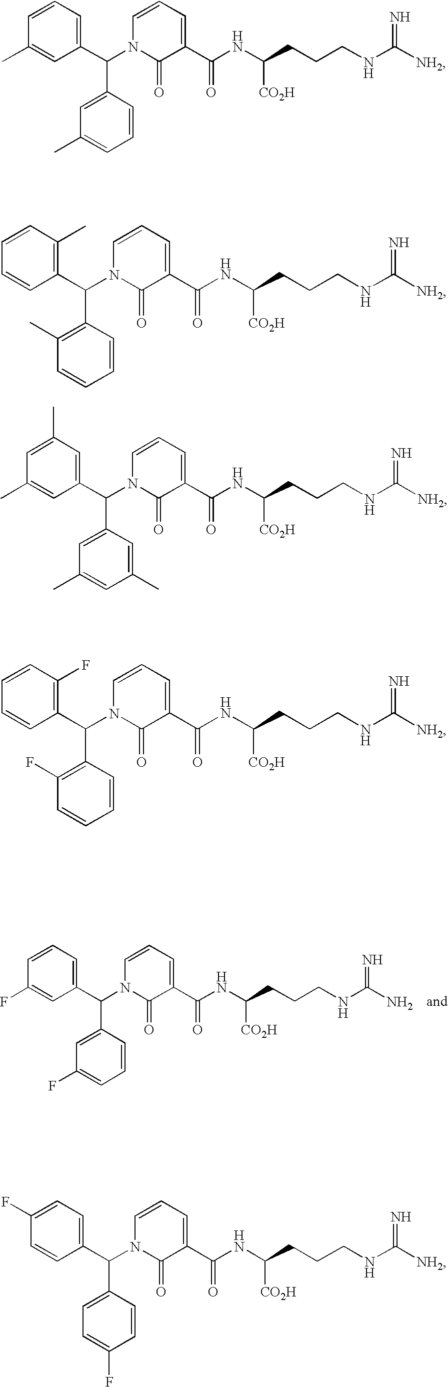

and pharmaceutically acceptable derivatives thereof.

In one embodiment, the compound is selected from:

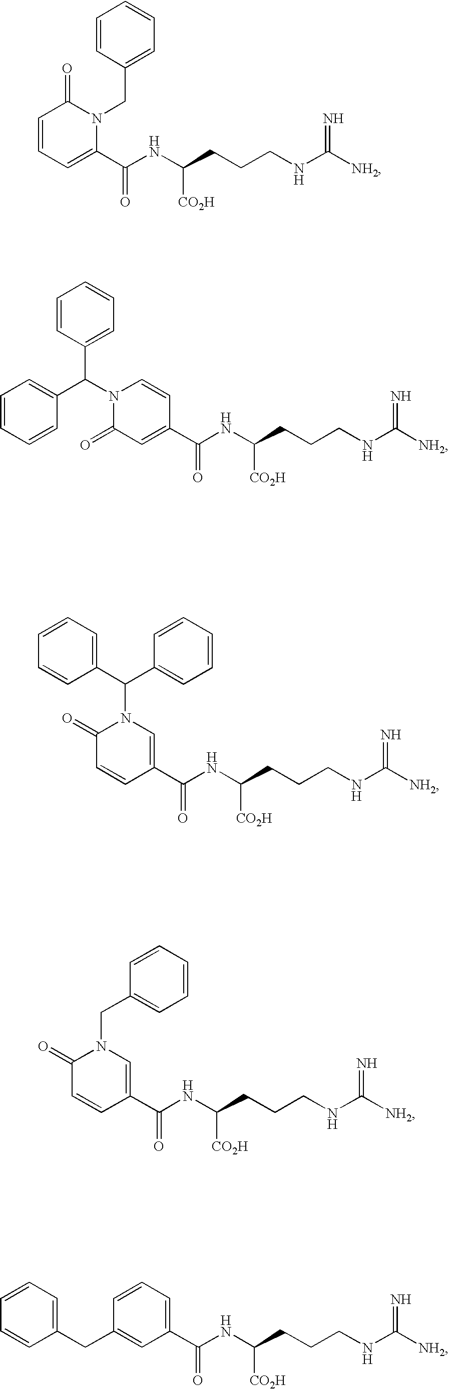

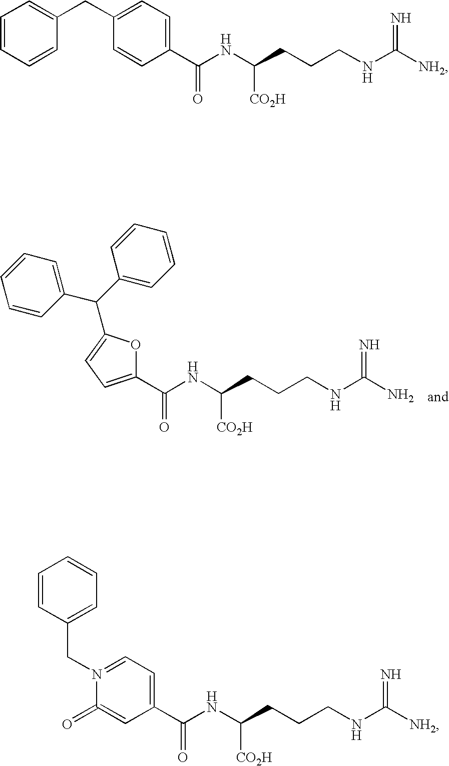

and pharmaceutically acceptable derivatives thereof.

In one embodiment, the compound is selected from:

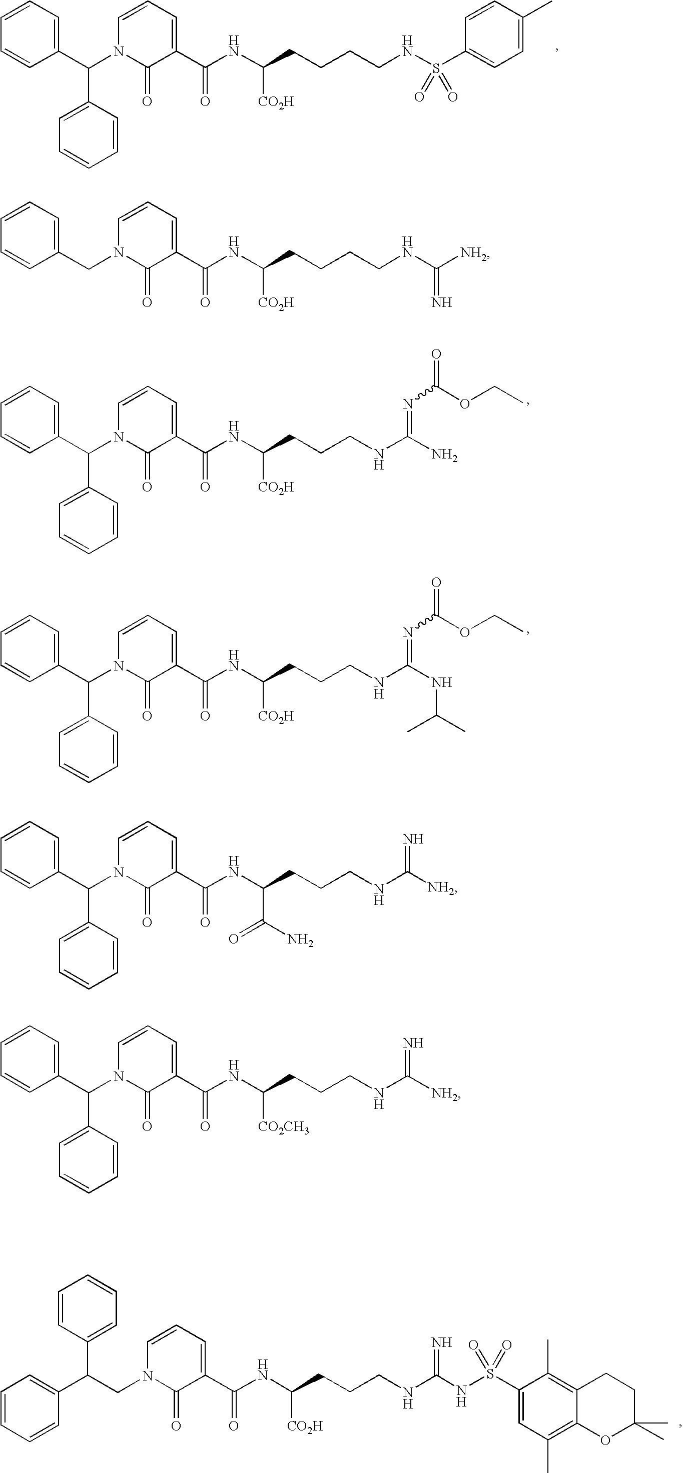

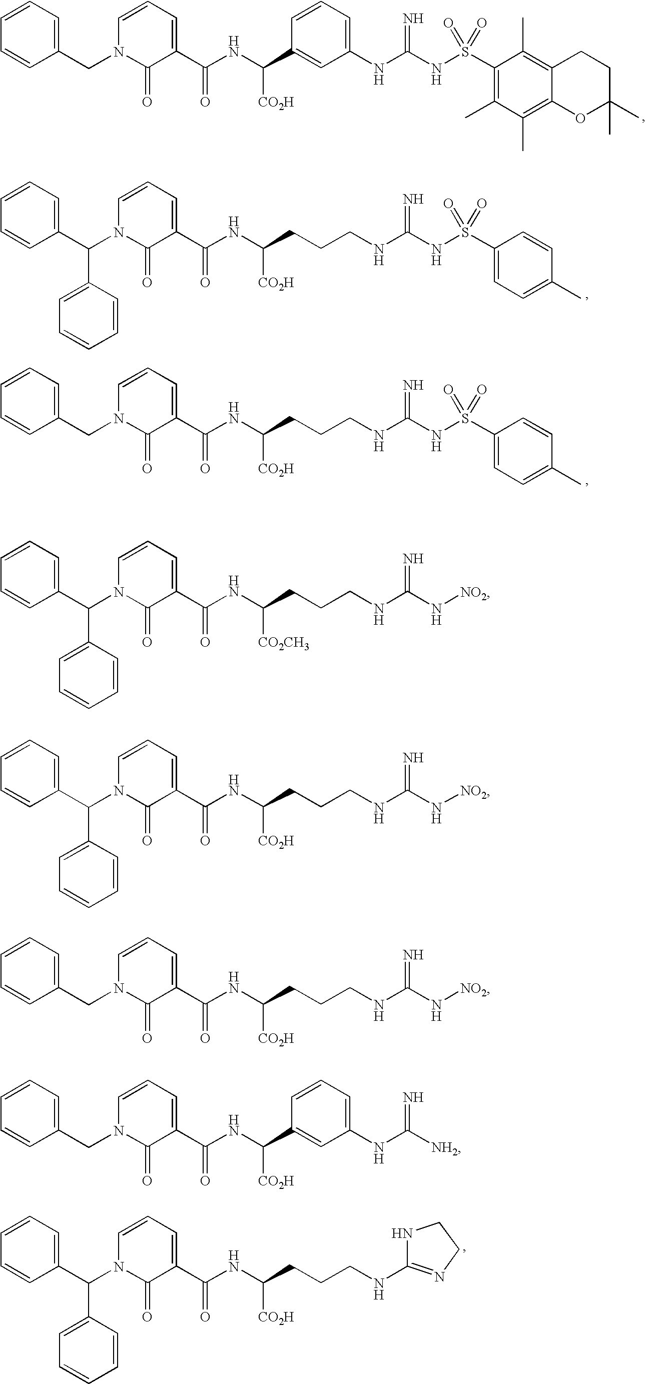

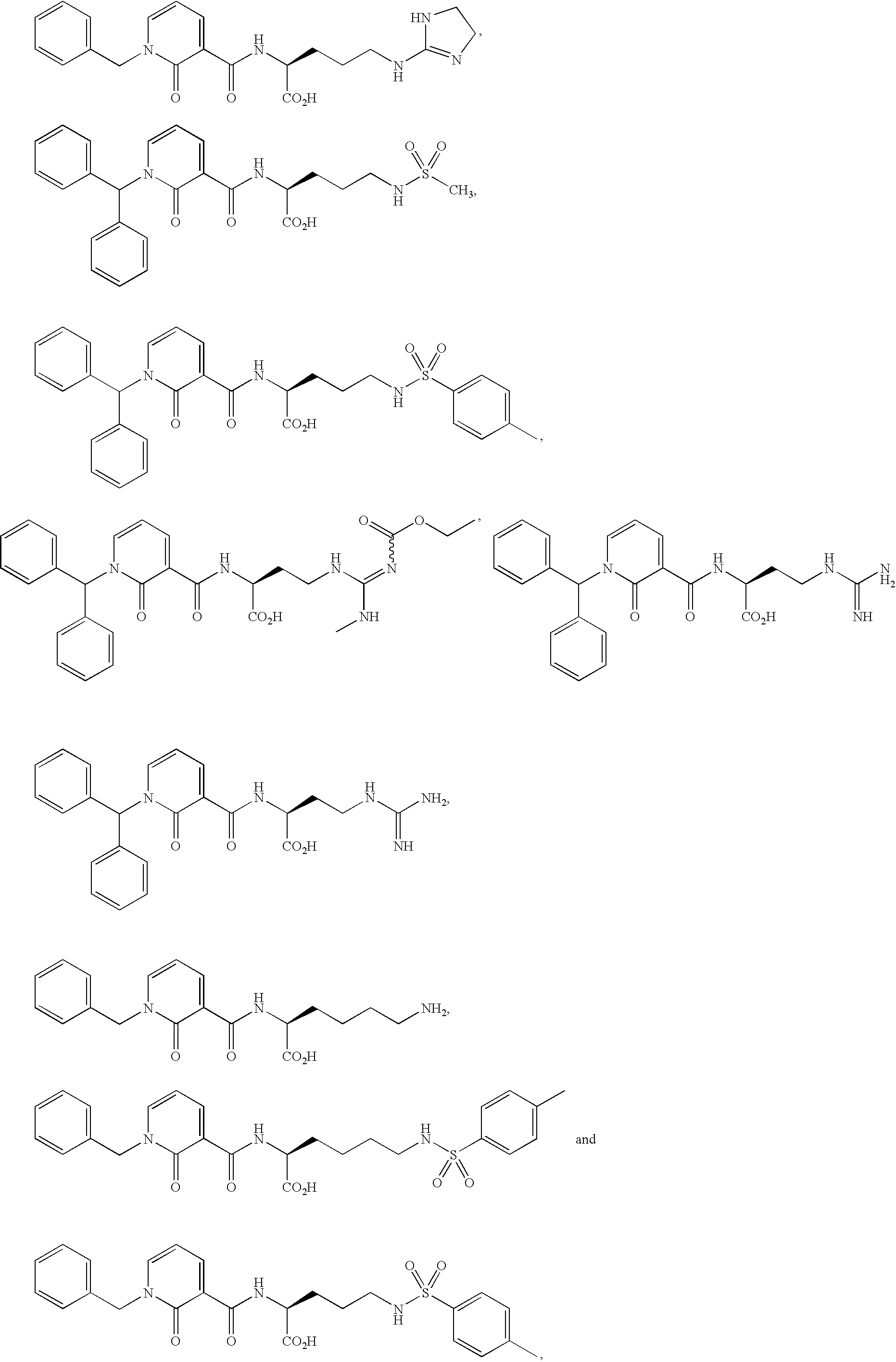

and pharmaceutically acceptable derivatives thereof.

In one embodiment, the compound is selected from:

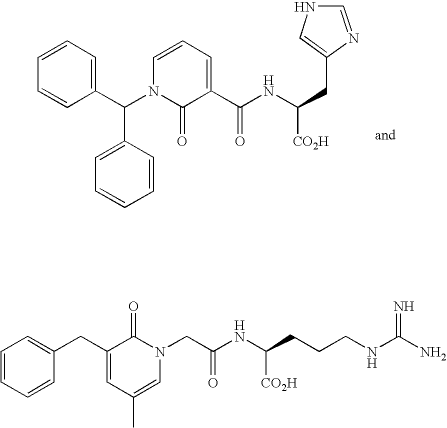

and pharmaceutically acceptable derivatives thereof.

In one embodiment, the compound is:

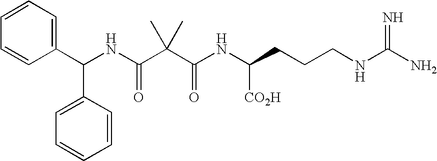

and pharmaceutically acceptable derivative thereof.

In one embodiment, the compound is:

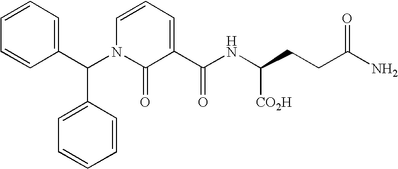

and pharmaceutically acceptable derivative thereof.

In one embodiment, the compound is:

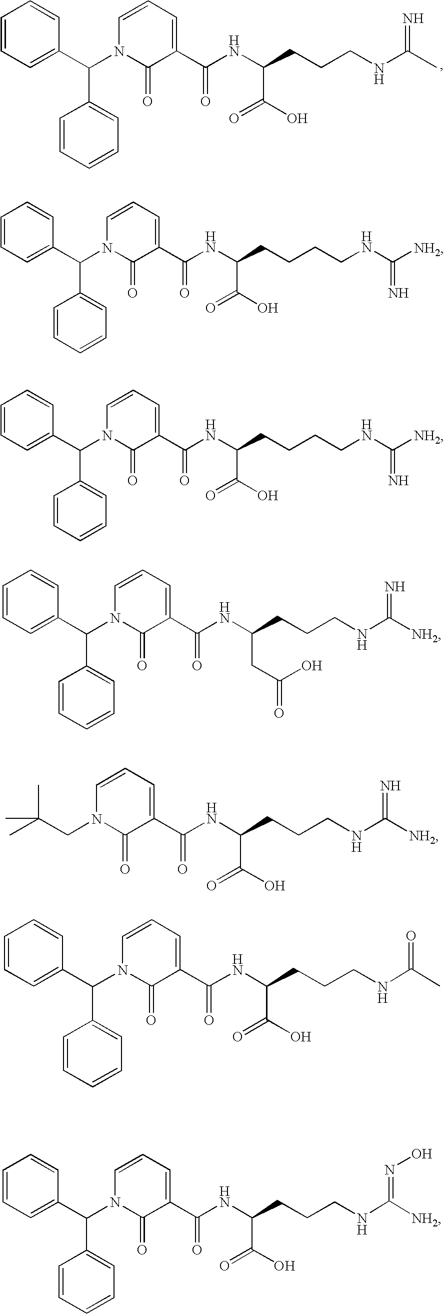

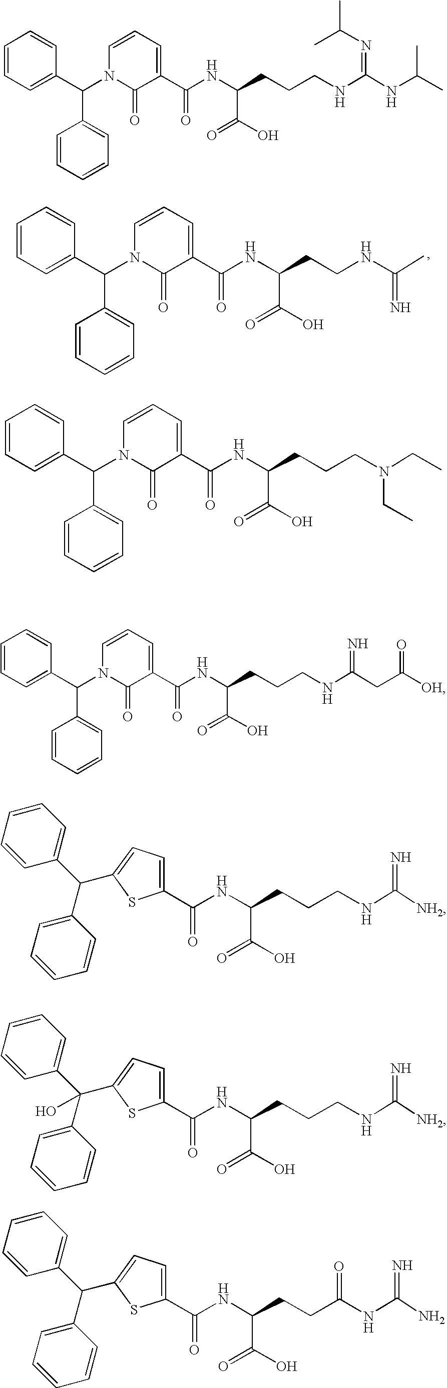

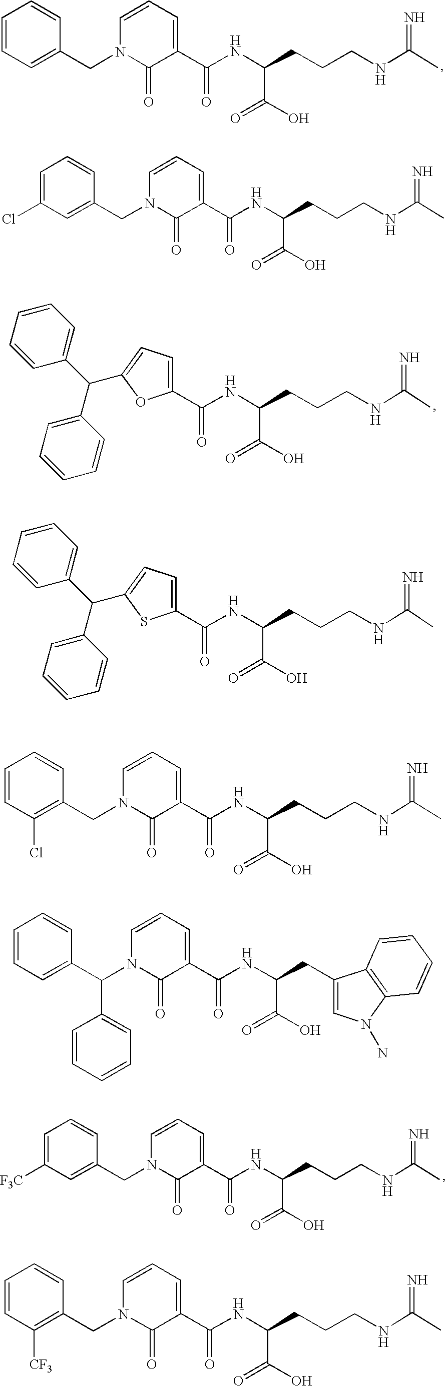

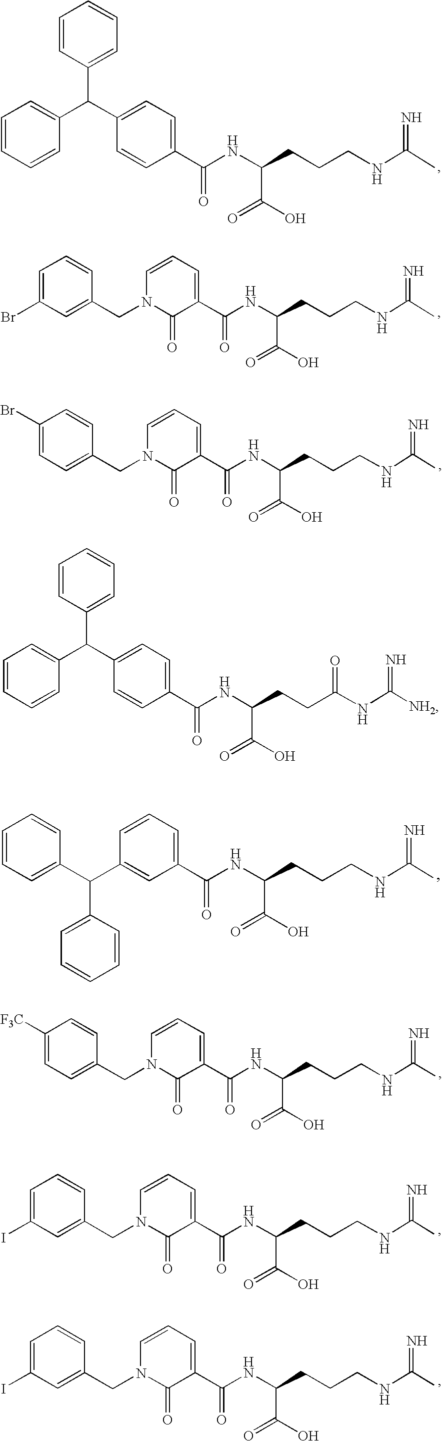

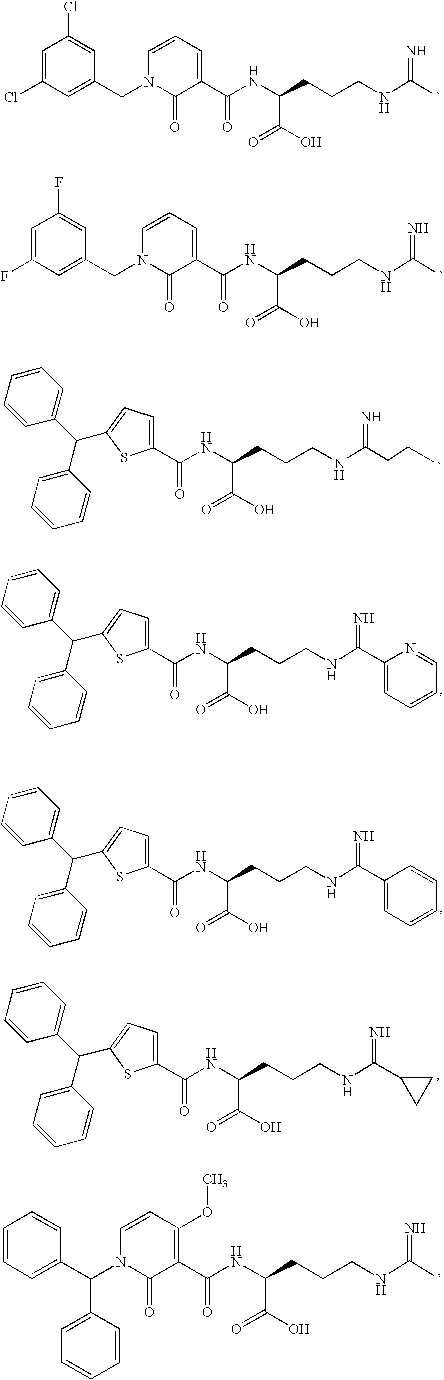

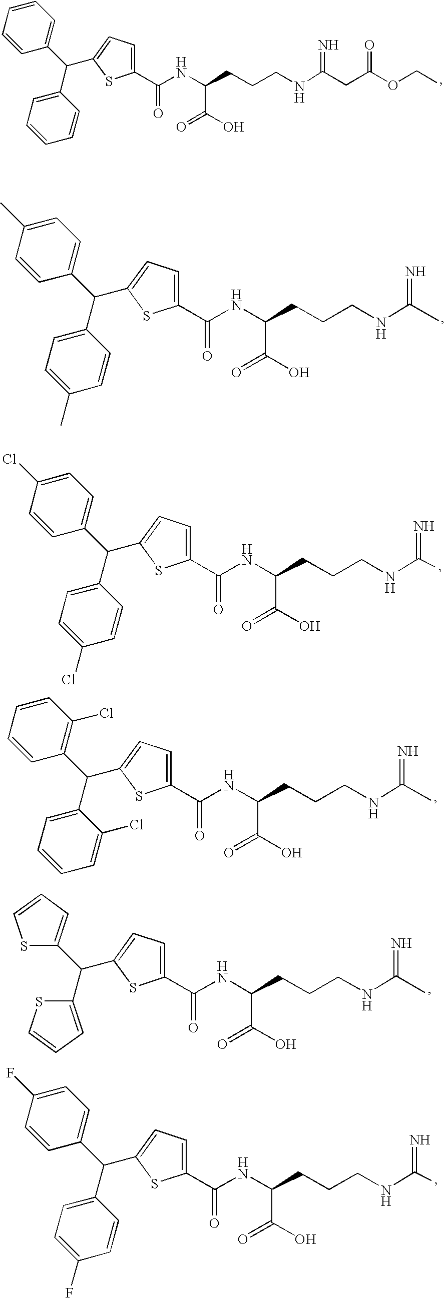

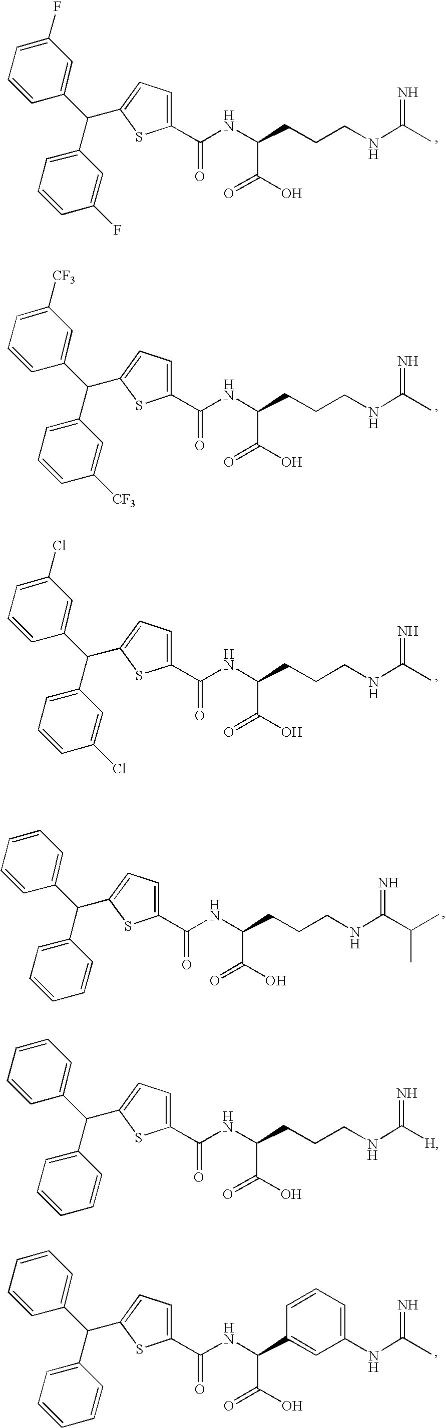

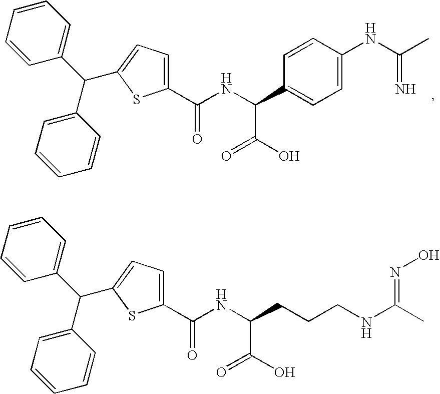

or a pharmaceutically acceptable derivative thereof.

5.2.1 Preparation of the Compounds

The compounds provided herein can be prepared by routine chemical reactions known to one of skill in the art. General schemes for preparation of exemplary compounds are illustrated below:

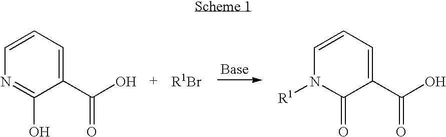

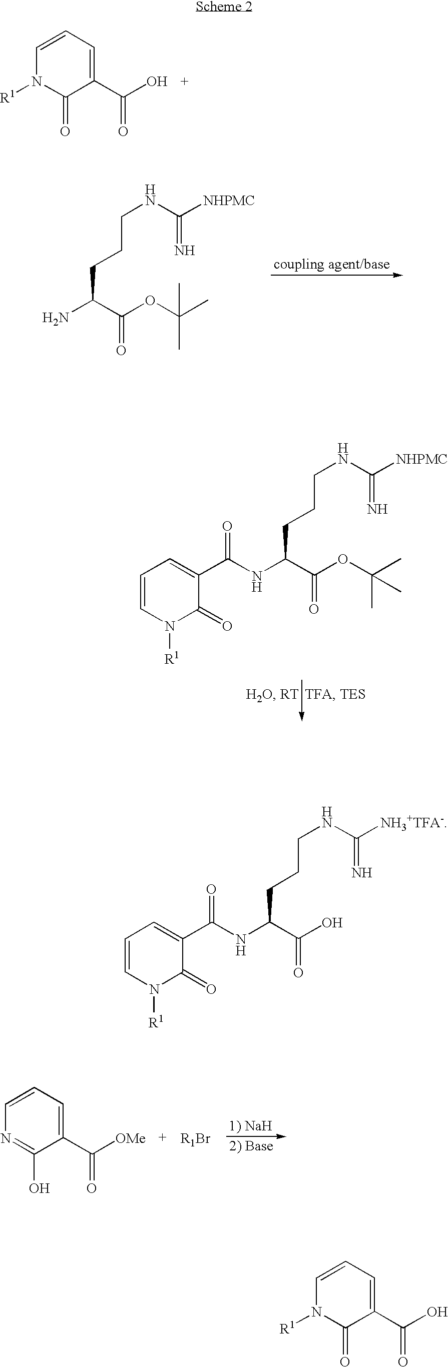

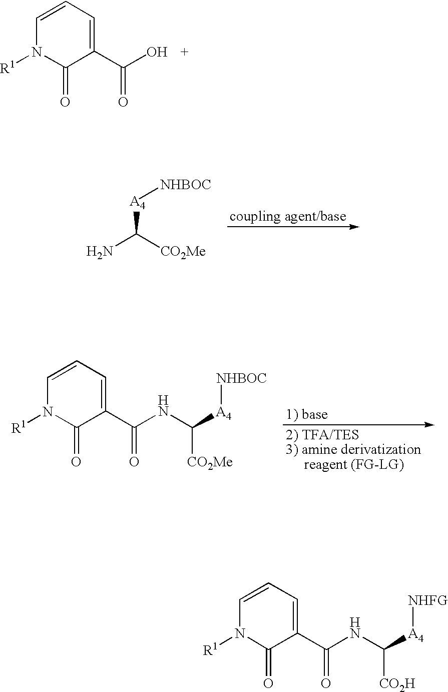

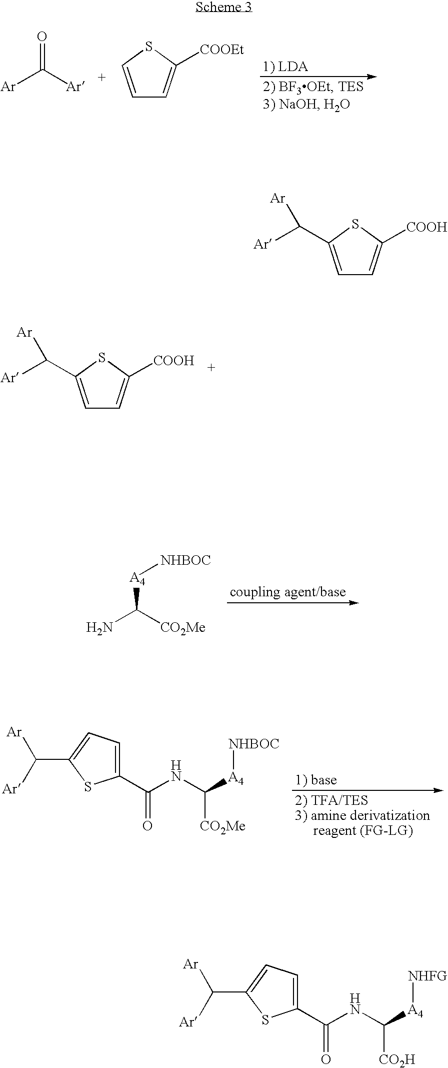

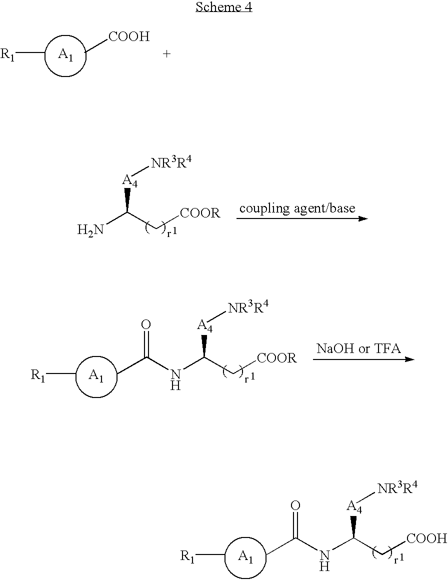

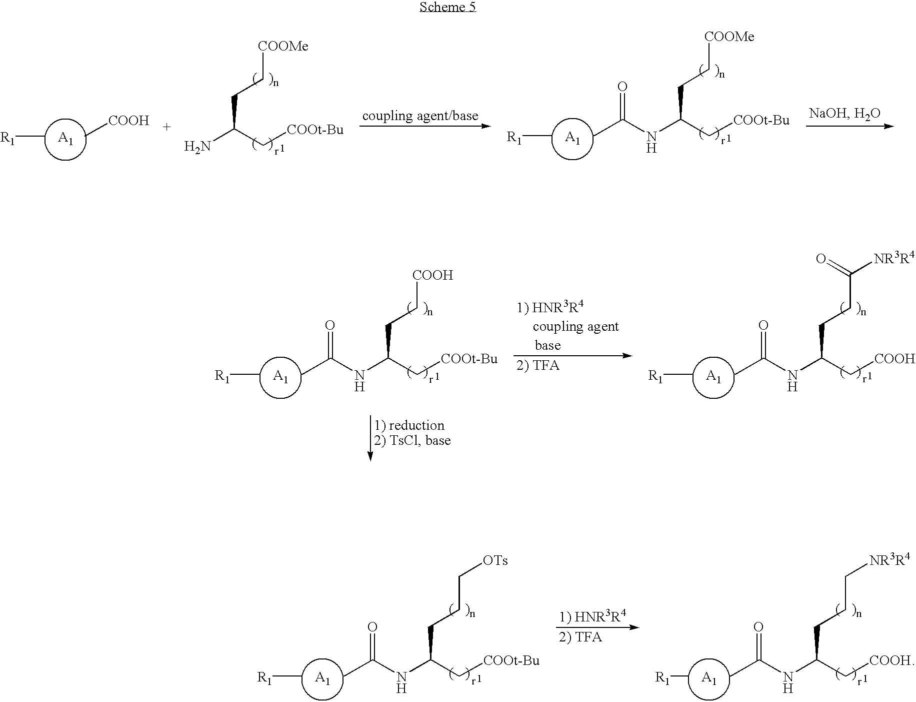

The coupling reaction between suitably substituted 2-oxo-1,2-dihydropyridine-3-carboxylic acid and (s)-tert-butyl 2-amino-5-[3-(2,2,5,7,8-pentamethylchroman-6-ylsulfonyl)guanidino]pentanoate can be carried out in presence of any coupling agent and base known to one of skill in the art. Exemplary coupling agents for use in the reaction include, but are not limited to HOBt(N-Hydroxybenzotriazole), HBTU (2-(1H-Benzotriazole-1-yl)-1,1,3,3-tetramethyluronium hexafluorophosphate), DCC(N,N′-dicyclohexylcarbodiimide), BOP (Benzotriazole-1-yl-oxy-tris-(dimethylamino)-phosphoniumhexafluorophosphate) and others known to one of skill in the art. Exemplary bases are DBU (diazabicyclo[5.4.0]undec-7-ene), DIEA DIPEA (diisopropylethylamine), TBAF (tetrabutylammonium fluoride) and piperidine. The Examples section provides reaction schemes and experimental details for preparation of exemplary compounds provided herein.

5.3 Formulation of Pharmaceutical Compositions

The pharmaceutical compositions provided herein contain therapeutically effective amounts of one or more of compounds provided herein that are useful in the prevention, treatment, or amelioration of one or more of the symptoms of C3a receptor mediated diseases.

The compositions contain one or more compounds provided herein. The compounds are formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers.

In one embodiment, the compounds described above are formulated into pharmaceutical compositions using techniques and procedures well known in the art (see, e.g., Remington's Pharmaceutical Sciences, 20^{th} eds., Mack Publishing, Easton Pa. (2000)).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that treats, prevents, or ameliorates one or more of the symptoms of C3a receptor mediated diseases.

In one embodiment, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as known in the art. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline (PBS) lacking divalent cations is added and the flask shaken until the lipid film is dispersed. The resulting vesicles are washed to remove unencapsulated compound, pelleted by centrifugation, and then resuspended in PBS.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in in vitro and in vivo systems described herein and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of C3a receptor mediated diseases.

In one embodiment, a therapeutically effective dosage should produce a serum concentration of active ingredient of from about 0.1 ng/ml to about 50-100 μg/ml. The pharmaceutical compositions, in certain embodiments, should provide a dosage of from about 0.001 mg to about 2000 mg of compound per kilogram of body weight per day. Pharmaceutical dosage unit forms are prepared to provide from about 1 mg to about 1000 mg and from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing C3a receptor mediated diseases. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets can be used. The compositions are in liquid, semi-liquid or solid form and are formulated in a manner suitable for each route of administration. In one embodiment, modes of administration include parenteral and oral modes of administration. In certain embodiments, oral administration is contemplated.

Solutions or suspensions used for parenteral, intradermal, subcutaneous, or topical application can include any of the following components: a sterile diluent, such as water for injection, saline solution, fixed oil, polyethylene glycol, glycerine, propylene glycol, dimethyl acetamide or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are formulated and administered in unit-dosage forms or multiple-dosage forms. Unit-dose forms as used herein refer to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

Sustained-release preparations can also be prepared. Suitable examples of sustained-release preparations include semipermeable matrices of solid hydrophobic polymers containing the compound provided herein, which matrices are in the form of shaped articles, e.g., films, or microcapsule. Examples of sustained-release matrices include polyesters, hydrogels (for example, poly(2-hydroxyethyl-methacrylate), or poly(vinylalcohol)), polylactides, copolymers of L-glutamic acid and ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid. While polymers such as ethylene-vinyl acetate and lactic acid-glycolic acid enable release of molecules for over 100 days, certain hydrogels release proteins for shorter time periods. When encapsulated compound remain in the body for a long time, they may denature or aggregate as a result of exposure to moisture at 37° C., resulting in a loss of biological activity and possible changes in their structure. Rational strategies can be devised for stabilization depending on the mechanism of action involved. For example, if the aggregation mechanism is discovered to be intermolecular S—S bond formation through thio-disulfide interchange stabilization may be achieved by modifying sulfhydryl residues, lyophilizing from acidic solutions, controlling moisture content, using appropriate additives, and developing specific polymer matrix compositions

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active ingredient, in one embodiment, 0.1-85% or 75-95% active ingredient.

The active compounds or pharmaceutically acceptable derivatives may be prepared with carriers that protect the compound against rapid elimination from the body, such as time release formulations or coatings.

The compositions may include other active compounds to obtain desired combinations of properties. The compounds provided herein, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as C3a receptor mediated diseases. It is to be understood that such combination therapy constitutes a further aspect of the compositions and methods of treatment provided herein.

5.3.1 Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, such as capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic acids include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, can be encapsulated in a gelatin capsule. Such solutions, and the preparation and encapsulation thereof, are disclosed in U.S. Pat. Nos. 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include, but are not limited to, those containing a compound provided herein, a dialkylated mono- or poly-alkylene glycol, including, but not limited to, 1,2-dimethoxymethane, diglyme, triglyme, tetraglyme, polyethylene glycol-350-dimethyl ether, polyethylene glycol-550-dimethyl ether, polyethylene glycol-750-dimethyl ether wherein 350, 550 and 750 refer to the approximate average molecular weight of the polyethylene glycol, and one or more antioxidants, such as butylated hydroxytoluene (BHT), butylated hydroxyanisole (BHA), propyl gallate, vitamin E, hydroquinone, hydroxycoumarins, ethanolamine, lecithin, cephalin, ascorbic acid, malic acid, sorbitol, phosphoric acid, thiodipropionic acid and its esters, and dithiocarbamates.

Other formulations include, but are not limited to, aqueous alcoholic solutions including a pharmaceutically acceptable acetal. Alcohols used in these formulations are any pharmaceutically acceptable water-miscible solvents having one or more hydroxyl groups, including, but not limited to, propylene glycol and ethanol. Acetals include, but are not limited to, di(lower alkyl)acetals of lower alkyl aldehydes such as acetaldehyde diethyl acetal.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

5.3.2 Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained is also contemplated herein. Briefly, a compound provided herein is dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The compound diffuses through the outer polymeric membrane in a release rate controlling step. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions includes EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. In certain embodiments, a therapeutically effective dosage is formulated to contain a concentration of at least about 0.1% w/w up to about 90% w/w or more, or more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

5.3.3 Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound provided herein, or a pharmaceutically acceptable derivative thereof, in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg or 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, 5-35 mg or about 9-30 mg of lyophilized powder, is added per mL of sterile water or other suitable carrier. The precise amount depends upon the selected compound. Such amount can be empirically determined.

5.3.4 Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment of inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will have diameters of less than 50 microns or less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5.3.5 Compositions for Other Routes of Administration

Other routes of administration, such as topical application, transdermal patches, and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. In certain embodiments, the weight of a rectal suppository is about 2 to 3 gm.

Tablets and capsules for rectal administration are manufactured using the same pharmaceutically acceptable substance and by the same methods as for formulations for oral administration.

5.3.6 Sustained Release Compositions

Active ingredients such as the compounds provided herein can be administered by controlled release means or by delivery devices that are well known to those of ordinary skill in the art. Examples include, but are not limited to, those described in U.S. Pat. Nos. 3,845,770; 3,916,899; 3,536,809; 3,598,123; and 4,008,719; 5,674,533; 5,059,595; 5,591,767; 5,120,548; 5,073,543; 5,639,476; 5,354,556; 5,639,480; 5,733,566; 5,739,108; 5,891,474; 5,922,356; 5,972,891; 5,980,945; 5,993,855; 6,045,830; 6,087,324; 6,113,943; 6,197,350; 6,248,363; 6,264,970; 6,267,981; 6,376,461; 6,419,961; 6,589,548; 6,613,358; 6,699,500 each of which is incorporated herein by reference. Such dosage forms can be used to provide slow or controlled release of one or more active ingredients using, for example, hydropropylmethyl cellulose, other polymer matrices, gels, permeable membranes, osmotic systems, multilayer coatings, microparticles, liposomes, microspheres, or a combination thereof to provide the desired release profile in varying proportions. Suitable controlled release formulations known to those of ordinary skill in the art, including those described herein, can be readily selected for use with the active ingredients provided herein. Thus, the compositions provided encompasse single unit dosage forms suitable for oral administration such as, but not limited to, tablets, capsules, gelcaps, and caplets that are adapted for controlled release.

All controlled release pharmaceutical products have a common goal of improving drug therapy over that achieved by their non controlled counterparts. Ideally, the use of an optimally designed controlled release preparation in medical treatment is characterized by a minimum of drug substance being employed to cure or control the condition in a minimum amount of time. Advantages of controlled release formulations include extended activity of the drug, reduced dosage frequency, and increased subject compliance. In addition, controlled release formulations can be used to affect the time of onset of action or other characteristics, such as blood levels of the drug, and can thus affect the occurrence of side (e.g., adverse) effects.

Most controlled release formulations are designed to initially release an amount of drug (active ingredient) that promptly produces the desired therapeutic effect, and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled release of an active ingredient can be stimulated by various conditions including, but not limited to, pH, temperature, enzymes, water, or other physiological conditions or compounds.

In certain embodiments, the drug may be administered using intravenous infusion, an implantable osmotic pump, a transdermal patch, liposomes, or other modes of administration. In one embodiment, a pump may be used (see, Sefton, CRC Crit. Ref. Biomed. Eng. 14:201 (1987); Buchwald et al., Surgery 88:507 (1980); Saudek et al., N. Engl. J. Med. 321:574 (1989)). In another embodiment, polymeric materials can be used. In yet another embodiment, a controlled release system can be placed in a subject at an appropriate site determined by a practitioner of skill, i.e., thus requiring only a fraction of the systemic dose (see, e.g., Goodson, Medical Applications of Controlled Release, vol. 2, pp. 115-138 (1984)). Other controlled release systems are discussed in the review by Langer (Science 249:1527-1533 (1990)). The active ingredient can be dispersed in a solid inner matrix, e.g., polymethylmethacrylate, polybutylmethacrylate, plasticized or unplasticized polyvinylchloride, plasticized nylon, plasticized polyethyleneterephthalate, natural rubber, polyisoprene, polyisobutylene, polybutadiene, polyethylene, ethylene-vinylacetate copolymers, silicone rubbers, polydimethylsiloxanes, silicone carbonate copolymers, hydrophilic polymers such as hydrogels of esters of acrylic and methacrylic acid, collagen, cross-linked polyvinylalcohol and cross-linked partially hydrolyzed polyvinyl acetate, that is surrounded by an outer polymeric membrane, e.g., polyethylene, polypropylene, ethylene/propylene copolymers, ethylene/ethyl acrylate copolymers, ethylene/vinylacetate copolymers, silicone rubbers, polydimethyl siloxanes, neoprene rubber, chlorinated polyethylene, polyvinylchloride, vinylchloride copolymers with vinyl acetate, vinylidene chloride, ethylene and propylene, ionomer polyethylene terephthalate, butyl rubber epichlorohydrin rubbers, ethylene/vinyl alcohol copolymer, ethylene/vinyl acetate/vinyl alcohol terpolymer, and ethylene/vinyloxyethanol copolymer, that is insoluble in body fluids. The active ingredient then diffuses through the outer polymeric membrane in a release rate controlling step. The percentage of active ingredient in such parenteral compositions is highly dependent on the specific nature thereof, as well as the needs of the subject.

5.3.7 Targeted Formulations

The compounds provided herein, or pharmaceutically acceptable derivatives thereof, may also be formulated to be targeted to a particular tissue, receptor, or other area of the body of the subject to be treated. Many such targeting methods are well known to those of skill in the art. All such targeting methods are contemplated herein for use in the instant compositions. For non-limiting examples of targeting methods, see, e.g., U.S. Pat. Nos. 6,316,652, 6,274,552, 6,271,359, 6,253,872, 6,139,865, 6,131,570, 6,120,751, 6,071,495, 6,060,082, 6,048,736, 6,039,975, 6,004,534, 5,985,307, 5,972,366, 5,900,252, 5,840,674, 5,759.542 and 5,709,874.

In one embodiment, liposomal suspensions, including tissue-targeted liposomes, such as tumor-targeted liposomes, may also be suitable as pharmaceutically acceptable carriers. These may be prepared according to methods known to those skilled in the art. For example, liposome formulations may be prepared as described in U.S. Pat. No. 4,522,811. Briefly, liposomes such as multilamellar vesicles (MLV's) may be formed by drying down egg phosphatidyl choline and brain phosphatidyl serine (7:3 molar ratio) on the inside of a flask. A solution of a compound provided herein in phosphate buffered saline lacking divalent cations (PBS) is added and the flask shaken until the lipid film is dispersed. The resulting vesicles are washed to remove unencapsulated compound, pelleted by centrifugation, and then resuspended in PBS.

5.3.8 Articles of Manufacture

The compounds or pharmaceutically acceptable derivatives can be packaged as articles of manufacture containing packaging material, a compound or pharmaceutically acceptable derivative thereof provided herein, which is used for treatment, prevention or amelioration of one or more symptoms associated with C3a activity, and a label that indicates that the compound or pharmaceutically acceptable derivative thereof is used for treatment, prevention or amelioration of one or more symptoms of C3a receptor mediated diseases.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,252. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated.

5.4 Evaluation of the Activity of the Compounds

The C3a receptor biological activity of the compounds is demonstrated by methods known to one of skill in the art. Exemplary methods are described in International Application Publication No. 99/15490 and U.S. Pat. No. 6,489,339, which are incorporated herein by reference. In certain embodiments, methods to demonstrate C3a receptor biological activity of the compounds include compound induced Ca²⁺ mobilization and compound inhibition of human C3a induced Ca²⁺ mobilization. Certain exemplary methods are described in details in Example 47.

5.5 Methods of Treatments and Prevention

C3a receptor is ubiquitous in the mammalian host and is responsible for many biological functions, including many pathologies. In certain embodiments, provided herein are methods for modulating the C3a receptor activity. The methods are accomplished by contacting the C3a receptor with a compound provided herein. In certain embodiments, the methods are for antagonizing the C3a receptor. In other embodiments, the methods are for agonizing C3a receptor.

In certain embodiments, provided herein are methods for treating, preventing or ameliorating diseases associated with C3a receptor modulation. Such diseases include, but are not limited to acute inflammatory disease, atherosclerosis, chronic polyarthritis, systemic vasculitis, multiple sclerosis, Alzheimer's Disease, CNS inflammatory disease, Crohn's Disease, food allergies, non-bronchial allergies, ostoartritis, osteoporosis, thyroid disease, coronary heart disease renal disease, for example, Systemic Lupus Erythematosis, SLE-associated nephritis, membranoproliferative GN, membranous nephritis; rheumatological diseases, for example, rheumatoid arthritis, SLE, Behcet's syndrome, juvenile rheumatoid arthritis, Sjogren's syndrome; neurological diseases, for example, myasthenia gravis, multiple sclerosis, cerebral lupus, Guillain-Barre syndrome, Alzheimer's disease; dermatological diseases, for example, pernphigus/pemphigoid, phototoxic reactions, vasculitis; biocompatibility/shock diseases, for example, post-bypass syndrome, catheter reactions, sepsis, ARDS, anaphylaxis, transplant rejection, pre-eclampsia; and other diseases, for example, atheroma, bowel inflammation, thyroiditis, and infertility, suseptibility to pyogenic infections, glomerulonephritis, suseptibility to neisserial infections, recurrent subcutaneous swelling and mucosal oedema, and recurrent episodes of thrombosis/haemolysis.

5.5.1 Combination Therapy with a Second Active Agent

The compounds provided herein may be administered as the sole active ingredient or in combination with other active ingredients. Other active ingredients that may be used in combination with the compounds provided herein include but are not limited to, compounds known to treat diseases associated with C3a receptor modulation or compounds known to modulate C3a receptor activity. Exemplary of such compounds are provided in U.S. Pat. Nos. 6,489,339; 5,472,939 and 5,942,405; and International Application Publication No. WO200009129 and WO 1999015490.

Administration of the active ingredient combination may take place either by separate administration of the active ingredients to the patient or in the form of combination products in which a plurality of active ingredients are present in one pharmaceutical preparation.

It will be appreciated that every suitable combination of the compounds provided herein with one or more of the aforementioned compounds and optionally one or more further pharmacologically active substances is contemplated herein.

It is understood that the foregoing detailed description and accompanying examples are merely illustrative, and are not to be taken as limitations upon the scope of the subject matter. Various changes and modifications to the disclosed embodiments will be apparent to those skilled in the art. Such changes and modifications, including without limitation those relating to the chemical structures, substituents, derivatives, intermediates, syntheses, formulations and/or methods of use provided herein, may be made without departing from the spirit and scope thereof. U.S. patents and publications referenced herein are incorporated by reference.

### 6. EXAMPLES

Certain embodiments of the claimed subject matter are illustrated by the following non-limiting examples.

### Example 1

### Compound 1

### Synthesis of (2S)-2-{[(1-benzyl-2-oxo-1,2-dihydropyridin-3-yl)carbonyl]amino}-5-carbamimidamidopentanoic acid•TFA

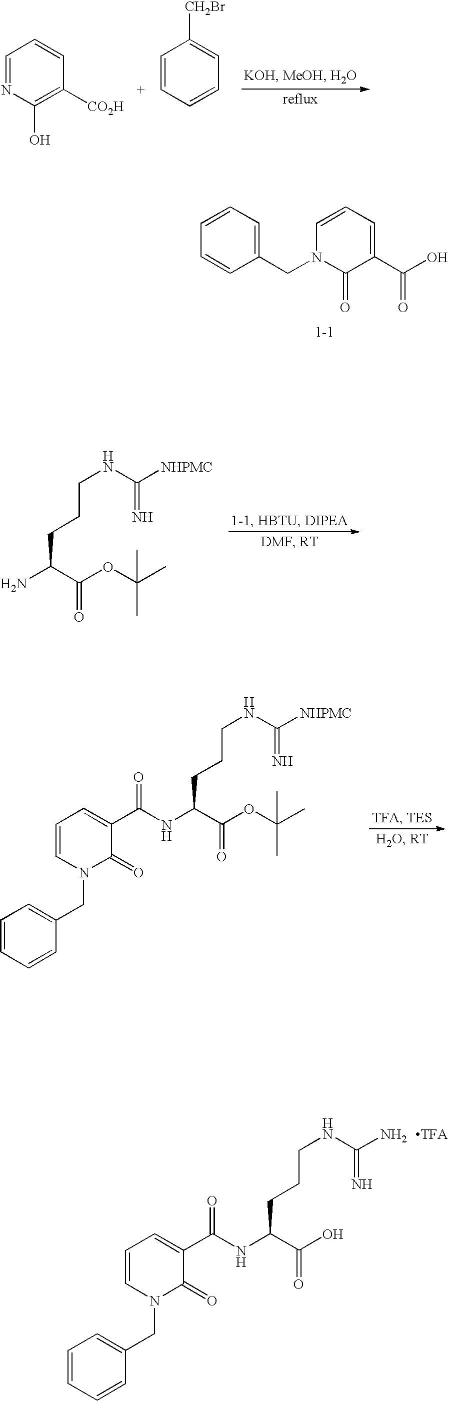

### I. 1-Benzyl-2-oxo-1,2-dihydropyridine-3-carboxylic acid

To a suspension of 2-hydroxynicotinic acid (1.00 g, 7.19 mmol) in water (3 mL) and methanol (10 mL) was added KOH (1.21 g, 21.57 mmol). The resulting solution was heated at reflux for 15 minutes and then benzyl bromide (1.8 mL, 15.1 mmol) was added. Heating was continued for an additional 90 minutes and then the reaction was cooled to room temperature and filtered. The filtrate was diluted with water and washed twice with diethyl ether. The organic phase was separated, extracted twice with 2M NaOH and then both aqueous extracts were added to the original aqueous phase. The precipitate isolated by filtration of the reaction was then dissolved in water and also added to the basic aqueous extract. The combined aqueous phase was acidified with 2M HCl, and the resulting white precipitate was isolated by vacuum filtration, washed with water and air dried to give a white solid (0.90 g, 55%).

### II. tert-Butyl (2S)-2-{[(1-benzyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (500 mg, 1.0 mmol), 1-benzyl-2-oxo-1,2-dihydropyridine-3-carboxylic acid (344 mg, 1.5 mmol), and HBTU (569 mg, 1.5 mmol) in anhydrous DMF (5 mL) was added DIPEA (0.7 mL, 4.0 mmol). The reaction mixture was stirred at room temperature overnight and then diluted with EtOAc. The organic layer was washed successively with saturated NaHCO₃, water and saturated NaCl, dried over Na₂SO₄ and filtered. The filtrate was concentrated under reduced pressure and the residue was purified by column chromatography on silica gel, eluting with a 1:1 EtOAc/hexanes to 100% EtOAc gradient to give a white foam (700 mg, 98%).

### III. (2S)-2-{[(1-Benzyl-2-oxo-1,2-dihydropyridin-3-yl)carbonyl]amino}-5-carbamimidamidopentanoic acid•TFA

To a solution of tert-butyl (2S)-2-{[(1-benzyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate (183 mg, 0.26 mmol) in TFA (2 mL), triethylsilane (0.2 mL) and water (0.2 mL) were added. After stirring the reaction mixture at room temperature for 3 hr, MTBE (25 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give a white solid (50 mg, 38%).

The following compounds were synthesized by modifications of the general procedure described in Example 1.

Compound 9: Step I was carried out using 1.00 g 2-hydroxynicotinic acid, 1.61 g KOH and 1.1 mL MeI in 10 mL MeOH/2 mL H₂O to provide 0.75 g 1-methyl-2-oxo-1,2-dihydropyridine-3-carboxylic acid. Step II was carried out using 30 mg 1-methyl-2-oxo-1,2-dihydropyridine-3-carboxylic acid, 107 mg tert-butyl (2S)-2-amino-5-{[(2,2,5,7,8-pentamethyl-3,4-dihydro-2H-chromen-6-yl)sulfonyl]carbamimidamido}pentanoate, 106 mg HBTU and 77 μL DIPEA in 2 mL DMF to provide 115 mg tert-butyl (2S)-2-{[(1-methyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate. Step III was carried out using 115 mg tert-butyl (2S)-2-{[(1-methyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate to provide 14.4 mg (2S)-5-carbamimidamido-2-{[(1-methyl-2-oxo-1,2-dihydropyridin-3-yl)carbonyl]amino}pentanoic acid•TFA following reversed phase HPLC purification.

Compound 35: Step I was carried out using 1.53 g 2-hydroxy-6-methylnicotinic acid, 1.96 g KOH and 2.7 mL benzyl bromide in 40 mL MeOH/20 mL H₂O over 3 days heating. An additional 2.81 g KOH and 4.7 mL benzyl bromide were added after the first day of heating. Following heating, the MeOH was removed under reduced pressure, and the aqueous phase was washed with Et₂O. After acidification with 6 M HCl, the product was extracted with EtOAc, and subsequent washings were performed (4 times with 2 M HCl, once with deionized H₂O, and once with saturated aqueous NaCl). The organic phase was dried over anhydrous Na₂SO₄, taken to dryness under reduced pressure and chromatographed on silica gel with 1:39 MeOH/CH₂Cl₂ to provide 368 mg 1-benzyl-6-methyl-2-oxo-1,2-dihydropyridine-3-carboxylic acid. Step II was carried out using 70 mg 1-benzyl-6-methyl-2-oxo-1,2-dihydropyridine-3-carboxylic acid, 143 mg tert-butyl (2S)-2-amino-5-{[(2,2,5,7,8-pentamethyl-3,4-dihydro-2H-chromen-6-yl)sulfonyl]carbamimidamido}pentanoate, 154 mg HBTU and 0.13 mL DIPEA in 3 mL DMF provide 188 mg tert-butyl (2S)-2-{[(1-benzyl-6-methyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate. Step III was carried out using 188 mg tert-butyl (2S)-2-{[(1-benzyl-6-methyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate to provide 45.8 mg (2S)-2-{[(1-benzyl-6-methyl-2-oxo-1,2-dihydropyridin-3-yl)carbonyl]amino}-5-carbamimidamidopentanoic acid•TFA following reversed phase HPLC purification.

Compound 60: Step II was carried out with 150 mg compound 1-1, 244 mg tert-butyl (2S)-2-amino-6-[(tert-butoxycarbonyl)amino]hexanoate hydrochloride, 380 mg HBTU, and 0.25 mL DIPEA in 3 mL DMF to provide 327 mg tert-butyl (2S)-2-{[(1-benzyl-2-oxo-1,2-dihydropyridin-3-yl)carbonyl]amino}-6-[(tert-butoxycarbonyl)amino]hexanoate. Step III was conducted with 327 mg tert-butyl (2S)-2-{[(1-benzyl-2-oxo-1,2-dihydropyridin-3-yl)carbonyl]amino}-6-[(tert-butoxycarbonyl)amino]hexanoate, 2 mL TFA, 0.2 mL triethylsilane, and 0.2 mL deionized H₂O to provide 121 mg (2S)-6-amino-2-{[(1-benzyl-2-oxo-1,2-dihydropyridin-3-yl)carbonyl]amino}hexanoic acid•TFA.

Compound 67: Step II was conducted with 76 mg compound 1-1, tert-butyl (2S)-2-amino-5-{[(4-methylphenyl)sulfonyl]amino}pentanoate [generated in situ from 135 mg tert-butyl (2S)-2-[(tert-butoxycarbonyl)amino]-5-{[(4-methylphenyl)sulfonyl]amino}pentanoate and 33 mg propionyl chloride in 3 mL MeOH), 137 mg HBTU, and 0.16 mL DIPEA in 2 mL DMF to provide 70 mg tert-butyl (2S)-2-{[(1-benzyl-2-oxo-1,2-dihydropyridin-3-yl)carbonyl]amino}-5-{[(4-methylphenyl)sulfonyl]amino}pentanoate. Step III was conducted with 68 mg tert-butyl (2S)-2-{[(1-benzyl-2-oxo-1,2-dihydropyridin-3-yl)carbonyl]amino}-5-{[(4-methylphenyl)sulfonyl]amino}pentanoate and 2 mL TFA in 5 mL CH₂Cl₂ to provide 15 mg (2S)-2-{[(1-benzyl-2-oxo-1,2-dihydropyridin-3-yl)carbonyl]amino}-5-{[(4-methylphenyl)sulfonyl]amino}pentanoic acid.

### Example 2

### Compound 2

### Synthesis of (2R)-2-{[(1-benzyl-2-oxo-1,2-dihydropyridin-3-yl)carbonyl]amino}-5-carbamimidamidopentanoic acid•TFA

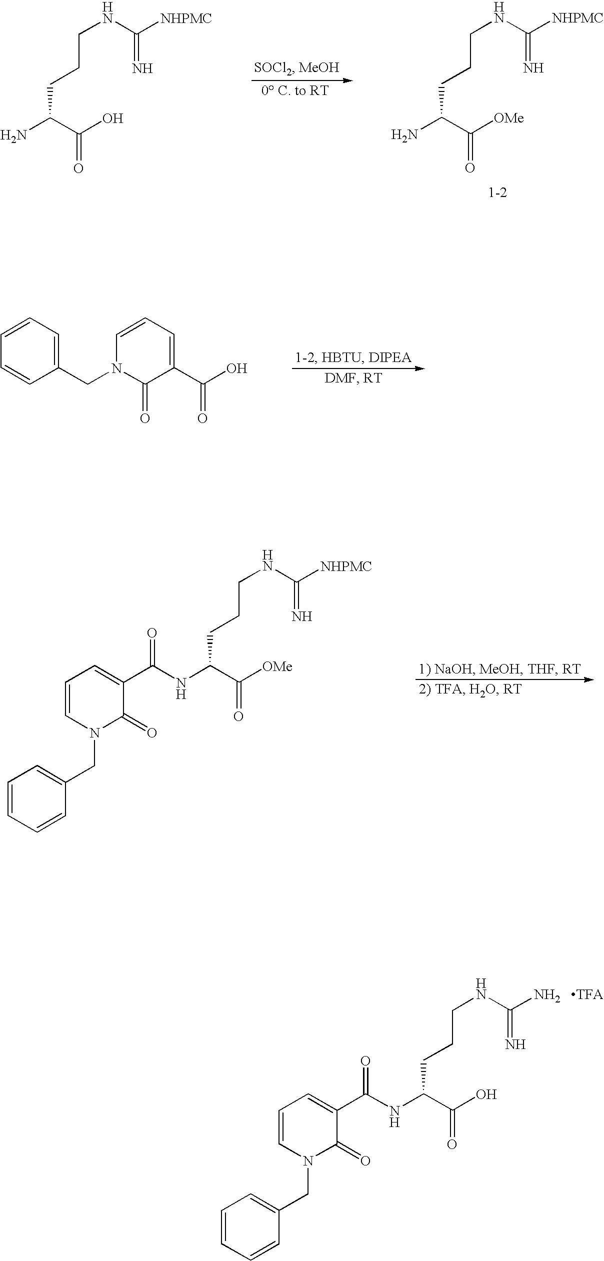

### I. Methyl (2R)-2-amino-5-{[(2,2,5,7,8-pentamethyl-3,4-dihydro-2H-chromen-6-yl)sulfonyl]carbamimidamido}pentanoate

Thionyl chloride (0.66 mL, 9.1 mmol) was added dropwise to a suspension of (2R)-2-amino-5-{[(2,2,5,7,8-pentamethyl-3,4-dihydro-2H-chromen-6-yl)sulfonyl]carbamimidamido}pentanoic acid (400 mg, 0.91 mmol) in 10 mL anhydrous methanol at 0° C. The reaction was stirred at 0° C. for 90 min, then at room temperature for 3 hr. The solvent was removed under reduced pressure and the crude product was purified by column chromatography on silica gel, eluting with 9:1 dichloromethane/methanol to give a pale yellow oil that solidified to a white solid under vacuum (239 mg, 58%).

### II. Methyl (2R)-2-{[(1-benzyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate

To a solution of methyl (2R)-2-amino-5-{[(2,2,5,7,8-pentamethyl-3,4-dihydro-2H-chromen-6-yl)sulfonyl]carbamimidamido}pentanoate (98 mg, 0.22 mmol), 1-benzyl-2-oxo-1,2-dihydropyridine-3-carboxylic acid (49 mg, 0.22 mmol), and HBTU (117 mg, 0.31 mmol) in anhydrous DMF (2 mL) was added DIPEA (0.14 mL, 0.77 mmol). The reaction mixture was stirred at room temperature for 2 days and then diluted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 100% EtOAc to give a white solid (121 mg, 82%).

### III. (2R)-2-{[(1-benzyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoic acid

To a solution of methyl (2R)-2-{[(1-benzyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate (121 mg, 0.18 mmol) in 1:1 THF/MeOH (4 mL) was added 2M NaOH (0.6 mL, 1.1 mmol) and the reaction stirred at room temperature for 2.5 hr. The reaction was then diluted with water, washed with diethyl ether (2 times) and the layers separated. The aqueous phase was acidified with 2M HCl and extracted with EtOAc. The organic layer was washed with water and saturated NaCl, dried over Na₂SO₄ and filtered. The solvent was removed under reduced pressure to give a white semi-solid (115 mg, 98%).

### IV. (2R)-2-{[(1-benzyl-2-oxo-1,2-dihydropyridin-3-yl)carbonyl]amino}-5-carbamimidamidopentanoic acid•TFA

To a solution of (2R)-2-{[(1-benzyl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoic acid (115 mg, 0.18 mmol) in TFA (2 mL) was added H₂O (0.4 mL). After stirring the reaction mixture at room temperature for 4 hr, MTBE (20 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized, purified by reversed-phase preparative HPLC using a 10 to 60% CH₃CN/0.1% TFA in H₂O gradient, and then lyophilized again to give a white solid (36 mg, 40%).

### Example 3

### Compound 13

### Synthesis of (2S)-5-carbamimidamido-2-({[1-(2-naphthylmethyl)-2-oxo-1,2-dihydropyridin-3-yl]carbonyl}amino)pentanoic acid•TFA

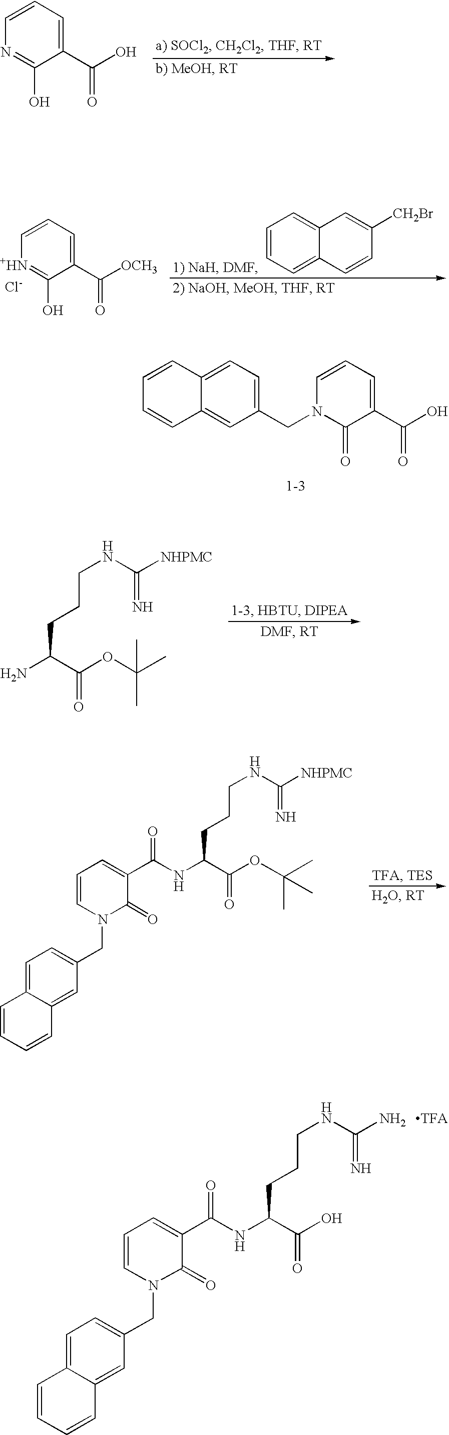

### I. Methyl 2-oxo-1,2-dihydropyridine-3-carboxylate hydrochloride

To a suspension of 2-hydroxynicotinic acid (10.00 g, 71.9 mmol) in anhydrous dichloromethane (150 mL) was added thionyl chloride (25.9 mL, 215.8 mmol) followed by anhydrous THF (150 mL). The reaction mixture was stirred at room temperature for 1 hr, and then excess methanol was added to the suspension until a homogenous solution was obtained. The reaction mixture was filtered and the filtrate concentrated under reduced pressure to give a pale yellow oil which solidified to an off-white solid under vacuum (13.96 g, 100%).

### II. Methyl 1-(2-naphthylmethyl)-2-oxo-1,2-dihydropyridine-3-carboxylate

Sodium hydride (60%, 46 mg, 1.13 mmol) was added to a solution of methyl 2-oxo-1,2-dihydropyridine-3-carboxylate hydrochloride (150 mg, 0.80 mmol) in DMF (5 mL). The resulting suspension was stirred at room temperature for 30 min and then a solution of 2-bromomethylnaphthalene (273 mg, 1.23 mmol) in anhydrous DMF (3 mL) was added via syringe. After stirring overnight at room temperature, the reaction was quenched with 2M HCl and diluted with ethyl acetate. The organic layer was washed with water (3 times) and saturated NaCl, dried over Na₂SO₄ and filtered. The filtrate was concentrated under reduced pressure and the residue was purified by column chromatography on silica gel, eluting with 60:40% EtOAc/hexanes to 100% EtOAc gradient to give a white solid (140 mg, 60%).

### II. 1-(2-Naphthylmethyl)-2-oxo-1,2-dihydropyridine-3-carboxylic acid

To a solution of methyl 1-(2-naphthylmethyl)-2-oxo-1,2-dihydropyridine-3-carboxylate (130 mg, 0.44 mmol) in 1:1 THF/MeOH (10 mL) was added 2M NaOH (1.3 mL, 2.6 mmol) and the reaction was stirred at room temperature for 5 hr. The reaction was diluted with water, and the resulting solution was washed twice with diethyl ether. Acidification of the aqueous phase produced a thick white precipitate. The solid was isolated by filtration and washed with water to give pure product (118 mg, 96%).

### IV. tert-Butyl (2S)-2-({[1-(2-naphthylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (107 mg, 0.22 mmol), 1-(2-naphthylmethyl)-2-oxo-1,2-dihydropyridine-3-carboxylic acid (60 mg, 0.22 mmol), and HBTU (117 mg, 0.31 mmol) in anhydrous DMF (2 mL) was added DIPEA (0.10 mL, 0.55 mmol). The reaction mixture was stirred at room temperature overnight and then diluted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 100% EtOAc (171 mg, 100%).

### V. (2S)-5-Carbamimidamido-2-({[1-(2-naphthylmethyl)-2-oxo-1,2-dihydropyridin-3-yl]carbonyl}amino)pentanoic acid•TFA

To a solution of tert-butyl (2S)-2-({[1-(2-naphthylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate (171 mg, 0.23 mmol) in TFA (2 mL), triethylsilane (0.2 mL) and water (0.2 mL) were added. After stirring the reaction mixture at room temperature for 3 hr, MTBE (25 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give a white solid (87 mg, 70%).

The following compounds were synthesized by modifications of the general procedure described in Example 3.

Compound 3: Step II was carried out using 372 mg methyl 2-oxo-1,2-dihydropyridine-3-carboxylate hydrochloride, 117 mg 60% NaH, and 840 mg 2-bromobenzyl bromide in 15 mL DMF to provide 529 mg methyl 1-(2-bromobenzyl)-2-oxo-1,2-dihydropyridine-3-carboxylate. Step III was carried out using 529 mg methyl 1-(2-bromobenzyl)-2-oxo-1,2-dihydropyridine-3-carboxylate, 0.25 mL 6 M NaOH, and 4 mL MeOH to provide 230 mg 1-(2-bromobenzyl)-2-oxo-1,2-dihydropyridine-3-carboxylic acid. Step IV was carried out using 230 mg 1-(2-bromobenzyl)-2-oxo-1,2-dihydropyridine-3-carboxylic acid, 330 mg tert-butyl (2S)-2-amino-5-{[(2,2,5,7,8-pentamethyl-3,4-dihydro-2H-chromen-6-yl)sulfonyl]carbamimidamido}pentanoate, 341 mg HBTU and 0.31 mL DIPEA in 10 mL DMF to provide 420 mg tert-butyl (2S)-2-({[1-(2-bromobenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 420 mg tert-butyl (2S)-2-({[1-(2-bromobenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 4 mL TFA, 0.1 mL triethylsilane and 0.1 mL H₂O to provide 180 mg (2S)-2-({[1-(2-bromobenzyl)-2-oxo-1,2-dihydropyridin-3-yl]carbonyl}amino)-5-carbamimidamidopentanoic acid•TFA.

Compound 4: Step II was carried out using 600 mg methyl 2-oxo-1,2-dihydropyridine-3-carboxylate hydrochloride, 246 mg 60% NaH, and 1.57 g 3-bromobenzyl bromide in 30 mL DMF to provide 0.61 g methyl 1-(3-bromobenzyl)-2-oxo-1,2-dihydropyridine-3-carboxylate. Step III was carried out using 0.61 g methyl 1-(3-bromobenzyl)-2-oxo-1,2-dihydropyridine-3-carboxylate, 0.25 mL 6 M NaOH, and 4 mL MeOH to provide 220 mg 1-(3-bromobenzyl)-2-oxo-1,2-dihydropyridine-3-carboxylic acid. Step IV was carried out using 220 mg 1-(3-bromobenzyl)-2-oxo-1,2-dihydropyridine-3-carboxylic acid, 309 mg tert-butyl (2S)-2-amino-5-{[(2,2,5,7,8-pentamethyl-3,4-dihydro-2H-chromen-6-yl)sulfonyl]carbamimidamido}pentanoate, 327 mg HBTU and 0.30 mL DIPEA in 10 mL DMF to provide 164 mg tert-butyl (2S)-2-({[1-(3-bromobenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 164 mg tert-butyl (2S)-2-({[1-(3-bromobenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 4 mL TFA, 0.1 mL triethylsilane and 0.1 mL H₂O to provide 42 mg (2S)-2-({[1-(3-bromobenzyl)-2-oxo-1,2-dihydropyridin-3-yl]carbonyl}amino)-5-carbamimidamidopentanoic acid•TFA.

Compound 5: Step II was carried out using 600 mg methyl 2-oxo-1,2-dihydropyridine-3-carboxylate hydrochloride, 220 mg 60% NaH, and 1.77 g 4-bromobenzyl bromide in 30 mL DMF to provide 428 mg methyl 1-(4-bromobenzyl)-2-oxo-1,2-dihydropyridine-3-carboxylate. Step III was carried out using 428 mg methyl 1-(4-bromobenzyl)-2-oxo-1,2-dihydropyridine-3-carboxylate, 0.25 mL 6 M NaOH, and 4 mL MeOH to provide 120 mg 1-(4-bromobenzyl)-2-oxo-1,2-dihydropyridine-3-carboxylic acid. Step IV was carried out using 120 mg 1-(4-bromobenzyl)-2-oxo-1,2-dihydropyridine-3-carboxylic acid, 213 mg tert-butyl (2S)-2-amino-5-{[(2,2,5,7,8-pentamethyl-3,4-dihydro-2H-chromen-6-yl)sulfonyl]carbamimidamido}pentanoate, 177 mg HBTU and 0.16 mL DIPEA in 10 mL DMF to provide 140 mg tert-butyl (2S)-2-({[1-(4-bromobenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 140 mg tert-butyl (2S)-2-({[1-(4-bromobenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 4 mL TFA, 0.1 mL triethylsilane and 0.1 mL H₂O to provide 67 mg (2S)-2-({[1-(4-bromobenzyl)-2-oxo-1,2-dihydropyridin-3-yl]carbonyl}amino)-5-carbamimidamidopentanoic acid•TFA.

Compound 6: Step I was carried out with 15.0 g 2-hydroxynicotinic acid and 23.6 mL SOCl₂ in 180 mL CH₂Cl₂/180 mL THF. Following quench with EtOH, 12.54 g ethyl 2-oxo-1,2-dihydropyridine-3-carboxylate hydrochloride was isolated following trituration with boiling hexanes. Step II was carried out using 430 mg 2-isopropylbenzyl bromide, 500 mg ethyl 2-oxo-1,2-dihydropyridine-3-carboxylate hydrochloride and 160 mg 60% NaH in 5 mL DMF to provide 460 mg ethyl 1-(2-isopropylbenzyl)-2-oxo-1,2-dihydropyridine-3-carboxylate. Step III was carried out with 460 mg ethyl 1-(2-isopropylbenzyl)-2-oxo-1,2-dihydropyridine-3-carboxylate and 10 drops 6 M NaOH in 5 mL MeOH to provide 1-(2-isopropylbenzyl)-2-oxo-1,2-dihydropyridine-3-carboxylic acid (yield not determined). Step IV was carried out using 1-(2-isopropylbenzyl)-2-oxo-1,2-dihydropyridine-3-carboxylic acid from Step III, 765 mg tert-butyl (2S)-2-amino-5-{[(2,2,5,7,8-pentamethyl-3,4-dihydro-2H-chromen-6-yl)sulfonyl]carbamimidamido}pentanoate, 700 mg HBTU and 0.51 mL DIPEA in 10 mL DMF to provide 925 mg tert-butyl (2S)-2-({[1-(2-isopropylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 925 mg tert-butyl (2S)-2-({[1-(2-isopropylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 5 mL TFA, 0.1 mL triethylsilane and 0.1 mL H₂O to provide 275 mg (2S)-5-carbamimidamido-2-({[1-(2-isopropylbenzyl)-2-oxo-1,2-dihydropyridin-3-yl]carbonyl}amino)pentanoic acid•TFA following reversed-phase HPLC purification.

Compound 7: Step I was carried as for compound 6 above. Step II was carried out using 470 mg 3-isopropylbenzyl bromide, 500 mg ethyl 2-oxo-1,2-dihydropyridine-3-carboxylate hydrochloride and 160 mg 60% NaH in 5 mL DMF to provide 310 mg ethyl 1-(3-isopropylbenzyl)-2-oxo-1,2-dihydropyridine-3-carboxylate. Step III was carried out with 310 mg ethyl 1-(3-isopropylbenzyl)-2-oxo-1,2-dihydropyridine-3-carboxylate and 10 drops 6 M NaOH in 5 mL MeOH to provide 1-(3-isopropylbenzyl)-2-oxo-1,2-dihydropyridine-3-carboxylic acid (yield not determined). Step IV was carried out using 1-(3-isopropylbenzyl)-2-oxo-1,2-dihydropyridine-3-carboxylic acid from Step III, 516 mg tert-butyl (2S)-2-amino-5-{[(2,2,5,7,8-pentamethyl-3,4-dihydro-2H-chromen-6-yl)sulfonyl]carbamimidamido}pentanoate, 473 mg HBTU and 0.35 mL DIPEA in 10 mL DMF to provide 540 mg tert-butyl (2S)-2-({[1-(3-isopropylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 540 mg tert-butyl (2S)-2-({[1-(3-isopropylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 5 mL TFA, 0.1 mL triethylsilane and 0.1 mL H₂O to provide 202 mg (2S)-5-carbamimidamido-2-({[1-(3-isopropylbenzyl)-2-oxo-1,2-dihydropyridin-3-yl]carbonyl}amino)pentanoic acid•TFA.

Compound 8: Step II was carried out using 405 mg 4-isopropylbenzyl bromide, 500 mg methyl 2-oxo-1,2-dihydropyridine-3-carboxylate hydrochloride and 188 mg 60% NaH in 10 mL DMF to provide methyl 1-(4-isopropylbenzyl)-2-oxo-1,2-dihydropyridine-3-carboxylate (yield not determined). Step III was carried out with methyl 1-(4-isopropylbenzyl)-2-oxo-1,2-dihydropyridine-3-carboxylate from Step II and 10 drops 6 M NaOH in 5 mL MeOH to provide 1-(4-isopropylbenzyl)-2-oxo-1,2-dihydropyridine-3-carboxylic acid (yield not determined). Step IV was carried out using 100 mg 1-(4-isopropylbenzyl)-2-oxo-1,2-dihydropyridine-3-carboxylic acid from Step III, 183 mg tert-butyl (2S)-2-amino-5-{[(2,2,5,7,8-pentamethyl-3,4-dihydro-2H-chromen-6-yl)sulfonyl]carbamimidamido}pentanoate, 170 mg HBTU and 0.17 mL DIPEA in 10 mL DMF to provide 311 mg tert-butyl (2S)-2-({[1-(4-isopropylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 311 mg tert-butyl (2S)-2-({[1-(4-isopropylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 5 mL TFA, 0.1 mL triethylsilane and 0.1 mL H₂O to provide 164 mg (2S)-5-carbamimidamido-2-([1-(4-isopropylbenzyl)-2-oxo-1,2-dihydropyridin-3-yl]carbonyl}amino)pentanoic acid-TFA following reversed-phase HPLC purification.

Compound 10: Step II was carried out using 360 mg methyl 2-oxo-1,2-dihydropyridine-3-carboxylate hydrochloride, 100 mg 60% NaH, and 400 mg 9-bromofluorene in 18 mL DMF to provide 0.80 g crude methyl 1-(9H-fluoren-9-yl)-2-oxo-1,2-dihydropyridine-3-carboxylate. Step III was carried out using 0.80 g methyl 1-(9H-fluoren-9-yl)-2-oxo-1,2-dihydropyridine-3-carboxylate and 3 mL 2 M NaOH in 6 mL THF/1 mL MeOH to provide 220 mg 1-(9H-fluoren-9-yl)-2-oxo-1,2-dihydropyridine-3-carboxylic acid. Step IV was carried out using 220 mg 1-(9H-fluoren-9-yl)-2-oxo-1,2-dihydropyridine-3-carboxylic acid, 300 mg tert-butyl (2S)-2-amino-5-{[(2,2,5,7,8-pentamethyl-3,4-dihydro-2H-chromen-6-yl)sulfonyl]carbamimidamido}pentanoate, 320 mg HBTU and 0.15 mL DIPEA in 4 mL DMF to provide 420 mg tert-butyl (2S)-2-({[1-(9H-fluoren-9-yl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 420 mg tert-butyl (2S)-2-({[1-(9H-fluoren-9-yl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 6 mL TFA, 0.6 mL triethylsilane and 0.6 mL H₂O to provide 170 mg (2S)-5-carbamimidamido-2-({[1-(9H-fluoren-9-yl)-2-oxo-1,2-dihydropyridin-3-yl]carbonyl}amino)pentanoic acid•TFA.

Compound 12: Step II was carried out using 241 mg methyl 2-oxo-1,2-dihydropyridine-3-carboxylate hydrochloride, 133 mg 60% NaH, and 435 mg 3,3-diphenylpropyl chloride in 7 mL DMF to provide 81 mg methyl 1-(3,3-diphenylpropyl)-2-oxo-1,2-dihydropyridine-3-carboxylate. Step III was carried out using 81 mg methyl 1-(3,3-diphenylpropyl)-2-oxo-1,2-dihydropyridine-3-carboxylate and 0.7 mL 2 M NaOH in 4 mL 1:1 THF/MeOH to provide 68 mg 1-(3,3-diphenylpropyl)-2-oxo-1,2-dihydropyridine-3-carboxylic acid. Step IV was carried out using 68 mg 1-(3,3-diphenylpropyl)-2-oxo-1,2-dihydropyridine-3-carboxylic acid, 101 mg tert-butyl (2S)-2-amino-5-{[(2,2,5,7,8-pentamethyl-3,4-dihydro-2H-chromen-6-yl)sulfonyl]carbamimidamido}pentanoate, 106 mg HBTU and 0.09 mL DIPEA in 2 mL DMF to provide 157 mg tert-butyl (2S)-2-({[1-(3,3-diphenylpropyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 157 mg tert-butyl (2S)-2-({[1-(3,3-diphenylpropyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 2 mL TFA, 0.2 mL triethylsilane and 0.2 mL H₂O to provide 82 mg (2S)-5-carbamimidamido-2-({[1-(3,3-diphenylpropyl)-2-oxo-1,2-dihydropyridin-3-yl]carbonyl}amino)pentanoic acid•TFA.

Compound 14: Step II was carried out using 400 mg methyl 2-oxo-1,2-dihydropyridine-3-carboxylate hydrochloride, 120 mg 60% NaH, and 460 mg 1-chloromethylnaphthalene in 26 mL DMF to provide 500 mg methyl 1-(naphthalen-1-ylmethyl)-2-oxo-1,2-dihydropyridine-3-carboxylate. Step III was carried out using 500 mg methyl 1-(naphthalen-1-ylmethyl)-2-oxo-1,2-dihydropyridine-3-carboxylate and 3 mL 2 M NaOH in 6 mL THF/1 mL MeOH to provide 500 mg 1-(naphthalen-1-ylmethyl)-2-oxo-1,2-dihydropyridine-3-carboxylic acid. Step IV was carried out using 500 mg 1-(naphthalen-1-ylmethyl)-2-oxo-1,2-dihydropyridine-3-carboxylic acid, 890 mg tert-butyl (2S)-2-amino-5-{[(2,2,5,7,8-pentamethyl-3,4-dihydro-2H-chromen-6-yl)sulfonyl]carbamimidamido}pentanoate, 950 mg HBTU and 0.40 mL DIPEA in 9 mL DMF to provide 910 mg tert-butyl (2S)-2-({[1-(1-naphthylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 900 mg tert-butyl (2S)-2-({[1-(1-naphthylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 12 mL TFA, 1.2 mL triethylsilane and 1.2 mL H₂O to provide 350 mg (2S)-5-carbamimidamido-2-({[1-(1-naphthylmethyl)-2-oxo-1,2-dihydropyridin-3-yl]carbonyl}amino)pentanoic acid•TFA following reversed-phase HPLC purification.

Compound 15: Step II was carried out using 175 mg methyl 2-oxo-1,2-dihydropyridine-3-carboxylate hydrochloride, 48 mg 60% NaH, and 224 mg 2-chloromethylquinoline in 5 mL DMF to provide 86 mg methyl 1-(quinolin-2-ylmethyl)-2-oxo-1,2-dihydropyridine-3-carboxylate. Step III was carried out using 86 mg methyl 1-(quinolin-2-ylmethyl)-2-oxo-1,2-dihydropyridine-3-carboxylate, 0.88 mL 2 M NaOH, and 4 1:1 THF/MeOH to provide 75 mg 1-(quinolin-2-ylmethyl)-2-oxo-1,2-dihydropyridine-3-carboxylic acid. Step IV was carried out using 75 mg 1-(quinol-2-ylmethyl)-2-oxo-1,2-dihydropyridine-3-carboxylic acid, 133 mg tert-butyl (2S)-2-amino-5-{[(2,2,5,7,8-pentamethyl-3,4-dihydro-21′-chromen-6-yl)sulfonyl]carbamimidamido}pentanoate, 143 mg HBTU and 0.12 mL DIPEA in 3 mL DMF to provide 136 mg tert-butyl (2S)-2-({[2-oxo-1-(quinolin-2-ylmethyl)-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 136 mg tert-butyl (2S)-2-({[2-oxo-1-(quinolin-2-ylmethyl)-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 2 mL TFA, 0.2 mL triethylsilane and 0.2 mL H₂O to provide 133 mg (2S)-5-carbamimidamido-2-({[2-oxo-1-(quinolin-2-ylmethyl)-1,2-dihydropyridin-3-yl]carbonyl}amino)pentanoic acid•TFA.

Compound 16: Step I was carried as for compound 6 above. Step II was carried out using 185 mg 6-(bromomethyl)-1,1,4,4-tetramethyl-1,2,3,4-tetrahydronaphthalene, 167 mg ethyl 2-oxo-1,2-dihydropyridine-3-carboxylate hydrochloride and 40 mg 60% NaH in 5 mL DMF to provide 100 mg ethyl 2-oxo-1-[(5,5,8,8-tetramethyl-5,6,7,8-tetrahydro-2-naphthyl)methyl]-1,2-dihydropyridine-3-carboxylate. Step III was carried out with 100 mg ethyl 2-oxo-1-[(5,5,8,8-tetramethyl-5,6,7,8-tetrahydro-2-naphthyl)methyl]-1,2-dihydropyridine-3-carboxylateand 10 drops 6 M NaOH in 5 mL MeOH to provide 90 mg 2-oxo-1-[(5,5,8,8-tetramethyl-5,6,7,8-tetrahydro-2-naphthyl)methyl]-1,2-dihydropyridine-3-carboxylic acid. Step IV was carried out using 90 mg 2-oxo-1-[(5,5,8,8-tetramethyl-5,6,7,8-tetrahydro-2-naphthyl)methyl]-1,2-dihydropyridine-3-carboxylic acid, 167 mg tert-butyl (2S)-2-amino-5-{[(2,2,5,7,8-pentamethyl-3,4-dihydro-2H-chromen-6-yl)sulfonyl]carbamimidamido}pentanoate, 160 mg HBTU and 0.14 mL DIPEA in 10 mL DMF to provide 220 mg tert-butyl (2S)-2-[({2-oxo-1-[(5,5,8,8-tetramethyl-5,6,7,8-tetrahydro-2-naphthyl)methyl]-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step V was carried out using 220 mg tert-butyl (2S)-2-[({2-oxo-1-[(5,5,8,8-tetramethyl-5,6,7,8-tetrahydro-2-naphthyl)methyl]-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 5 mL TFA, 0.1 mL triethylsilane and 0.1 mL H₂O to provide 110 mg (2S)-5-carbamimidamido-2-[({2-oxo-1-[(5,5,8,8-tetramethyl-5,6,7,8-tetrahydro-2-naphthyl)methyl]-1,2-dihydropyridin-3-yl}carbonyl)amino]pentanoic acid•TFA following reversed-phase HPLC purification.

Compound 17: Step I was carried as for compound 6 above. Step II was carried out using 256 mg 2-bromomethyl-6-fluoronaphthalene, 270 mg ethyl 2-oxo-1,2-dihydropyridine-3-carboxylate hydrochloride and 65 mg 60% NaH in 5 mL DMF to provide 165 mg ethyl 1-[(6-fluoro-2-naphthyl)methyl]-2-oxo-1,2-dihydropyridine-3-carboxylate. Step III was carried out with 165 mg ethyl 1-[(6-fluoro-2-naphthyl)methyl]-2-oxo-1,2-dihydropyridine-3-carboxylate and 10 drops 6 M NaOH in 5 mL MeOH to provide 140 mg 1-[(6-fluoro-2-naphthyl)methyl]-2-oxo-1,2-dihydropyridine-3-carboxylic acid. Step IV was carried out using 140 mg 1-[(6-fluoro-2-naphthyl)methyl]-2-oxo-1,2-dihydropyridine-3-carboxylic acid, 316 mg tert-butyl (2S)-2-amino-5-{[(2,2,5,7,8-pentamethyl-3,4-dihydro-2H-chromen-6-yl)sulfonyl]carbamimidamido}pentanoate, 300 mg HBTU and 0.28 mL DIPEA in 10 mL DMF to provide 220 mg tert-butyl (2S)-2-[({1-[(6-fluoro-2-naphthyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step V was carried out using 220 mg tert-butyl (2S)-2-[({1-[(6-fluoro-2-naphthyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 5 mL TFA, 0.1 mL triethylsilane and 0.1 mL H₂O to provide 107 mg (2S)-5-carbamimidamido-2-[({1-[(6-fluoro-2-naphthyl)methyl]-2-oxo-1,2-dihydropyridin-3-yl}carbonyl)amino]pentanoic acid•TFA following reversed-phase HPLC purification.

Compound 18: Step I was carried as for compound 6 above. Step II was carried out using 186 mg 2-bromomethyl-3-methoxynaphthalene, 250 mg ethyl 2-oxo-1,2-dihydropyridine-3-carboxylate hydrochloride and 60 mg 60% NaH in 5 mL DMF to provide 220 mg ethyl 1-[(3-methoxy-2-naphthyl)methyl]-2-oxo-1,2-dihydropyridine-3-carboxylate. Step III was carried out with 220 mg ethyl 1-[(3-methoxy-2-naphthyl)methyl]-2-oxo-1,2-dihydropyridine-3-carboxylate and 10 drops 6 M NaOH in 5 mL MeOH to provide 200 mg 1-[(3-methoxy-2-naphthyl)methyl]-2-oxo-1,2-dihydropyridine-3-carboxylic acid. Step IV was carried out using 200 mg 1-[(3-methoxy-2-naphthyl)methyl]-2-oxo-1,2-dihydropyridine-3-carboxylic acid, 317 mg tert-butyl (2S)-2-amino-5-{[(2,2,5,7,8-pentamethyl-3,4-dihydro-2H-chromen-6-yl)sulfonyl]carbamimidamido}pentanoate, 295 mg HBTU and 0.35 mL DIPEA in 10 mL DMF to provide 270 mg tert-butyl (2S)-2-[({1-[(3-methoxy-2-naphthyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step V was carried out using 270 mg tert-butyl (2S)-2-[({1-[(3-methoxy-2-naphthyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 2 mL TFA, 0.1 mL triethylsilane and 0.1 mL H₂O to provide 30 mg (2S)-5-carbamimidamido-2-[({1-[(3-methoxy-2-naphthyl)methyl]-2-oxo-1,2-dihydropyridin-3-yl}carbonyl)amino]pentanoic acid•TFA following reversed-phase HPLC purification.

Compound 19: Step I was carried as for compound 6 above. Step II was carried out using 130 mg 2-bromomethyl-6-methoxynaphthalene, 167 mg ethyl 2-oxo-1,2-dihydropyridine-3-carboxylate hydrochloride and 40 mg 60% NaH in 5 mL DMF to provide 310 mg ethyl 1-[(6-methoxy-2-naphthyl)methyl]-2-oxo-1,2-dihydropyridine-3-carboxylate. Step III was carried out with 310 mg ethyl 1-[(6-methoxy-2-naphthyl)methyl]-2-oxo-1,2-dihydropyridine-3-carboxylate and 0.5 mL 6 M NaOH in 5 mL MeOH to provide 260 mg 1-[(6-methoxy-2-naphthyl)methyl]-2-oxo-1,2-dihydropyridine-3-carboxylic acid. Step IV was carried out using 260 mg 1-[(6-methoxy-2-naphthyl)methyl]-2-oxo-1,2-dihydropyridine-3-carboxylic acid, 417 mg tert-butyl (2S)-2-amino-5-{[(2,2,5,7,8-pentamethyl-3,4-dihydro-2H-chromen-6-yl)sulfonyl]carbamimidamido}pentanoate, 382 mg HBTU and 0.45 mL DIPEA in 10 mL DMF to provide 160 mg tert-butyl (2S)-2-[({1-[(6-methoxy-2-naphthyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step V was carried out using 160 mg tert-butyl (2S)-2-[({1-[(6-methoxy-2-naphthyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 2 mL TFA, 0.1 mL triethylsilane and 0.1 mL H₂O to provide 30 mg (2S)-5-carbamimidamido-2-[({1-[(6-methoxy-2-naphthyl)methyl]-2-oxo-1,2-dihydropyridin-3-yl}carbonyl)amino]pentanoic acid•TFA following reversed-phase HPLC purification.

Compound 37: Step II was carried out using 950 mg 2-bromo-N,N-diphenylacetamide, 626 mg methyl 2-oxo-1,2-dihydropyridine-3-carboxylate hydrochloride and 288 mg 60% NaH in 15 mL DMF to provide 130 mg methyl 1-[2-(diphenylamino)-2-oxoethyl]-2-oxo-1,2-dihydropyridine-3-carboxylate. Step III was carried out with 130 mg methyl 1-[2-(diphenylamino)-2-oxoethyl]-2-oxo-1,2-dihydropyridine-3-carboxylate and 0.5 mL 6 M NaOH in 4 mL MeOH to provide 90 mg 1-[2-(diphenylamino)-2-oxoethyl]-2-oxo-1,2-dihydropyridine-3-carboxylic acid. Step IV was carried out using 90 mg 1-[2-(diphenylamino)-2-oxoethyl]-2-oxo-1,2-dihydropyridine-3-carboxylic acid, 197 mg tert-butyl (2S)-2-amino-5-{[(2,2,5,7,8-pentamethyl-3,4-dihydro-2H-chromen-6-yl)sulfonyl]carbamimidamido}pentanoate, 164 mg HBTU and 0.15 mL DIPEA in 5 mL DMF to provide 204 mg tert-butyl (2S)-2-[({1-[2-(diphenylamino)-2-oxoethyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step V was carried out using 204 mg tert-butyl (2S)-2-[({1-[2-(diphenylamino)-2-oxoethyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 1.5 mL TFA, 0.1 mL triethylsilane and 0.1 mL H₂O to provide 28 (2S)-5-carbamimidamido-2-[({1-[2-(diphenylamino)-2-oxoethyl]-2-oxo-1,2-dihydropyridin-3-yl}carbonyl)amino]pentanoic acid•TFA following reversed-phase HPLC purification.

Compound 38: Step II was carried out using 0.4 mL 3-chlorobenzyl bromide, 330 mg methyl 2-oxo-1,2-dihydropyridine-3-carboxylate hydrochloride and 220 mg 60% NaH in 11 mL DMF to provide 690 mg methyl 1-(3-chlorobenzyl)-2-oxo-1,2-dihydro-pyridine-3-carboxylate. Step III was carried out with 690 mg methyl 1-(3-chlorobenzyl)-2-oxo-1,2-dihydro-pyridine-3-carboxylate and 3 mL 2 M NaOH in 6 mL THF/1 mL MeOH to provide 460 mg 1-(3-chlorobenzyl)-2-oxo-1,2-dihydro-pyridine-3-carboxylic acid. Step IV was carried out using 150 mg 1-(3-chlorobenzyl)-2-oxo-1,2-dihydro-pyridine-3-carboxylic acid, 250 mg tert-butyl (2S)-2-amino-5-{[(2,2,5,7,8-pentamethyl-3,4-dihydro-2H-chromen-6-yl)sulfonyl]carbamimidamido}pentanoate, 300 mg HBTU and 0.13 mL DIPEA in 3 mL DMF to provide 370 mg tert-butyl (2S)-2-({[1-(3-chlorobenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step V was carried out using 370 mg tert-butyl (2S)-2-({[1-(3-chlorobenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 6 mL TFA, 0.6 mL triethylsilane and 0.6 mL H₂O to provide 200 mg (2S)-5-carbamimidamido-2-({[1-(3-chlorobenzyl)-2-oxo-1,2-dihydropyridin-3-yl]carbonyl}amino)pentanoic acid•TFA.

Compound 39: Step II was carried out using 0.4 mL 3-fluorobenzyl bromide, 330 mg methyl 2-oxo-1,2-dihydropyridine-3-carboxylate hydrochloride and 220 mg 60% NaH in 11 mL DMF to provide 660 mg methyl 1-(3-fluorobenzyl)-2-oxo-1,2-dihydro-pyridine-3-carboxylate. Step III was carried out with 660 mg methyl 1-(3-fluorobenzyl)-2-oxo-1,2-dihydro-pyridine-3-carboxylate and 3 mL 2 M NaOH in 6 mL THF/1 mL MeOH to provide 380 mg 1-(3-fluorobenzyl)-2-oxo-1,2-dihydro-pyridine-3-carboxylic acid. Step IV was carried out using 140 mg 1-(3-fluorobenzyl)-2-oxo-1,2-dihydro-pyridine-3-carboxylic acid, 250 mg tert-butyl (2S)-2-amino-5-{[(2,2,5,7,8-pentamethyl-3,4-dihydro-2H-chromen-6-yl)sulfonyl]carbamimidamido}pentanoate, 300 mg HBTU and 0.13 mL DIPEA in 3 mL DMF to provide 430 mg tert-butyl (2S)-2-(f{[1-(3-fluorobenzyl)-2-oxo-1,2-dihydropyridin-3-yl]carbonyl amino)-5-[(2,2,5,7,8-pentamethyl-3,4-dihydro-2H-chromen-6-yl)sulfonyl]carbamimidamido}pentanoate. Step V was carried out using 430 mg tert-butyl (2S)-2-({[1-(3-fluorobenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 6 mL TFA, 0.6 mL triethylsilane and 0.6 mL H₂O to provide 140 mg (2S)-5-carbamimidamido-2-({[1-(3-fluorobenzyl)-2-oxo-1,2-dihydropyridin-3-yl]carbonyl}amino)pentanoic acid•TFA.

### Example 4

### Compound 28

### Synthesis of (2S)-5-carbamimidamido-2-[({1-[(2′-isopropylbiphenyl-2-yl)methyl]-2-oxo-1,2-dihydropyridin-3-yl}carbonyl)amino]pentanoic acid•TFA

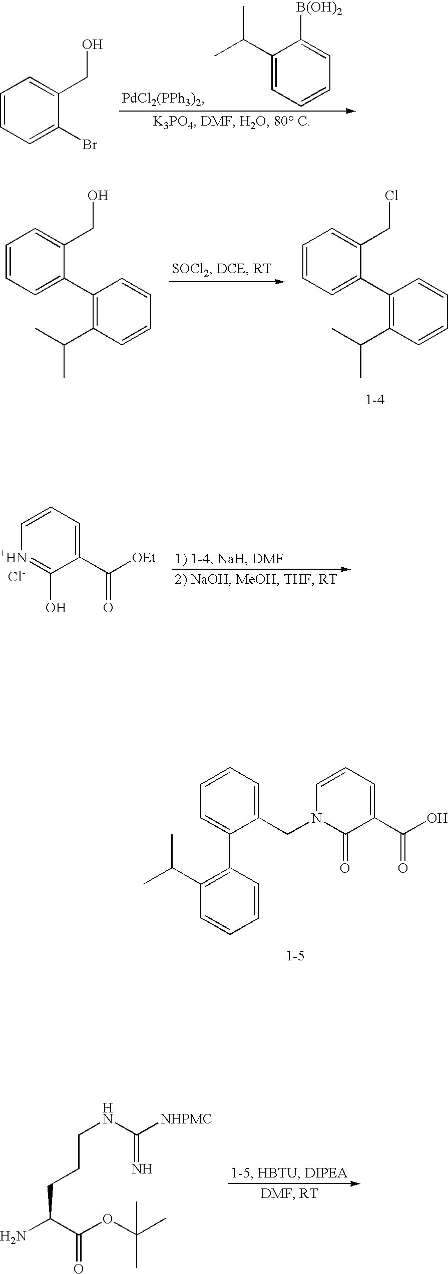

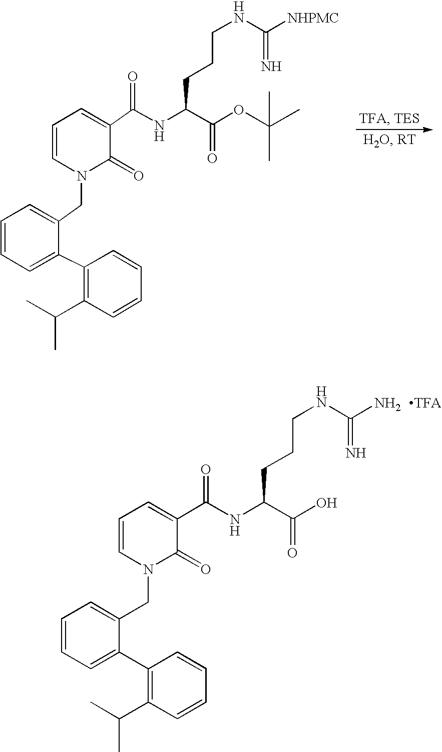

### I. (2′-Isopropylbiphenyl-2-yl)methanol

In a round bottom flask, 2-isopropylphenylboronic acid (0.58 g, 3.57 mmol), 2-bromobenzyl alcohol (0.66 g, 3.57 mmol), K₃PO₄ (1.9 g, 8.93 mmol), and PdCl₂(PPh₃)₂ (0.13 g, 0.18 mmol) were combined. To the mixture of reactants was added DMF (16 mL) and H₂O (4 mL). The reaction mixture was heated at 80° C. under N₂ overnight, then diluted with H₂O and extracted with EtOAc. The EtOAc extract was washed sequentially with H₂O and saturated NaCl, dried over anhydrous MgSO₄ and filtered. The filtrate was concentrated under reduced pressure and the residue was purified by column chromatography on silica gel, eluting with 20:1 EtOAc/hexanes followed by 10:1 EtOAc/hexanes to give an off-white solid (0.22 g, 27%).

### II. 2-(Chloromethyl)-2′-isopropylbiphenyl

To a solution of (2′-isopropylbiphenyl-2-yl)methanol (0.22 g, 0.97 mmol) in 1,2-dichloroethane (5 mL) was added thionyl chloride (0.08 mL, 1.07 mmol). The reaction was stirred overnight at room temperature and then evaporated to dryness under reduced pressure. Addition of hexanes to the residue followed by removal of the solvent under reduced pressure was repeated three times and then the product was dried under vacuum to give a yellow oil (0.25 g, 100%).

### III. Ethyl 1-[(2′-isopropylbiphenyl-2-yl)methyl]-2-oxo-1,2-dihydropyridine-3-carboxylate

Sodium hydride (60%, 80 mg, 2.0 mmol) was added to a solution of 3-(ethoxycarbonyl)-2-hydroxypyridinium chloride (200 mg, 0.94 mmol) in DMF (2.5 mL). The resulting suspension was stirred at room temperature for 30 min and then a solution of 2-(chloromethyl)-2′-isopropylbiphenyl (230 mg, 0.94 mmol) in anhydrous DMF (2.5 mL) was added via syringe. After stirring overnight at room temperature, the reaction was diluted with water and extracted into ethyl acetate. The organic layer was washed sequentially with water and saturated NaCl, dried over Na₂SO₄ and filtered. The filtrate was concentrated under reduced pressure to give the crude product which was used without further purification (0.40 mg, >100%).

### IV. 1-[(2′-Isopropylbiphenyl-2-yl)methyl]-2-oxo-1,2-dihydropyridine-3-carboxylic acid

To a solution of ethyl 1-[(2′-isopropylbiphenyl-2-yl)methyl]-2-oxo-1,2-dihydropyridine-3-carboxylate (0.40 mg, 1.07 mmol theoretical) in 6:1 THF/MeOH (7 mL) was added 2M NaOH (3 mL, 6 mmol) and the reaction stirred at room temperature for 2 hr. The reaction was diluted with water, and the resulting solution was washed twice with diethyl ether. Acidification of the aqueous phase produced a thick white precipitate which was extracted into EtOAc. The organic phase was washed sequentially with H₂O and with saturated NaCl, dried over Na₂SO₄ and filtered. The filtrate was concentrated under reduced pressure to give a yellow oil (0.15 g, 40%).

### V. tert-Butyl (2S)-2-[({1-[(2′-isopropylbiphenyl-2-yl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (160 mg, 0.33 mmol), 1-[2′-isopropylbiphenyl-2-yl)methyl]-2-oxo-1,2-dihydropyridine-3-carboxylic acid (150 mg, 0.43 mmol), and HBTU (170 mg, 0.45 mmol) in anhydrous DMF (2 mL) was added DIPEA (0.07 mL, 0.42 mmol). The reaction mixture was stirred at room temperature overnight, then diluted with water and extracted into EtOAc. The organic layer was washed sequentially with water and saturated NaCl, dried over Na₂SO₄ and filtered. The filtrate was concentrated under reduced pressure and the residue was purified by column chromatography on silica gel, eluting 1:2 EtOAc/hexanes followed by 1:1 EtOAc/hexanes to give a white foam (150 mg, 60%).

### VI. (2S)-5-Carbamimidamido-2-[({1-[(2′-isopropylbiphenyl-2-yl)methyl]-2-oxo-1,2-dihydropyridin-3-yl}carbonyl)amino]pentanoic acid•TFA

To a solution of tert-butyl (2S)-2-[({[1-(2′-isopropylbiphenyl-2-yl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate (150 mg, 0.18 mmol) in TFA (3 mL), triethylsilane (0.3 mL) and water (0.3 mL) were added. After stirring the reaction mixture at room temperature for 3 hr, MTBE (25 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give an off-white solid (60 mg, 54%).

The following compounds were synthesized by modifications of the general procedure described in Example 4.

Compound 20: Step I was carried out using 1.00 g 2-bromobenzyl alcohol, 0.72 g phenylboronic acid, 75 mg PdCl₂(PPh₃)₂ and 2.84 g K₃PO₄ in 20 mL DMF/4 mL H₂O to provide 0.87 g biphenyl-2-yl-methanol. Step II was carried out using 300 mg biphenyl-2-yl-methanol, 0.59 mL SOCl₂ and 10 mL CH₂Cl₂ to provide 292 mg 2-chloromethyl-biphenyl following elution with 1:9 EtOAc/hexanes through a short plug of silica gel. Step III was carried out using 292 mg 2-chloromethyl-biphenyl, 202 mg methyl 2-oxo-1,2-dihydropyridine-3-carboxylate hydrochloride and 55 mg 60% NaH in 15 mL DMF to provide 241 mg methyl 1-(biphenyl-2-ylmethyl)-2-oxo-1,2-dihydropyridine-3-carboxylate. Step IV was carried out with 241 mg methyl 1-(biphenyl-2-ylmethyl)-2-oxo-1,2-dihydropyridine-3-carboxylate and 2.3 mL 2 M NaOH in 10 mL 1:1 THF/MeOH to provide 211 mg 1-(biphenyl-2-ylmethyl)-2-oxo-1,2-dihydropyridine-3-carboxylic acid. Step V was carried out using 80 mg 1-(biphenyl-2-ylmethyl)-2-oxo-1,2-dihydropyridine-3-carboxylic acid, 130 mg tert-butyl (2S)-2-amino-5-{[(2,2,5,7,8-pentamethyl-3,4-dihydro-2H-chromen-6-yl)sulfonyl]carbamimidamido}pentanoate, 138 mg HBTU and 0.12 mL DIPEA in 2 mL DMF to provide 191 mg tert-butyl (2S)-2-({[1-(biphenyl-2-ylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step VI was carried out using 191 mg tert-butyl (2S)-2-({[1-(biphenyl-2-ylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 2 mL TFA, 0.2 mL triethylsilane and 0.2 mL H₂O to provide 51 mg (2S)-2-({[1-(biphenyl-2-ylmethyl)-2-oxo-1,2-dihydropyridin-3-yl]carbonyl}amino)-5-carbamimidamidopentanoic acid•TFA following reversed-phase HPLC purification.

Compound 21: Step I was carried out using 1.00 g 3-bromobenzyl alcohol, 0.72 g phenylboronic acid, 75 mg PdCl₂(PPh₃)₂ and 2.84 g K₃PO₄ in 20 mL DMF/4 mL H₂O to provide 0.84 g biphenyl-3-ylmethanol. Step II was carried out using 300 mg biphenyl-3-ylmethanol, 0.60 mL SOCl₂ and 10 mL CH₂Cl₂ to provide 196 mg 3-chloromethylbiphenyl following elution with 1:9 EtOAc/hexanes through a short plug of silica gel. Step III was carried out using 196 mg 3-chloromethylbiphenyl, 135 mg methyl 2-oxo-1,2-dihydropyridine-3-carboxylate hydrochloride and 37 mg 60% NaH in 10 mL DMF to provide 143 mg methyl 1-(biphenyl-3-ylmethyl)-2-oxo-1,2-dihydropyridine-3-carboxylate. Step IV was carried out with 143 mg methyl 1-(biphenyl-3-ylmethyl)-2-oxo-1,2-dihydropyridine-3-carboxylate and 1.3 mL 2 M NaOH in 8 mL 1:1 THF/MeOH to provide 127 mg 1-(biphenyl-3-ylmethyl)-2-oxo-1,2-dihydropyridine-3-carboxylic acid. Step V was carried out using 66 mg 1-(biphenyl-3-ylmethyl)-2-oxo-1,2-dihydropyridine-3-carboxylic acid, 107 mg tert-butyl (2S)-2-amino-5-{[(2,2,5,7,8-pentamethyl-3,4-dihydro-2H-chromen-6-yl)sulfonyl]carbamimidamido}pentanoate, 117 mg HBTU and 0.10 mL DIPEA in 2 mL DMF to provide 162 mg tert-butyl (2S)-2-({[1-(biphenyl-3-ylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step VI was carried out using 162 mg tert-butyl (2S)-2-({[1-(biphenyl-3-ylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 2 mL TFA, 0.2 mL triethylsilane and 0.2 mL H₂O to provide 61 mg (2S)-2-({[1-(biphenyl-3-ylmethyl)-2-oxo-1,2-dihydropyridin-3-yl]carbonyl}amino)-5-carbamimidamidopentanoic acid•TFA following reversed-phase HPLC purification.

Compound 22: Step I was carried out using 1.00 g 4-bromobenzyl alcohol, 0.72 g phenylboronic acid, 75 mg PdCl₂(PPh₃)₂ and 2.84 g K₃PO₄ in 20 mL DMF/4 mL H₂O to provide 0.79 g biphenyl-4-ylmethanol. Step II was carried out using 300 mg biphenyl-4-ylmethanol, 0.60 mL SOCl₂ and 10 mL CH₂Cl₂ to provide 196 mg 4-chloromethylbiphenyl following elution with 1:9 EtOAc/hexanes through a short plug of silica gel. Step III was carried out using 196 mg 4-chloromethylbiphenyl, 135 mg methyl 2-oxo-1,2-dihydropyridine-3-carboxylate hydrochloride and 37 mg 60% NaH in 15 mL DMF to provide 108 mg methyl 1-(biphenyl-4-ylmethyl)-2-oxo-1,2-dihydropyridine-3-carboxylate. Step IV was carried out with 108 mg methyl 1-(biphenyl-4-ylmethyl)-2-oxo-1,2-dihydropyridine-3-carboxylate and 1.01 mL 2 M NaOH in 6 mL 1:1 THF/MeOH to provide 98 mg 1-(biphenyl-4-ylmethyl)-2-oxo-1,2-dihydropyridine-3-carboxylic acid. Step V was carried out using 83 mg 1-(biphenyl-4-ylmethyl)-2-oxo-1,2-dihydropyridine-3-carboxylic acid, 135 mg tert-butyl (2S)-2-amino-5-{[(2,2,5,7,8-pentamethyl-3,4-dihydro-2H-chromen-6-yl)sulfonyl]carbamimidamido}pentanoate, 143 mg HBTU and 0.12 mL DIPEA in 3 mL DMF to provide 213 mg tert-butyl (2S)-2-({[1-(biphenyl-4-ylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step VI was carried out using 191 mg tert-butyl (2S)-2-({[1-(biphenyl-4-ylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 2 mL TFA, 0.2 mL triethylsilane and 0.2 mL H₂O to provide 35 mg (2S)-2-({[1-(biphenyl-4-ylmethyl)-2-oxo-1,2-dihydropylidin-3-yl]carbonyl}amino)-5-carbamimidamidopentanoic acid following reversed-phase HPLC purification.

Compound 23: Step I was carried out using 0.33 mL benzyl bromide, 0.34 g 2-hydroxymethylphenylboronic acid dihydrate, 89 mg PdCl₂(PPh₃)₂ and 1.35 g K₃PO₄ in 10 mL DMF/2.5 mL H₂O to provide 0.42 g 2-benzylbenzyl alcohol. Step II was carried out using 0.42 g 2-benzylbenzyl alcohol, 0.17 mL SOCl₂ and 6 mL CH₂Cl₂ to provide 0.38 g 2-benzylbenzyl chloride. Step III was carried out using 0.38 g 2-benzylbenzyl chloride, 0.27 g methyl 2-oxo-1,2-dihydropyridine-3-carboxylate hydrochloride and 78 mg 60% NaH in 9 mL DMF to provide 0.55 g methyl 1-(2-benzylbenzyl)-2-oxo-1,2-dihydropyridine-3-carboxylate. Step IV was carried out with 0.55 g methyl 1-(2-benzylbenzyl)-2-oxo-1,2-dihydropyridine-3-carboxylate and 1.5 mL 2 M NaOH in 3 mL THF/0.5 mL MeOH to provide 0.30 g 1-(2-benzylbenzyl)-2-oxo-1,2-dihydropyridine-3-carboxylic acid. Step V was carried out using 130 mg 1-(2-benzylbenzyl)-2-oxo-1,2-dihydropyridine-3-carboxylic acid, 150 mg tert-butyl (2S)-2-amino-5-{[(2,2,5,7,8-pentamethyl-3,4-dihydro-2H-chromen-6-yl)sulfonyl]carbamimidamido}pentanoate, 160 mg HBTU and 0.07 mL DIPEA in 2 mL DMF to provide 300 mg tert-butyl (2S)-2-({[1-(2-benzylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step VI was carried out using 300 mg tert-butyl (2S)-2-({[1-(2-benzylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 6 mL TFA, 0.6 mL triethylsilane and 0.6 mL H₂O to provide 28 mg (2S)-2-({[1-(2-benzylbenzyl)-2-oxo-1,2-dihydropyridin-3-yl]carbonyl}amino)-5-carbamimidamidopentanoic acid•TFA following reversed-phase HPLC purification.

Compound 24: Step I was carried out using 0.51 mL benzyl bromide, 0.60 g 3-hydroxymethylphenylboronic acid, 140 mg PdCl₂(PPh₃)₂ and 2.1 g K₃PO₄ in 16 mL DMF/4 mL H₂O to provide 0.33 g 3-benzylbenzyl alcohol. Step II was carried out using 0.33 g 3-benzylbenzyl alcohol, 0.13 mL SOCl₂ and 5 mL 1,2-dichloroethane to provide 0.30 g 3-benzylbenzyl chloride. Step III was carried out using 0.30 g 3-benzylbenzyl chloride, 0.21 g methyl 2-oxo-1,2-dihydropyridine-3-carboxylate hydrochloride and 60 mg 60% NaH in 7 mL DMF to provide 0.45 g methyl 1-(3-benzylbenzyl)-2-oxo-1,2-dihydropyridine-3-carboxylate. Step IV was carried out with 0.45 g methyl 1-(3-benzylbenzyl)-2-oxo-1,2-dihydropyridine-3-carboxylate and 1.5 mL 2 M NaOH in 3 mL THF/0.5 mL MeOH to provide 0.28 g 1-(3-benzylbenzyl)-2-oxo-1,2-dihydropyridine-3-carboxylic acid. Step V was carried out using 130 mg 1-(3-benzylbenzyl)-2-oxo-1,2-dihydropyridine-3-carboxylic acid, 150 mg tert-butyl (2S)-2-amino-5-{[(2,2,5,7,8-pentamethyl-3,4-dihydro-2H-chromen-6-yl)sulfonyl]carbamimidamido}pentanoate, 160 mg HBTU and 0.07 mL DIPEA in 2 mL DMF to provide 270 mg tert-butyl (2S)-2-({[1-(3-benzylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step VI was carried out using 270 mg tert-butyl (2S)-2-({[1-(3-benzylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 6 mL TFA, 0.6 mL triethylsilane and 0.6 mL H₂O to provide 107 mg (2S)-2-({[1-(3-benzylbenzyl)-2-oxo-1,2-dihydropyridin-3-yl]carbonyl}amino)-5-carbamimidamidopentanoic acid•TFA following reversed-phase HPLC purification.

Compound 25: Step I was carried out using 0.51 mL benzyl bromide, 0.60 g 4-hydroxymethylphenylboronic acid, 140 mg PdCl₂(PPh₃)₂ and 2.1 g K₃PO₄ in 16 mL DMF/4 mL H₂O to provide 0.60 g 4-benzylbenzyl alcohol. Step II was carried out using 0.60 g 4-benzylbenzyl alcohol, 0.24 mL SOCl₂ and 9 mL 1,2-dichloroethane to provide 0.48 g 4-benzylbenzyl chloride. Step III was carried out using 0.48 g 4-benzylbenzyl chloride, 0.34 g methyl 2-oxo-1,2-dihydropyridine-3-carboxylate hydrochloride and 98 mg 60% NaH in 11 mL DMF to provide 0.70 g methyl 1-(4-benzylbenzyl)-2-oxo-1,2-dihydropyridine-3-carboxylate. Step IV was carried out with 0.70 g methyl 1-(4-benzylbenzyl)-2-oxo-1,2-dihydropyridine-3-carboxylate and 1.5 mL 2 M NaOH in 3 mL THF/0.5 mL MeOH to provide 0.50 g 1-(4-benzylbenzyl)-2-oxo-1,2-dihydropyridine-3-carboxylic acid. Step V was carried out using 130 mg 1-(4-benzylbenzyl)-2-oxo-1,2-dihydropyridine-3-carboxylic acid, 150 mg tert-butyl (2S)-2-amino-5-{[(2,2,5,7,8-pentamethyl-3,4-dihydro-2H-chromen-6-yl)sulfonyl]carbamimidamido}pentanoate, 160 mg HBTU and 0.07 mL DIPEA in 2 mL DMF to provide 100 mg tert-butyl (2S)-2-({[1-(4-benzylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step VI was carried out using 100 mg tert-butyl (2S)-2-({[1-(4-benzylbenzyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 3 mL TFA, 0.3 mL triethylsilane and 0.3 mL H₂O to provide 28 mg (2S)-2-({[1-(4-benzylbenzyl)-2-oxo-1,2-dihydropyridin-3-yl]carbonyl}amino)-5-carbamimidamidopentanoic acid•TFA following reversed-phase HPLC purification.

Compound 26: Step I was carried out using 0.43 mL 2-bromobenzyl alcohol, 0.40 g 1-naphthaleneboronic acid, 82 mg PdCl₂(PPh₃)₂ and 1.2 g K₃PO₄ in 8 mL DMF/2 mL H₂O to provide 0.35 g 2-(1-naphthyl)benzyl alcohol. Step II was carried out using 0.35 g 2-(1-naphthyl)benzyl alcohol, 0.12 mL SOCl₂ and 5 mL 1,2-dichloroethane to provide 0.34 g 2-(1-naphthyl)benzyl chloride. Step III was carried out using 0.33 g 2-(1-naphthyl)benzyl chloride. 0.20 g methyl 2-oxo-1,2-dihydropyridine-3-carboxylate hydrochloride and 110 mg 60% NaH in 6.5 mL DMF to provide 0.50 g methyl 1-[2-(1-naphthyl)benzyl]-2-oxo-1,2-dihydropyridine-3-carboxylate. Step IV was carried out with 0.50 g methyl 1-[2-(1-naphthyl)benzyl]-2-oxo-1,2-dihydropyridine-3-carboxylate and 3 mL 2 M NaOH in 6 mL THF/1 mL MeOH to provide 90 mg 1-[2-(1-naphthyl)benzyl]-2-oxo-1,2-dihydropyridine-3-carboxylic acid. Step V was carried out using 80 mg 1-[2-(1-naphthyl)benzyl]-2-oxo-1,2-dihydropyridine-3-carboxylic acid, 110 mg tert-butyl (2S)-2-amino-5-{[(2,2,5,7,8-pentamethyl-3,4-dihydro-2H-chromen-6-yl)sulfonyl]carbamimidamido}pentanoate, 120 mg HBTU and 0.05 mL DIPEA in 2 mL DMF to provide 100 mg tert-butyl (2S)-2-[({1-[2-(1-naphthyl)benzyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step VI was carried out using 100 mg tert-butyl (2S)-2-[({1-[2-(1-naphthyl)benzyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 3 mL TFA, 0.3 mL triethylsilane and 0.3 mL H₂O to provide 20 mg (2S)-5-carbamimidamido-2-[({1-[2-(1-naphthyl)benzyl]-2-oxo-1,2-dihydropyridin-3-yl}carbonyl)amino]pentanoic acid•TFA following reversed-phase HPLC purification.

Compound 27: Step I was carried out using 0.43 mL 2-bromobenzyl alcohol, 0.40 g 2-naphthaleneboronic acid, 82 mg PdCl₂(PPh₃)₂ and 1.2 g K₃PO₄ in 4 mL DMF/1 mL H₂O to provide 0.21 g 2-(2-naphthyl)benzyl alcohol. Step II was carried out using 0.21 g 2-(2-naphthyl)benzyl alcohol, 0.07 mL SOCl₂ and 5 mL 1,2-dichloroethane to provide 0.20 g 2-(2-naphthyl)benzyl chloride. Step III was carried out using 0.20 g 2-(2-naphthyl)benzyl chloride, 0.12 g methyl 2-oxo-1,2-dihydropyridine-3-carboxylate hydrochloride and 66 mg 60% NaH in 4 mL DMF to provide 0.30 g methyl 1-[2-(2-naphthyl)benzyl]-2-oxo-1,2-dihydropyridine-3-carboxylate. Step IV was carried out with 0.30 g methyl 1-[2-(2-naphthyl)benzyl]-2-oxo-1,2-dihydropyridine-3-carboxylate and 1.5 mL 2 M NaOH in 3 mL THF/0.5 mL MeOH to provide 190 mg 1-[2-(2-naphthyl)benzyl]-2-oxo-1,2-dihydropyridine-3-carboxylic acid. Step V was carried out using 190 mg 1-[2-(2-naphthyl)benzyl]-2-oxo-1,2-dihydropyridine-3-carboxylic acid, 260 mg tert-butyl (2S)-2-amino-5-{[(2,2,5,7,8-pentamethyl-3,4-dihydro-2H-chromen-6-yl)sulfonyl]carbamimidamido}pentanoate, 280 mg HBTU and 0.12 mL DIPEA in 3 mL DMF to provide 310 mg tert-butyl (2S)-2-[({1-[2-(2-naphthyl)benzyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step VI was carried out using 300 mg tert-butyl (2S)-2-[({1-[2-(2-naphthyl)benzyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 6 mL TFA, 0.6 mL triethylsilane and 0.6 mL 1-120 to provide 120 mg (2S)-5-carbamimidamido-2-[({1-[2-(2-naphthyl)benzyl]-2-oxo-1,2-dihydropyridin-3-yl}carbonyl)amino]pentanoic acid•TFA following reversed-phase HPLC purification.

Compound 29: Step I was carried out using 0.66 g 2-bromobenzyl alcohol, 0.58 g 3-isopropylphenylboronic acid, 130 mg PdCl₂(PPh₃)₂ and 1.9 g K₃PO₄ in 16 mL DMF/4 mL H₂O to provide 0.44 g (3′-isopropylbiphenyl-2-yl)methanol. Step II was carried out using 0.44 g (3′-isopropylbiphenyl-2-yl)methanol, 0.16 mL SOCl₂ and 10 mL 1,2-dichloroethane to provide 0.42 g 2-(chloromethyl)-3′-isopropylbiphenyl. Step III was carried out using 0.40 g 2-(chloromethyl)-3′-isopropylbiphenyl, 0.33 g ethyl 2-oxo-1,2-dihydropyridine-3-carboxylate hydrochloride and 180 mg 60% NaH in 2.5 mL DMF to provide 0.48 g ethyl 1-[(3′-isopropylbiphenyl-2-yl)methyl]-2-oxo-1,2-dihydropyridine-3-carboxylate. Step IV was carried out with 0.48 g ethyl 1-[(3′-isopropylbiphenyl-2-yl)methyl]-2-oxo-1,2-dihydropyridine-3-carboxylateand 3 mL 2 M NaOH in 6 mL THF/1 mL MeOH to provide 0.18 g 1-[(3′-isopropylbiphenyl-2-yl)methyl]-2-oxo-1,2-dihydropyridine-3-carboxylic acid. Step V was carried out using 150 mg 1-[(3′-isopropylbiphenyl-2-yl)methyl]-2-oxo-1,2-dihydropyridine-3-carboxylic acid, 160 mg tert-butyl (2S)-2-amino-5-{[(2,2,5,7,8-pentamethyl-3,4-dihydro-2H-chromen-6-yl)sulfonyl]carbamimidamido}pentanoate, 170 mg HBTU and 0.07 mL DIPEA in 2 mL DMF to provide 150 mg tert-butyl (2S)-2-[({1-[(3′-isopropylbiphenyl-2-yl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step VI was carried out using 150 mg tert-butyl (2S)-2-[({1-[(3′-isopropylbiphenyl-2-yl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 3 mL TFA, 0.3 mL triethylsilane and 0.3 mL H₂O to provide 30 mg (2S)-5-carbamimidamido-2-[({1-[(3′-isopropylbiphenyl-2-yl)methyl]-2-oxo-1,2-dihydropyridin-3-yl}carbonyl)amino]pentanoic acid•TFA.

Compound 30: Step I was carried out using 0.66 g 2-bromobenzyl alcohol, 0.58 g 4-isopropylphenylboronic acid, 130 mg PdCl₂(PPh₃)₂ and 1.9 g K₃PO₄ in 16 mL DMF/4 mL H₂O to provide 0.60 g (4′-isopropylbiphenyl-2-yl)methanol. Step II was carried out using 0.60 g (4′-isopropylbiphenyl-2-yl)methanol, 0.21 mL SOCl₂ and 13 mL 1,2-dichloroethane to provide 0.54 g 2-(chloromethyl)-4′-isopropylbiphenyl. Step III was carried out using 0.52 g 2-(chloromethyl)-4′-isopropylbiphenyl, 0.43 g ethyl 2-oxo-1,2-dihydropyridine-3-carboxylate hydrochloride and 180 mg 60% NaH in 2.5 mL DMF to provide 0.55 g ethyl 1-[(4′-isopropylbiphenyl-2-yl)methyl]-2-oxo-1,2-dihydropyridine-3-carboxylate. Step IV was carried out with 0.55 g ethyl 1-[(4′-isopropylbiphenyl-2-yl)methyl]-2-oxo-1,2-dihydropyridine-3-carboxylate and 3 mL 2 M NaOH in 6 mL THF/1 mL MeOH to provide 0.17 g 1-[(4′-isopropylbiphenyl-2-yl)methyl]-2-oxo-1,2-dihydropyridine-3-carboxylic acid. Step V was carried out using 150 mg 1-[(4′-isopropylbiphenyl-2-yl)methyl]-2-oxo-1,2-dihydropyridine-3-carboxylic acid, 160 mg tert-butyl (2S)-2-amino-5-{[(2,2,5,7,8-pentamethyl-3,4-dihydro-2H-chromen-6-yl)sulfonyl]carbamimidamido pentanoate, 170 mg HBTU and 0.07 mL DIPEA in 2 mL DMF to provide 150 mg tert-butyl (2S)-2-[({1-[(4′-isopropylbiphenyl-2-yl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step VI was carried out using 150 mg tert-butyl (2S)-2-[({1-[(4′-isopropylbiphenyl-2-yl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 3 mL TFA, 0.3 mL triethylsilane and 0.3 mL H₂O to provide 30 mg (2S)-5-carbamimidamido-2-[({1-[(4′-isopropylbiphenyl-2-yl)methyl]-2-oxo-1,2-dihydropyridin-3-yl}carbonyl)amino]pentanoic acid•TFA following reversed phase HPLC purification.

Compound 31: Step I was carried out using 1.3 g 2-bromobenzyl alcohol, 1.0 g 4-fluorophenylboronic acid, 250 mg PdCl₂(PPh₃)₂ and 3.8 g K₃PO₄ in 32 mL DMF/8 mL H₂O to provide 1.2 g (4′-fluorobiphenyl-2-yl)methanol. Step TI was carried out using 1.2 g (4′-fluorobiphenyl-2-yl)methanol, 0.48 mL SOCl₂ and 30 mL CH₂Cl₂ to provide 1.1 g 2-(chloromethyl)-4′-fluorobiphenyl. Step III was carried out using 1.0 g 2-(chloromethyl)-4′-fluorobiphenyl, 0.70 g methyl 2-oxo-1,2-dihydropyridine-3-carboxylate hydrochloride and 0.38 g 60% NaH in 8 mL DMF to provide 0.95 g methyl 1-[(4′-fluorobiphenyl-2-yl)methyl]-2-oxo-1,2-dihydropyridine-3-carboxylate. Step IV was carried out with 0.95 g methyl 1-[(4′-fluorobiphenyl-2-yl)methyl]-2-oxo-1,2-dihydropyridine-3-carboxylate and 5 mL 2 M NaOH in 10 mL THF/1.5 mL MeOH to provide 0.82 g 1-[(4′-fluorobiphenyl-2-yl)methyl]-2-oxo-1,2-dihydropyridine-3-carboxylic acid. Step V was carried out using 400 mg 1-[(4′-fluorobiphenyl-2-yl)methyl]-2-oxo-1,2-dihydropyridine-3-carboxylic acid, 620 mg tert-butyl (2S)-2-amino-5-{[(2,2,5,7,8-pentamethyl-3,4-dihydro-2H-chromen-6-yl)sulfonyl]carbamimidamido}pentanoate, 660 mg HBTU and 0.28 mL DIPEA in 6 mL DMF to provide 380 mg tert-butyl (2S)-2-[({1-[(4′-fluorobiphenyl-2-yl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step VI was carried out using 380 mg tert-butyl (2S)-2-[({1-[(4′-fluorobiphenyl-2-yl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 6 mL TFA, 0.6 mL triethylsilane and 0.6 mL H₂O to provide 180 mg (2S)-5-carbamimidamido-2-[({1-[(4′-fluorobiphenyl-2-yl)methyl]-2-oxo-1,2-dihydropyridin-3-yl}carbonyl)amino]pentanoic acid•TFA.

### Example 5

### Compound 32

### Synthesis of (2S)-5-carbamimidamido-2-({[1-(2-naphthyl)-2-oxo-1,2-dihydropyridin-3-yl]carbonyl}amino)pentanoic acid•TFA

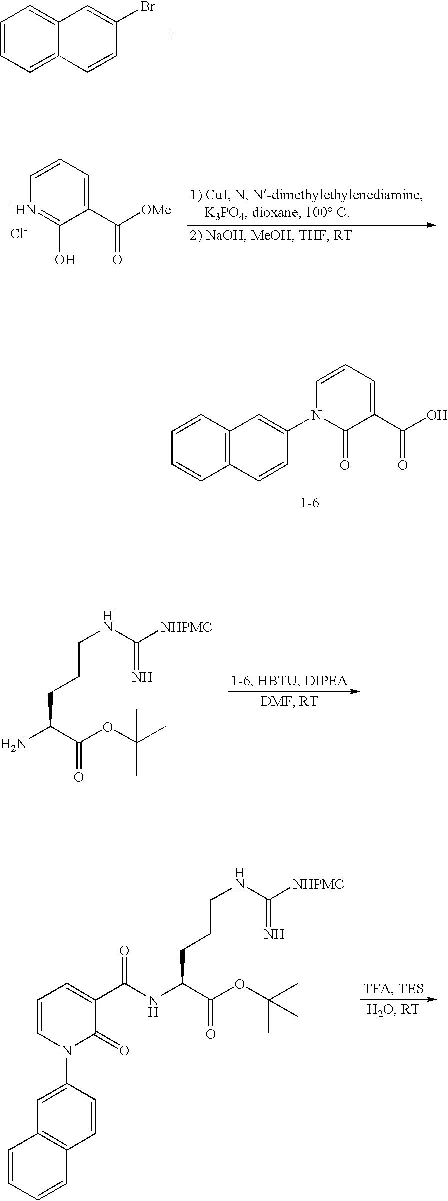

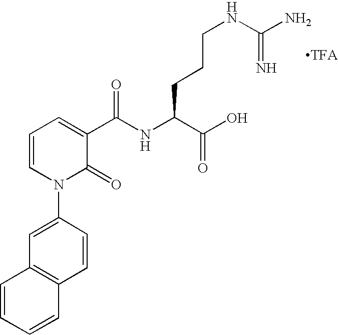

### I. Methyl 1-(2-naphthyl)-2-oxo-1,2-dihydropyridine-3-carboxylate

In a roundbottom flask were combined methyl 2-oxo-1,2-dihydropyridine-3-carboxylate hydrochloride (500 mg, 2.65 mmol), 2-bromonaphthalene (800 mg, 3.86 mmol), CuI (131 mg, 0.69 mmol), and finely powdered K₃PO₄ (1.76 g, 8.28 mmol). The flask was purged with a stream of N₂ for 5 min. To the solid reactants was then added anhydrous 1,4-dioxane (25 mL), followed by N,N′-dimethylethylendiamine (0.15 mL, 1.38 mmol). The suspension was purged with a stream of N₂ for a further 5 min and then heated to 100° C. overnight. The reaction was quenched with 2M HCl, and the product was extracted into EtOAc. The organic phase was washed sequentially with H₂O and with saturated NaCl, dried over Na₂SO₄ and filtered. The filtrate was concentrated under reduced pressure and the residue was purified by column chromatography on silica gel, eluting with 60:40% EtOAc/hexanes to 100% EtOAc gradient to give a white solid (243 mg, 33%).

### II. 1-(2-naphthyl)-2-oxo-1,2-dihydropyridine-3-carboxylic acid

To a solution methyl 1-(2-naphthyl)-2-oxo-1,2-dihydropyridine-3-carboxylate (243 mg, 0.87 mmol) in 1:1 THF/MeOH (12 mL) was added 2M NaOH (2.6 mL, 5.2 mmol) and the reaction stirred at room temperature for 2 hr. The reaction was diluted with water, and the resulting solution was washed twice with diethyl ether. Acidification of the aqueous phase produced a yellow precipitate which was extracted into EtOAc. The organic phase was washed sequentially with H₂O and with saturated NaCl, dried over Na₂SO₄ and filtered. The filtrate was concentrated under reduced pressure to give a yellow solid (237 mg, 100%).

### III. tert-Butyl (2S)-2-({[1-(2-naphthyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (141 mg, 0.28 mmol), 1-(2-naphthyl)-2-oxo-1,2-dihydropyridine-3-carboxylic acid (75 mg, 0.28 mmol), and HBTU (149 mg, 0.39 mmol) in anhydrous DMF (2 mL) was added DIPEA (0.13 mL, 0.70 mmol). The reaction mixture was stirred at room temperature overnight, then diluted with water and extracted into EtOAc. The organic layer was washed with water (3 times) and saturated NaCl, dried over Na₂SO₄ and filtered. The filtrate was concentrated under reduced pressure and the residue was purified by column chromatography on silica gel, eluting with 3:1 EtOAc/hexanes followed by 100% EtOAc to give a yellow solid (194 mg, 93%).

### IV. (2S)-5-Carbamimidamido-2-({[1-(2-naphthyl)-2-oxo-1,2-dihydropyridin-3-yl]carbonyl}amino)pentanoic acid•TFA

To a solution of tert-butyl (2S)-2-({[1-(2-naphthyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate (194 mg, 0.26 mmol) in TFA (2 mL), triethylsilane (0.2 mL) and water (0.2 mL) were added. After stirring the reaction mixture at room temperature for 4.5 hr, MTBE (20 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give a pale yellow solid (88 mg, 63%).

The following compounds were synthesized by modifications of the general procedure described in Example 5.

Compound 33: Step I was conducted with 500 mg methyl 2-oxo-1,2-dihydropyridine-3-carboxylate hydrochloride, 0.41 mL bromobenzene, 131 mg CuI, 0.15 N,N′-dimethylethylenediamine, and 1.76 g K₃PO₄ in 25 mL dioxane at 100° C. in a sealed tube to provide 62 mg methyl 2-oxo-1-phenyl-1,2-dihydropyridine-3-carboxylate. Step II was conducted with 62 mg 2-oxo-1-phenyl-1,2-dihydropyridine-3-carboxylate and 0.84 mL 2 M NaOH in 6 mL 1:1 THF/MeOH to provide 52 mg 2-oxo-1-phenyl-1,2-dihydropyridine-3-carboxylic acid. Step III was conducted using 52 mg 2-oxo-1-phenyl-1,2-dihydropyridine-3-carboxylic acid, 120 mg tert-butyl (2S)-2-amino-5-{[(2,2,5,7,8-pentamethyl-3,4-dihydro-2H-chromen-6-yl)sulfonyl]carbamimidamido}pentanoate, 127 mg HBTU, and 0.11 DIPEA to provide 151 mg tert-butyl (2S)-2-{[(2-oxo-1-phenyl-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate. Step IV was conducted using 151 mg tert-butyl (2S)-2-{[(2-oxo-1-phenyl-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate, 2 mL TFA, 0.2 mL triethylsilane, and 0.2 mL deionized H₂O to provide 11 mg (2S)-5-carbamimidamido-2-{[(2-oxo-1-phenyl-1,2-dihydropyridin-3-yl)carbonyl]amino}pentanoic acid•TFA following reversed phase HPLC purification.

Compound 34: Step I was conducted with 500 mg ethyl 2-oxo-1,2-dihydropyridine-3-carboxylate hydrochloride, 804 mg 4-bromobiphenyl, 190 mg CuI, 0.13 N,N′-dimethylethylenediamine, and 1.57 g K₃PO₄ in 25 mL dioxane heated at 100° C. to provide 425 mg ethyl 1-(biphenyl-4-yl)-2-oxo-1,2-dihydropyridine-3-carboxylate. Step II was conducted with 383 mg ethyl 1-(biphenyl-4-yl)-2-oxo-1,2-dihydropyridine-3-carboxylate and 3.6 mL 2 M NaOH in 16 mL 1:1 THF/MeOH to provide 312 mg 1-(biphenyl-4-yl)-2-oxo-1,2-dihydropyridine-3-carboxylic acid. Step III was conducted using 70 mg 1-(biphenyl-4-yl)-2-oxo-1,2-dihydropyridine-3-carboxylic acid, 120 mg tert-butyl (2S)-2-amino-5-{[(2,2,5,7,8-pentamethyl-3,4-dihydro-2H-chromen-6-yl)sulfonyl]carbamimidamido}pentanoate, 127 mg HBTU, and 0.11 DIPEA to provide 191 mg tert-butyl (2S)-2-{[(1-biphenyl-4-yl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate. Step IV was conducted using 191 mg tert-butyl (2S)-2-{[(1-biphenyl-4-yl-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate, 2 mL TFA, 0.2 mL triethylsilane, and 0.2 mL deionized H₂O to provide 86 mg (2S)-2-{[(1-biphenyl-4-yl-2-oxo-1,2-dihydropyridin-3-yl)carbonyl]amino}-5-carbamimidamidopentanoic acid•TFA following reversed phase HPLC purification.

### Example 6

### Compound 36

### Synthesis of (2S)-2-{[(1-benzyl-2-oxo-6-phenyl-1,2-dihydropyridin-3-yl)carbonyl]amino}-5-carbamimidamidopentanoic acid•TFA

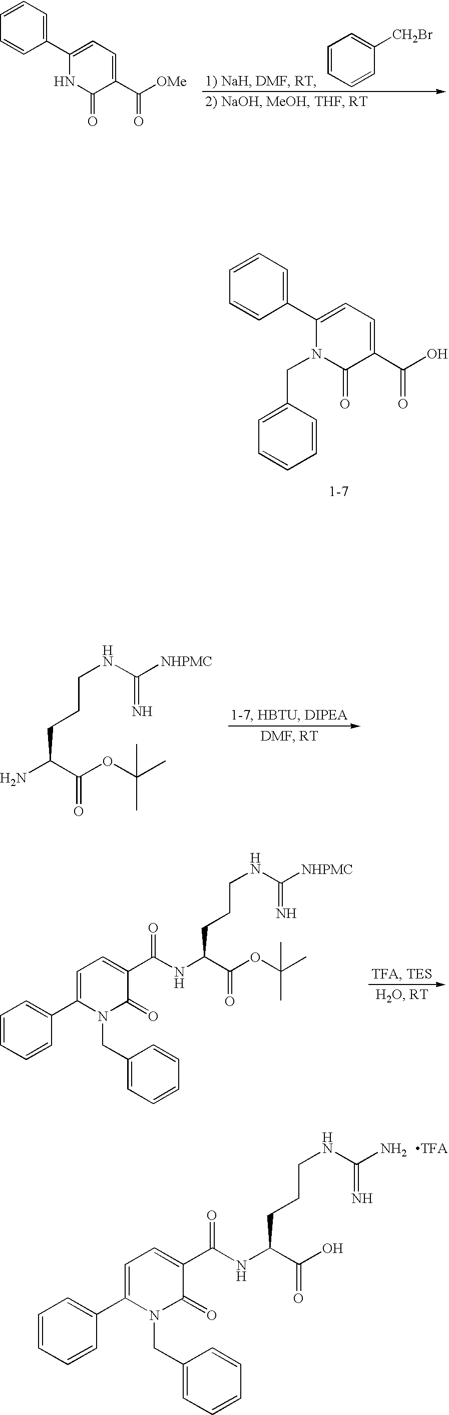

### I. Methyl 1-benzyl-2-oxo-6-phenyl-1,2-dihydropyridine-3-carboxylate

Sodium hydride (60%, 63 mg, 1.57 mmol) was added to a solution of methyl 2-oxo-6-phenyl-1,2-dihydropyridine-3-carboxylate (300 mg, 1.31 mmol) in DMF (8 mL). The resulting suspension was stirred at room temperature for 30 min and then benzyl bromide (0.23 mL, 1.97 mmol) was added. After stirring overnight at room temperature, the reaction was quenched with 2M HCl and diluted with ethyl acetate. The organic layer was washed with water (3 times) and saturated NaCl, dried over Na₂SO₄ and filtered. The filtrate was concentrated under reduced pressure to give a mixture of O- and N-alkylated products. The desired N-alkylation product was obtained by column chromatography purification on silica gel, eluting with a 1:4 to 3:2 EtOAc/hexanes gradient to give a white solid (75 mg, 18%).

### II. 1-Benzyl-2-oxo-6-phenyl-1,2-dihydropyridine-3-carboxylic acid

To a solution of methyl 1-benzyl-2-oxo-6-phenyl-1,2-dihydropyridine-3-carboxylate (75 mg, 0.24 mmol) in 1:1 THF/MeOH (4 mL) was added 2M NaOH (0.7 mL, 1.4 mmol) and the reaction stirred at room temperature for 2 hr. The reaction was diluted with water, and the resulting solution was washed twice with diethyl ether. Acidification of the aqueous phase produced a white precipitate which was extracted into EtOAc. The organic phase was washed sequentially with H₂O and with saturated NaCl, dried over Na₂SO₄ and filtered. The filtrate was concentrated under reduced pressure to give a white solid (67 mg, 93%).

### III. tert-Butyl (2S)-2-{[(1-benzyl-2-oxo-6-phenyl-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (93 mg, 0.19 mmol), 1-benzyl-2-oxo-6-phenyl-1,2-dihydropyridine-3-carboxylic acid (57 mg, 0.19 mmol), and HBTU (101 mg, 0.27 mmol) in anhydrous DMF (2 mL) was added DIPEA (0.12 mL, 0.67 mmol). The reaction mixture was stirred 2 days at room temperature, then diluted with water and extracted into EtOAc. The organic layer was washed with water (3 times) and saturated NaCl, dried over Na₂SO₄ and filtered. The filtrate was concentrated under reduced pressure and the residue was purified by column chromatography on silica gel, eluting with a 70:30% EtOAc/hexanes to 100% EtOAc gradient to give a white foam (140 mg, 94%).

### IV. (2S)-2-{[(1-Benzyl-2-oxo-6-phenyl-1,2-dihydropyridin-3-yl)carbonyl]amino}-5-carbamimidamidopentanoic acid•TFA

To a solution of tert-butyl (2S)-2-{[(1-benzyl-2-oxo-6-phenyl-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate (140 mg, 0.18 mmol) in TFA (2 mL), triethylsilane (0.2 mL) and water (0.2 mL) were added. After stirring the reaction mixture at room temperature for 4.5 hr, MTBE (20 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give a white solid (61 mg, 59%).

### Example 7

### Compound 11

### Synthesis of (2S)-5-carbamimidamido-2-({[1-(2,2-diphenylethyl)-2-oxo-1,2-dihydropyridin-3-yl]carbonyl}amino)pentanoic acid•TFA

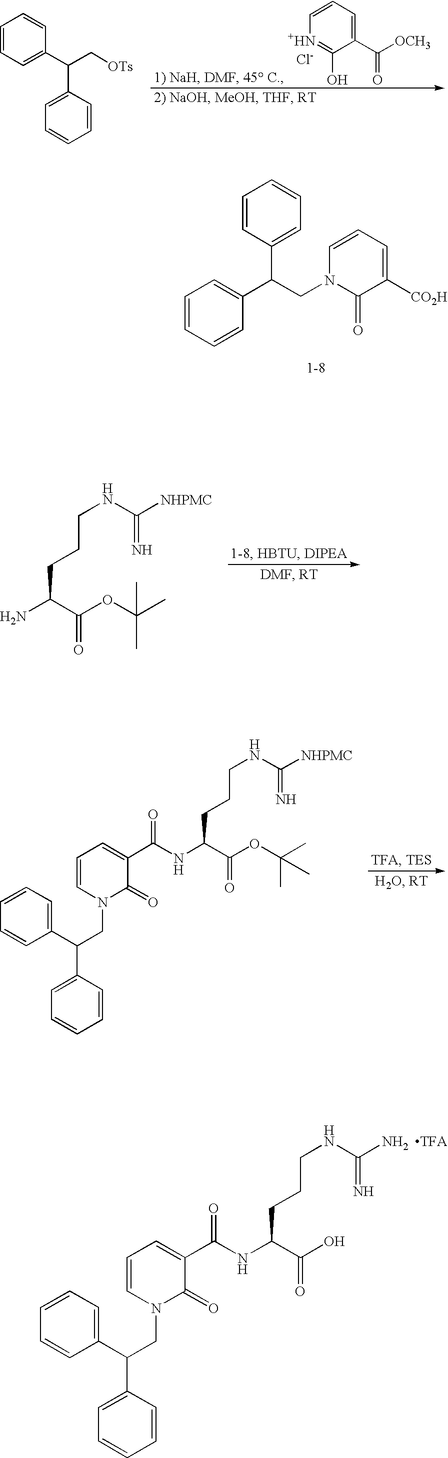

### I. Methyl 1-(2,2-diphenylethyl)-2-oxo-1,2-dihydropyridine-3-carboxylate

Sodium hydride (60%, 142 mg, 3.56 mmol) was added to a solution of methyl 2-oxo-1,2-dihydropyridine-3-carboxylate hydrochloride (519 mg, 2.76 mmol) in DMF (20 mL). The resulting suspension was stirred at room temperature for 30 min and then a solution of 1,1-diphenyl-2-(toluenesulfonyloxy)ethane (1.55 g, 4.40 mmol) in DMF (10 mL) was added via syringe. The reaction was heated at 40° C. for 2.5 hr and then overnight at 45° C. The reaction was quenched with 2M HCl and diluted with ethyl acetate. The organic layer was washed with water (3 times) and saturated NaCl, dried over Na₂SO₄ and filtered. The filtrate was concentrated under reduced pressure and the residue was purified by column chromatography on silica gel, eluting with 1:4 EtOAc/hexanes followed by 100% EtOAc to give the product (43 mg, 5%).

### II. 1-(2,2-Diphenylethyl)-2-oxo-1,2-dihydropyridine-3-carboxylic acid

To a solution of methyl 1-(2,2-diphenylethyl)-2-oxo-1,2-dihydropyridine-3-carboxylate (117 mg, 0.35 mmol) in 1:1 THF/MeOH (6 mL) was added 2M NaOH (1.1 mL, 2.2 mmol) and the reaction stirred at room temperature for 6 hr. The reaction was diluted with water, and the resulting solution was washed twice with diethyl ether. Acidification of the aqueous phase produced a thick white precipitate which was extracted into EtOAc. The organic layer was separated, washed successively with water and saturated NaCl, dried over Na₂SO₄ and filtered. The filtrate was concentrated under reduced pressure to give a white solid (81 mg, 72%).

### III. tert-Butyl (2S)-2-({[1-(2,2-diphenylethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (126 mg, 0.25 mmol), 1-(2,2-diphenylethyl)-2-oxo-1,2-dihydropyridine-3-carboxylic acid (81 mg, 0.25 mmol), and HBTU (133 mg, 0.31 mmol) in anhydrous DMF (2 mL) was added DIPEA (0.11 mL, 0.63 mmol). The reaction mixture was stirred at room temperature overnight and then diluted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 4:1 EtOAc/hexanes to give the product (224 mg).

### IV. (2S)-5-Carbamimidamido-2-({[1-(2,2-diphenylethyl)-2-oxo-1,2-dihydropyridin-3-yl]carbonyl}amino)pentanoic acid•TFA

To a solution of tert-butyl (2S)-2-({[1-(2,2-diphenylethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate (224 mg, 0.25 mmol theoretical) in TFA (2 mL), triethylsilane (0.2 mL) and water (0.2 mL) were added. After stirring the reaction mixture at room temperature for 4.5 hr, MTBE (25 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give a white solid (98 mg, 67%).

The following compound was synthesized by modifications of the general procedure described in Example 7

Compound 79, (2S)-2-({[1-(2,2-diphenylethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoic acid•TFA, was obtained as a byproduct from Step IV from Example 7.

### Example 8

### Compound 41

### Synthesis of (2R)-5-carbamimidamido-2-({[1-(diphenylmethyl)-2-oxo-1,2-dihydropyridin-3-yl]carbonyl}amino)pentanoic acid•TFA

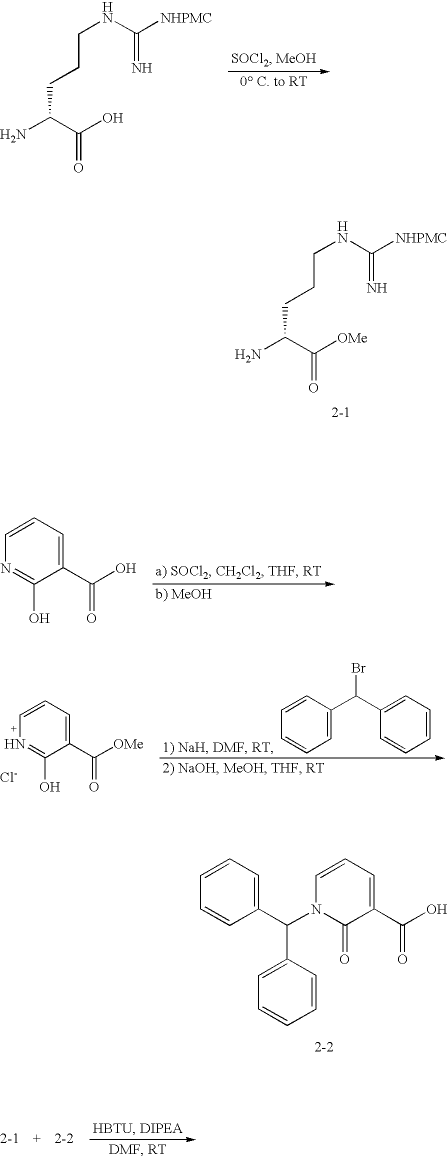

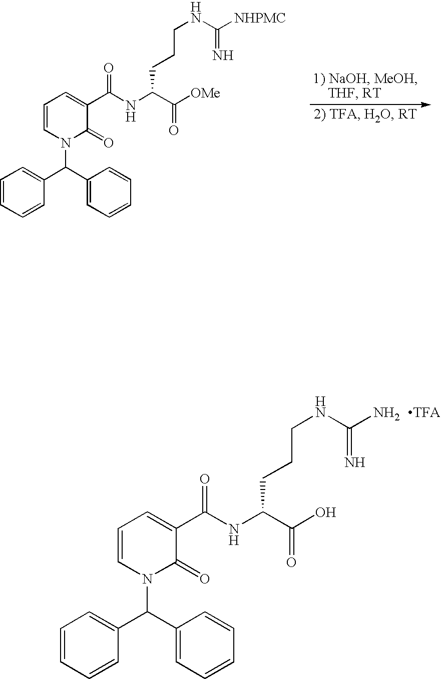

### I. Methyl (2R)-2-amino-5-{[(2,2,5,7,8-pentamethyl-3,4-dihydro-2H-chromen-6-yl)sulfonyl]carbamimidamido}pentanoate

Thionyl chloride (0.66 mL, 9.1 mmol) was added dropwise to a suspension of (2R)-2-amino-5-{[(2,2,5,7,8-pentamethyl-3,4-dihydro-2H-chromen-6-yl)sulfonyl]carbarnimidamido}pentanoic acid (400 mg, 0.91 mmol) in 10 mL anhydrous methanol at 0° C. The reaction was stirred at 0° C. for 90 min, then at room temperature for 3 hr. The solvent was removed under reduced pressure and the crude product was purified by column chromatography on silica gel, eluting with 9:1 dichloromethane:methanol to give a pale yellow oil that solidified to a white solid under vacuum (239 mg, 58%).

### II. Methyl 2-oxo-1,2-dihydropyridine-3-carboxylate hydrochloride

To a suspension of 2-hydroxynicotinic acid (10.00 g, 71.9 mmol) in anhydrous dichloromethane (150 mL) was added thionyl chloride (25.9 mL, 215.8 mmol) followed by anhydrous THF (150 mL). The reaction mixture was stirred at room temperature for 1 hr, and then excess methanol was added to the suspension until a homogenous solution was obtained. The reaction mixture was filtered and the filtrate concentrated under reduced pressure to give a pale yellow oil which solidified to an off-white solid under vacuum (13.96 g, 100%).

### III. Methyl 1-(diphenylmethyl)-2-oxo-1,2-dihydropyridine-3-carboxylate

Sodium hydride (60%, 275 mg, 6.87 mmol) was added to a solution of methyl 2-oxo-1,2-dihydropyridine-3-carboxylate hydrochloride (500 mg, 2.65 mmol) in DMF (15 mL). The resulting thick white suspension was vigorously stirred at room temperature for 30 min and then a solution of benzhydryl bromide (1.13 g, 4.58 mmol) in anhydrous DMF (15 mL) was added via syringe. After stirring 2 days at room temperature, the reaction was quenched with 2M HCl and diluted with ethyl acetate. The organic layer was washed with water (3 times) and saturated NaCl, dried over Na₂SO₄ and filtered. The filtrate was concentrated under reduced pressure and the residue was purified by column chromatography on silica gel, eluting with 40-60% EtOAc:hexanes gradient to give a yellow foam (503 mg, 60%).

### IV. 1-(Diphenylmethyl)-2-oxo-1,2-dihydropyridine-3-carboxylic acid

To a solution of methyl 1-(diphenylmethyl)-2-oxo-1,2-dihydropyridine-3-carboxylate (776 mg, 2.43 mmol) in 1:1 THF:MeOH (24 mL) was added 2M NaOH (7.3 mL, 14.6 mmol) and then stirred at room temperature for 5 hr. The reaction was diluted with water, and the resulting solution was washed twice with diethyl ether. The aqueous phase was acidified and extracted with EtOAc. The organic layer was washed with water and saturated NaCl, dried over Na₂SO₄ and filtered. The solvent was removed under reduced pressure to give a pale yellow solid (713 mg, 96%).

### V. Methyl (2R)-2-({[1-(diphenylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate

To a solution of methyl (2R)-2-amino-5-{[(2,2,5,7,8-pentamethyl-3,4-dihydro-2H-chromen-6-yl)sulfonyl]carbamimidamido}pentanoate (141 mg, 0.31 mmol), 1-(diphenylmethyl)-2-oxo-1,2-dihydropyridine-3-carboxylic acid (95 mg, 0.31 mmol), and HBTU (165 mg, 0.43 mmol) in anhydrous DMF (3 mL) was added DIPEA (0.19 mL, 1.1 mmol). The reaction mixture was stirred at room temperature for 2 days and then diluted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 9:1 EtOAc:hexanes to give a brown foam (211 mg, 92%).

### VI. (2R)-2-({[1-(Diphenylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoic acid

To a solution of methyl (2R)-2-({[1-(diphenylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido pentanoate (211 mg, 0.28 mmol) in 1:1 THF/MeOH (8 mL) was added 2M NaOH (0.85 mL, 1.71 mmol) and stirred at room temperature for 2.5 hr. The reaction was then diluted with water, washed with diethyl ether (2 times) and the layers separated. The aqueous phase was acidified with 2M HCl and extracted with EtOAc. The organic layer was washed with water and saturated NaCl, dried over Na₂SO₄ and filtered. The solvent was removed under reduced pressure to give a white semi-solid (180 mg, 87%).

### VII. (2R)-5-Carbamimidamido-2-({[1-(diphenylmethyl)-2-oxo-1,2-dihydropyridin-3-yl]carbonyl}amino)pentanoic acid•TFA

To a solution of (2R)-2-({[1-(diphenylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoic acid (180 mg, 0.25 mmol) in TFA (3 mL) was added H₂O (0.4 mL). After stirring the reaction mixture at room temperature for 4 hr. MTBE (20 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized, purified by reversed-phase preparative HPLC using a 10 to 60% CH₃CN:0.1% TFA in H₂O gradient, and then lyophilized again to give a white solid (40 mg, 28%).

### Example 9

### Compound 44

### Synthesis of (2S)-2-[({1-[bis(4-fluorophenyl)methyl]-2-oxo-1,2-dihydropyridin-3-yl}carbonyl)amino]-5-carbamimidamidopentanoic acid•TFA

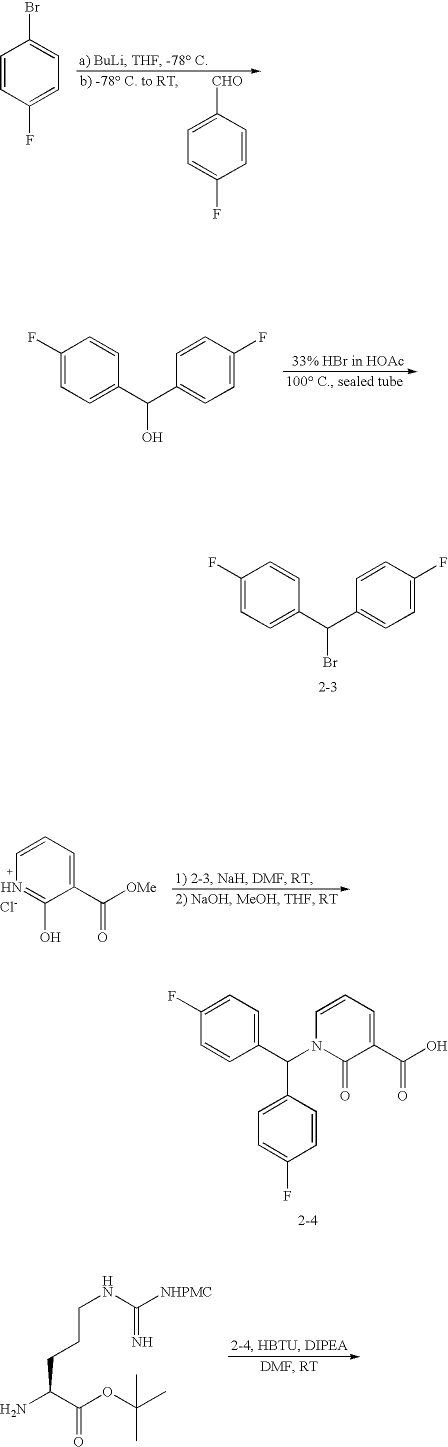

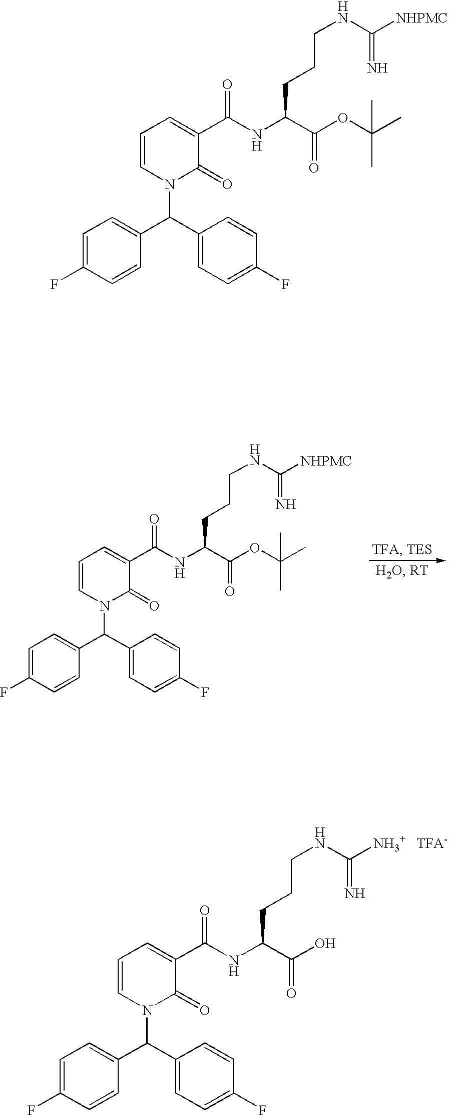

### I. Bis(4-fluorophenyl)methanol

To a solution of 1-bromo-4-fluorobenzene (1.00 g, 5.71 mmol) in anhydrous THF (7 mL) at −78° C. was added n-BuLi (2.5M in hexanes, 2.5 mL, 6.25 mmol) dropwise over 1 min. After 20 minutes, a solution of 4-fluorobenzaldehyde (709 mg, 5.71 mmol) in anhydrous THF (3 mL) was added via syringe. The reaction was stirred at −78° C. for 75 minutes, quenched with glacial acetic acid (1 mL) and then allowed to warm to room temperature. The reaction mixture was poured into H₂O, and the product was extracted into diethyl ether. The organic layer was washed with saturated NaCl, dried over Na₂SO₄ and filtered. The filtrate was concentrated under reduced pressure and the resulting residue was purified by column chromatography on silica gel eluting with 1:9 EtOAc/hexanes (1.19 g, 94%).

### II. 4,4′-(bromomethylene)bis(fluorobenzene)

In a sealed tube, a solution of bis(4-fluorophenyl)methanol (700 mg) in HBr (33% in acetic acid, 6 mL) was stirred at room temperature for 1.75 hr and then heated to 100° C. for 3 hours. After cooling to room temperature, the reaction mixture was quenched with saturated aqueous NaHCO₃. The product was extracted into diethyl ether, the layers were separated and the organic phase was dried over Na₂SO₄ and filtered. Following solvent removal under reduced pressure, the residue was purified by rapid elution through a short column of silica gel using 1:3 EtOAc/hexanes as the eluent to give a pale yellow oil (690 mg, 76%).

### III. Methyl 1-[bis(4-fluorophenyl)methyl]-2-oxo-1,2-dihydropyridine-3-carboxylate

A solution of methyl 2-oxo-1,2-dihydropyridine-3-carboxylate hydrochloride (389 mg, 2.06 mmol), 4,4′-(bromomethylene)bis(fluorobenzene) (690 mg, 2.4 mmol), and sodium hydride (60%, 120 mg, 3 mmol) in anhydrous DMF (8 mL) was stirred at room temperature overnight, and the reaction then diluted with saturated aqueous NaHCO₃ and extracted with diethyl ether. The organic layer separated, dried over Na₂SO₄ and filtered. The solvent was removed under reduced pressure and the resulting residue was purified by column chromatography on silica gel eluting with 1:99 MeOH/EtOAc to give a colorless oil (205 mg, 28%).

### IV. 1-[Bis(4-fluorophenyl)methyl]-2-oxo-1,2-dihydropyridine-3-carboxylic acid

To a solution of methyl 1-[bis(4-fluorophenyl)methyl]-2-oxo-1,2-dihydropyridine-3-carboxylate (200 mg, 0.56 mmol) in MeOH (8 mL) was added 6 M NaOH (0.5 mL, 3 mmol). The reaction mixture was stirred at room temperature overnight, acidified with 1 M HCl, and extracted with diethyl ether. The organic phase was dried over Na₂SO₄ and filtered. The solvent was removed under reduced pressure and the resulting residue was purified by column chromatography on silica gel eluting with 1:1 EtOAc/hexanes to give a white solid (180 mg, 94%).

### V. tert-Butyl (2S)-2-[({1-[bis(4-fluorophenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (313 mg, 0.63 mmol), 1-[bis(4-fluorophenyl)methyl]-2-oxo-1,2-dihydropyridine-3-carboxylic acid (180 mg, 0.53 mmol), and HBTU (240 mg, 0.63 mmol) in anhydrous DMF (5 mL) was added DIPEA (0.11 mL, 0.63 mmol). The reaction mixture was stirred at room temperature overnight, diluted with water and then extracted with diethyl ether. The organic layer was washed with saturated NaCl, dried over Na₂SO₄ and filtered. The filtrate was concentrated under reduced pressure and the residue was purified by column chromatography on silica gel, eluting with 1:99 MeOH/EtOAc to give a colorless oil (260 mg, 60%).

### VI. (2S)-2-[({1-[Bis(4-fluorophenyl)methyl]-2-oxo-1,2-dihydropyridin-3-yl}carbonyl)amino]-5-carbamimidamidopentanoic acid•TFA

To a solution of tert-butyl (2S)-2-[({1-[bis(4-fluorophenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate (137 mg, 0.17 mmol) in TFA (2 mL), triethylsilane (0.1 mL) and water (0.1 mL) were added. After stirring the reaction mixture at room temperature for 2 hr, MTBE (45 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give a white solid (21 mg, 21%).

The following compounds were synthesized by modifications of the general procedure described in Example 9.

Compound 40: Step V was conducted conducted using 0.55 g compound 2-2 from Example 8, 0.90 g tert-butyl (2S)-2-amino-5-{[(2,2,5,7,8-pentamethyl-3,4-dihydro-2H-chromen-6-yl)sulfonyl]carbamimidamido}pentanoate, 0.96 g HBTU, and 0.80 mL DIIPEA in 10 mL DMF to provide 1.40 g tert-butyl (2S)-2-({[1-(diphenylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step VI was conducted using 1.40 g tert-butyl (2S)-2-({[1-(diphenylmethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 14 mL TFA, 1.4 mL triethylsilane, and 1.4 mL deionized H₂O to provide 0.76 g (2S)-5-carbamimidamido-2-({[1-(diphenylmethyl)-2-oxo-1,2-dihydropyridin-3-yl]carbonyl}amino)pentanoic acid•TFA.

Compound 42: Step I was conducted with 2.0 g 2-bromotoluene, 1.41 g o-tolualdehyde, and 5.15 mL 2.5 M BuLi in 25 mL THF to provide 1.72 g bis(2-methylphenyl)methanol. Step II was conducted with 400 mg bis(2-methylphenyl)methanol and 4 mL 33% HBr in acetic acid to provide 350 mg 1,1′-(bromomethylene)bis(2-methylbenzene). Step 111 was conducted using 350 mg 2,2′-(bromomethylene)bis(methylbenzene), 174 mg methyl 2-oxo-1,2-dihydropyridine-3-carboxylate hydrochloride and 61 mg 60% NaH in 5 mL DMF to provide 80 mg methyl 1-[bis(2-methylphenyl)methyl]-2-oxo-1,2-dihydropyridine-3-carboxylate. Step IV was conducted using 80 mg methyl 1-[bis(2-methylphenyl)methyl]-2-oxo-1,2-dihydropyridine-3-carboxylate and 10 drops 6 M NaOH in 3 mL MeOH/0.5 mL THF to provide 88 mg 1-[bis(2-methylphenyl)methyl]-2-oxo-1,2-dihydropyridine-3-carboxylic acid. Step V was conducted with 88 mg 1-[bis(2-methylphenyl)methyl]-2-oxo-1,2-dihydropyridine-3-carboxylic acid, 158 mg tert-butyl (2S)-2-amino-5-{[(2,2,5,7,8-pentamethyl-3,4-dihydro-2H-chromen-6-yl)sulfonyl]carbamimidamido}pentanoate, 120 mg HBTU, and 0.06 mL DIPEA in 3 mL DMF to provide 180 mg tert-butyl (2S)-2-[({1-[bis(2-methylphenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step VI was conducted using 180 mg tert-butyl (2S)-2-[({1-[bis(2-methylphenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 2 mL TFA, 0.1 mL triethylsilane and 0.1 mL deionized H₂O to provide 35 mg (2S)-2-[({1-[bis(2-methylphenyl)methyl]-2-oxo-1,2-dihydropyridin-3-yl}carbonyl)amino]-5-carbamimidamidopentanoic acid•TFA following reversed-phase HPLC purification.

Compound 43: Step I was conducted with 2.0 g 3-bromotoluene, 1.41 g m-tolualdehyde, and 5.15 mL 2.5 M BuLi in 25 mL THF to provide 2.2 g bis(3-methylphenyl)methanol. Step II was conducted with 1.13 g bis(3-methylphenyl)methanol and 2 mL 33% HBr in acetic acid to provide 350 mg 3,3′-(bromomethylene)bis(methylbenzene). Step III was conducted using 300 mg 3,3′-(bromomethylene)bis(methylbenzene), 157 mg methyl 2-oxo-1,2-dihydropyridine-3-carboxylate hydrochloride and 60 mg 60% NaH in 10 mL DMF to provide 60 mg methyl 1-[bis(3-methylphenyl)methyl]-2-oxo-1,2-dihydropyridine-3-carboxylate. Step IV was conducted using 60 mg methyl 1-[bis(3-methylphenyl)methyl]-2-oxo-1,2-dihydropyridine-3-carboxylate and 10 drops 6 M NaOH in 3 mL MeOH/0.5 mL THF to provide 1-[bis(3-methylphenyl)methyl]-2-oxo-1,2-dihydropyridine-3-carboxylic acid. Step V was conducted with 1-[bis(3-methylphenyl)methyl]-2-oxo-1,2-dihydropyridine-3-carboxylic acid (crude material from previous step), 167 mg tert-butyl (2S)-2-amino-5-{[(2,2,5,7,8-pentamethyl-3,4-dihydro-2H-chromen-6-yl)sulfonyl]carbamimidamido}pentanoate, 127 mg HBTU, and 0.06 mL DIPEA in 3 mL DMF to provide 205 mg tert-butyl (2S)-2-[({1-[bis(3-methylphenyl)methyl]-2-oxo-1,2-dihydropyridin-3-yl}carbonyl)amino]-5-{[(2,2,5,7,8-pentamethyl-3,4-dihydro-2,3-chromen-6-yl)sulfonyl]carbamimidamido}pentanoate. Step VI was conducted using 200 mg tert-butyl (2S)-2-[({1-[bis(3-methylphenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 2 mL TFA, 0.1 mL triethylsilane and 0.1 mL deionized H₂O to provide 16 mg (2S)-2-[({1-[bis(3-methylphenyl)methyl]-2-oxo-1,2-dihydropyridin-3-yl}carbonyl)amino]-5-carbamimidamidopentanoic acid following reversed-phase HPLC purification.

Compound 45: Step I was conducted with 1.5 g 1-bromo-3-fluorobenzene, 1.41 g 3-fluorobenzaldehyde, and 3.75 mL 2.5 M BuLi in 10 mL THF to provide 1.7 g bis(3-fluorophenyl)methanol. Step II was conducted with 1.7 g bis(3-fluorophenyl)methanol and ˜4 mL 33% HBr in acetic acid to provide 3,3′-(bromomethylene)bis(fluorobenzene) (yield not determined). Step III was conducted using 3,3′-(bromomethylene)bis(fluorobenzene) from Step 11,490 mg methyl 2-oxo-1,2-dihydropyridine-3-carboxylate hydrochloride and 250 mg 60% NaH in 10 mL DMF to provide 160 mg methyl 1-[bis(3-fluorophenyl)methyl]-2-oxo-1,2-dihydropyridine-3-carboxylate. Step IV was conducted using 160 mg methyl 1-[bis(3-fluorophenyl)methyl]-2-oxo-1,2-dihydropyridine-3-carboxylate and 10 drops 6 M NaOH in 3 mL MeOH/0.5 mL THF to provide 110 mg 1-[bis(3-fluorophenyl)methyl]-2-oxo-1,2-dihydropyridine-3-carboxylic acid. Step V was conducted with 110 mg 1-[bis(3-fluorophenyl)methyl]-2-oxo-1,2-dihydropyridine-3-carboxylic acid, 192 mg tert-butyl (2S)-2-amino-5-{[(2,2,5,7,8-pentamethyl-3,4-dihydro-2H-chromen-6-yl)sulfonyl]carbamimidamido}pentanoate, 148 mg HBTU, and 0.15 mL DIPEA in 5 mL DMF to provide 288 mg tert-butyl (2S)-2-[({1-[bis(3-fluorophenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step VI was conducted using 288 mg tert-butyl (2S)-2-[({1-[bis(3-fluorophenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 2 mL TFA, 0.1 mL triethylsilane and 0.1 mL deionized H₂O to provide 98 mg (2S)-2-[({1-[bis(3-fluorophenyl)methyl]-2-oxo-1,2-dihydropyridin-3-yl}carbonyl)amino]-5-carbamimidamidopentanoic acid•TFA following reversed-phase HPLC purification.

Compound 46: Step I was conducted with 1.0 g 1-bromo-2-fluorobenzene, 0.71 g 2-fluorobenzaldehyde, and 2.5 mL 2.5 M BuLi in 10 mL THF to provide 1.32 g bis(2-fluorophenyl)methanol. Step II was conducted with 0.70 g bis(2-fluorophenyl)methanol and ˜4 mL 33% HBr in acetic acid to provide 760 mg 2,2′-(bromomethylene)bis(fluorobenzene). Step III was conducted using 760 mg 2,2′-(bromomethylene)bis(fluorobenzene), 370 mg methyl 2-oxo-1,2-dihydropyridine-3-carboxylate hydrochloride and 150 mg 60% NaH in 10 mL DMF to provide 470 mg methyl 1-[bis(2-fluorophenyl)methyl]-2-oxo-1,2-dihydropyridine-3-carboxylate. Step IV was conducted using 470 mg methyl 1-[bis(2-fluorophenyl)methyl]-2-oxo-1,2-dihydropyridine-3-carboxylate and 10 drops 6 M NaOH in 5 mL MeOH to provide 430 mg 1-[bis(2-fluorophenyl)methyl]-2-oxo-1,2-dihydropyridine-3-carboxylic acid. Step V was conducted with 180 mg 1-[bis(2-fluorophenyl)methyl]-2-oxo-1,2-dihydropyridine-3-carboxylic acid, 300 mg tert-butyl (2S)-2-amino-5-{[(2,2,5,7,8-pentamethyl-3,4-dihydro-2H-chromen-6-yl)sulfonyl]carbamimidamido}pentanoate, 228 mg HBTU, and 0.17 mL DIPEA in 10 mL DMF to provide 420 mg tert-butyl (2S)-2-[({1-[bis(2-fluorophenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step VI was conducted using 420 mg tert-butyl (2S)-2-[({1-[bis(2-fluorophenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 2 mL TFA, 0.1 mL triethylsilane and 0.1 mL deionized H₂O to provide 63 mg (2S)-2-[({1-[bis(2-fluorophenyl)methyl]-2-oxo-1,2-dihydropyridin-3-yl}carbonyl)amino]-5-carbamimidamidopentanoic acid•TFA following reversed-phase HPLC purification.

Compound 47: Step I was conducted with 1.0 g 5-bromo-m-xylene, 0.73 g 3,5-dimethylbenzaldehyde, and 2.4 mL 2.5 M BuLi in 10 mL THF to provide 0.88 g bis(3,5-dimethylphenyl)methanol. Step II was conducted with 370 mg bis(3,5-dimethylphenyl)methanol and 4 mL 33% HBr in acetic acid to provide 460 mg bis(3,5-dimethylphenyl)methyl bromide. Step III was conducted using 460 mg bis(3,5-dimethylphenyl)methyl bromide, 208 mg methyl 2-oxo-1,2-dihydropyridine-3-carboxylate hydrochloride and 100 mg 60% NaH in 10 mL DMF to provide 64 mg methyl 1-[bis(3,5-dimethylphenyl)methyl]-2-oxo-1,2-dihydropyridine-3-carboxylate. Step IV was conducted using 64 mg methyl 1-[bis(3,5-dimethylphenyl)methyl]-2-oxo-1,2-dihydropyridine-3-carboxylate and 5 drops 6 M NaOH in 2 mL MeOH to provide 60 mg 1-[bis(3,5-dimethylphenyl)methyl]-2-oxo-1,2-dihydropyridine-3-carboxylic acid. Step V was conducted with 60 mg 1-[bis(3,5-dimethylphenyl)methyl]-2-oxo-1,2-dihydropyridine-3-carboxylic acid, 100 mg tert-butyl (2S)-2-amino-5-{[(2,2,5,7,8-pentamethyl-3,4-dihydro-2H-chromen-6-yl)sulfonyl]carbamimidamido}pentanoate, 78 mg HBTU, and 0.07 mL DIPEA in 3 mL DMF to provide 84 mg tert-butyl (2S)-2-[({1-[bis(3,5-dimethylphenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate. Step VI was conducted using 84 mg tert-butyl (2S)-2-[({1-[bis(3,5-dimethylphenyl)methyl]-2-oxo-1,2-dihydropyridin-3-yl}carbonyl)amino]-5-{[(2,2,5,7,8-pentamethyl-3,4-dihydro-2H-chromen-6-yl)sulfonyl]carbamimidamido}pentanoate, 1.5 mL TFA, 0.1 mL triethylsilane and 0.1 mL deionized H₂O to provide 10 mg (2S)-2-[({1-[bis(3,5-dimethylphenyl)methyl]-2-oxo-1,2-dihydropyridin-3-yl}carbonyl)amino]-5-carbamimidamidopentanoic acid•TFA following reversed-phase HPLC purification.

Compound 48: Step I was conducted with 1.0 g 1-bromo-3-trifluoromethylbenzene, 0.77 g 3-trifluoromethylbenzaldehyde, and 1.8 mL 2.5 M BuLi in 15 mL THF to provide 0.77 g bis[3-(trifluoromethyl)phenyl]methanol. Step II was conducted with 760 mg bis[3-(trifluoromethyl)phenyl]methanol and 4 mL 33% HBr in acetic acid to provide 505 mg 1,1′-(bromomethylene)bis[3-(trifluoromethyl)benzene]. Step III was conducted using 505 mg 1,1′-(bromomethylene)bis[3-(trifluoromethyl)benzene], 217 mg methyl 2-oxo-1,2-dihydropyridine-3-carboxylate hydrochloride and 64 mg 60% NaH in 10 mL DMF to provide 310 mg methyl 1-{bis[3-(trifluoromethyl)phenyl]methyl}-2-oxo-1,2-dihydropyridine-3-carboxylate. Step IV was conducted using 310 mg methyl 1-{bis[3-(trifluoromethyl)phenyl]methyl}-2-oxo-1,2-dihydropyridine-3-carboxylate and 10 drops 6 M NaOH in 5 mL MeOH to provide 300 mg 1-{bis[3-(trifluoromethyl)phenyl]methyl}-2-oxo-1,2-dihydropyridine-3-carboxylic acid. Step V was conducted with 300 mg 1-{bis[3-(trifluoromethyl)phenyl]methyl}-2-oxo-1,2-dihydropyridine-3-carboxylic acid, 338 mg tert-butyl (2S)-2-amino-5-{[(2,2,5,7,8-pentamethyl-3,4-dihydro-2H-chromen-6-yl)sulfonyl]carbamimidamido}pentanoate, 258 mg HBTU, and 0.37 mL DIPEA in 10 mL DMF to provide 440 mg tert-butyl (2S)-2-{[(1-{bis[3-(trifluoromethyl)phenyl]methyl}-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate. Step VI was conducted using 440 mg tert-butyl (2S)-2-{[(1-{bis[3-(trifluoromethyl)phenyl]methyl}-2-oxo-1,2-dihydropyridin-3-yl)carbonyl]amino}-5-{[(2,2,5,7,8-pentamethyl-3,4-dihydro-2H-chromen-6-yl)sulfonyl]carbamimidamido}pentanoate, 3 mL TFA, 0.1 mL triethylsilane and 0.1 mL deionized H₂O to provide 128 mg (2S)-2-{[(1-{bis[3-(trifluoromethyl)phenyl]methyl}-2-oxo-1,2-dihydropyridin-3-yl)carbonyl]amino}-5-carbamimidamidopentanoic acid•TFA following reversed-phase HPLC purification.

Compound 49: Step I was conducted with 1.0 g 1-bromo-4-trifluoromethylbenzene, 0.77 g 4-trifluoromethylbenzaldehyde, and 1.8 mL 2.5 M BuLi in 15 mL THF to provide 1.06 g bis[4-(trifluoromethyl)phenyl]methanol. Step II was conducted with 1.0 g bis[4-(trifluoromethyl)phenyl]methanol and ˜4 mL 33% HBr in acetic acid to provide 543 mg 1,1′-(bromomethylene)bis[4-(trifluoromethyl)benzene]. Step III was conducted using 543 mg 1,1′-(bromomethylene)bis[4-(trifluoromethyl)benzene], 233 mg methyl 2-oxo-1,2-dihydropyridine-3-carboxylate hydrochloride and 70 mg 60% NaH in 10 mL DMF to provide 240 mg methyl 1-{bis[4-(trifluoromethyl)phenyl]methyl}-2-oxo-1,2-dihydropyridine-3-carboxylate. Step IV was conducted using 240 mg methyl 1-{bis[4-(trifluoromethyl)phenyl]methyl}-2-oxo-1,2-dihydropyridine-3-carboxylate and 10 drops 6 M NaOH in 5 mL MeOH to provide 220 mg 1-{bis[4-(trifluoromethyl)phenyl]methyl}-2-oxo-1,2-dihydropyridine-3-carboxylic acid. Step V was conducted with 220 mg 1-{bis[4-(trifluoromethyl)phenyl]methyl}-2-oxo-1,2-dihydropyridine-3-carboxylic acid, 263 mg tert-butyl (2S)-2-amino-5-{[(2,2,5,7,8-pentamethyl-3,4-dihydro-2H-chromen-6-yl)sulfonyl]carbamimidamido}pentanoate, 201 mg HBTU, and 0.27 mL DIPEA in 10 mL DMF to provide 320 mg tert-butyl (2S)-2-{[(1-{bis[4-(trifluoromethyl)phenyl]methyl}-2-oxo-1,2-dihydropyridin-3-yl)carbonyl]amino 1-5-{[(2,2,5,7,8-pentamethyl-3,4-dihydro-2H-chromen-6-yl)sulfonyl]carbamimidamido}pentanoate. Step VI was conducted using 320 mg tert-butyl (2S)-2-{[(1-{bis[4-(trifluoromethyl)phenyl]methyl}-2-oxo-1,2-dihydropyridin-3-yl)carbonyl]amino 3-5-{[(2,2,5,7,8-pentamethyl-3,4-dihydro-2H-chromen-6-yl)sulfonyl]carbamimidamido}pentanoate, 3 mL TFA, 0.1 mL triethylsilane and 0.1 mL deionized H₂O to provide 178 mg (2S)-2-{[(1-{bis[4-(trifluoromethyl)phenyl]methyl}-2-oxo-1,2-dihydropyridin-3-yl)carbonyl]amino}-5-carbamimidamidopentanoic acid•TFA following reversed-phase HPLC purification.

Compound 50: Step III was conducted using 250 mg methyl 2-oxo-1,2-dihydropyridine-3-carboxylate hydrochloride, 0.45 mL α-methylbenzyl bromide, and 68 mg 60% NaH in 16 mL DMF to provide 69 mg methyl 2-oxo-1-(1-phenylethyl)-1,2-dihydropyridine-3-carboxylate. Step IV was conducted using 69 mg methyl 2-oxo-1-(1-phenylethyl)-1,2-dihydropyridine-3-carboxylate and 0.8 mL 2 M NaOH in 6 mL 1:1 THF/MeOH to provide 58 mg 2-oxo-1-(1-phenylethyl)-1,2-dihydropyridine-3-carboxylic acid. Step V was conducted using 58 mg 2-oxo-1-(1-phenylethyl)-1,2-dihydropyridine-3-carboxylic acid, 119 mg mg tert-butyl (2S)-2-amino-5-{[(2,2,5,7,8-pentamethyl-3,4-dihydro-2H-chromen-6-yl)sulfonyl]carbamimidamido}pentanoate, 127 mg HBTU and 0.11 mL DIPEA in 2 mL DMF to provide 174 mg tert-butyl (2S)-2-({[2-oxo-1-(1-phenylethyl)-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate. Step VI was conducted using 174 mg tert-butyl (2S)-2-({[2-oxo-1-(1-phenylethyl)-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate, 2 mL TFA, 0.2 mL triethylsilane, and 0.2 mL deionized H₂O to provide 60 mg (2S)-5-carbamimidamido-2-({[2-oxo-1-(1-phenylethyl)-1,2-dihydropyridin-3-yl]carbonyl}amino)pentanoic acid•TFA following reversed-phase HPLC purification.

Compound 90: Step III was conducted using sodium hydride (60%, 325 mg), methyl 2-oxo-1,2-dihydropyridine-3-carboxylate hydrochloride (500 mg) in DMF (16 mL) and 1-iodo-2,2-dimethylpropane (0.7 mL), the reaction mixture was heated to 160° C. in a microwave reactor for 10 minutes and the crude material was not purified. This reaction gave methyl 1-(2,2-dimethylpropyl)-2-oxo-1,2-dihydropyridine-3-carboxylate (0.85 g) as a light yellow oil. Step IV was conducted using methyl methyl 1-(2,2-dimethylpropyl)-2-oxo-1,2-dihydropyridine-3-carboxylate (0.80 g, 2.43), THF (12 mL) MeOH (2 mL) and aqueous NaOH (2M, 6 mL) to give 1-(2,2-dimethylpropyl)-2-oxo-1,2-dihydropyridine-3-carboxylic acid (160 mg) as a white solid. Step V was conducted using tert-butyl (2S)-2-amino-5-{[(2,2,5,7,8-pentamethyl-3,4-dihydro-2H-chromen-6-yl)sulfonyl]carbamimidamido}pentanoate (380 mg), 1-(2,2-dimethylpropyl)-2-oxo-1,2-dihydropyridine-3-carboxylic acid (160 mg), and HBTU (410 mg), DMF (4 mL) and DIPEA (0.2 mL) to give tert-butyl (2S)-2-({[1-(2,2-dimethylpropyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate (360 mg) as a light yellow oil. Step VI was conducted using tert-butyl (2S)-2-({[1-(2,2-dimethylpropyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoate (360 mg), TFA (6 mL), H₂O (0.6 mL), trietylsilane (0.6 mL) to give (2S)-5-carbamimidamido-2-({[1-(2,2-dimethylpropyl)-2-oxo-1,2-dihydropyridin-3-yl]carbonyl}amino)pentanoic acid•TFA (90, 150 mg) as an off-white solid.

### Example 10

### Compound 52

### Synthesis of (2S)-2-[(3-benzylbenzoyl)amino]-5-carbamimidamidopentanoic acid•TFA

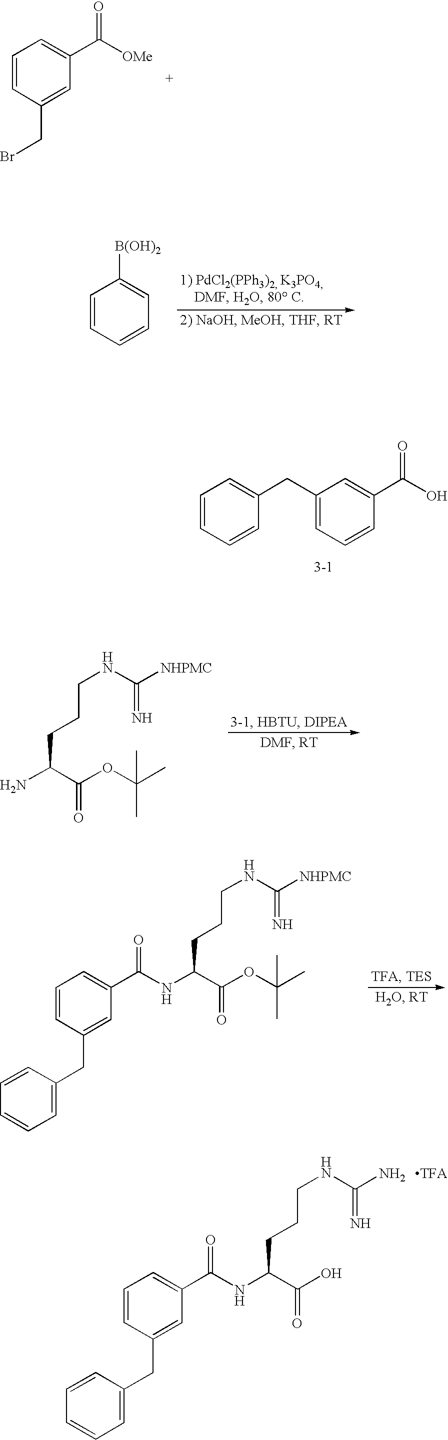

### I. Methyl 3-benzylbenzoate

A solution of 3-bromomethylbenzoate (500 mg, 2.18 mmol), phenylboronic acid (290 mg, 2.40 mmol), PdCl₂(PPh₃)₂ (46 mg, 0.07 mmol) and K₃PO₄ (1.16 g, 5.45 mmol) in DMF (10 mL) and water (2 mL) was heated at 80° C. for 1 hr and then cooled to room temperature and diluted with EtOAc. The reaction mixture was washed with water (3 times) and saturated NaCl, dried over Na₂SO₄ and filtered. The filtrate was concentrated under reduced pressure and the residue was purified by column chromatography on silica gel, eluting with 1:19 EtOAc:hexanes to give a colorless oil (353 mg, 72%).

### II. 3-Benzylbenzoic acid

To a solution methyl 3-benzylbenzoate (353 mg, 1.56 mmol) in MeOH (20 mL) was added 2M NaOH (3.1 mL, 6.2 mmol) and the reaction stirred at room temperature for 24 hr. The reaction was diluted with water, and the resulting solution was washed twice with diethyl ether. Acidification of the aqueous phase produced a white suspension which was extracted into EtOAc. The organic phase was washed sequentially with H₂O and saturated NaCl, dried over Na₂SO₄ and filtered. The filtrate was concentrated under reduced pressure to give a solid (313 mg, 95%).

### III. tert-Butyl (2S)-2-[(3-benzylbenzoyl)amino]-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (98 mg, 0.20 mmol), 3-benzylbenzoic acid (40 mg, 0.19 mmol) and HBTU (101 mg, 0.27 mmol) in anhydrous DMF (1 mL) was added DIPEA (0.07 mL, 0.42 mmol). The reaction mixture was stirred at room temperature overnight, then diluted with 2M HCl and extracted into EtOAc. The organic layer was washed with water (2 times) and saturated NaCl, dried over Na₂SO₄ and filtered. The filtrate was concentrated under reduced pressure and the residue was purified by column chromatography on silica gel, eluting with 3:2 EtOAc:hexanes to give a colorless oil (103 mg, 79%).

### IV. (2S)-2-[(3-Benzylbenzoyl)amino]-5-carbamimidamidopentanoic acid•TFA

To a solution of tert-butyl (2S)-2-[(3-benzylbenzoyl)amino]-5-{[(2,2,5,7,8-pentamethyl-3,4-dihydro-2H-chromen-6-yl)sulfonyl]carbamimidamido}pentanoate (103 mg, 0.15 mmol) in TFA (2 mL), triethylsilane (0.1 mL) and water (0.1 mL) were added. After stirring the reaction mixture at room temperature for 3 hr, MTBE (25 mL) was added resulting in formation of a white precipitate. The product was extracted into water and the resulting solution was lyophilized to give a brown solid (11 mg, 15%).

The following compound was synthesized by modifications of the general procedure described in Example 10.

Compound 51: Step III was carried out using 27 mg 4-benzylbenzoic acid, 70 mg tert-butyl (2S)-2-amino-5-{[(2,2,5,7,8-pentamethyl-3,4-dihydro-2H-chromen-6-yl)sulfonyl]carbamimidamido}pentanoate, 69 mg HBTU, and 50 μL DIPEA in 1 mL DMF to provide 85 mg tert-butyl (2S)-2-[(4-benzylbenzoyl)amino]-5-{[(2,2,5,7,8-pentamethyl-3,4-dihydro-2H-chromen-6-yl)sulfonyl]carbamimidamido}pentanoate. Step IV was conducted using 85 mg tert-butyl (2S)-2-[(4-benzylbenzoyl)amino]-5-{[(2,2,5,7,8-pentamethyl-3,4-dihydro-2H-chromen-6-yl)sulfonyl]carbamimidamido}pentanoate, 2 mL TFA, 0.1 mL triethylsilane and 0.1 mL deionized H₂O to provide 17 mg (2S)-2-[(4-benzylbenzoyl)amino]-5-carbamimidamidopentanoic acid•TFA.

### Example 11

### Compound 54

### Synthesis of (2S)-2-{[(1-benzyl-6-oxo-1,6-dihydropyridin-3-yl)carbonyl]amino}-5-carbamimidamidopentanoic acid•TFA

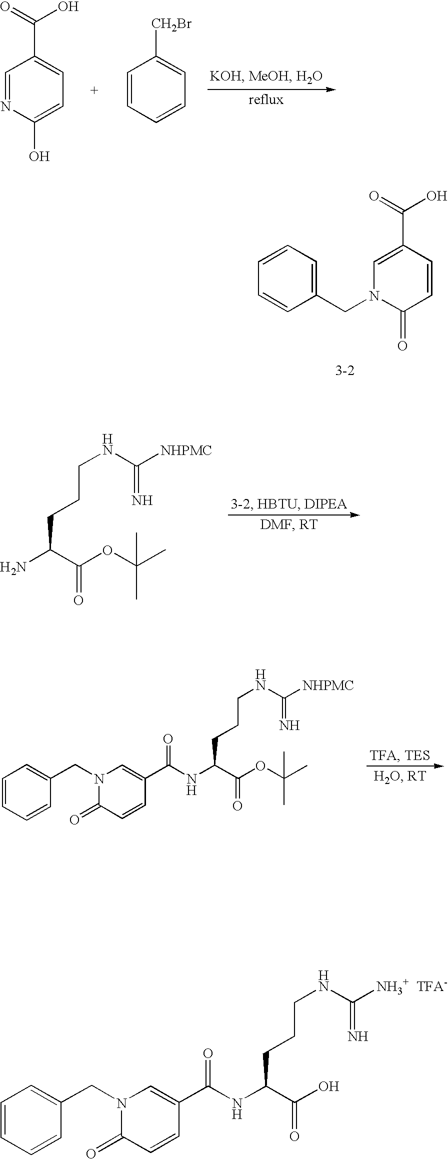

### I. 1-Benzyl-6-oxo-1,6-dihydropyridine-3-carboxylic acid

To a suspension of 6-hydroxynicotinic acid (0.50 g, 3.59 mmol) in water (1 mL) and methanol (5 mL) was added KOH (0.71 g, 12.57 mmol). The resulting solution was heated at reflux for 5 minutes and then benzyl bromide (0.85 mL, 7.19 mmol) was added. Heating was continued for an additional 90 minutes, the reaction was then cooled to room temperature and the methanol removed under reduced pressure. The resulting residue was diluted with water and washed twice with diethyl ether. The aqueous phase was acidified with 2 M HCl, and the resulting white precipitate was isolated by vacuum filtration, washed with water and air dried to give a white solid (0.49 g, 60%).

### II. tert-Butyl (2S)-2-{[(1-benzyl-6-oxo-1,6-dihydropyridin-3-yl)carbonyl]amino}-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (100 mg, 0.2 mmol), 1-benzyl-6-oxo-1,6-dihydropyridine-3-carboxylic acid (46 mg, 0.2 mmol), and HBTU (106 mg, 0.28 mmol) in anhydrous DMF (2 mL) was added DIPEA (0.09 mL, 0.50 mmol). The reaction mixture was stirred at room temperature for 4 days and then diluted with EtOAc. The organic layer was washed successively with water (3 times) and saturated NaCl, dried over Na₂SO₄ and filtered. The filtrate was concentrated under reduced pressure and the residue was purified by column chromatography on silica gel, eluting with 100% EtOAc to give a pale yellow foam (120 mg, 85%).

### III. (2S)-2-{[(1-Benzyl-6-oxo-1,6-dihydropyridin-3-yl)carbonyl]amino}-5-carbamimidamidopentanoic acid•TFA

To a solution of tert-butyl (2S)-2-{[(1-benzyl-6-oxo-1,6-dihydropyridin-3-yl)carbonyl]amino}-5-{[(2,2,5,7,8-pentamethyl-3,4-dihydro-2H-chromen-6-yl)sulfonyl]carbamimidamido}pentanoate (120 mg, 0.17 mmol) in TFA (2 mL), triethylsilane (0.2 mL) and water (0.2 mL) were added. After stirring the reaction mixture at room temperature for 4 hr, MTBE (20 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give a white solid (64 mg, 75%).

The following compounds were synthesized by modifications of the general procedure described in Example 11.

Compound 53: Step I was conducted with 0.30 g 2-hydroxyisonicotinic acid, 0.42 g KOH and 0.52 mL benzyl bromide in 3 mL MeOH/1 mL H₂O to provide 264 mg 1-benzyl-2-oxo-1,2-dihydropyridine-4-carboxylic acid. Step II was conducted using 46 mg 1-benzyl-2-oxo-1,2-dihydropyridine-4-carboxylic acid, 100 mg (S)-tert-butyl 2-amino-5-[3-(2,2,5,7,8-pentamethylchroman-6-ylsulfonyl)guanidino]pentanoate, 106 mg HBTU and 0.09 mL DIPEA in 2 mL DMF to provide 130 mg tert-butyl (2S)-2-{[(1-benzyl-2-oxo-1,2-dihydropyridin-4-yl)carbonyl]amino}-5-{[(2,2,5,7,8-pentamethyl-3,4-dihydro-2H-chromen-6-yl)sulfonyl]carbamimidamido}pentanoate. Step III was conducted using 130 mg tert-butyl (2S)-2-{[(1-benzyl-2-oxo-1,2-dihydropyridin-4-yl)carbonyl]amino}-5-{[(2,2,5,7,8-pentamethyl-3,4-dihydro-2H-chromen-6-yl)sulfonyl]carbamimidamido}pentanoate, 2 mL TFA, 0.2 mL triethylsilane, and 0.2 mL deionized H₂O to provide 65 mg (2S)-2-{[(1-benzyl-2-oxo-1,2-dihydropyridin-4-yl)carbonyl]amino}-5-carbamimidamidopentanoic acid•TFA.

Compound 55: Step I was conducted using 0.50 g 6-hydroxypyridine-2-carboxylic acid, 0.71 g KOH, and 0.85 mL benzyl bromide in 5 mL MeOH/1.5 mL deionized H₂O to provide 0.29 g 1-benzyl-6-oxo-1,6-dihydropyridine-2-carboxylic acid. Step II was conducted using 92 mg 1-benzyl-6-oxo-1,6-dihydropyridine-2-carboxylic acid, 200 mg mg (s)-tert-butyl 2-amino-5-[3-(2,2,5,7,8-pentamethylchroman-6-ylsulfonyl)guanidino]pentanoate, 212 mg HBTU, and 0.18 mL DIPEA in 4 mL DMF to provide 178 mg tert-butyl (2S)-2-{[(1-benzyl-6-oxo-1,6-dihydropyridin-2-yl)carbonyl]amino}-5-{[(2,2,5,7,8-pentamethyl-3,4-dihydro-2H-chromen-6-yl)sulfonyl]carbamimidamido}pentanoate. Step III was conducted with 178 mg tert-butyl (2S)-2-{[(1-benzyl-6-oxo-1,6-dihydropyridin-2-yl)carbonyl]amino}-5-{[(2,2,5,7,8-pentamethyl-3,4-dihydro-2H-chromen-6-yl)sulfonyl]carbamimidamido}pentanoate, 2 mL TFA, 0.2 mL triethylsilane, and 0.2 mL deionized H₂O to provide 16 mg (2S)-2-{[(1-benzyl-6-oxo-1,6-dihydropyridin-2-yl)carbonyl]amino}-5-carbamimidamidopentanoic acid•TFA following reversed-phase HPLC purification.

### Example 12

### Compound 57

### Synthesis of (2S)-5-carbamimidamido-2-({[1-(diphenylmethyl)-6-oxo-1,6-dihydropyridin-3-yl]carbonyl}amino)pentanoic acid•TFA

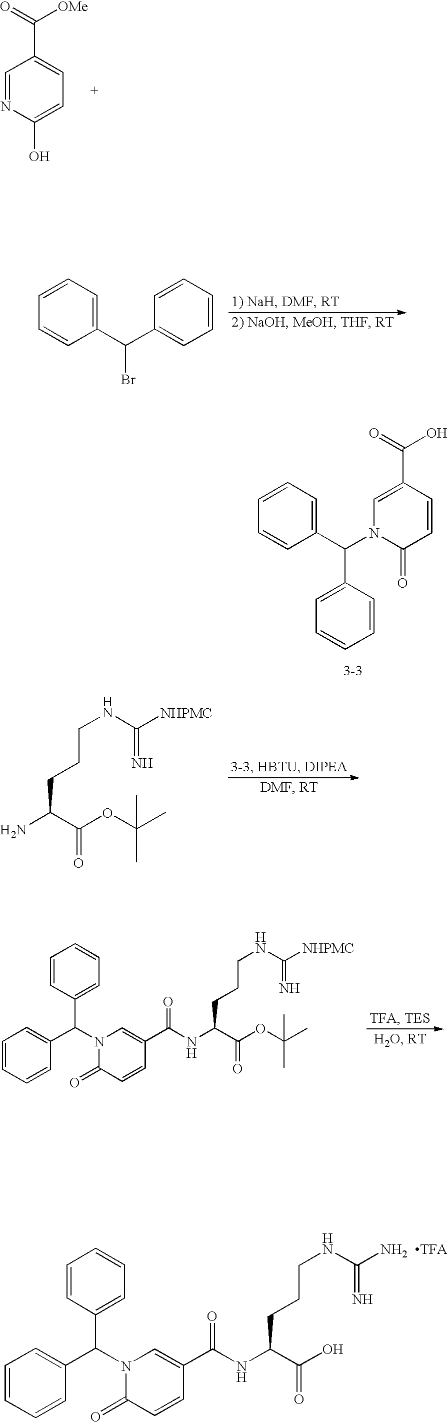

### I. Methyl 1-(diphenylmethyl)-6-oxo-1,6-dihydropyridine-3-carboxylate

Sodium hydride (60%, 96 mg, 2.40 mmol) was added to a solution of methyl 6-hydroxynicotinate (350 mg, 2.29 mmol) in DMF (15 mL). The resulting suspension was stirred at room temperature for 30 min and then a solution of benzhydryl bromide (0.71 mL, 2.86 mmol) in DMF (5 mL) was added by syringe. After stirring at room temperature for 6 days, the reaction was quenched with 2M HCl, diluted with water and extracted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 3:7 EtOAc:hexanes followed by 2:3 EtOAc:hexanes to give a white foam (426 mg, 58%).

### II. 1-(Diphenylmethyl)-6-oxo-1,6-dihydropyridine-3-carboxylic acid

To a solution of methyl 1-(diphenylmethyl)-6-oxo-1,6-dihydropyridine-3-carboxylate (426 mg, 1.34 mmol) in 1. THF:MeOH (10 mL) was added 2M NaOH (4.0 mL, 8.0 mmol) and the reaction stirred at room temperature for 3 hr. The reaction was diluted with water, and the resulting solution was washed twice with diethyl ether. The aqueous phase was acidified with 2 M HCl, and the resulting white precipitate was isolated by vacuum filtration, washed with water and air dried to give a white solid (0.41 g, 100%).

### III. tert-butyl (2S)-2-({[1-(diphenylmethyl)-6-oxo-1,6-dihydropyridin-3-yl]carbonyl}amino)-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (100 mg, 0.20 mmol), 1-(diphenylmethyl)-6-oxo-1,6-dihydropyridine-3-carboxylic acid (61 mg, 0.20 mmol), and HBTU (106 mg, 0.28 mmol) in anhydrous DMF (2 mL) was added DIPEA (0.09 mL, 0.50 mmol). The reaction mixture was stirred 2 days at room temperature and then diluted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 4:1 EtOAc:hexanes followed by 100% EtOAc to give a colorless oil (177 mg).

### IV. (2S)-5-Carbamimidamido-2-({[1-(diphenylmethyl)-6-oxo-1,6-dihydropyridin-3-yl]carbonyl}amino)pentanoic acid•TFA

To a solution of tert-butyl (2S)-2-({[1-(diphenylmethyl)-6-oxo-1,6-dihydropyridin-3-yl]carbonyl}amino)-5-{[(2,2,5,7,8-pentamethyl-3,4-dihydro-2H-chromen-6-yl)sulfonyl]carbamimidamido}pentanoate (177 mg, 0.23 mmol theoretical) in TFA (2 mL), triethylsilane (0.2 mL) and water (0.2 mL) were added. After stirring the reaction mixture at room temperature for 4 hr, MTBE (20 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give a white solid. Purification by reverse-phase HPLC eluting with a 1:9 CH₃CN:0.1% aqueous TFA to 3:2 CH₃CN:0.1% aqueous TFA gradient gave a white solid after lyophilization (34 mg, 26%).

The following compound was synthesized by modifications of the general procedure described in Example 11.

Compound 56: Step I was conducted with 300 mg methyl 2-hydroxyisonicotinate, 82 mg 60% NaH, and 605 benzhydryl bromide in 10 ml, DMF to provide 283 mg methyl methyl 1-(diphenylmethyl)-2-oxo-1,2-dihydropyridine-4-carboxylate. Step II was conducted with 283 mg methyl 1-(diphenylmethyl)-2-oxo-1,2-dihydropyridine-4-carboxylate and 2.7 mL 2 M NaOH in 8 mL 1:1 THF/MeOH to provide 220 mg 1-(diphenylmethyl)-2-oxo-1,2-dihydropyridine-4-carboxylic acid. Step III was conducted with 61 mg 1-(diphenylmethyl)-2-oxo-1,2-dihydropyridine-4-carboxylic acid, 100 mg tert-butyl (2S)-2-amino-5-{[(2,2,5,7,8-pentamethyl-3,4-dihydro-2H-chromen-6-yl)sulfonyl]carbamimidamido}pentanoate, 106 mg HBTU, and 0.09 mL DIPEA in 2 mL DMF to provide 168 mg tert-butyl (2S)-2-({[1-(diphenylmethyl)-2-oxo-1,2-dihydropyridin-4-yl]carbonyl}amino)-5-{[(2,2,5,7,8-pentamethyl-3,4-dihydro-2H-chromen-6-yl)sulfonyl]carbamimidamido}pentanoate. Step IV was conducted with 168 mg tert-butyl (2S)-2-({[1-(diphenylmethyl)-2-oxo-1,2-dihydropyridin-4-yl]carbonyl}amino)-5-{[(2,2,5,7,8-pentamethyl-3,4-dihydro-2H-chromen-6-yl)sulfonyl]carbamimidamido}pentanoate, 2 mL TFA, 0.2 mL triethylsilane, and 0.2 mL deionized H₂O to provide 56 mg (2S)-5-carbamimidamido-2-({[1-(diphenylmethyl)-2-oxo-1,2-dihydropyridin-4-yl]carbonyl}amino)pentanoic acid•TFA following reverse phase HPLC purification.

### Example 13

### Compound 58

### Synthesis of (2S)-5-carbamimidamido-2-({3-[(diphenylmethyl)amino]-2,2-dimethyl-3-oxopropanoyl}amino)pentanoic acid•TFA

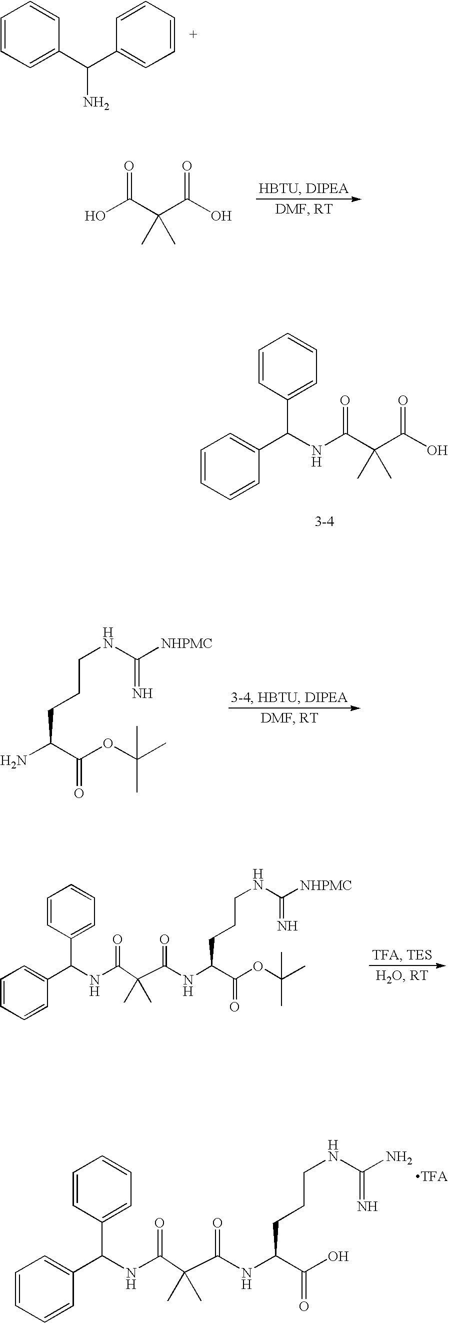

1,3-[(Diphenylmethyl)amino]-2,2-dimethyl-3-oxopropanoic acid

To a solution of benzhydrylamine (0.47 mL, 2.73 mmol), 2,2-dimethylmalonic acid (720 mg, 5.46 mmol) and HBTU (1.14 g, 3.0 mmol) in anhydrous DMF (sufficient quantity to dissolve reactants) was added DIPEA (0.53 mL, 3.0 mmol). The reaction mixture was stirred at room temperature overnight and then diluted with diethyl ether and water. The organic layer was washed with successively with 2M HCl and saturated NaCl, dried over Na₂SO₄ and filtered. The filtrate was concentrated under reduced pressure and the residue was purified by column chromatography on silica gel, eluting with 1% MeOH in EtOAc to give a colorless oil (300 mg, 39%).

### II. tert-butyl (2S)-2-({3-[(diphenylmethyl)amino]-2,2-dimethyl-3-oxopropanoyl} amino)-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (500 mg, 1.0 mmol), 3-[(diphenylmethyl)amino]-2,2-dimethyl-3-oxopropanoic acid (300 mg, 1.0 mmol) and HBTU (380 mg, 1.0 mmol) in anhydrous DMF (10 mL) was added DIPEA (0.35 mL, 2.0 mmol). The reaction mixture was stirred at room temperature overnight, then diluted with diethyl ether. The organic layer was washed with water and saturated NaCl, dried over Na₂SO₄ and filtered. The filtrate was concentrated under reduced pressure and the residue was purified by column chromatography on silica gel, eluting with 1:1 EtOAc:hexanes to give the product (200 mg, 26%).

### III. (2S)-5-Carbamimidamido-2-({3-[(diphenylmethyl)amino]-2,2-dimethyl-3-oxopropanoyl}amino)pentanoic acid•TFA

To a solution of tert-butyl (2S)-2-({3-[(diphenylmethyl)amino]-2,2-dimethyl-3-oxopropanoyl}amino)-5-{[(2,2,5,7,8-pentamethyl-3,4-dihydro-2H-chromen-6-yl)sulfonyl]carbamimidamido}pentanoate (200 mg, 0.26 mmol) in TFA (2 mL), triethylsilane (0.1 mL) and water (0.1 mL) were added. After stirring the reaction mixture at room temperature for 3 hr, MTBE (45 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was purified by reverse-phase HPLC eluting with a 1:9 CH₃CN:0.1% aqueous TFA to 3:2 CH₃CN:0.1% aqueous TFA gradient to give a white solid after lyophilization (66 mg, 46%).

### Example 14

### Compound 62

### Synthesis of (2S)-2-({[1-(diphenylmethyl)-2-oxo-1,2-dihydropyridin-3-yl]carbonyl}amino)-6-{[(4-methylphenyl)sulfonyl]amino}hexanoic acid

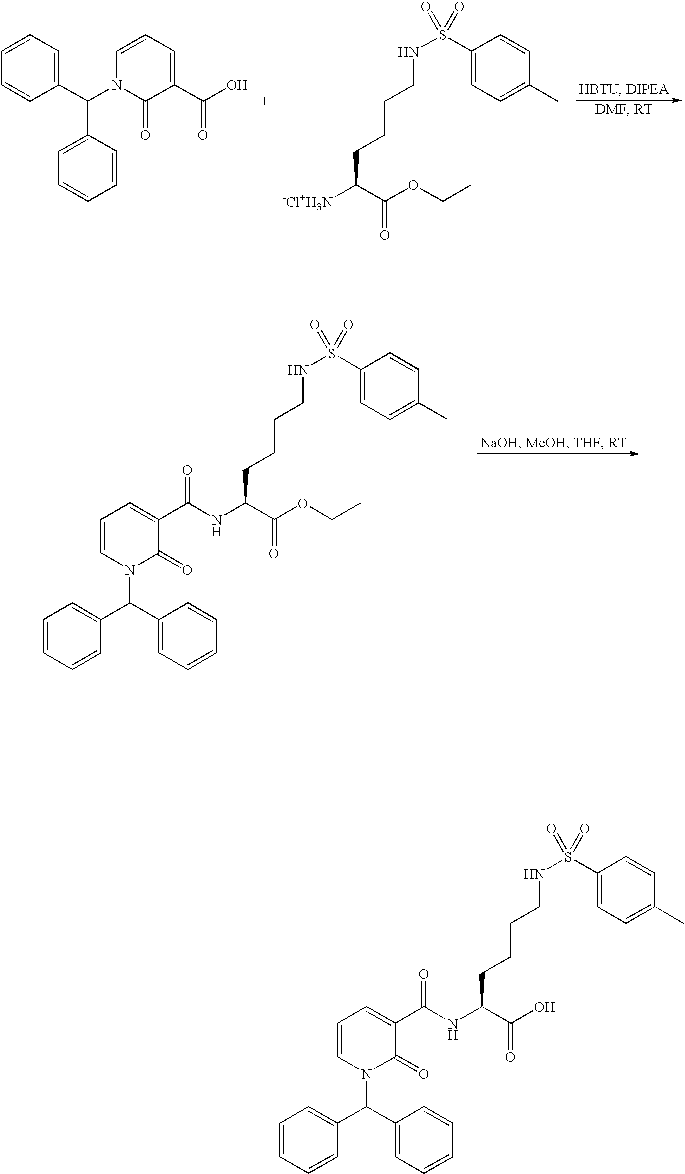

### I. Ethyl (2S)-2-({[1-(diphenylmethyl)-2-oxo-1,2-dihydropyridin-3-yl]carbonyl}amino)-6-{[(4-methylphenyl)sulfonyl]amino} hexanoate

To a solution of ethyl (2S)-2-amino-6-{[(4-methylphenyl)sulfonyl]amino}hexanoate hydrochloride (120 mg, 0.33 mmol), 1-(diphenylmethyl)-2-oxo-1,2-dihydropyridine-3-carboxylic acid (100 mg, 0.33 mmol), and HBTU (170 mg, 0.46 mmol) in DMF (2 mL) was added DIPEA (0.14 mL, 0.82 mmol). The reaction mixture was stirred at room temperature overnight, diluted with water and extracted into EtOAc. The EtOAc extract was washed successively with water and saturated NaCl, dried over MgSO₄ and filtered. The filtrate was concentrated under reduced pressure and the residue was purified by column chromatography on silica gel, eluting with 1:2 EtOAc/hexanes followed by 1:1 EtOAc/hexanes to give a white foam (100 mg, 50%).

### II. (2S)-2-({[1-(Diphenylmethyl)-2-oxo-1,2-dihydropyridin-3-yl]carbonyl}amino)-6-{[(4-methylphenyl)sulfonyl]amino} hexanoic acid

To a solution of ethyl (2S)-2-({[1-(diphenylmethyl)-2-oxo-1,2-dihydropyridin-3-yl]carbonyl}amino)-6-{[(4-methylphenyl)sulfonyl]amino} hexanoate (100 mg, 0.16 mmol) in 6:1 THF/MeOH (3.5 mL) was added 2M NaOH (1.5 mL, 3.0 mmol) and stirred at room temperature for 2 hr. The reaction was concentrated to dryness under reduced pressure, diluted with water, washed with diethyl ether (2 times) and the layers separated. The aqueous phase was acidified with 2M HCl and extracted with EtOAc. The organic layer was washed with water and saturated NaCl, dried over MgSO₄, filtered and the solvent was removed under reduced pressure. The solid was dissolved in CH₃CN/H₂O and the resulting solution was lyophilized lyophilized to give a white solid (89 mg, 94%).

The following compounds were synthesized by modifications of the general procedure described in Example 14.

Compound 61: Step I was conducted with 40 mg compound 1-1, 70 mg ethyl (2S)-2-amino-6-{[(4-methylphenyl)sulfonyl]amino} hexanoate hydrochloride, 90 mg HBTU, and 0.12 mL DIPEA in 2 mL DMF to provide 90 mg ethyl (2S)-2-{[(1-benzyl-2-oxo-1,2-dihydropyridin-3-yl)carbonyl]amino}-6-{[(4-methylphenyl)sulfonyl]amino}hexanoate. Step II was conducted using 90 mg ethyl (2S)-2-{[(1-benzyl-2-oxo-1,2-dihydropyridin-3-yl)carbonyl]amino}-6-{[(4-methylphenyl)sulfonyl]amino}hexanoate and 0.33 mL 2 M NaOH in 3 mL EtOH to provide 52 mg (2S)-2-{[(1-benzyl-2-oxo-1,2-dihydropyridin-3-yl)carbonyl]amino}-6-{[(4-methylphenyl)sulfonyl]amino} hexanoic acid.

### Example 15

### Compound 63

### Synthesis of (2S)-2-{[(1-benzyl-2-oxo-1,2-dihydropyridin-3-yl)carbonyl]amino}-6-carbamimidamidohexanoic acid•TFA

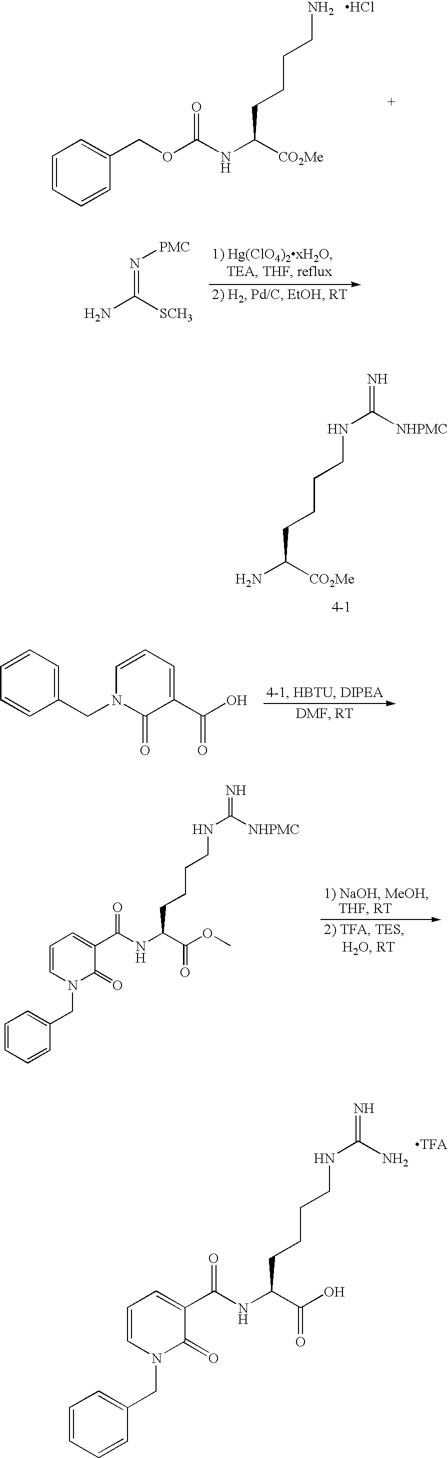

### I. Methyl (2S)-2-{[(benzyloxy)carbonyl]amino}-6-{[(2,2,5,7,8-pentamethyl-3,4-dihydro-2H-chromen-6-yl)sulfonyl]carbamimidamido} hexanoate

To a suspension of methyl (2S)-6-amino-2-{[(benzyloxy)carbonyl]amino}hexanoate hydrochloride (225 mg, 0.68 mmol), methyl N-[(2,2,5,7,8-pentamethyl-3,4-dihydro-2H-chromen-6-yl)sulfonyl]imidothiocarbamate (330 mg, 0.95 mmol) and Hg(ClO₄).xH₂O (407 mg) in THF (7 mL) was added TEA (0.28 mL, 2.0 mmol). The reaction mixture was heated at reflux under a nitrogen atmosphere overnight, cooled to room temperature, filtered through Celite, then concentrated under reduced pressure. The resulting residue was dissolved with EtOAc, washed successively with water and saturated NaCl, dried over Na₂SO₄ and filtered. The filtrate was concentrated under reduced pressure and the residue was purified by column chromatography on silica gel, eluting with 1:1 EtOAc:hexanes to 100% EtOAc to give the product (54 mg, 13%).

### II. Methyl (2S)-2-amino-6-{[(2,2,5,7,8-pentamethyl-3,4-dihydro-2H-chromen-6-yl)sulfonyl]carbamimidamido} hexanoate

To a solution of methyl (2S)-2-{[(benzyloxy)carbonyl]amino}-6-{[(2,2,5,7,8-pentamethyl-3,4-dihydro-2H-chromen-6-yl)sulfonyl]carbamimidamido}hexanoate (214 mg, 0.36 mmol) in EtOH (20 mL) was added 10% Palladium on carbon (71 mg, 0.33 wt eq). The reaction mixture was placed under 1 atm of H₂ and stirred at room temperature. After stirring overnight, an additional 10% Palladium on carbon (100 mg) was added, and stirring under 1 atm H₂ was continued for an additional 7 hr. The reaction mixture was filtered through Celite, and the solvent was removed under reduced pressure. The residue was purified by column chromatography on silica gel, eluting with 1:19 MeOH/CH₂Cl₂ followed by 1:9 MeOH/CH₂Cl₂ to give the product (107 mg, 64%).

### III. Methyl (2S)-2-{[(1-benzyl-2-oxo-1,2-dihydropyridin-3-yl)carbonyl]amino}-6-{[(2,2,5,7,8-pentamethyl-3,4-dihydro-2H-chromen-6-yl)sulfonyl]carbamimidamido} hexanoate

To a solution of methyl (2S)-2-amino-6-{[(2,2,5,7,8-pentamethyl-3,4-dihydro-2H-chromen-6-yl)sulfonyl]carbamimidamido}hexanoate (56 mg, 0.12 mmol), 1-benzyl-2-oxo-1,2-dihydropyridine-3-carboxylic acid (29 mg, 0.13 mmol), and HBTU (64 mg, 0.17 mmol) in DMF (2 mL) was added DIPEA (0.047 mL, 0.26 mmol). The reaction mixture was stirred overnight at room temperature and then diluted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 1:19 MeOH/CH₂Cl₂ to give a colorless oil (85 mg, 100%).

### IV. (2S)-2-{[(1-Benzyl-2-oxo-1,2-dihydropyridin-3-yl)carbonyl]amino}-6-{[(2,2,5,7,8-pentamethyl-3,4-dihydro-2H-chromen-6-yl)sulfonyl]carbamimidamido} hexanoic acid

To a solution of methyl (2S)-2-{[(1-benzyl-2-oxo-1,2-dihydropyridin-3-yl)carbonyl]amino}-6-{[(2,2,5,7,8-pentamethyl-3,4-dihydro-2H-chromen-6-yl)sulfonyl]carbamimidamido}hexanoate (85 mg, 0.13 mmol) in MeOH (4 mL) was added 2M NaOH (0.38 mL, 0.75 mmol) and then stirred at room temperature for 6 hr. The reaction mixture was diluted with water, washed with diethyl ether (2 times) and the layers separated. The aqueous phase was acidified with 2M HCl and extracted with EtOAc. The organic layer was washed with water and saturated NaCl, dried over MgSO₄, filtered and the solvent was removed under reduced pressure. The resulting semi-solid was dissolved in CH₃CN/H₂O and lyophilized to give a white solid (74 mg, 89%).

### V. (2S)-2-{[(1-Benzyl-2-oxo-1,2-dihydropyridin-3-yl)carbonyl]amino}-6-carbamimidamidohexanoic acid•TFA

To a solution of (2S)-2-{[(1-benzyl-2-oxo-1,2-dihydropyridin-3-yl)carbonyl]amino}-6-{[(2,2,5,7,8-pentamethyl-3,4-dihydro-2H-chromen-6-yl)sulfonyl]carbamimidamido}hexanoic acid (74 mg, 0.11 mmol) in TFA (2 mL), triethylsilane (0.1 mL) and water (0.1 mL) were added. After stirring the reaction mixture at room temperature for 3.5 hr, MTBE (25 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized to give a white solid (18 mg, 32%).

The following compounds were synthesized by modifications of the general procedure described in Example 15.

Compound 85: Step III was conducted with 245 mg compound 2-2, 360 mg methyl (2S)-2-amino-3-(1-trityl-1H-imidazol-4-yl)propanoate, 364 mg HBTU, and 0.56 mL DIPEA in 10 mL DMF to provide 480 mg methyl (2S)-2-({[1-(diphenylmethyl)-2-oxo-1,2-dihydropyridin-3-yl]carbonyl}amino)-3-(1-trityl-1H-imidazol-4-yl)propanoate. Step IV was conducted using 480 mg methyl (2S)-2-({[1-(diphenylmethyl)-2-oxo-1,2-dihydropyridin-3-yl]carbonyl}amino)-3-(1-trityl-1H-imidazol-4-yl)propanoate and 2 mL 6 M NaOH in 10 mL methanol to provide 450 mg (2S)-2-({[1-(diphenylmethyl)-2-oxo-1,2-dihydropyridin-3-yl]carbonyl}amino)-3-(1-trityl-1H-imidazol-4-yl)propanoic acid. Step V was conducted with 450 mg (2S)-2-({[1-(diphenylmethyl)-2-oxo-1,2-dihydropyridin-3-yl]carbonyl}amino)-3-(1-trityl-1H-imidazol-4-yl)propanoic acid, 3 mL TFA, 0.1 mL triethylsilane and 0.1 mL water to provide 140 mg (2S)-2-({[1-(diphenylmethyl)-2-oxo-1,2-dihydropyridin-3-yl]carbonyl}amino)-3-(1H-imidazol-4-yl)propanoic acid•TFA after reverse phase HPLC purification.

### Example 16

### Compound 64

### Synthesis of (2S)-5-amino-2-({[1-(diphenylmethyl)-2-oxo-1,2-dihydropyridin-3-yl]carbonyl}amino)-5-oxopentanoic acid

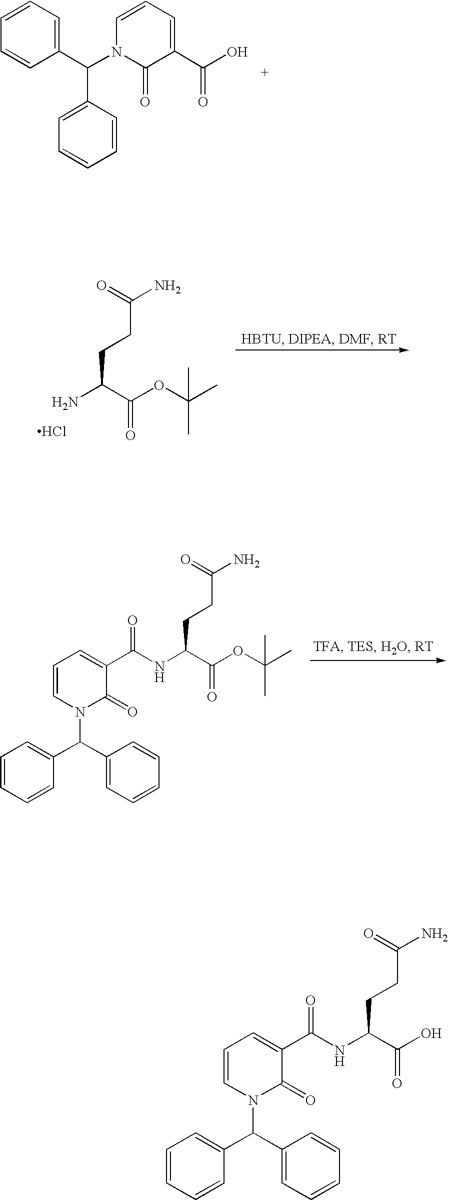

### I. tert-Butyl (2S)-5-amino-2-({[1-(diphenylmethyl)-2-oxo-1,2-dihydropyridin-3-yl]carbonyl}amino)-5-oxopentanoate

To a solution of tert-butyl (2S)-2,5-diamino-5-oxopentanoate hydrochloride (130 mg, 0.54 mmol), 1-(diphenylmethyl)-2-oxo-1,2-dihydropyridine-3-carboxylic acid (183 mg, 0.60 mmol), and HBTU (246 mg, 0.65 mmol) in DMF (5 mL) was added DIPEA (0.31 mL, 1.8 mmol). The reaction mixture was stirred at room temperature overnight, diluted with EtOAc then washed successively with water (3 times) and saturated NaCl, dried over Na₂SO₄ and filtered. The filtrate was concentrated under reduced pressure and the residue was purified by column chromatography on silica gel, eluting with 1:9 EtOAc/hexanes followed by 100% EtOAc to give a white foam (320 mg).

### II. (2S)-5-Amino-2-({[1-(diphenylmethyl)-2-oxo-1,2-dihydropyridin-3-yl]carbonyl}amino)-5-oxopentanoic acid

To a solution of tert-butyl (2S)-5-amino-2-({[1-(diphenylmethyl)-2-oxo-1,2-dihydropyridin-3-yl]carbonyl}amino)-5-oxopentanoate (320 mg, 0.54 mmol theoretical) in TFA (2 mL), triethylsilane (0.1 mL) and water (0.1 mL) were added and the reaction mixture was stirred at room temperature for 1 hr and then concentrated under reduced pressure. The resulting residue was purified by column chromatography on silica gel, eluting with a 100% EtOAc to 2:3 MeOH/EtOAc gradient to give a white solid (150 mg, 64%).

### Example 17

### Compound 65

### Synthesis of (2S)-4-carbamimidamido-2-({[1-(diphenylmethyl)-2-oxo-1,2-dihydropyridin-3-yl]carbonyl}amino)butanoic acid•TFA

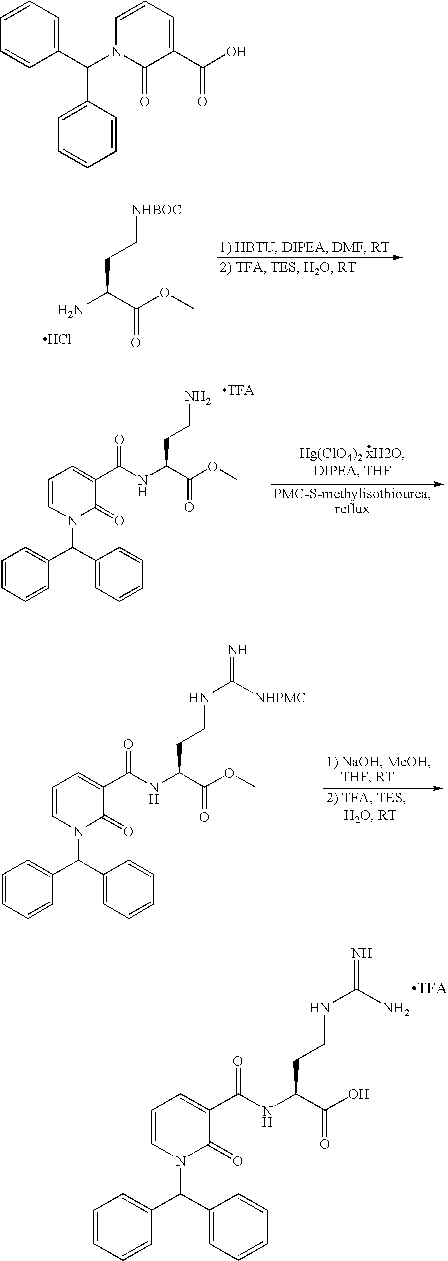

### I. Methyl (2S)-4-[(tert-butoxycarbonyl)amino]-2-({[1-(diphenylmethyl)-2-oxo-1,2-dihydropyridin-3-yl]carbonyl}amino)butanoate

To a solution of methyl (2S)-2-amino-4-[(tert-butoxycarbonyl)amino]butanoate hydrochloride (291 mg, 1.1 mmol), 1-(diphenylmethyl)-2-oxo-1,2-dihydropyridine-3-carboxylic acid (330 mg, 1.11 mmol), and HBTU (491 mg, 1.3 mmol) in DMF (10 mL) was added DIPEA (0.56 mL, 3.2 mmol). The reaction mixture was stirred overnight at room temperature and then diluted with EtOAc. The organic layer was washed with water and saturated NaCl, dried over Na₂SO₄ and filtered. The filtrate was concentrated under reduced pressure and the residue was purified by column chromatography on silica gel, eluting with a 1:9 EtOAc:hexanes to 100% EtOAc gradient to give a white foam (420 mg, 75%).

### II. Methyl (2S)-4-amino-2-({[1-(diphenylmethyl)-2-oxo-1,2-dihydropyridin-3-yl]carbonyl}amino)butanoate•TFA

To a solution of methyl (2S)-4-[(tert-butoxycarbonyl)amino]-2-({[1-(diphenylmethyl)-2-oxo-1,2-dihydropyridin-3-yl]carbonyl}amino)butanoate (420 mg, 0.81 mmol) in TFA (4 mL), triethylsilane (0.2 mL) and water (0.2 mL) were added. After stirring the reaction mixture at room temperature for 2.5 hr, the solvent was removed under reduced pressure. The resulting residue was purified by reverse-phase HPLC eluting with a 10% to 60% CH₃CN:0.1% aqueous TFA gradient to give a white solid (194 mg, 44%).

### III. Methyl (2S)-2-({[1-(diphenylmethyl)-2-oxo-1,2-dihydropyridin-3-yl]carbonyl}amino)-4-{[(2,2,5,7,8-pentamethyl-3,4-dihydro-2H-chromen-6-yl)sulfonyl]carbamimidamido}butanoate

To a solution of methyl (2S)-4-amino-2-({[1-(diphenylmethyl)-2-oxo-1,2-dihydropyridin-3-yl]carbonyl}amino)butanoate-TFA (53 mg, 0.1 mmol) and methyl N-[(2,2,5,7,8-pentamethyl-3,4-dihydro-2H-chromen-6-yl)sulfonyl]imidothiocarbamate (71 mg, 0.2 mmol) in THF (6 mL) was added DIPEA (0.035 mL) and Hg(ClO₄).xH₂O (44 mg). The reaction mixture was heated at reflux under a nitrogen atmosphere for 2 hr, and then additional PMC-S-methylisothiourea (100 mg, 0.28 mmol) was added. After heating at reflux for 2 hr more, the reaction mixture was allowed to stand at room temperature overnight and then concentrated under reduced pressure. The resulting residue was purified by column chromatography on silica gel, eluting with a 1:9 EtOAc:hexanes to 100% EtOAc gradient to give a colorless oil (35 mg, 48%).

### IV. (2S)-2-({[1-(Diphenylmethyl)-2-oxo-1,2-dihydropyridin-3-yl]carbonyl}amino)-4-{[(2,2,5,7,8-pentamethyl-3,4-dihydro-2H-chromen-6-yl)sulfonyl]carbamimidamido}butanoic acid

To a solution of methyl (2S)-2-({[1-(diphenylmethyl)-2-oxo-1,2-dihydropyridin-3-yl]carbonyl}amino)-4-{[(2,2,5,7,8-pentamethyl-3,4-dihydro-2H-chromen-6-yl)sulfonyl]carbamimidamido}butanoate (35 mg, 0.05 mmol) in MeOH (3 mL) was added 2M NaOH (5 drops, excess) and then stirred at room temperature overnight. The reaction was acidified with 1 M HCl and the product was extracted into EtOAc, dried over Na₂SO₄ and filtered. The filtrate was concentrated under reduced pressure to give the product (30 mg, 88%).

### V. (2S)-4-carbamimidamido-2-({[1-(diphenylmethyl)-2-oxo-1,2-dihydropyridin-3-yl]carbonyl}amino)butanoic acid•TFA

To a solution of (2S)-2-({[1-(diphenylmethyl)-2-oxo-1,2-dihydropyridin-3-yl]carbonyl}amino)-4-{[(2,2,5,7,8-pentamethyl-3,4-dihydro-2H-chromen-6-yl)sulfonyl]carbamimidamido}butanoic acid (30 mg, 0.04 mmol) in TFA (2 mL), triethylsilane (0.1 mL) and water (0.1 mL) were added. After stirring the reaction mixture at room temperature for 4 hr, the reaction mixture was concentrated under reduced pressure and purified by reverse-phase HPLC eluting with a 10% to 60% CH₃CN:0.1% aqueous TFA gradient to give a white solid after lyophilization (6 mg, 27%).

### Example 18

### Compound 66

### Synthesis of (2S)-2-({[1-(diphenylmethyl)-2-oxo-1,2-dihydropyridin-3-yl]carbonyl}amino)-4-{[[(ethoxycarbonyl)amino](methylamino)methylene]amino}butanoic acid

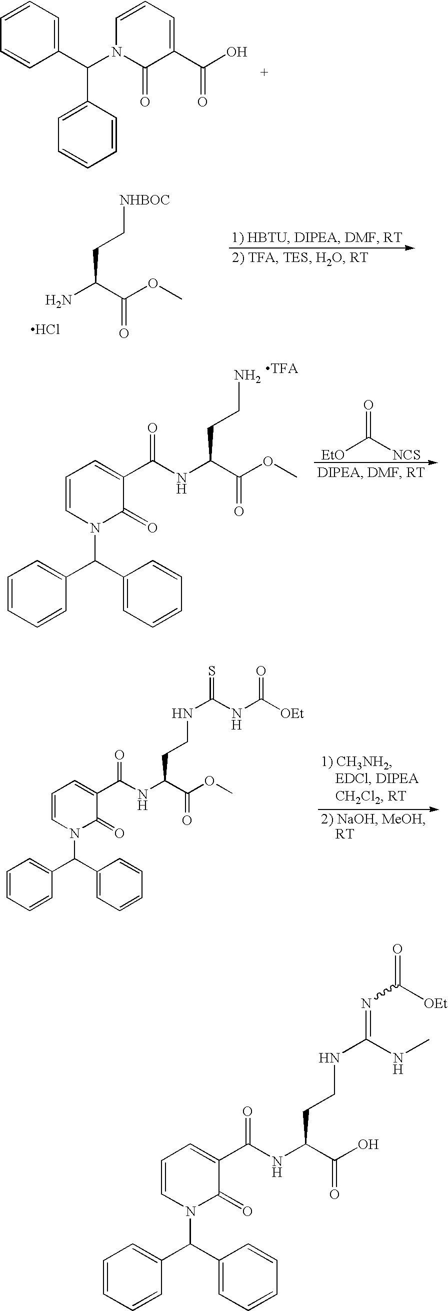

### I. Methyl (2S)-4-[(tert-butoxycarbonyl)amino]-2-({[1-(diphenylmethyl)-2-oxo-1,2-dihydropyridin-3-yl]carbonyl}amino)butanoate

To a solution of methyl (2S)-2-amino-4-[(tert-butoxycarbonyl)amino]butanoate hydrochloride (100 mg, 0.37 mmol), 1-(diphenylmethyl)-2-oxo-1,2-dihydropyridine-3-carboxylic acid (125 mg, 0.41 mmol), and HBTU (182 mg, 0.48 mmol) in DMF (sufficient quantity to dissolve reactants) was added DIPEA (0.19 mL, 1.11 mmol). The reaction mixture was stirred 2 hr at room temperature and then diluted with EtOAc. The organic layer was washed with water (3 times) and saturated NaCl, dried over Na₂SO₄ and filtered. The filtrate was concentrated under reduced pressure and the residue was purified by column chromatography on silica gel, eluting with a 1:9 EtOAc:hexanes to 100% EtOAc gradient to give a white foam (150 mg, 78%).

### II. Methyl (2S)-4-amino-2-({[1-(diphenylmethyl)-2-oxo-1,2-dihydropyridin-3-yl]carbonyl}amino)butanoate-TFA

To a solution of methyl (2S)-4-[(tert-butoxycarbonyl)amino]-2-({[1-(diphenylmethyl)-2-oxo-1,2-dihydropyridin-3-yl]carbonyl}amino)butanoate (140 mg, 0.27 mmol) in TFA (2 mL), triethylsilane (0.1 mL) and water (0.1 mL) were added. After stirring the reaction mixture at room temperature for 2 hr, the solvent was removed under reduced pressure. The resulting residue was purified by reverse-phase HPLC eluting with a 20% to 80% CH₃CN:0.1% aqueous TFA gradient to give a white solid after lyophilization (113 mg, 78%).

### III. Methyl (2S)-2-({[1-(diphenylmethyl)-2-oxo-1,2-dihydropyridin-3-yl]carbonyl}amino)-4-{[(ethoxycarbonyl)carbamothioyl]amino}butanoate

To a solution of methyl (2S)-4-amino-2-({[1-(diphenylmethyl)-2-oxo-1,2-dihydropyridin-3-yl]carbonyl}amino)butanoate•TFA (113 mg, 0.21 mmol) in CH₂Cl₂ (sufficient amount to dissolve reactant), ethoxycarbonyl isothiourea (0.37 mL, 0.32 mmol) and DIPEA (0.55 mL, 0.32 mmol) were added. The reaction was stirred at room temperature for 5 minutes and then directly purified by column chromatography on silica gel eluting with a 1:4 EtOAc/hexanes to 100% EtOAc gradient to give a colorless oil (115 mg, 98%).

### IV. Methyl (2S)-2-({[1-(diphenylmethyl)-2-oxo-1,2-dihydropyridin-3-yl]carbonyl}amino)-4-{[[(ethoxycarbonyl)amino](methylamino)methylene]amino}butanoate

To a solution of methyl (2S)-2-({[1-(diphenylmethyl)-2-oxo-1,2-dihydropyridin-3-yl]carbonyl}amino)-4-{[(ethoxycarbonyl)carbamothioyl]amino}butanoate (110 mg, 0.20 mmol) and EDCI (58 mg, 0.30 mmol) in CH₂Cl₂ (10 mL) was added methylamine (2M solution in THF, 0.4 mL, excess) and DIPEA (0.07 mL, 0.40 mmol) and the reaction stirred at room temperature overnight. The reaction mixture was diluted with EtOAc and washed successively with water and saturated NaCl, dried over Na₂SO₄ and filtered. The filtrate was concentrated under reduced pressure and the residue was purified by column chromatography on silica gel, eluting with a 1:9 EtOAc:hexanes to 100% EtOAc gradient to give a white foam (66 mg, 61%).

### V. (2S)-2-({[1-(Diphenylmethyl)-2-oxo-1,2-dihydropyridin-3-yl]carbonyl}amino)-4-{[{(ethoxycarbonyl)amino}(methylamino)methylene]amino}butanoic acid

To a solution of methyl (2S)-2-({[1-(diphenylmethyl)-2-oxo-1,2-dihydropyridin-3-yl]carbonyl}amino)-4-{[[(ethoxycarbonyl)amino](methylamino)methylene]amino}butanoate (66 mg, 0.12 mmol) in MeOH (5 mL) was added 2M NaOH (0.6 mL, 1.2 mmol) and then stirred at room temperature overnight. The reaction mixture was directly purified by reverse-phase HPLC eluting with a 10% to 60% CH₃CN:0.1% aqueous TFA gradient to give a white solid after lyophilization (58 mg, 91%).

### Example 19

### Compound 71

### Synthesis of (2S)-5-(4,5-dihydro-1H-imidazol-2-ylamino)-2-({[1-(diphenylmethyl)-2-oxo-1,2-dihydropyridin-3-yl]carbonyl}amino)pentanoic acid•TFA

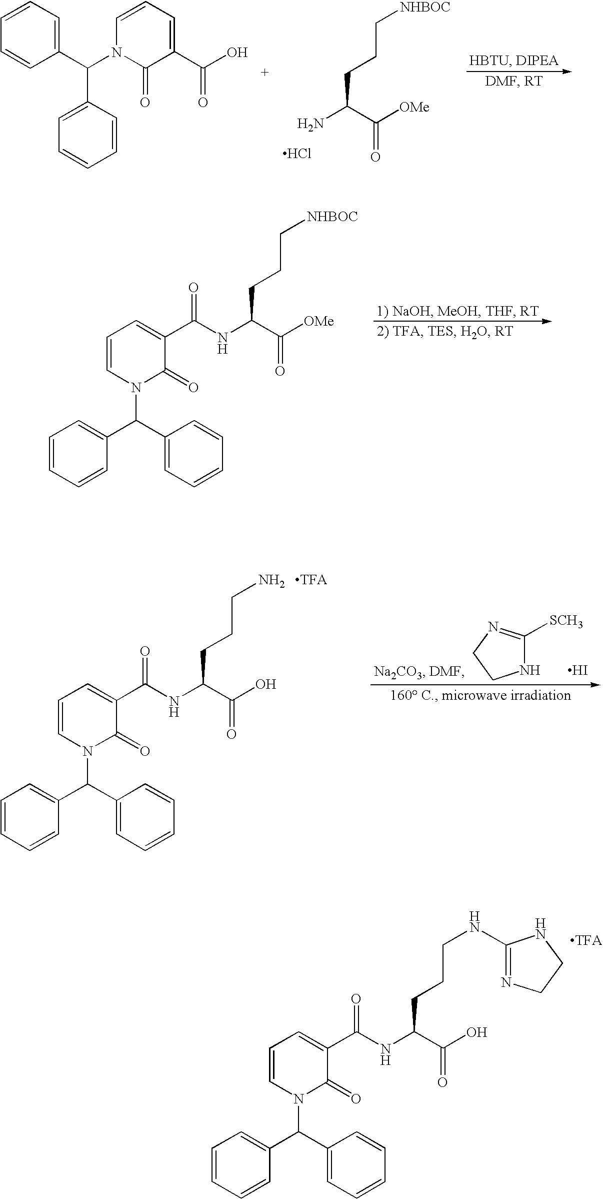

### I. Methyl (2S)-5-[(tert-butoxycarbonyl)amino]-2-({[1-(diphenylmethyl)-2-oxo-1,2-dihydropyridin-3-yl]carbonyl}amino)pentanoate

To a solution of methyl (2S)-2-amino-5-[(tert-butoxycarbonyl)amino]pentanoate hydrochloride (0.24 g, 0.86 mmol), 1-(diphenylmethyl)-2-oxo-1,2-dihydropyridine-3-carboxylic acid (0.25 g, 0.82 mmol), and HBTU (0.43 g, 1.1 mmol) in DMF (4 mL) was added DIPEA (0.43 mL, 2.5 mmol). The reaction mixture was stirred at room temperature overnight, diluted with water and extracted into EtOAc. The EtOAc extract was washed successively with water and saturated NaCl, dried over MgSO₄ and filtered. The filtrate was concentrated under reduced pressure to give a yellow oil which was used in the next step without purification (0.60 g).

### II. (2S)-5-[(tert-Butoxycarbonyl)amino]-2-({[1-(diphenylmethyl)-2-oxo-1,2-dihydropyridin-3-yl]carbonyl}amino)pentanoic acid

To a solution of methyl (2S)-5-[(tert-butoxycarbonyl)amino]-2-({[1-(diphenylmethyl)-2-oxo-1,2-dihydropyridin-3-yl]carbonyl}amino)pentanoate (0.60 g, 0.82 mmol theoretical) in 6:1 THF/MeOH (7 mL) was added 2M NaOH (3.0 mL, 6.0 mmol) and stirred at room temperature for 2 hr. The reaction was concentrated to dryness under reduced pressure and then diluted with water, washed with diethyl ether (2 times) and the layers separated. The aqueous phase was acidified with 2M HCl and extracted with EtOAc. The organic layer was washed with water and saturated NaCl, dried over MgSO₄ and filtered. The solvent was removed under reduced pressure to give a pale yellow foam which was used in the next step without purification (0.58 g).

### III. (2S)-5-Amino-2-({[1-(diphenylmethyl)-2-oxo-1,2-dihydropyridin-3-yl]carbonyl}amino)pentanoic acid•TFA

To a solution of (2S)-5-[(tert-butoxycarbonyl)amino]-2-({[1-(diphenylmethyl)-2-oxo-1,2-dihydropyridin-3-yl]carbonyl}amino)pentanoic acid (0.58 g, 0.82 mmol theoretical) in TFA (8 mL), triethylsilane (0.8 mL) and the reaction mixture was stirred at room temperature for 2 hr and then concentrated under reduced pressure. Excess EtOAc was added to the residue resulting in a white solid which was collected by filtration (0.41 g, 94% over 3 steps).

### IV. (2S)-5-(4,5-Dihydro-1H-imidazol-2-ylamino)-2-({[1-(diphenylmethyl)-2-oxo-1,2-dihydropyridin-3-yl]carbonyl}amino)pentanoic acid•TFA

To a solution of (2S)-5-amino-2-({[1-(diphenylmethyl)-2-oxo-1,2-dihydropyridin-3-yl]carbonyl}amino)pentanoic acid•TFA (0.15 g, 0.36 mmol) in DMF (2 mL) was added 2-(methylthio)-4,5-dihydro-1H-imidazole hydroiodide (0.12 g, 0.50 mmol) and Na₂CO₃ (0.19 g, 1.79 mmol) and then the reaction mixture was heated at 160° C. for 6 minutes in a Biotage Initiator microwave reactor. The solvent was removed under reduced pressure followed by purification of the resulting residue by reverse-phase HPLC eluting with a 10 to 60% CH₃CN:0.1% aqueous TFA gradient to give a white solid after lyophilization (33 mg, 20%).

The following compound was synthesized by modifications of the general procedure described in Example 19.

Compound 68: Step IV was conducted using 250 mg (2S)-5-amino-2-({[1-(diphenylmethyl)-2-oxo-1,2-dihydropyridin-3-yl]carbonyl}amino)pentanoic acid•TFA, 110 mg p-toluenesulfonyl chloride, and 0.3 mL DIPEA in 3 mL 1,2-dichloroethane at room temperature to provide 45 mg of (2S)-2-({[1-(diphenylmethyl)-2-oxo-1,2-dihydropyridin-3-yl]carbonyl}amino)-5-{[(4-methylphenyl)sulfonyl]amino}pentanoic acid following reversed-phase HPLC purification.

Compound 70: The title compound was synthesized analogously to Compound 71, except (S)-5-amio-2-(1-benzyl-2-oxo-1,2-dihydropyridine-3-carboxamido)pentanoic acid•TFA was used in place of (S)-5-amio-2-(1-benzhydryl-2-oxo-1,2-dihydropyridine-3-carboxamido)pentanoic acid•TFA. To a solution of 100 mg (2S)-5-amino-2-{[(1-benzyl-2-oxo-1,2-dihydropyridin-3-yl)carbonyl]amino}pentanoic acid•TFA in 5 mL MeOH was added 225 mg 2-(methylthio)-4,5-dihydro-1H-imidazole hydroiodide and 1 mL DIPEA. Following heating at 80° C. overnight, the solvent removed under reduced pressure and 41 mg (2S)-2-{[(1-benzyl-2-oxo-1,2-dihydropyridin-3-yl)carbonyl]amino}-5-(4,5-dihydro-1H-imidazol-2-ylamino)pentanoic acid•TFA was isolated following chromatography on silica gel with MeOH/CH₂Cl₂.

### Example 20

### Compound 72

### Synthesis of (2S)-{[(1-benzyl-2-oxo-1,2-dihydropyridin-3-yl)carbonyl]amino}(3-carbamimidamidophenyl)acetic acid•TFA

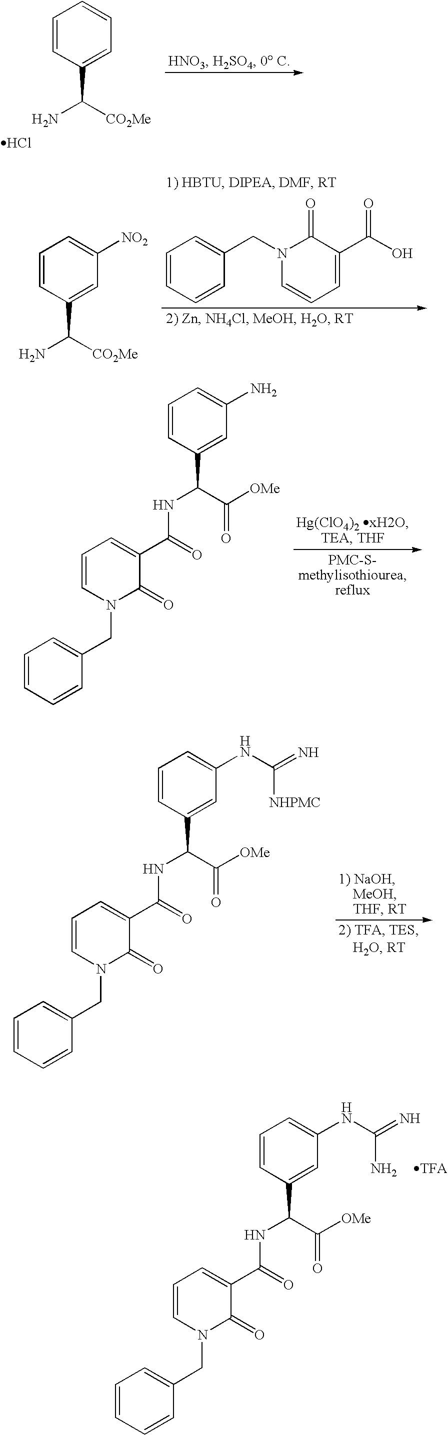

### I. Methyl (2S)-amino(3-nitrophenyl)acetate

To a solution of methyl (2S)-amino(phenyl)acetate hydrochloride (20.0 g, 99.2 mmol) in concentrated H₂SO₄ (100 mL), chilled to 0° C., was added fuming HNO₃ (7.4 mL). The reaction mixture was stirred at 0° C. for 4 hr and then poured onto ice. The product was extracted with EtOAc, and the EtOAc extract was cooled to 0° C. and washed successively with saturated NaHCO₃ (2 times) and saturated NaCl, dried over Na₂SO₄ and filtered. The filtrate was concentrated under reduced pressure and the residue was purified by column chromatography on silica gel, eluting with a 1:20 EtOAc/hexanes to 2:1 EtOAc/hexanes gradient to give a brown oil (0.4 g, 2%).

### II. Methyl (2S)-{[(1-benzyl-2-oxo-1,2-dihydropyridin-3-yl)carbonyl]amino}-(3-nitrophenyl)acetate

To a solution of methyl (2S)-amino(3-nitrophenyl)acetate (1.7 g, 8.1 mmol), 1-benzyl-2-oxo-1,2-dihydropyridine-3-carboxylic acid (1.6 g, 7.6 mmol), and HBTU (4.3 g, 11.3 mmol) in DMF (35 mL) was added DIPEA (1.8 mL, 10.5 mmol). The reaction mixture was stirred overnight at room temperature and then diluted with EtOAc. The organic layer was washed with water and saturated NaCl, dried over MgSO₄ and filtered. The filtrate was concentrated under reduced pressure and the residue was purified by column chromatography on silica gel, eluting with a 1:10 EtOAc/hexanes to 1:2 EtOAc/hexanes gradient to give a mixture of (S)-methyl 2-(1-benzyl-2-oxo-1,2-dihydropyridine-3-carboxamido)-2-(3-nitrophenyl)acetate and unreacted 1-benzyl-2-oxo-1,2-dihydropyridine-3-carboxylic acid (5.2 g).

### III. Methyl (2S)-(3-aminophenyl) {[(1-benzyl-2-oxo-1,2-dihydropyridin-3-yl)carbonyl]amino}acetate

To a solution of the mixture of methyl (2S)-{[(1-benzyl-2-oxo-1,2-dihydropyridin-3-yl)carbonyl]amino}-(3-nitrophenyl)acetate and unreacted 1-benzyl-2-oxo-1,2-dihydropyridine-3-carboxylic acid (5.2 g, ˜12.3 mmol) in MeOH (40 mL) was added a solution of NH₄Cl (1.5 g, 27.1 mmol) in water (30 mL), followed by Zn dust (5.4 g 82.7 mmol). The reaction mixture was stirred 2 hr at room temperature and then filtered through Celite. The filtrate was concentrated under reduced pressure and the resulting residue was dissolved with EtOAc, washed successively with saturated NaHCO₃, water and saturated NaCl, dried over MgSO₄ and filtered. The filtrate was concentrated under reduced pressure to give a yellow oil (2.5 g, 52% over 2 steps).

### IV. Methyl (2S)-{[(1-benzyl-2-oxo-1,2-dihydropyridin-3-yl)carbonyl]amino}(3-{[(2,2,5,7,8-pentamethyl-3,4-dihydro-2H-chromen-6-yl)sulfonyl]carbamimidamido} phenyl)acetate

To a solution of methyl (2S)-(3-aminophenyl){[(1-benzyl-2-oxo-1,2-dihydropyridin-3-yl)carbonyl]amino}acetate (2.4 g, 6.1 mmol), (Z)-2-methyl-1-(2,2,5,7,8-pentamethylchroman-6-ylsulfonyl)isothiouronium (2.8 g, 8.0 mmol) and Hg(ClO₄).xH₂O (3.4 g) in THF (30 mL) was added TEA (2.6 mL, 18.4 mmol). The reaction mixture was heated at reflux under a nitrogen atmosphere for 2 days. After heating at reflux for 2 days, additional (Z)-2-methyl-1-(2,2,5,7,8-pentamethylchroman-6-ylsulfonyl)isothiouronium (0.5 g, 1.4 mmol) was added and the reaction was heated at reflux overnight again. The reaction was then cooled to room temperature and concentrated under reduced pressure. The resulting residue was dissolved in EtOAc and filtered through Celite. The filtrate was washed successively with water, saturated NaHCO₃ and saturated NaCl, dried over MgSO₄ and filtered. The filtrate was concentrated under reduced pressure and the residue was purified twice by column chromatography on silica gel; first eluting with a 1:100 MeOH/CH₂Cl₂ to 1:10 MeOH/CH₂Cl₂ gradient and second eluting with a 1:5 EtOAc/hexanes to 5:1 EtOAc/hexanes to give a yellow foam (0.48 g, 11%).

### V. (2S)-{[(1-Benzyl-2-oxo-1,2-dihydropyridin-3-yl)carbonyl]amino}(3-{[(2,2,5,7,8-pentamethyl-3,4-dihydro-2H-chromen-6-yl)sulfonyl]carbamimidamido}phenyl)acetic acid

To a solution of methyl (2S)-{[(1-benzyl-2-oxo-1,2-dihydropyridin-3-yl)carbonyl]amino}(3-{[(2,2,5,7,8-pentamethyl-3,4-dihydro-2H-chromen-6-yl)sulfonyl]carbamimidamido}phenyl)acetate (0.48 g, 0.69 mmol) in 1:6 MeOH/THF (3.5 mL) was added 2M NaOH (1.5 mL, 3.0 mmol) and then the reaction was stirred at room temperature for 2 hr. The reaction mixture was diluted with water, washed with diethyl ether (2 times) and the layers separated. The aqueous phase was acidified with 2M HCl and extracted with EtOAc. The organic layer was washed with water and saturated NaCl, dried over MgSO₄, filtered. The filtrate was concentrated under reduced pressure and the residue was purified by column chromatography on silica gel, eluting with a 1:1 EtOAc/hexanes to 5:1 EtOAc/hexanes gradient, followed sequentially by 1:20 MeOH/EtOAc, 1: 10 MeOH/EtOAc, 1:20 MeOH/CHCl₃, and finally 1:10 MeOH/CHCl₃ to give an off-white solid (0.20 g, 43%).

### VI. (2S)-{[(1-Benzyl-2-oxo-1,2-dihydropyridin-3-yl)carbonyl]amino}(3-carbamimidamidophenyl)acetic acid•TFA

To a solution of (2S)-{[(1-benzyl-2-oxo-1,2-dihydropyridin-3-yl)carbonyl]amino}(3-{[(2,2,5,7,8-pentamethyl-3,4-dihydro-2H-chromen-6-yl)sulfonyl]carbamimidamido}phenyl)acetic acid (0.16 g, 0.23 mmol) in TFA (3 mL), triethylsilane (0.3 mL) and water (0.3 mL) were added. After stirring the reaction mixture at room temperature for 3 hr, MTBE (30 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized. The resulting solid was purified by reverse-phase HPLC eluting with a 10 to 60% CH₃CN:0.1% aqueous TFA gradient to give an off-white solid after lyophilization (35 mg, 28%).

The following compound was synthesized by modifications of the general procedure described in Example 20.

Compound 78, (2S)-{[(1-benzyl-2-oxo-1,2-dihydropyridin-3-yl)carbonyl]amino}(3-{[(2,2,5,7,8-pentamethyl-3,4-dihydro-2H-chromen-6-yl)sulfonyl]carbamimidamido}phenyl)acetic acid, was obtained as a by-product from Step VI of Example 20.

### Example 21

### Compound 77

### Synthesis of (2S)-2-({[1-(diphenylmethyl)-2-oxo-1,2-dihydropyridin-3-yl]carbonyl}amino)-5-{[(4-methylphenyl)sulfonyl]carbamimidamido}pentanoic acid

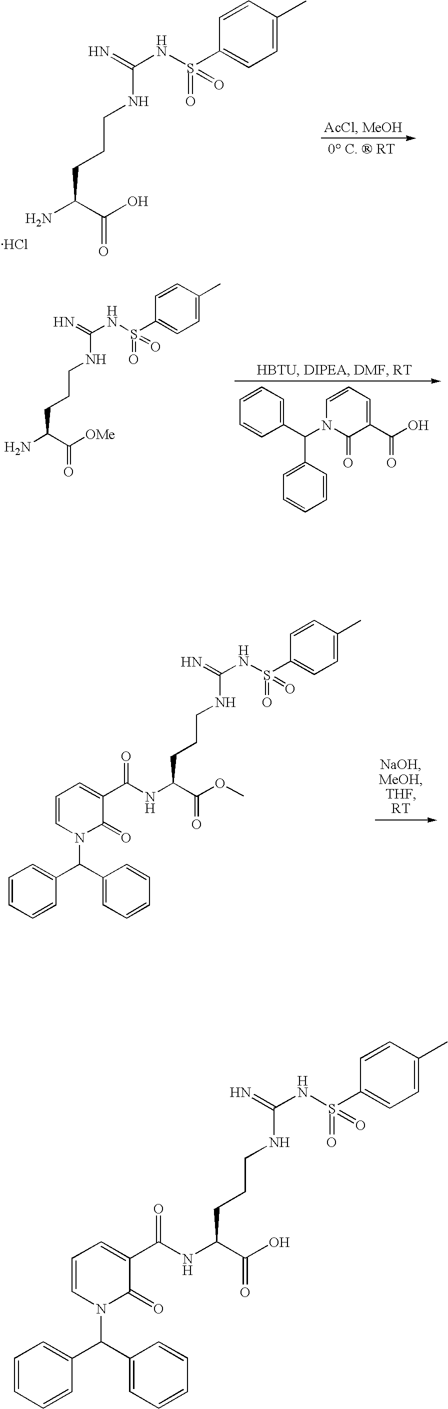

### I. Methyl (2S)-2-amino-5-{[(4-methylphenyl)sulfonyl]carbamimidamido}pentanoate

A solution of acetyl chloride (4.1 mL, 57.7 mmol) in methanol (75 mL) cooled to 0° C. was added via syringe to solid (2S)-2-amino-5-{[(4-methylphenyl)sulfonyl]carbamimidamido}pentanoic acid hydrochloride (1.00 g, 02.89 mmol). The reaction was gradually warmed room temperature and stirred overnight. The solvent was removed under reduced pressure and the crude product was purified by column chromatography on silica gel, eluting with 9:1 dichloromethane:methanol to give a pale yellow oil (yield not determined).

### II. Methyl (2S)-2-({[1-(diphenylmethyl)-2-oxo-1,2-dihydropyridin-3-yl]carbonyl}amino)-5-{[(4-methylphenyl)sulfonyl]carbamimidamido}pentanoate

To a solution of methyl (2S)-2-amino-5-{[(4-methylphenyl)sulfonyl]carbamimidamido}pentanoate (198 mg, 0.55 mmol), 1-(diphenylmethyl)-2-oxo-1,2-dihydropyridine-3-carboxylic acid (152 mg, 0.50 mmol), and HBTU (265 mg, 0.70 mmol) in DMF (5 mL) was added DIPEA (0.36 mL, 2.0 mmol). The reaction mixture was stirred at room temperature overnight and then diluted with EtOAc. The organic solution was washed successively with water and saturated NaCl, dried over Na₂SO₄ and filtered. The filtrate was concentrated under reduced pressure and the residue was purified by column chromatography on silica gel, eluting with 100% EtOAc to give a yellow oil (278 mg, 88% over 2 steps).

### III. (2S)-2-({[1-(Diphenylmethyl)-2-oxo-1,2-dihydropyridin-3-yl]carbonyl}amino)-5-{[(4-methylphenyl)sulfonyl]carbamimidamido} pentanoic acid

To a solution of methyl (2S)-2-({[1-(diphenylmethyl)-2-oxo-1,2-dihydropyridin-3-yl]carbonyl}amino)-5-{[(4-methylphenyl)sulfonyl]carbamimidamido}pentanoate (278 mg, 0.44 mmol) in 1:1 THF/MeOH (12 mL) was added 2M NaOH (1.3 mL, 2.6 mmol) and stirred at room temperature for 3 hr. The reaction was diluted with water, washed with diethyl ether (2 times) and the layers separated. The aqueous phase was acidified with 2M HCl and extracted with EtOAc. The organic layer was washed with water and saturated NaCl, dried over Na₂SO₄, filtered and the solvent was removed under reduced pressure. Purification of the resulting solid by reverse-phase HPLC eluting with a 1:9 CH₃CN:0.1% aqueous TFA gave a white solid after lyophilization (166 mg, 61%).

The following compounds were synthesized by modifications of the general procedure described in Example 21.

Compound 73: Step II was conducted using 225 mg compound 1-1, 265 mg L-nitroarginine methyl ester hydrochloride, 520 mg HBTU, and 0.61 mL DIPEA in 5 mL DMF to provide 207 mg methyl (2S)-2-{[(1-benzyl-2-oxo-1,2-dihydropyridin-3-yl)carbonyl]amino}-5-(nitrocarbamimidamido)pentanoate. Step III was conducted using 207 mg methyl (2S)-2-{[(1-benzyl-2-oxo-1,2-dihydropyridin-3-yl)carbonyl]amino}-5-(nitrocarbamimidamido)pentanoate and 1.9 mL 2 M NaOH in 8 mL 1:1 THF/MeOH to provide 89 mg (2S)-2-{[(1-benzyl-2-oxo-1,2-dihydropyridin-3-yl)carbonyl]amino}-5-(nitrocarbamimidamido)pentanoic acid following aqueous workup and reversed-phase HPLC purification.

Compound 74 and 75: Synthesized analogously as for Compound 73, except 100 mg compound 2-2 (used in place of 1-1), 107 mg L-nitroarginine methyl ester hydrochloride, 117 mg HBTU, and 0.10 mL DIPEA in 2 mL DMF were used to conduct the synthesis and provide 108 mg methyl (2S)-2-({[1-(diphenylmethyl)-2-oxo-1,2-dihydropyridin-3-yl]carbonyl}amino)-5-(nitrocarbamimidamido)pentanoate, 75. Step III was conducted using 76 mg methyl (2S)-2-({[1-(diphenylmethyl)-2-oxo-1,2-dihydropyridin-3-yl]carbonyl}amino)-5-(nitrocarbamimidamido)pentanoate and 0.6 mL 2 M NaOH in 6 mL 1:1 THF/MeOH to provide 21 mg (2S)-2-({[1-(diphenylmethyl)-2-oxo-1,2-dihydropyridin-3-yl]carbonyl}amino)-5-(nitrocarbamimidamido)pentanoic acid, 74 following aqueous workup.

Compound 76: Step II was conducted using 116 mg Compound 1-1, 200 mg methyl (2S)-2-amino-5-{[(4-methylphenyl)sulfonyl]carbamimidamido}pentanoate, 269 mg HBTU and 0.36 mL DIPEA in 5 mL DMF to provide 184 mg methyl (2S)-2-{[(1-benzyl-2-oxo-1,2-dihydropyridin-3-yl)carbonyl]amino}-5-{[(4-methylphenyl)sulfonyl]carbamimidamido}pentanoate. Step III was conducted using 184 mg methyl (2S)-2-{[(1-benzyl-2-oxo-1,2-dihydropyridin-3-yl)carbonyl]amino}-5-{[(4-methylphenyl)sulfonyl]carbamimidamido}pentanoate and 1.0 mL 2 M NaOH in 10 mL 1:1 THF/MeOH to provide 109 mg (2S)-2-{[(1-benzyl-2-oxo-1,2-dihydropyridin-3-yl)carbonyl]amino}-5-{[(4-methylphenyl)sulfonyl]carbamimidamido} pentanoic acid following reversed-phase HPLC purification.

### Example 22

### Compound 80

### Synthesis of methyl (2S)-5-carbamimidamido-2-({[1-(diphenylmethyl)-2-oxo-1,2-dihydropyridin-3-yl]carbonyl}amino)pentanoate•TFA

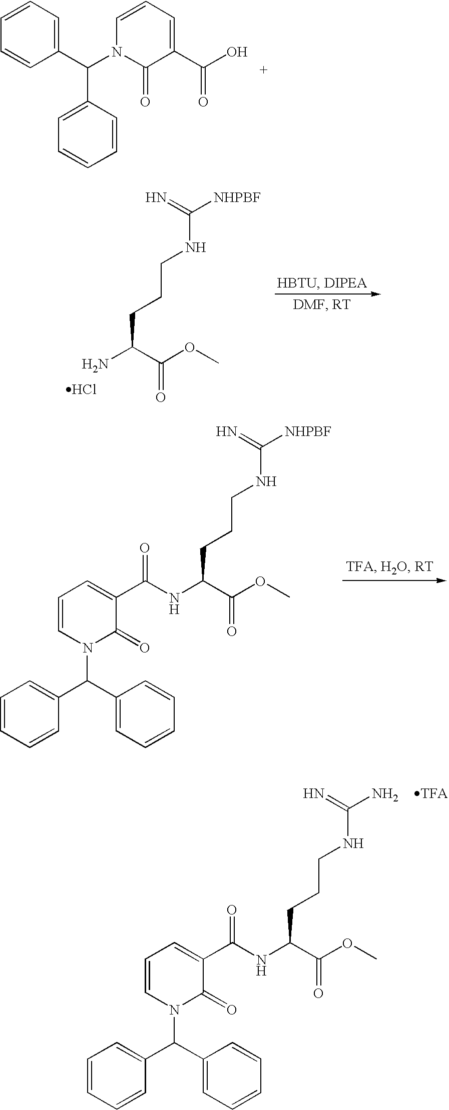

### I. Methyl (2S)-2-({[1-(diphenylmethyl)-2-oxo-1,2-dihydropyridin-3-yl]carbonyl}amino)-5-({2,2,4,6,7-pentamethyl-2,3-dihydrobenzofuran-5-yl}sulfonyl) carbamimidamido}pentanoate

To a solution of methyl (2S)-2-amino-5-[({2,2,4,6,7-pentamethyl-2,3-dihydrobenzofuran-5-yl}sulfonyl]carbamimidamido}pentanoate hydrochloride (156 mg, 0.33 mmol), 1-(diphenylmethyl)-2-oxo-1,2-dihydropyridine-3-carboxylic acid (100 mg, 0.33 mmol), and HBTU (175 mg, 0.46 mmol) in DMF (2 mL) was added DIPEA (0.21 mL, 1.16 mmol). The reaction mixture was stirred at room temperature overnight, diluted with EtOAc, washed successively with water and saturated NaCl, dried over Na₂SO₄ and filtered. The filtrate was concentrated under reduced pressure and the residue was purified by column chromatography on silica gel, eluting with 3:1 EtOAc/hexanes followed by 100% EtOAc (267 mg). The product was used in the next step without further purification.

### II. Methyl (2S)-5-carbamimidamido-2-({[1-(diphenylmethyl)-2-oxo-1,2-dihydropyridin-3-yl]carbonyl}amino)pentanoate•TFA

To a solution of methyl (2S)-2-({[1-(diphenylmethyl)-2-oxo-1,2-dihydropyridin-3-yl]carbonyl}amino)-5-({2,2,4,6,7-pentamethyl-2,3-dihydrobenzofuran-5-yl}sulfonyl) carbamimidamido}pentanoate (267 mg, 0.22 mmol theoretical) in TFA (2 mL) was added water (0.2 mL). After stirring at room temperature for 5 hr, MTBE (30 mL) was added resulting in formation of a white precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized. The resulting solid was purified by reverse-phase HPLC eluting with a 10 to 60% CH₃CN:0.1% aqueous TFA gradient to give a white solid after lyophilization (78 mg, 29% over 2 steps).

### Example 23

### Compound 81

### Synthesis of N-[(1S)-4-carbamimidamido-1-carbamoylbutyl]-1-(diphenylmethyl)-2-oxo-1,2-dihydropyridine-3-carboxamide-TFA

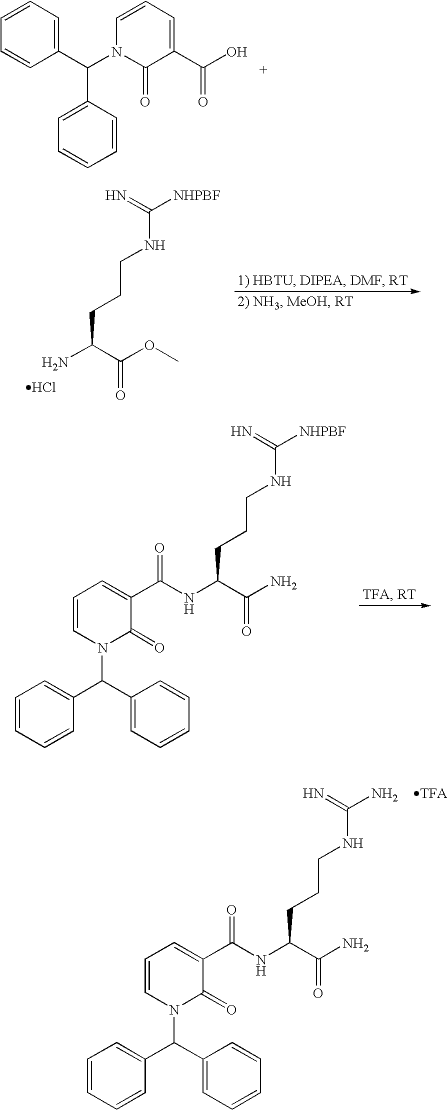

### I. Methyl (2S)-2-({[1-(diphenylmethyl)-2-oxo-1,2-dihydropyridin-3-yl]carbonyl}amino)-5-{[(2,2,4,6,7-pentamethyl-2,3-dihydro-1-benzofuran-5-yl)sulfonyl]carbamimidamido}pentanoate

To a solution of methyl (2S)-2-amino-5-{[(2,2,4,6,7-pentamethyl-2,3-dihydro-1-benzofuran-5-yl)sulfonyl]carbamimidamido}pentanoate hydrochloride (400 mg, 0.84 mmol), 1-(diphenylmethyl)-2-oxo-1,2-dihydropyridine-3-carboxylic acid (256 mg, 0.84 mmol), and HBTU (446 mg, 1.18 mmol) in DMF (4 mL) was added DIPEA (0.52 mL, 2.94 mmol). The reaction mixture was stirred at room temperature overnight, diluted with EtOAc, washed successively with water and saturated NaCl, dried over Na₂SO₄ and filtered. The filtrate was concentrated under reduced pressure and the residue was purified by column chromatography on silica gel, eluting with a 4:1 EtOAc/texanes to 100% EtOAc gradient to give a pale yellow oil (637 mg). The product was used in the next step without further purification.

### II. N-[(1S)-1-Carbamoyl-4-{[(2,2,4,6,7-pentamethyl-2,3-dihydro-1-benzofuran-5-yl)sulfonyl]carbamimidamido}butyl]-1-(diphenylmethyl)-2-oxo-1,2-dihydropyridine-3-carboxamide

A solution of methyl (2S)-2-({[1-(diphenylmethyl)-2-oxo-1,2-dihydropyridin-3-yl]carbonyl}amino)-5-{[(2,2,4,6,7-pentamethyl-2,3-dihydro-1-benzofuran-5-yl)sulfonyl]carbamimidamido}pentanoate (164 mg, 0. mmol) was prepared in MeOH (5 mL). Anhydrous NH₃ gas was then bubbled into the solution for 5 minutes, and then the reaction mixture was stirred at room temperature overnight and then concentrated to dryness under reduced pressure. The residue was purified by column chromatography on silica gel, eluting successively with 100% EtOAc, 1:99 MeOH/CH₂Cl₂, and finally 1:19 MeOH/CH₂Cl₂ to give a white foam (158 mg, 98% over 2 steps).

### III. N-[(1S)-4-carbamimidamido-1-carbamoylbutyl]-1-(diphenylmethyl)-2-oxo-1,2-dihydropyridine-3-carboxamide-TFA

A solution of N-[(1S)-1-carbamoyl-4-{[(2,2,4,6,7-pentamethyl-2,3-dihydro-1-benzofuran-5-yl)sulfonyl]carbamimidamido}butyl]-1-(diphenylmethyl)-2-oxo-1,2-dihydropyridine-3-carboxamide (158 mg, 0.22 mmol) in TFA (2 mL) was stirred at room temperature for 1.5 hr, then MTBE (20 mL) was added resulting in formation of a yellow precipitate. The solid was isolated by centrifugation, and the MTBE supernatant was removed by decantation. The remaining solid was triturated with additional MTBE and centrifuged again, and the MTBE was removed by decantation. The solid was dissolved in CH₃CN/H₂O and the resulting solution was lyophilized. The resulting solid was purified by reverse-phase HPLC eluting with a 10 to 60% CH₃CN:0.1% aqueous TFA gradient to give a white solid after lyophilization (37 mg, 29%).

### Example 24

### Compound 82

### Synthesis of (2S)-2-({[1-(diphenylmethyl)-2-oxo-1,2-dihydropyridin-3-yl]carbonyl}amino)-5-{[{(ethoxycarbonyl)amino}(isopropylamino)methylene]amino}pentanoic acid

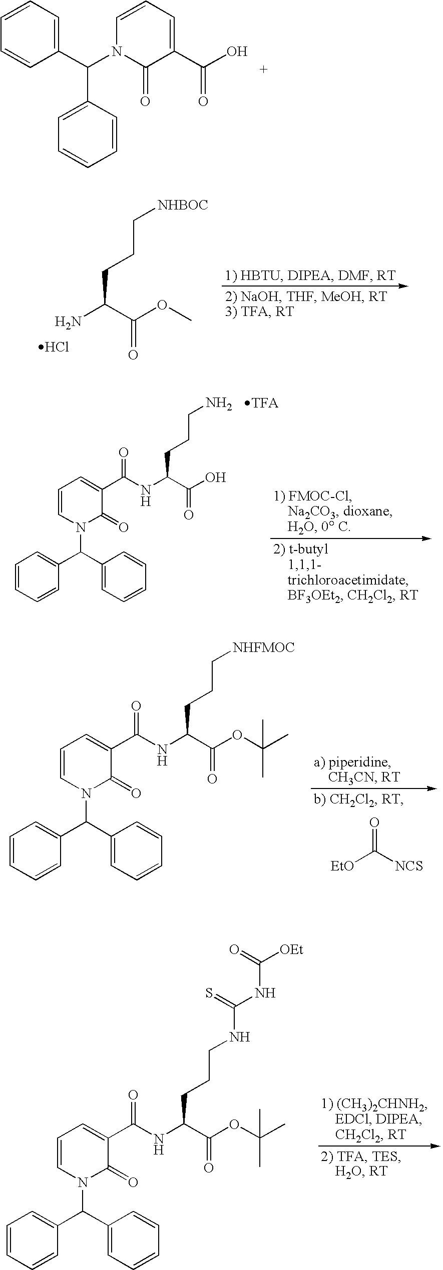

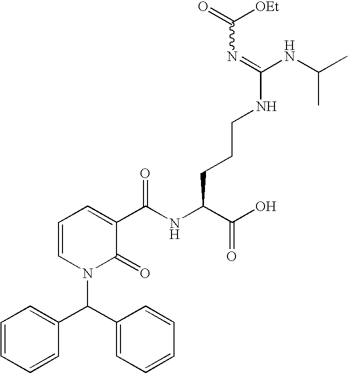

### I. Methyl (2S)-5-[(tert-butoxycarbonyl)amino]-2-({[1-(diphenylmethyl)-2-oxo-1,2-dihydropyridin-3-yl]carbonyl}amino)pentanoate

To a solution of methyl (2S)-2-amino-5-[(tert-butoxycarbonyl)amino]pentanoate hydrochloride chloride (1.00 g, 3.54 mmol), 1-benzyl-2-oxo-1,2-dihydropyridine-3-carboxylic acid (1.08 g, 3.54 mmol), and HBTU (1.88 g, 4.96 mmol) in DMF (20 mL) was added DIPEA (202 mL, 12.4 mmol). The reaction mixture was stirred overnight at room temperature and then diluted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 1:1 EtOAc/hexanes to give a pale yellow foam (1.81 g, 96%).

### II. (2S)-5-[(tert-Butoxycarbonyl)amino]-2-({[1-(diphenylmethyl)-2-oxo-1,2-dihydropyridin-3-yl]carbonyl}amino)pentanoic acid

To a solution of methyl (2S)-5-[(tert-butoxycarbonyl)amino]-2-({[1-(diphenylmethyl)-2-oxo-1,2-dihydropyridin-3-yl]carbonyl}amino)pentanoate (1.81 g, 3.4 mmol) in 1:1 MeOH/THF (40 mL) was added 2M NaOH (10.2 mL, 20.4 mmol) and then stirred at room temperature for 4 hr. The reaction mixture was diluted with water, washed with diethyl ether (2 times) and the layers separated. The aqueous phase was acidified with 2M HCl and extracted with EtOAc. The organic layer was washed with water and saturated NaCl, dried over Na₂SO₄, and filtered. The filtrate was concentrated under reduced pressure to give a pale yellow foam (1.89 g).

### III. (2S)-5-Amino-2-({[1-(diphenylmethyl)-2-oxo-1,2-dihydropyridin-3-yl]carbonyl}amino)pentanoic acid•TFA

To a solution of (2S)-5-[(tert-butoxycarbonyl)amino]-2-({[1-(diphenylmethyl)-2-oxo-1,2-dihydropyridin-3-yl]carbonyl}amino)pentanoic acid (390 mg, 0.75 mmol theoretical) in TFA (3 mL) was added triethylsilane (0.3 mL) and water (0.4 mL). After stirring at room temperature for 2 hr, the reaction mixture was concentrated to dryness under reduced pressure. The residual oil was treated with excess diethyl ether, resulting in formation of a white precipitate. The precipitate was isolated by filtration, washed with Et₂O, and dried under vacuum to give a white solid (360 mg, 90%).

### IV. (2S)-2-({[1-(Diphenylmethyl)-2-oxo-1,2-dihydropyridin-3-yl]carbonyl}amino)-5-{[(9H-fluoren-9-ylmethoxy)carbonyl]amino}pentanoic acid

To a suspension of (2S)-5-amino-2-({[1-(diphenylmethyl)-2-oxo-1,2-dihydropyridin-3-yl]carbonyl}amino)pentanoic acid•TFA (1.05 g, 1.97 mmol) in water (11 mL) was added solid Na₂CO₃ (0.98 g, 11.82 mmol), and the reaction mixture was cooled to 0° C. in an ice bath. A solution of FMOC chloride (607 mg, 2.26 mmol) in 1,4-dioxane (18 mL) was added dropwise to the reaction mixture, and the combined solution was stirred 1.5 hr at 0° C. and then diluted with water. The aqueous solution was washed with diethyl ether (2 times) and acidified with 2 M HCl forming a white precipitate which was extracted into EtOAc. The EtOAc extract was washed successively with water and saturated NaCl, dried over Na₂SO₄ and filtered. The filtrate was concentrated under reduced pressure to give the product (1.46 g).

### V. tert-Butyl (2S)-2-({[1-(diphenylmethyl)-2-oxo-1,2-dihydropyridin-3-yl]carbonyl}amino)-5-{[(9H-fluoren-9-ylmethoxy)carbonyl]amino}pentanoate

To a solution of (2S)-2-({[1-(diphenylmethyl)-2-oxo-1,2-dihydropyridin-3-yl]carbonyl}amino)-5-{[(9H-fluoren-9-ylmethoxy)carbonyl]amino}pentanoic acid (1.20 g, 1.88 mmol) in CH₂Cl₂ (12 mL) was added t-butyl 1,1,1-trichloroacetimidate (0.51 mL, 2.85 mmol) and BF₃.OEt₂ (0.054 mL, 0.47 mmol). After stirring over 2 days at room temperature, the reaction was quenched with water, and the product was extracted into EtOAc. The EtOAc extract was washed successively with water and saturated NaCl, dried over Na₂SO₄ and filtered. The filtrate was concentrated under reduced pressure and the residue was purified by column chromatography on silica gel eluting with 1:1 EtOAc/hexanes to give a white solid (1.03 g, 79%).

### VI. tert-Butyl (2S)-2-({[1-(diphenylmethyl)-2-oxo-1,2-dihydropyridin-3-yl]carbonyl}amino)-5-{[(ethoxycarbonyl)carbamothioyl]amino}pentanoate

To a solution of tert-butyl (2S)-2-({[1-(diphenylmethyl)-2-oxo-1,2-dihydropyridin-3-yl]carbonyl}amino)-5-{[(9H-fluoren-9-ylmethoxy)carbonyl]amino}pentanoate (500 mg, 0.72 mmol) in CH₃CN (5 mL) was added piperidine (1 mL), and the reaction was stirred at room temperature for 1 hr. The reaction mixture was then taken to dryness under reduced pressure and then redissolved in CH₂Cl₂. The CH₂Cl₂ was removed under reduced pressure, and the redissolution/evaporation sequence was repeated twice more. The crude product was then dried under high vacuum for several hours until the odor of residual piperidine was no longer present. The crude amine product was redissolved in CH₂Cl₂ (5 mL), and to the solution was added ethoxycarbonyl isothiocyanate (0.12 mL, 1.08 mmol). The reaction was stirred at room temperature overnight, diluted with water, and the product was extracted into EtOAc. The EtOAc extract was washed successively with water and saturated NaCl, dried over Na₂SO₄ and filtered. The filtrate was concentrated under reduced pressure and the residue was purified by column chromatography on silica gel with a 1:3 EtOAc/hexanes to 100% EtOAc gradient to give an orange oil (158 mg, 36%).

### VII. tert-Butyl (2S)-2-({[1-(diphenylmethyl)-2-oxo-1,2-dihydropyridin-3-yl]carbonyl}amino)-5-{[{(ethoxycarbonyl)amino}(isopropylamino)methylene]amino}pentanoate

To a solution of tert-butyl (2S)-2-({[1-(diphenylmethyl)-2-oxo-1,2-dihydropyridin-3-yl]carbonyl}amino)-5-{[(ethoxycarbonyl)carbamothioyl]amino}pentanoate (79 mg, 0.13 mmol) in CH₂Cl₂ (2 mL) was added isopropylamine (0.017 mL, 0.20 mmol) and DIPEA (0.025 mL, 0.14 mmol), followed by EDCI (58 mg, 0.30 mmol). The reaction mixture was stirred at room temperature overnight and then diluted with EtOAc. The EtOAc solution was washed sequentially with water (2 times) and saturated NaCl, dried over Na₂SO₄ and filtered. The filtrate was concentrated under reduced pressure and the residue was purified by chromatography on silica gel eluting with a 1:1 EtOAc/hexanes to 100% EtOAc gradient to give a colorless oil (56 mg, 68%).

### VIII. (2S)-2-({[1-(Diphenylmethyl)-2-oxo-1,2-dihydropyridin-3-yl]carbonyl}amino)-5-{[{(ethoxycarbonyl)amino}(isopropylamino)methylene]amino}pentanoic acid

To a solution of tert-butyl (2S)-2-({[1-(diphenylmethyl)-2-oxo-1,2-dihydropyridin-3-yl]carbonyl}amino)-5-{[{(ethoxycarbonyl)amino}(isopropylamino)methylene]amino}pentanoate (0.1 mL) and water (0.1 mL) were added. After stirring the at room temperature for 1.5 hr, and the reaction was diluted with water and extracted with diethyl ether. The organic layer was separated, washed with water (2 times) and extracted with 2M NaOH. The basic aqueous extract was washed with diethyl ether (2 times), and the aqueous phase was then acidified with 2 M HCl, resulting in formation of a white precipitate. The product was extracted into EtOAc, and the extract was washed successively with water and saturated NaCl, dried over Na₂SO₄ and filtered. The filtrate was concentrated under reduced pressure to give a colorless oil. The oil was redissolved in CH₃CN/H₂O and lyophilized to give a white solid (47 mg, 92%).

The following compounds were synthesized by modifications of the general procedure described in Example 24

Compound 69: Step VI was conducted using 187 mg tert-butyl (2S)-2-({[1-(diphenylmethyl)-2-oxo-1,2-dihydropyridin-3-yl]carbonyl}amino)-5-{[(9H-fluoren-9-ylmethoxy)carbonyl]amino}pentanoate and 0.4 mL piperidine in 2 mL CH₃CN. Following removal of the CH₃CN solvent and piperidine, the residue was redissolved in 2 mL anhydrous CH₂Cl₂ and treated with 31 μL methanesulfonyl chloride and 0.15 mL DIPEA to provide 36 mg tert-butyl (2S)-2-({[1-(diphenylmethyl)-2-oxo-1,2-dihydropyridin-3-yl]carbonyl}amino)-5-[(methylsulfonyl)amino]pentanoate. Step VII was not performed for this analog. Step VIII was conducted using 36 mg tert-butyl (2S)-2-({[1-(diphenylmethyl)-2-oxo-1,2-dihydropyridin-3-yl]carbonyl}amino)-5-[(methylsulfonyl)amino]pentanoate, 1 mL TFA, 0.1 mL triethylsilane, and 0.1 mL H₂O. The reaction mixture was diluted with deionized H₂O and made basic with 2 M NaOH. The aqueous solution was washed twice with Et₂O and then acidified with 2 M HCl. The product was extracted into EtOAc, and the EtOAc phase was washed twice with deionized H₂O and once with saturated aqueous NaCl. The organic layer was dried over anhydrous Na₂SO₄, filtered and concentrated, to provide 20 mg (S)-2-(1-benzhydryl-2-oxo-1,2-dihydropyridine-3-carboxamido)-5-(methylsulfonamido)pentanoic acid.

Compound 83: Step VII was conducted with 49 mg tert-butyl (2S)-2-({[1-(diphenylmethyl)-2-oxo-1,2-dihydropyridin-3-yl]carbonyl}amino)-5-{[(ethoxycarbonyl)carbamothioyl]amino}pentanoate, 23 mg EDCI and 16 μL DIPEA in 2 mL CH₂Cl₂. Excess NH₃ gas was bubbled into the solution. After overnight stirring, 26 mg tert-butyl (2S)-2-({[1-(diphenylmethyl)-2-oxo-1,2-dihydropyridin-3-yl]carbonyl}amino)-5-[(ethoxycarbonyl)carbamimidamido]pentanate was isolated. Step VIII was conducted using 26 mg mg tert-butyl (2S)-2-({[1-(diphenylmethyl)-2-oxo-1,2-dihydropyridin-3-yl]carbonyl}amino)-5-[(ethoxycarbonyl)carbamimidamido]pentanoate, 1 mL TFA, 0.1 mL triethylsilane and 0.1 mL deionized H₂O to provide 11 mg (2S)-2-({[1-(diphenylmethyl)-2-oxo-1,2-dihydropyridin-3-yl]carbonyl}amino)-5-[(ethoxycarbonyl)carbamimidamido]pentanoic acid following reversed-phase HPLC purification.

### Example 25

### Compound 84

### Synthesis of (2S)-2-[{(3-benzyl-5-methyl-2-oxopyridin-1(2H)-yl)acetyl]amino}-5-carbamimidamidopentanoic acid•TFA

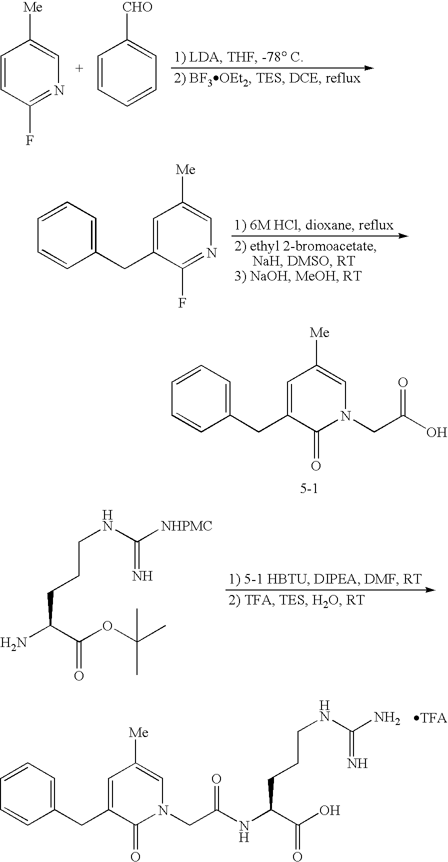

### I. (2-Fluoro-5-methylpyridin-3-yl)(phenyl)methanol

To a solution of LDA (1.8M in THF/heptane/ethylbenzene, 75 mL, 135 mmol) in THF (75 mL) cooled to −78° C. was added a solution of 2-fluoro-5-methylpyridine (9.3 mL, 90 mmol) in THF (150 mL) slowly via canula over 20 min. Stirring was continued for 2.75 h at −78° C. and then benzaldehyde (9.1 mL, 90 mmol) was rapidly added to the reaction mixture. After stirring an additional 1.5 h at −78° C., the reaction was quenched with water, allowed to warm to room temperature and then the product was extracted into EtOAc. The organic layer was separated and washed successively with water and saturated NaCl, dried over Na₂SO₄ and filtered. The filtrate was concentrated under reduced pressure to give an orange oil. The crude product was without purification (18.7 g, 96%)

### II. 3-Benzyl-2-fluoro-5-methylpyridine

To a solution of (2-fluoro-5-methylpyridin-3-yl)(phenyl)methanol (18.7 g, 86.2 mmol) in 1,2-dichloroethane (120 mL) was added BF₃—OEt₂ (55 mL, 431 mmol) and triethylsilane (25 mL, 155 mmol). The reaction mixture was heated at reflux for 2 hr, cooled to room temperature, quenched with water and extracted with EtOAc. The organic layer was washed successively with water (3 times) and saturated NaCl, dried over Na₂SO₄ and filtered. The filtrate was concentrated under reduced pressure to give a red-orange oil (15.1 g, 87%).

### III. 3-Benzyl-5-methylpyridin-2(1H)-one

To a solution of 3-benzyl-2-fluoro-5-methylpyridine (15.1 g, 75 mmol) in 1,4-dioxane (60 mL) was added 6M HCl (210 mL). The reaction mixture was heated at reflux overnight, cooled to room temperature, diluted with water and extracted with EtOAc. The organic layer was washed successively with water (2 times) and saturated NaCl, dried over Na₂SO₄ and filtered. The filtrate was concentrated under reduced pressure and the residue was purified by column chromatography on silica gel, eluting with 9:1 EtOAc:hexanes followed by 100% EtOAc to give an orange solid (6.27 g, 42%).

### IV. Ethyl 2-(3-benzyl-5-methyl-2-oxopyridin-1(2H)-yl)acetate

Sodium hydride (60%, 82 mg, 2.06 mmol) was added to a solution of 3-benzyl-5-methylpyridin-2(1H)-one (0.39 g, 1.96 mmol) in DMSO (8 mL). The resulting suspension was stirred at room temperature for 30 min and then ethyl 2-bromoacetate (0.26 mL, 2.35 mmol) was added. After stirring at room temperature for 2 days, the reaction was quenched with 2M HCl and extracted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 2:3 EtOAc:hexanes followed by 1:1 EtOAc:hexanes to give a yellow oil (0.35 g, 63%).

### V. 2-(3-Benzyl-5-methyl-2-oxopyridin-1(2H)-yl)acetic acid

To a solution of ethyl 2-(3-benzyl-5-methyl-2-oxopyridin-1(2H)-yl)acetate (0.35 g, 1.23 mmol) in methanol (4 mL) was added 2M NaOH (1.5 mL, 3.0 mmol) and the reaction was stirred at room temperature for 2 hr. The reaction was diluted with water, and the resulting solution was washed twice with diethyl ether. Acidification of the aqueous phase produced a white precipitate which was extracted into EtOAc. The organic phase was washed sequentially with H₂O and with saturated NaCl, dried over Na₂SO₄ and filtered. The filtrate was concentrated under reduced pressure to give a white solid (0.25 g, 78%).

### VI. tert-Butyl (2S)-2-{[(3-benzyl-5-methyl-2-oxopyridin-1(2H)-yl)acetyl]amino}-5-{[(2,2,5,7,8-pentamethyl-3,4-dihydro-2H-chromen-6-yl)sulfonyl]carbamimidamido}pentanoate

To a solution of tert-butyl (2S)-2-amino-5-{[(2,2,5,7,8-pentamethyl-3,4-dihydro-2H-chromen-6-yl)sulfonyl]carbamimidamido}pentanoate (85 mg, 0.17 mmol), 2-(3-benzyl-5-methyl-2-oxopyridin-1(2H)-yl)acetic acid (40 mg, 0.16 mmol), and HBTU (83 mg, 0.22 mmol) in anhydrous DMF (1 mL) was added DIPEA (0.06 mL, 0.34 mmol). The reaction mixture was stirred 2 days at room temperature, quenched with 2M HCl and then extracted with EtOAc. The organic layer was washed with water (3 times) and saturated NaCl, dried over Na₂SO₄ and filtered. The filtrate was concentrated under reduced pressure and the residue was purified by column chromatography on silica gel, eluting with 3:7 EtOAc:hexanes followed by 100% EtOAc to give a white semi-solid (90 mg, 79%).

### VII. (2S)-2-{[(3-benzyl-5-methyl-2-oxopyridin-1(2H)-yl)acetyl]amino}-5-carbamimidamidopentanoic acid•TFA

To a solution of tert-butyl (2S)-2-{[(3-benzyl-5-methyl-2-oxopyridin-1(2H)-yl)acetyl]amino}-5-{[(2,2,5,7,8-pentamethyl-3,4-dihydro-2H-chromen-6-yl)sulfonyl]carbamimidamido}pentanoate (90 mg, 0.12 mmol) in TFA (0.9 mL), triethylsilane (0.05 mL) and water (0.05 mL) were added. After stirring the reaction mixture at room temperature for 2.5 hr, MTBE (20 mL) was added resulting in formation of a precipitate. The solid was isolated by filtration and washed with MTBE. The solid was dissolved in CH₃CN/H₂O and the resulting solution was lyophilized to give a solid. Purification by reverse-phase HPLC eluting with a 1:9 CH₃CN:0.1% aqueous TFA to 3:2 CH₃CN:0.1% aqueous TFA gradient gave a white solid after lyophilization (7 mg, 11%).

### Example 26

### Compound 86

### Synthesis of (2S)-2-({[1-(diphenylmethyl)-2-oxo-1,2-dihydropyridin-3-yl]carbonyl}amino)-5-(ethanimidoylamino)pentanoic acid•TFA

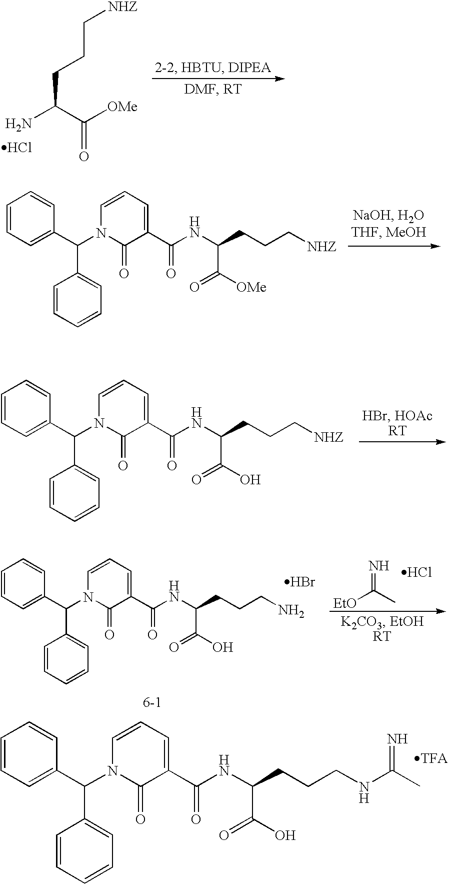

### I. Methyl (2S)-5-{[(benzyloxy)carbonyl]amino}-2-({[1-(diphenylmethyl)-2-oxo-1,2-dihydropyridin-3-yl]carbonyl}amino)pentanoate

A solution of methyl (2S)-2-amino-5-{[(benzyloxy)carbonyl]amino}pentanoate (1.56 g), 1-(diphenylmethyl)-2-oxo-1,2-dihydropyridine-3-carboxylic acid (1.50 g), diisopropylethylamine (3.07 g) and HBTU (2.61 g) in DMF(25 mL) were stirred at room temperature overnight. The resulting mixture was diluted with ethyl acetate, washed with water (3 times) and with brine. The organic layer was dried over MgSO₄, filtered and concentrated under reduced pressure. The residue was purified by automated silica gel column chromatography (Biotage®) eluting with 60% ethyl acetate/hexanes increasing to 100% ethyl acetate to give the title compound (2.783 g) as a white foam.

### II. (2S)-5-{[(Benzyloxy)carbonyl]amino}-2-({[1-(diphenylmethyl)-2-oxo-1,2-dihydropyridin-3-yl]carbonyl}amino)pentanoic acid

To a solution of methyl (2S)-5-{[(benzyloxy)carbonyl]amino}-2-({[1-(diphenylmethyl)-2-oxo-1,2-dihydropyridin-3-yl]carbonyl}amino)pentanoate (2.78 g) in THF (30 mL) and MeOH (30 mL), aqueous NaOH (2.0 M, 15 mL) was added. The resulting mixture was stirred at room temperature for 3 h, then was diluted with ether and water. The aqueous layer was washed with ether and then acidified with HCl (2M) and extracted with ethy acetate. The ethyl acetate layer was washed with water and brine, dried over Na₂SO₄, filtered and concentrated under reduced pressure to give the title compound (2.69 g) as a white foam.

### III. (2S)-5-Amino-2-({[1-(diphenylmethyl)-2-oxo-1,2-dihydropyridin-3-yl]carbonyl}amino)pentanoic acid hydrobromide

A suspension of (2S)-5-{[(benzyloxy)carbonyl]amino}-2-({[1-(diphenylmethyl)-2-oxo-1,2-dihydropyridin-3-yl]carbonyl}amino)pentanoic acid (2.69 g) in HBr in HOAc (33%, 20 mL) was stirred for 1.5 h at room temperature, by which time all solids had dissolved. The resulting mixture was diluted with water and extracted with ether (2 times). The aqueous layer was lyophilized to give the title compound (2.32 g).

### IV. (2S)-2-({[1-(Diphenylmethyl)-2-oxo-1,2-dihydropyridin-3-yl]carbonyl}amino)-5-(ethanimidoylamino)pentanoic acid•TFA

To a solution of (2S)-5-amino-2-({[1-(diphenylmethyl)-2-oxo-1,2-dihydropyridin-3-yl]carbonyl}amino)pentanoic acid hydrobromide (400 mg) in ethanol (10 mL) at room temperature, ethyl acetimidate hydrochloride (148 mg) and K₂CO₃ (480 mg) were added sequentially. The resulting suspension was stirred overnight and the resulting was filtered through Celite® and concentrated under reduced pressure. The residue was purifiec by reverse phase HPLC, eluting with acetonitrile/water/TFA mixture and the fractions containing the desired product were lyophilized to give the title compound (73.5 mg) as a white solid.

### Example 27

### Compound 88

### Synthesis of (2S)-2-({[1-(diphenylmethyl)-2-oxo-1,2-dihydropyridin-3-yl]carbonyl}amino)-6-(ethanimidoylamino)hexanoic acid•TFA

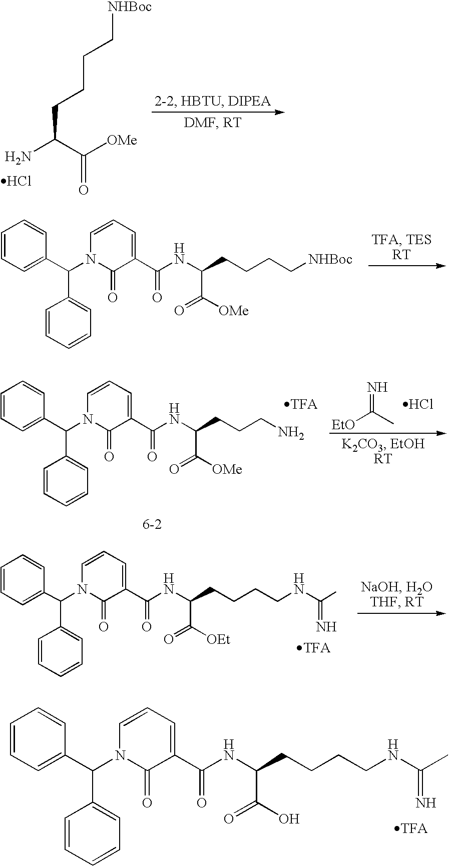

### I. Methyl (2S)-6-[(tert-butoxycarbonyl)amino]-2-({[1-(diphenylmethyl)-2-oxo-1,2-dihydropyridin-3-yl]carbonyl}amino)hexanoate

A solution of methyl (2S)-2-amino-6-[(tert-butoxycarbonyl)amino]hexanoatehydrochloride (546 mg), 1-(diphenylmethyl)-2-oxo-1,2-dihydropyridine-3-carboxylic acid (2-2, 560 mg), diisopropylethylamine (1.15 mL) and HBTU (976 mg) in DMF(9 mL) were stirred at room temperature overnight. The resulting mixture was diluted with ethyl acetate and washed with water (3 times) and brine. The organic layer was dried over Na₂SO₄, filtered and concentrated under reduced pressure. The residue was purified by automated silica gel column chromatography (Biotage™) to give the title compound (910 mg) as a yellow foam.

### II. Methyl (2S)-6-amino-2-({[1-(diphenylmethyl)-2-oxo-1,2-dihydropyridin-3-yl]carbonyl}amino)hexanoate-TFA

To a solution of methyl (2S)-6-[(tert-butoxycarbonyl)amino]-2-({[1-(diphenylmethyl)-2-oxo-1,2-dihydropyridin-3-yl]carbonyl}amino)hexanoate (910 mg) in TFA (5 mL), triethylsilane (0.5 mL) was added. The resulting mixture was stirred at room temperature for 1.5 hour and the reaction mixture was diluted with ether and concentrated under reduced pressure to give a yellow oil. This material was taken up in ether and concentrated to give a the title compound (840 mg) as an off-white solid.

### III. Ethyl (2S)-2-({[1-(diphenylmethyl)-2-oxo-1,2-dihydropyridin-3-yl]carbonyl}amino)-6-(ethanimidoylamino)hexanoate•TFA

To a solution of methyl (2S)-6-amino-2-({[1-(diphenylmethyl)-2-oxo-1,2-dihydropyridin-3-yl]carbonyl}amino)hexanoate•TFA (200 mg) in ethanol (4 mL) at room temperature, ethyl acetimidate hydrochloride (53 mg) was added and the reaction mixture was stirred at room temperature for 20 minutes. To the resulting mixture, K₂CO₃ (109 mg) was added and the resulting suspension was stirred overnight. Additional ethyl acetimidate hydrochloride (50 mg) and K₂CO₃ (100 mg) were added, the mixture was stirred an additional 6 hours, diluted with water and extracted with ether (3 times). The organic layers were combined, dried over Na₂SO₄, filtered and concentrated under reduced pressure. The aqueous layer was acidifid with HCl (2M) and lyophilized. The residue from both the organic layer and aquous layer were separately purified by reverse phase HPLC, eluting with acetonitrile/water/TFA mixture and the fractions from both purifications containing the desired product were combined and lyophilized to give the title compound (99 mg) as a white solid.

### IV. (2S)-2-({[1-(Diphenylmethyl)-2-oxo-1,2-dihydropyridin-3-yl]carbonyl}amino)-6-(ethanimidoylamino)hexanoic acid•TFA

To a solution of ethyl (2S)-2-({[1-(diphenylmethyl)-2-oxo-1,2-dihydropyridin-3-yl]carbonyl}amino)-6-(ethanimidoylamino)hexanoate•TFA (6-2, 99 mg) in THF (2 mL) and MeOH (2 mL), aqueous NaOH (2.0 M, 0.5 mL) was added. The resulting mixture was stirred at room temperature for 3 h, then was diluted with acetonitrile and water and was lyophilized. The resulting solid was purified by reverse phase HPLC, eluting with acetonitrile/water/TFA mixture and the fractions containing the desired product were combined and lyophilized to give the title compound (20.9 mg) as a white solid.

### Example 28

### Compound 94

### Synthesis of (2S)-2-({[1-(diphenylmethyl)-2-oxo-1,2-dihydropyridin-3-yl]carbonyl}amino)-4-(ethanimidoylamino)butanoic acid•TFA

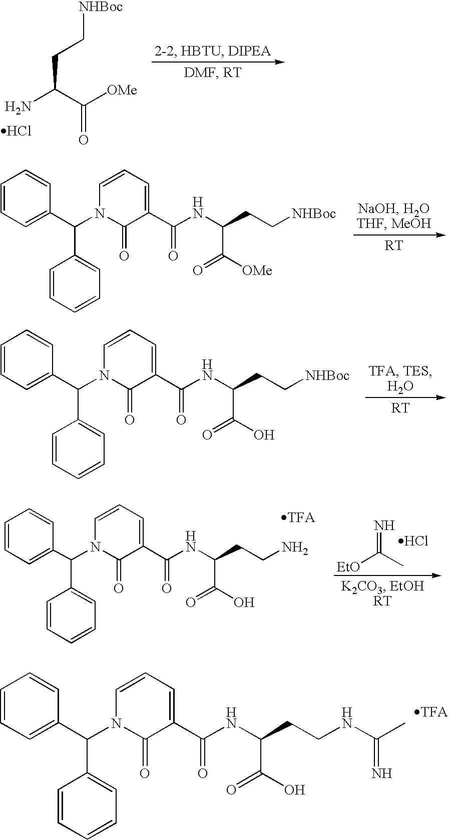

### I. Methyl (2S)-4-[(tert-butoxycarbonyl)amino]-2-({[1-(diphenylmethyl)-2-oxo-1,2-dihydropyridin-3-yl]carbonyl}amino)butanoate

A solution of methyl (2S)-2-amino-4-[(tert-butoxycarbonyl)amino]butanoate hydrochloride (1.00 g), 1-(diphenylmethyl)-2-oxo-1,2-dihydropyridine-3-carboxylic acid (2-2, 1.13 g), diisopropylethylamine (2.3 mL) and HBTU (1.98 g) in DMF(20 mL) was stirred at room temperature overnight. The resulting mixture was diluted with ethyl acetate, washed with water (3 times) and with brine. The organic layer was dried over MgSO₄, filtered and concentrated under reduced pressure. The residue was purified by automated silica gel column chromatography (Biotage™ eluting with 40% ethyl acetate/hexanes increasing to 50% ethyl acetate/hexanes to give the title compound (1.95 g) as a white foam.

### II. (2S)-4-[(tert-Butoxycarbonyl)amino]-2-({[1-(diphenylmethyl)-2-oxo-1,2-dihydropyridin-3-yl]carbonyl}amino)butanoic acid

To a solution of methyl (2S)-4-[(tert-butoxycarbonyl)amino]-2-({[1-(diphenylmethyl)-2-oxo-1,2-dihydropyridin-3-yl]carbonyl}amino)butanoate (1.20 g) in THF (15 mL) and MeOH (15 mL), aqueous NaOH (2.0 M, 6.9 mL) was added. The resulting mixture was stirred at room temperature for 3 h, then was diluted with ether and water. The aqueous layer was washed with ether and then acidified with HCl (2M) and extracted with ethy acetate. The ethyl acetate layer was washed with water and brine, dried over Na₂SO₄, filtered and concentrated under reduced pressure to give the title compound (1.16 g) as a white solid.

### III. (2S)-4-Amino-2-({[1-(diphenylmethyl)-2-oxo-1,2-dihydropyridin-3-yl]carbonyl}amino)butanoic acid•TFA

To a solution of (2S)-4-[(tert-butoxycarbonyl)amino]-2-({[1-(diphenylmethyl)-2-oxo-1,2-dihydropyridin-3-yl]carbonyl}amino)butanoic acid (1.16 mg) in TFA (4 mL), triethylsilane (0.4 mL) and water (0.4 mL) were added. The resulting mixture was stirred at room temperature for 2 hours and the reaction mixture was concentrated under reduced pressure to give a pale pink oil oil. This material was taken up in acetonitrile and water and lyophilized to give the title compound as a white solid.

### IV. (2S)-2-({[1-(Diphenylmethyl)-2-oxo-1,2-dihydropyridin-3-yl]carbonyl}amino)-4-(ethanimidoylamino)butanoic acid•TFA

To a solution of (2S)-4-amino-2-({[1-(diphenylmethyl)-2-oxo-1,2-dihydropyridin-3-yl]carbonyl}amino)butanoic acid•TFA (200 mg) in ethanol (5 mL) at room temperature, ethyl acetimidate hydrochloride (72 mg) and K₂CO₃ (215 mg) were added sequentially. The resulting suspension was stirred overnight and the resulting was filtered through Celite and concentrated under reduced pressure. The residue was purified by reverse phase HPLC, eluting with acetonitrile/water/TFA mixture and the fractions containing the desired product were lyophilized to give the title compound (73.5 mg) as a white solid.

### Example 29

### Compound 96

### Synthesis of (2S)-5-[(2-carboxyethanimidoyl)amino]-2-({[1-(diphenylmethyl)-2-oxo-1,2-dihydropyridin-3-yl]carbonyl}amino)pentanoic acid•TFA

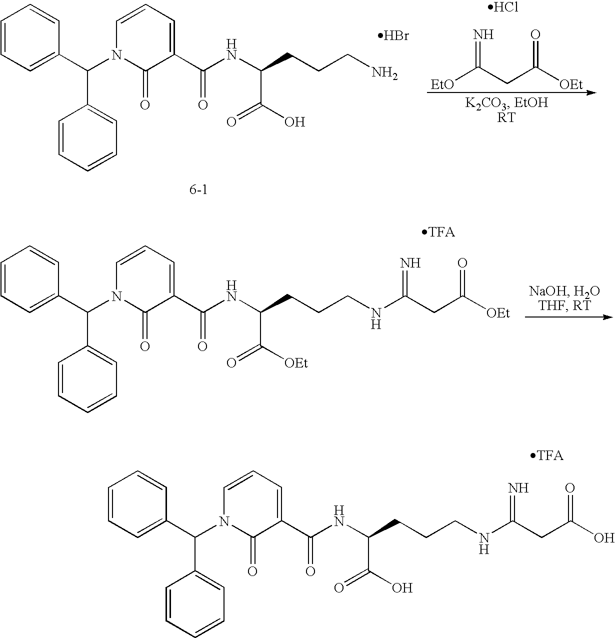

### I. (Ethyl (2S)-2-({[1-(diphenylmethyl)-2-oxo-1,2-dihydropyridin-3-yl]carbonyl}amino)-5-[(3-ethoxy-3-oxopropanimidoyl)amino]pentanoate-TFA.

To a solution of (S)-5-amino-2-(1-benzhydryl-2-oxo-1,2-dihydropyridine-3-carboxamido)pentanoic acid hydrobromide (6-1,600 mg) in ethanol (15 mL) at room temperature, ethyl 3-ethoxy-3-iminopropanoate hydrochloride (352 mg) and K₂CO₃ (660 mg) were added sequentially. The resulting suspension was stirred overnight and the resulting mixture was filtered through Celite® and concentrated under reduced pressure. The residue was purified by reverse phase HPLC, eluting with acetonitrile/water/TFA mixture and the fractions containing the desired product were lyophilized to give the title compound (160 mg) as a white solid.

### II. (2S)-5-[(2-Carboxyethanimidoyl)amino]-2-({[1-(diphenylmethyl)-2-oxo-1,2-dihydropyridin-3-yl]carbonyl}amino)pentanoic acid•TFA

To a solution of ethyl (2S)-2-({[1-(diphenylmethyl)-2-oxo-1,2-dihydropyridin-3-yl]carbonyl}amino)-5-[(3-ethoxy-3-oxopropanimidoyl)amino]pentanoate•TFA(160 mg) in THF (4 mL) and MeOH (4 mL), aqueous NaOH (2.0 M, 1.5 mL) was added. The resulting mixture was stirred at room temperature for 3 h, then was diluted with water and extracted with ether (twice). The aqueous layer was acidified with HCl (2M) and was extracted with ethyl acetate. The aqueous layer was lyophilized and the resulting solid was purified by reverse phase HPLC, eluting with acetonitrile/water/TFA mixture and the fractions containing the desired product were combined and lyophilized to give the title compound (48 mg) as a white solid.

### Example 30

### Compound 101

### Synthesis of (2S)-2-({[1-(3-chlorobenzyl)-2-oxo-1,2-dihydropyridin-3-yl]carbonyl}amino)-5-(ethanimidoylamino)pentanoic acid•TFA

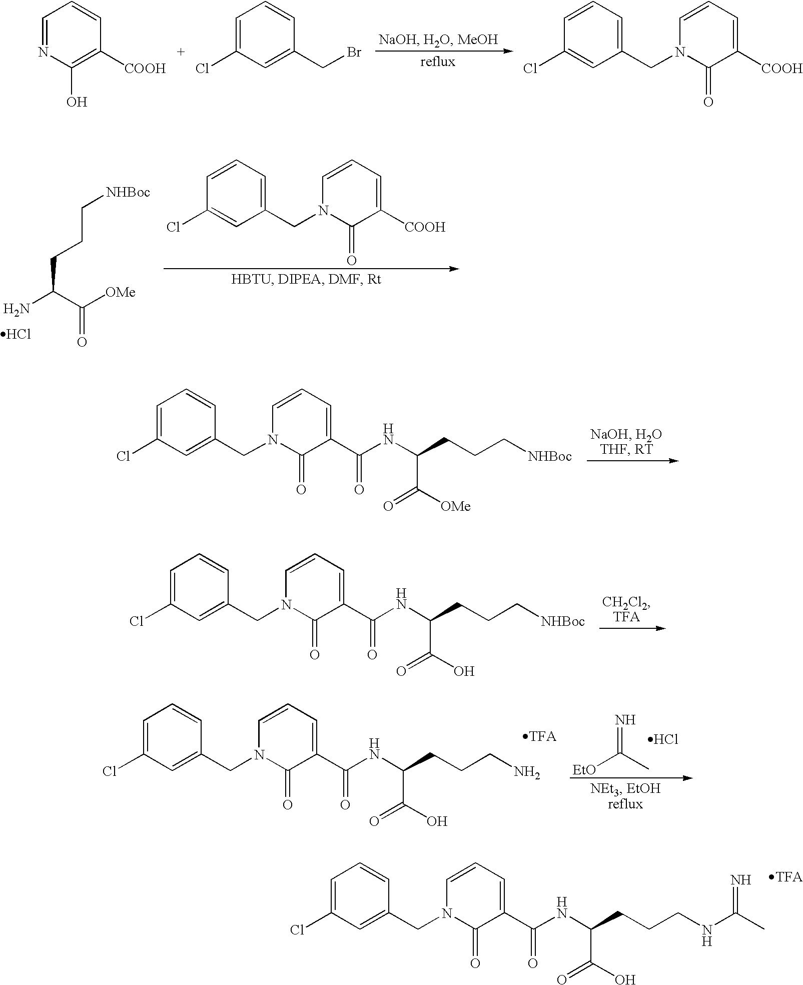

### I. 1-(3-Chlorobenzyl)-2-oxo-1,2-dihydropyridine-3-carboxylic acid

To a solution of 2-hydroxynicotinic acid (2.0 g) in a water:methanol mixture (3:10, 20 mL), NaOH (1.72 g) was added and the mixture was heated to reflux. To the resulting mixture, 3-chlorobenzyl bromide (5.9 g) was added and the mixture was refluxed overnight. The mixture was cooled to room temperature and the methanol was removed under reduced pressure. The resulting mixture was diluted with ethyl acetate and water and HCl (2 M) was added. The resulting precipitate was collected by filtration and dried under vacuum overnight to give the title compound (2.68 g) as a white solid.

### II. Methyl (2S)-5-[(tert-butoxycarbonyl)amino]-2-({[1-(3-chlorobenzyl)-2-oxo-1,2-dihydropyridin-3-yl]carbonyl}amino)pentanoate

A solution of methyl (2S)-2-amino-5-[(tert-butoxycarbonyl)amino]pentanoate hydrochloride (470 mg), 1-(3-chlorobenzyl)-2-oxo-1,2-dihydropyridine-3-carboxylic acid (6-2, 520 mg), diisopropylethylamine (1.2 mL) and HBTU (920 mg) in DMF(9 mL) was stirred at room temperature overnight. The resulting mixture was diluted with ethyl acetate and washed with water (3 times) and brine. The organic layer was dried over Na₂SO₄, filtered and concentrated under reduced pressure. The residue was purified by automated silica gel column chromatography (Biotage®), eluting with hexanes increasing to 50% ethyl acetate/hexanes and finally to 100% ethyl acetate to give the title compound (650 mg) as a yellow solid.

### III. (2S)-5-[(tert-Butoxycarbonyl)amino]-2-({[1-(3-chlorobenzyl)-2-oxo-1,2-dihydropyridin-3-yl]carbonyl}amino)pentanoic acid

To a solution of methyl (2S)-5-[(tert-butoxycarbonyl)amino]-2-({[1-(3-chlorobenzyl)-2-oxo-1,2-dihydropyridin-3-yl]carbonyl}amino)pentanoate (650 mg) in THF (2 mL), aqueous NaOH (2.0 M, 4 mL) was added. The resulting mixture was stirred at room temperature 4 hours, then was diluted with water and was extracted with ether. The aqueous layer was acidified with HCl (2 M) and extracted with ethyl acetate. The ethyl acetate layer was dried over MgSO₄, filtered and concentrated under reduced pressure to give the title compound (600 mg) as an off-white solid.

### IV. (2S)-5-Amino-2-({[1-(3-chlorobenzyl)-2-oxo-1,2-dihydropyridin-3-yl]carbonyl}amino)pentanoic acid•TFA

To a solution of (2S)-5-[(tert-butoxycarbonyl)amino]-2-({[1-(3-chlorobenzyl)-2-oxo-1,2-dihydropyridin-3-yl]carbonyl}amino)pentanoic acid (600 mg) in dichloromethane (3 mL), TFA (3 mL) was added. The resulting mixture was stirred at room temperature overnight and the reaction mixture was concentrated under reduced pressure to give the title compound (600 mg) as a yellow oil. This material was taken to the next step without further purification.

V. (2S)-2-({[1-(3-Chlorobenzyl)-2-oxo-1,2-dihydropyridin-3-yl]carbonyl}amino)-5-(ethanimidoylamino)pentanoic acid•TFA

To a solution of (2S)-5-amino-2-({[1-(3-chlorobenzyl)-2-oxo-1,2-dihydropyridin-3-yl]carbonyl}amino)pentanoic acid•TFA(400 mg) in ethanol (5 mL) at room temperature, ethyl acetimidate hydrochloride (250 mg) and NEt₃ (0.8 mL) were added sequentially. The resulting suspension was heated to reflux and was stirred for 3 hours. The resulting mixture was concentrated under reduced pressure and the residue was purified by reverse phase HPLC, eluting with an acetonitrile/water/TFA gradient mixture. Fractions containing the desired product were lyophilized to give the title compound (200 mg) as a white solid.

The following compounds were synthesized by modifications of the general procedure described in Example 30.

Compound 104: Step I was performed using 2-hydroxynicotinic acid (1.3 g), NaOH (1.2 g) and 2-chlorobenzyl chloride (3.0 g) to give 1-(2-chlorobenzyl)-2-oxo-1,2-dihydropyridine-3-carboxylic acid (0.95 g) as a white solid. Step II was performed using 1-(2-chlorobenzyl)-2-oxo-1,2-dihydropyridine-3-carboxylic acid (0.60 g), methyl (2S)-2-amino-5-[(tert-butoxycarbonyl)amino]pentanoatehydrochloride (0.5 g), diisopropylethylamine (0.95 mL) and HBTU (1.0 g) to give methyl (2S)-5-[(tert-butoxycarbonyl)amino]-2-({[1-(2-chlorobenzyl)-2-oxo-1,2-dihydropyridin-3-yl]carbonyl}amino)pentanoate (0.70 g) as a yellow solid. Step III was performed using methyl (2S)-5-[(tert-butoxycarbonyl)amino]-2-({[1-(2-chlorobenzyl)-2-oxo-1,2-dihydropyridin-3-yl]carbonyl}amino)pentanoate (0.70 g), THF (3 mL) and aqueous NaOH (2 M, 3 mL) and the mixture was stirred for 3 hours to give (2S)-5-[(tert-butoxycarbonyl)amino]-2-({[1-(2-chlorobenzyl)-2-oxo-1,2-dihydropyridin-3-yl]carbonyl}amino)pentanoic acid (0.6 g) as a white solid. Step IV was performed using (2S)-5-[(tert-butoxycarbonyl)amino]-2-({[1-(2-chlorobenzyl)-2-oxo-1,2-dihydropyridin-3-yl]carbonyl}amino)pentanoic acid (0.6 g), TFA (3 mL) and dichloromethane (3 mL) to give (2S)-5-amino-2-({[1-(2-chlorobenzyl)-2-oxo-1,2-dihydropyridin-3-yl]carbonyl}amino)pentanoic acid•TFA (0.7 g) was an off-white solid. Step V was performed using (2S)-5-amino-2-({[1-(2-chlorobenzyl)-2-oxo-1,2-dihydropyridin-3-yl]carbonyl}amino)pentanoic acid•TFA (0.7 g), ethanol (5 mL), ethyl acetimidate hydrochloride (370 mg) and NEt₃ (1.1 mL) and the reaction mixture was heated to 90° C. for 2 hours to give (2S)-2-({[1-(2-chlorobenzyl)-2-oxo-1,2-dihydropyridin-3-yl]carbonyl}amino)-5-(ethanimidoylamino)pentanoic acid•TFA (104, 300 mg) as a white solid.

Compound 106: Step I was performed using 2-hydroxynicotinic acid (1.0 g), NaOH (0.9 g) and 3-trifluoromethylbenzyl bromide (2.23 g) to give 2-oxo-1-[3-(trifluoromethyl)benzyl]-1,2-dihydropyridine-3-carboxylic acid as a white solid. Step II was performed using 2-oxo-1-[3-(trifluoromethyl)benzyl]-1,2-dihydropyridine-3-carboxylic acid (0.40 g), methyl (2S)-2-amino-5-[(tert-butoxycarbonyl)amino]pentanoatehydrochloride (0.3 g), triethylamine (0.5 mL) and HBTU (0.60 g) to give methyl (2S)-5-[(tert-butoxycarbonyl)amino]-2-[({2-oxo-1-[3-(trifluoromethyl)benzyl]-1,2-dihydropyridin-3-yl}carbonyl)amino]pentanoate (0.55 g) as a white solid. Step III was performed using methyl (2S)-5-[(tert-butoxycarbonyl)amino]-2-[({2-oxo-1-[3-(trifluoromethyl)benzyl]-1,2-dihydropyridin-3-yl}carbonyl)amino]pentanoate (0.55 g), THF (2 mL) and aqueous NaOH (2 M, 3 mL) and the mixture was stirred for 2 hours to give 2S)-5-[(tert-butoxycarbonyl)amino]-2-[({2-oxo-1-[3-(trifluoromethyl)benzyl]-1,2-dihydropyridin-3-yl}carbonyl)amino]pentanoic acid (0.5 g) as a white solid. Step IV was performed using 2S)-5-[(tert-butoxycarbonyl)amino]-2-[({2-oxo-1-[3-(trifluoromethyl)benzyl]-1,2-dihydropyridin-3-yl]carbonyl)amino}pentanoic acid (0.5 g), TFA (1.5 mL) and dichloromethane (2 mL) and the mixture was concentrated after stirring at room temperature for one hour to give (2S)-5-amino-2-[({2-oxo-1-[3-(trifluoromethyl)benzyl]-1,2-dihydropyridin-3-yl}carbonyl)amino]pentanoic acid•TFA (0.5 g) as an off-white solid. Step V was performed using (2S)-5-amino-2-[({2-oxo-1-[3-(trifluoromethyl)benzyl]-1,2-dihydropyridin-3-yl}carbonyl)amino]pentanoic acid•TFA (0.5 g), ethanol (5 mL), ethyl acetimidate hydrochloride (234 mg) and NEt₃ (0.5 mL) and the reaction mixture was heated to 90° C. for 2 hours to give (2S)-5-(ethanimidoylamino)-2-[({2-oxo-1-[3-(trifluoromethyl)benzyl]-1,2-dihydropyridin-3-yl}carbonyl)amino]pentanoic acid•TFA (106,0.060 g) as a white solid.

Compound 107: Step I was performed using 2-hydroxynicotinic acid (1.0 g), NaOH (0.9 g) and 2-trifluoromethylbenzyl bromide (2.23 g) to give 2-oxo-1-[2-(trifluoromethyl)benzyl]-1,2-dihydropyridine-3-carboxylic acid (1.6 g) as a white solid. Step II was performed using 2-oxo-1-[2-(trifluoromethyl)benzyl]-1,2-dihydropyridine-3-carboxylic acid (0.40 g), methyl (2S)-2-amino-5-[(tert-butoxycarbonyl)amino]pentanoatehydrochloride (0.3 g), triethylamine (0.5 mL) and HBTU (0.60 g) to give methyl (2S)-5-[(tert-butoxycarbonyl)amino]-2-[({2-oxo-1-[2-(trifluoromethyl)benzyl]-1,2-dihydropyridin-3-yl}carbonyl)amino]pentanoate (0.6 g) as an off-white solid. Step III was performed using methyl (2S)-5-[(tert-butoxycarbonyl)amino]-2-[({2-oxo-1-[2-(trifluoromethyl)benzyl]-1,2-dihydropyridin-3-yl}carbonyl)amino]pentanoate (0.6 g), THF (2 mL) and aqueous NaOH (2 M, 3 mL) and the mixture was stirred for 2 hours to give (2S)-5-[(tert-butoxycarbonyl)amino]-2-[({2-oxo-1-[2-(trifluoromethyl)benzyl]-1,2-dihydropyridin-3-yl}carbonyl)amino]pentanoic acid (0.56 g) as a white solid. Step IV was performed using (2S)-5-[(tert-butoxycarbonyl)amino]-2-[({2-oxo-1-[2-(trifluoromethyl)benzyl]-1,2-dihydropyridin-3-yl}carbonyl)amino]pentanoic acid (0.56 g), TFA (1.5 mL) and dichloromethane (2 mL) and the mixture was concentrated after stirring at room temperature for one hour to give (2S)-5-amino-2-[({2-oxo-1-[2-(trifluoromethyl)benzyl]-1,2-dihydropyridin-3-yl}carbonyl)amino]pentanoic acid•TFA (0.5 g). Step V was performed using (2S)-5-amino-2-[({2-oxo-1-[2-(trifluoromethyl)benzyl]-1,2-dihydropyridin-3-yl}carbonyl)amino]pentanoic acid•TFA (0.5 g), ethanol (5 mL), ethyl acetimidate hydrochloride (234 mg) and NEt₃ (0.5 mL) and the reaction mixture was heated to 90° C. for 2 hours to give (2S)-5-(ethanimidoylamino)-2-[({2-oxo-1-[2-(trifluoromethyl)benzyl]-1,2-dihydropyridin-3-yl}carbonyl)amino]pentanoic acid•TFA (107, 0.170 g) as a white solid.

Compound 109: Step I was performed using 2-hydroxynicotinic acid (1.0 g), NaOH (0.9 g) and 3-bromobenzyl bromide (2.23 g) to give 1-(3-bromobenzyl)-2-oxo-1,2-dihydropyridine-3-carboxylic acid (1.7 g) as a white solid. Step II was performed using 1-(3-bromobenzyl)-2-oxo-1,2-dihydropyridine-3-carboxylic acid (0.56 g), methyl (2S)-2-amino-5-[(tert-butoxycarbonyl)amino]pentanoatehydrochloride (0.4 g), triethylamine (1.0 mL) and HBTU (0.8 g) to give methyl (2S)-2-({[1-(3-bromobenzyl)-2-oxo-1,2-dihydropyridin-3-yl]carbonyl}amino)-5-[(tert-butoxycarbonyl)amino]pentanoate (0.6 g) as a white solid. Step III was performed using methyl (2S)-2-({[1-(3-bromobenzyl)-2-oxo-1,2-dihydropyridin-3-yl]carbonyl}amino)-5-[(tert-butoxycarbonyl)amino]pentanoate (0.6 g), THF (2 mL) and aqueous NaOH (2 M, 3 mL) and the mixture was stirred for 2 hours to give (2S)-2-({[1-(3-bromobenzyl)-2-oxo-1,2-dihydropyridin-3-yl]carbonyl}amino)-5-[(tert-butoxycarbonyl)amino]pentanoic acid (0.55 g) as a white solid. Step IV was performed using (2S)-2-({[1-(3-bromobenzyl)-2-oxo-1,2-dihydropyridin-3-yl]carbonyl}amino)-5-[(tert-butoxycarbonyl)amino]pentanoic acid (0.55 g), TFA (1 mL) and dichloromethane (2 mL) and the mixture was concentrated after stirring at room temperature for one hour to give (2S)-5-amino-2-({[1-(3-bromobenzyl)-2-oxo-1,2-dihydropyridin-3-yl]carbonyl}amino)pentanoic acid•TFA (0.5 g). Step V was performed using (2S)-5-amino-2-({[1-(3-bromobenzyl)-2-oxo-1,2-dihydropyridin-3-yl]carbonyl}amino)pentanoic acid•TFA (0.5 g), ethanol (5 mL), ethyl acetimidate hydrochloride (230 mg) and NEt₃ (0.6 mL) and the reaction mixture was heated to 90° C. for 2 hours to give (2S)-2-({[1-(3-bromobenzyl)-2-oxo-1,2-dihydropyridin-3-yl]carbonyl}amino)-5-(ethanimidoylamino)pentanoic acid•TFA (109, 0.12 g) as a white solid.

Compound 110: Step I was performed using 2-hydroxynicotinic acid (1.0 g), NaOH (0.9 g) and 4-bromobenzyl bromide (2.3 g) to give 2-oxo-1-(4-bromobenzyl)-1,2-dihydropyridine-3-carboxylic acid (1.6 g) as a white solid. Step II was performed using 2-oxo-1-(4-bromobenzyl)-1,2-dihydropyridine-3-carboxylic acid (0.60 g), methyl (2S)-2-amino-5-[(tert-butoxycarbonyl)amino]pentanoatehydrochloride (0.43 g), triethylamine (1.6 mL) and HBTU (0.85 g) to give methyl (2S)-2-({[1-(4-bromobenzyl)-2-oxo-1,2-dihydropyridin-3-yl]carbonyl}amino)-5-[(tert-butoxycarbonyl)amino]pentanoate (0.62 g) as a white solid. Step III was performed using methyl (2S)-2-({[1-(4-bromobenzyl)-2-oxo-1,2-dihydropyridin-3-yl]carbonyl}amino)-5-[(tert-butoxycarbonyl)amino]pentanoate (0.60 g) to give (2S)-2-({[1-(4-bromobenzyl)-2-oxo-1,2-dihydropyridin-3-yl]carbonyl}amino)-5-[(tert-butoxycarbonyl)amino]pentanoic acid (0.55 g) as a white solid. Step IV was performed using (2S)-2-({[1-(4-bromobenzyl)-2-oxo-1,2-dihydropyridin-3-yl]carbonyl}amino)-5-[(tert-butoxycarbonyl)amino]pentanoic acid (0.55 g), TFA (1 mL) and dichloromethane (2 mL) and the mixture was concentrated after stirring at room temperature for 2 hours to give (2S)-5-amino-2-({[1-(4-bromobenzyl)-2-oxo-1,2-dihydropyridin-3-yl]carbonyl}amino)pentanoic acid•TFA (0.56 g) as an off-white solid. Step V was performed using (2S)-5-amino-2-({[1-(4-bromobenzyl)-2-oxo-1,2-dihydropyridin-3-yl]carbonyl}amino)pentanoic acid•TFA (0.56 g), ethanol (5 mL), ethyl acetimidate hydrochloride (260 mg) and NEt₃ (0.6 mL) and the reaction mixture was heated to 90° C. for 2 hours to give (2S)-2-({[1-(4-bromobenzyl)-2-oxo-1,2-dihydropyridin-3-yl]carbonyl}amino)-5-(ethanimidoylamino)pentanoic acid•TFA, (110, 0.21 g) as a white solid.

Compound 113: Step I was performed using 2-hydroxynicotinic acid (1.0 g), NaOH (0.9 g) and 4-trifluoromethylbenzyl bromide (2.33 g) to give 2-oxo-1-[4-(trifluoromethyl)benzyl]-1,2-dihydropyridine-3-carboxylic acid (1.3 g) as a white solid. Step II was performed using 2-oxo-1-[4-(trifluoromethyl)benzyl]-1,2-dihydropyridine-3-carboxylic acid (0.58 g), methyl (2S)-2-amino-5-[(tert-butoxycarbonyl)amino]pentanoate hydrochloride (0.43 g), triethylamine (1.0 mL) and HBTU (0.75 g) to give methyl (2S)-5-[(tert-butoxycarbonyl)amino]-2-[({2-oxo-1-[4-(trifluoromethyl)benzyl]-1,2-dihydropyridin-3-yl]carbonyl)amino}pentanoate (0.4 g) as a white solid. Step III was performed methyl (2S)-5-[(tert-butoxycarbonyl)amino]-2-[({2-oxo-1-[4-(trifluoromethyl)benzyl]-1,2-dihydropyridin-3-yl}carbonyl)amino]pentanoate (0.4 g), to give (2S)-5-[(tert-butoxycarbonyl)amino]-2-[({2-oxo-1-[4-(trifluoromethyl)benzyl]-1,2-dihydropyridin-3-yl}carbonyl)amino]pentanoic acid (0.36 g) as a white solid. Step IV was performed using (2S)-5-[(tert-butoxycarbonyl)amino]-2-[({2-oxo-1-[4-(trifluoromethyl)benzyl]-1,2-dihydropyridin-3-yl}carbonyl)amino]pentanoic acid (0.35 g) to give (2S)-5-amino-2-[({2-oxo-1-[4-(trifluoromethyl)benzyl]-1,2-dihydropyridin-3-yl}carbonyl)amino]pentanoic acid•TFA (0.35 g). Step V was performed using (2S)-5-amino-2-[({2-oxo-1-[4-(trifluoromethyl)benzyl]-1,2-dihydropyridin-3-yl}carbonyl)amino]pentanoic acid•TFA (0.35 g), ethanol (5 mL), ethyl acetimidate hydrochloride (164 mg) and NEt₃ (0.39 mL) and the reaction mixture was heated to 70° C. for 3 hours to give (2S)-5-(ethanimidoylamino)-2-[({2-oxo-1-[4-(trifluoromethyl)benzyl]-1,2-dihydropyridin-3-yl}carbonyl)amino]pentanoic acid•TFA, (113, 0.110 g) as a white solid.

Compound 114: Step I was performed using 2-hydroxynicotinic acid (0.2 g), H₂O (1 mL), MeOH (3 mL), KOH (0.24 g) and 3-iodobenzyl bromide (0.90 g) and the mixture was heated to 65° C. overnight to give 1-(3-iodobenzyl)-2-oxo-1,2-dihydropyridine-3-carboxylic acid (0.56 g) as an off-white solid. Step II was performed using 1-(3-iodobenzyl)-2-oxo-1,2-dihydropyridine-3-carboxylic acid (0.50 g), DMF (7.0 mL), methyl (2S)-2-amino-5-[(tert-butoxycarbonyl)amino]pentanoate hydrochloride (0.40 g), disopropylethylamine (0.32 mL) and HBTU (0.75 g) to give methyl (2S)-5-[(tert-butoxycarbonyl)amino]-2-({[1-(3-iodobenzyl)-2-oxo-1,2-dihydropyridin-3-yl]carbonyl}amino)pentanoate (0.80 g) as a light yellow oil. Step III was performed using methyl (2S)-5-[(tert-butoxycarbonyl)amino]-2-({[1-(3-iodobenzyl)-2-oxo-1,2-dihydropyridin-3-yl]carbonyl}amino)pentanoate (0.80 g), THF (10 mL), methanol (1.5 mL) and aqueous NaOH (2 M, 5 mL) and the mixture was stirred for 2 hours to give (2S)-5-[(tert-butoxycarbonyl)amino]-2-({[1-(3-iodobenzyl)-2-oxo-1,2-dihydropyridin-3-yl]carbonyl}amino)pentanoic acid (0.80 g) as an off-white foam. Step IV was performed using (2S)-5-[(tert-butoxycarbonyl)amino]-2-({[1-(3-iodobenzyl)-2-oxo-1,2-dihydropyridin-3-yl]carbonyl}amino)pentanoic acid (0.78 g), TFA (4 mL) and dichloromethane (4 mL) and the mixture was concentrated after stirring at room temperature for two hours to give (2S)-5-amino-2-({[1-(3-iodobenzyl)-2-oxo-1,2-dihydropyridin-3-yl]carbonyl}amino)pentanoic acid•TFA (0.7 g) as an off-white solid. Step V was performed using (2S)-5-amino-2-({[1-(3-iodobenzyl)-2-oxo-1,2-dihydropyridin-3-yl]carbonyl}amino)pentanoic acid•TFA (0.3 g), ethanol (6 mL), ethyl acetimidate hydrochloride (160 mg) and NEt₃ (1.3 mL) and the reaction mixture was stirred at room temperature overnight to give (2S)-5-(ethanimidoylamino)-2-({[1-(3-iodobenzyl)-2-oxo-1,2-dihydropyridin-3-yl]carbonyl}amino)pentanoic acid•TFA (114, 73 mg) as a white solid.

Compound 115: Step I was performed using 2-hydroxynicotinic acid (0.2 g), H₂O (1 mL), MeOH (3 mL), KOH (0.24 g) and 3,5-dibromobenzyl bromide (1.0 g) and the mixture was heated to 65° C. overnight to give 1-(3,5-dibromobenzyl)-2-oxo-1,2-dihydropyridine-3-carboxylic acid (0.85 g) as an off-white solid. Step II was performed using 1-(3,5-dibromobenzyl)-2-oxo-1,2-dihydropyridine-3-carboxylic acid (0.54 g), DMF (7.0 mL), methyl (2S)-2-amino-5-[(tert-butoxycarbonyl)amino]pentanoate hydrochloride (0.40 g), disopropylethylamine (0.32 mL) and HBTU (0.75 g) to give methyl (2S)-5-[(tert-butoxycarbonyl)amino]-2-({[1-(3,5-dibromobenzyl)-2-oxo-1,2-dihydropyridin-3-yl]carbonyl}amino)pentanoate (0.85 g) as a light yellow oil. Step III was performed using methyl (2S)-5-[(tert-butoxycarbonyl)amino]-2-({[1-(3,5-dibromobenzyl)-2-oxo-1,2-dihydropyridin-3-yl]carbonyl}amino)pentanoate (0.85 g), THF (10 mL), methanol (1.5 mL) and aqueous NaOH (2 M, 5 mL) and the mixture was stirred for 2 hours to give (2S)-5-[(tert-butoxycarbonyl)amino]-2-({[1-(3,5-dibromobenzyl)-2-oxo-1,2-dihydropyridin-3-yl]carbonyl}amino)pentanoic acid (0.83 g) as an off-white foam. Step IV was performed using (2S)-5-[(tert-butoxycarbonyl)amino]-2-({[1-(3,5-dibromobenzyl)-2-oxo-1,2-dihydropyridin-3-yl]carbonyl}amino)pentanoic acid (0.82 g), TFA (4 mL) and dichloromethane (4 mL) and the mixture was concentrated after stirring at room temperature for two hours to give (2S)-5-amino-2-({[1-(3,5-dibromobenzyl)-2-oxo-1,2-dihydropyridin-3-yl]carbonyl}amino)pentanoic acid•TFA (0.8 g). Step V was performed using (2S)-5-amino-2-({[1-(3,5-dibromobenzyl)-2-oxo-1,2-dihydropyridin-3-yl]carbonyl}amino)pentanoic acid•TFA (0.35 g), ethanol (7 mL), ethyl acetimidate hydrochloride (170 mg) and NEt₃ (1.5 mL) and the reaction mixture was stirred at room temperature overnight to give (2S)-2-({[1-(3,5-dibromobenzyl)-2-oxo-1,2-dihydropyridin-3-yl]carbonyl}amino)-5-(ethanimidoylamino)pentanoic acid•TFA (115, 65 mg) as a white solid.

Compound 116: Step I was performed using 2-hydroxynicotinic acid (0.32 g), H₂O (4 mL), MeOH (12 mL), KOH (0.4 g) and 3,5-dichlorobenzyl chloride (1.0 g) and the mixture was heated to reflux for 1 hour to give 1-(3,5-dichlorobenzyl)-2-oxo-1,2-dihydropyridine-3-carboxylic acid (0.46 g) as a white solid. Step II was performed using 1-(3,5-dichlorobenzyl)-2-oxo-1,2-dihydropyridine-3-carboxylic acid (0.46 g), DMF (8 mL), methyl (2S)-2-amino-5-[(tert-butoxycarbonyl)amino]pentanoate hydrochloride (0.44 g), disopropylethylamine (0.35 mL) and HBTU (0.82 g) to give methyl (2S)-5-[(tert-butoxycarbonyl)amino]-2-({[1-(3,5-dichlorobenzyl)-2-oxo-1,2-dihydropyridin-3-yl]carbonyl}amino)pentanoate (0.80 g) as a light yellow oil. Step III was performed using methyl (2S)-5-[(tert-butoxycarbonyl)amino]-2-({[1-(3,5-dichlorobenzyl)-2-oxo-1,2-dihydropyridin-3-yl]carbonyl}amino)pentanoate (0.80 g), THF (10 mL), methanol (1.5 mL) and aqueous NaOH (2 M, 5 mL) and the mixture was stirred for 2 hours to give (2S)-5-[(tert-Butoxycarbonyl)amino]-2-({[1-(3,5-dichlorobenzyl)-2-oxo-1,2-dihydropyridin-3-yl]carbonyl}amino)pentanoic acid (0.80 g) as an off-white foam. Step IV was performed using (2S)-5-[(tert-Butoxycarbonyl)amino]-2-({[1-(3,5-dichlorobenzyl)-2-oxo-1,2-dihydropyridin-3-yl]carbonyl}amino)pentanoic acid (0.76 g), TFA (4.5 mL) and dichloromethane (4.5 mL) and the mixture was concentrated after stirring at room temperature for two hours to give (2S)-5-amino-2-({[1-(3,5-dichlorobenzyl)-2-oxo-1,2-dihydropyridin-3-yl]carbonyl}amino)pentanoic acid•TFA (0.7 g) as a light brown oil. Step V was performed using (2S)-5-amino-2-({[1-(3,5-dichlorobenzyl)-2-oxo-1,2-dihydropyridin-3-yl]carbonyl}amino)pentanoic acid•TFA (0.30 g), ethanol (7 mL), ethyl acetimidate hydrochloride (180 mg) and NEt₃ (1.5 mL) and the reaction mixture was stirred at room temperature overnight to give (2S)-2-({[1-(3,5-dichlorobenzyl)-2-oxo-1,2-dihydropyridin-3-yl]carbonyl}amino)-5-(ethanimidoylamino)pentanoic acid•TFA, (116, 73 mg) as a white solid.

Compound 117: Step I was performed using 2-hydroxynicotinic acid (0.32 g), H₂O (4 mL), MeOH (12 mL), KOH (0.4 g) and 3,5-difluorobenzyl bromide (1.0 g) and the mixture was heated to reflux for 1 hour to give 1-(3,5-difluorobenzyl)-2-oxo-1,2-dihydropyridine-3-carboxylic acid (0.56 g) as a white solid. Step II was performed using 1-(3,5-difluorobenzyl)-2-oxo-1,2-dihydropyridine-3-carboxylic acid (0.56 g), DMF (11 mL), methyl (2S)-2-amino-5-[(tert-butoxycarbonyl)amino]pentanoate hydrochloride (0.60 g), disopropylethylamine (0.5 mL) and HBTU (1.1 g) to give methyl (2S)-5-[(tert-butoxycarbonyl)amino]-2-({[1-(3,5-difluorobenzyl)-2-oxo-1,2-dihydropyridin-3-yl]carbonyl}amino)pentanoate (1.0 g) as a light yellow oil. Step III was performed using methyl (2S)-5-[(tert-butoxycarbonyl)amino]-2-({[1-(3,5-difluorobenzyl)-2-oxo-1,2-dihydropyridin-3-yl]carbonyl}amino)pentanoate (1.0 g), THF (12 mL), methanol (2 mL) and aqueous NaOH (2 M, 6 mL) and the mixture was stirred for 2 hours to give (2S)-5-[(tert-butoxycarbonyl)amino]-2-({[1-(3,5-difluorobenzyl)-2-oxo-1,2-dihydropyridin-3-yl]carbonyl}amino)pentanoic acid (1.0 g) as an off-white foam. Step IV was performed using (2S)-5-[(tert-butoxycarbonyl)amino]-2-({[1-(3,5-difluorobenzyl)-2-oxo-1,2-dihydropyridin-3-yl]carbonyl}amino)pentanoic acid (0.95 g), TFA (6 mL) and dichloromethane (6 mL) and the mixture was concentrated after stirring at room temperature for two hours to give (2S)-5-amino-2-({[1-(3,5-difluorobenzyl)-2-oxo-1,2-dihydropyridin-3-yl]carbonyl}amino)pentanoic acid•TFA (0.8 g) as a light brown oil. Step V was performed using (2S)-5-amino-2-({[1-(3,5-difluorobenzyl)-2-oxo-1,2-dihydropyridin-3-yl]carbonyl}amino)pentanoic acid•TFA (0.40 g), ethanol (11 mL), ethyl acetimidate hydrochloride (260 mg) and NEt₃ (2.2 mL) and the reaction mixture was stirred at room temperature overnight to give (2S)-2-({[1-(3,5-difluorobenzyl)-2-oxo-1,2-dihydropyridin-3-yl]carbonyl}amino)-5-(ethanimidoylamino)pentanoic acid•TFA (117, 41 mg) as a white solid.

### Example 31

### Compound 100

### Synthesis of (2S)-2-{[(1-benzyl-2-oxo-1,2-dihydropyridin-3-yl)carbonyl]amino}-5-(ethanimidoylamino)pentanoic acid•TFA

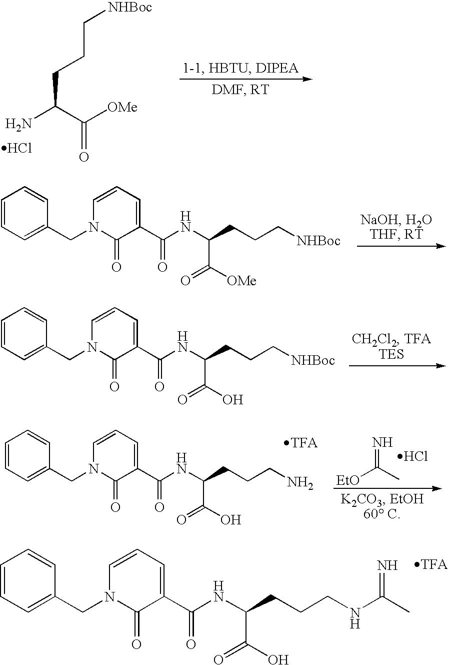

### I. Methyl (2S)-2-{[(1-benzyl-2-oxo-1,2-dihydropyridin-3-yl)carbonyl]amino}-5-[(tert-butoxycarbonyl)amino]pentanoate

A solution of methyl (2S)-2-amino-5-[(tert-butoxycarbonyl)amino]pentanoate hydrochloride (480 mg), 1-benzyl-2-oxo-1,2-dihydropyridine-3-carboxylic acid (1-1, 440 mg), diisopropylethylamine (1.25 mL) and HBTU (0.96 g) in DMF(7 mL) was stirred at room temperature overnight. The resulting mixture was diluted with ethyl acetate and washed with water (3 times) and brine. The organic layer was dried over Na₂SO₄, filtered and concentrated under reduced pressure. The residue was purified by automated silica gel column chromatography (Biotage®) to give the title compound (710 mg) as a pale yellow solid.

### II. (2S)-2-{[(1-Benzyl-2-oxo-1,2-dihydropyridin-3-yl)carbonyl]amino}-5-[(tert-butoxycarbonyl)amino]pentanoic acid

To a solution of methyl (2S)-2-{[(1-benzyl-2-oxo-1,2-dihydropyridin-3-yl)carbonyl]amino}-5-[(tert-butoxycarbonyl)amino]pentanoate (700 mg) in THF (3 mL), aqueous NaOH (2.0 M, 3 mL) was added. The resulting mixture was stirred at room temperature overnight, then was acidified with HCl (2M) and extracted with ethyl acetate. The ethyl acetate layer was dried over MgSO₄, filtered and concentrated under reduced pressure to give the title compound (500 mg) as an off-white solid.

### III. (2S)-5-Amino-2-{[(1-benzyl-2-oxo-1,2-dihydropyridin-3-yl)carbonyl]amino}pentanoic acid•TFA

To a solution of (2S)-2-{[(1-benzyl-2-oxo-1,2-dihydropyridin-3-yl)carbonyl]amino}-5-[(tert-butoxycarbonyl)amino]pentanoic acid (500 mg) in dichloromethane (3 mL), TFA (2 mL) was added. The resulting mixture was stirred at room temperature for 5 minutes and triethylsilane (0.2 mL) was added. The resulting mixture was stirred at room temperature for 2 hours and the reaction mixture was concentrated under reduced pressure to give the title compound as a yellow oil. This material was taken to the next step without further purification.

### IV. (2S)-2-{[(1-Benzyl-2-oxo-1,2-dihydropyridin-3-yl)carbonyl]amino}-5-(ethanimidoylamino)pentanoic acid•TFA

To a solution of (2S)-5-amino-2-{[(1-benzyl-2-oxo-1,2-dihydropyridin-3-yl)carbonyl]amino}pentanoic acid•TFA (470 mg) in ethanol (10 mL) at room temperature, ethyl acetimidate hydrochloride (190 mg) and K₂CO₃ (600 mg) were added sequentially. The resulting suspension was heated to 60° C. and was stirred overnight. The resulting mixture was filtered through Celite and concentrated under reduced pressure and the residue was purified by reverse phase HPLC, eluting with an acetonitrile/water/TFA gradient mixture. Fractions containing the desired product were lyophilized to give the title compound (70 mg) as a white solid.

### Example 32

### Compound 122

### Synthesis of (2S)-2-({[1-(diphenylmethyl)-4-methoxy-2-oxo-1,2-dihydropyridin-3-yl]carbonyl}amino)-5-(ethanimidoylamino)pentanoic acid•TFA

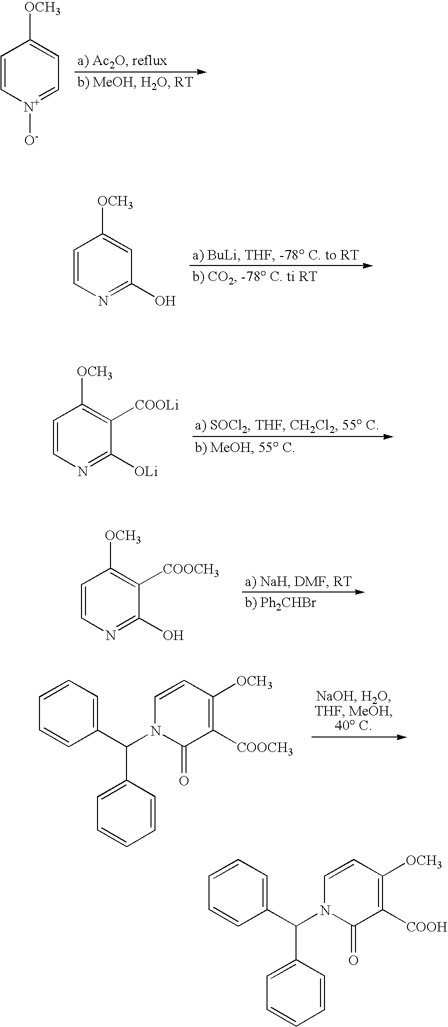

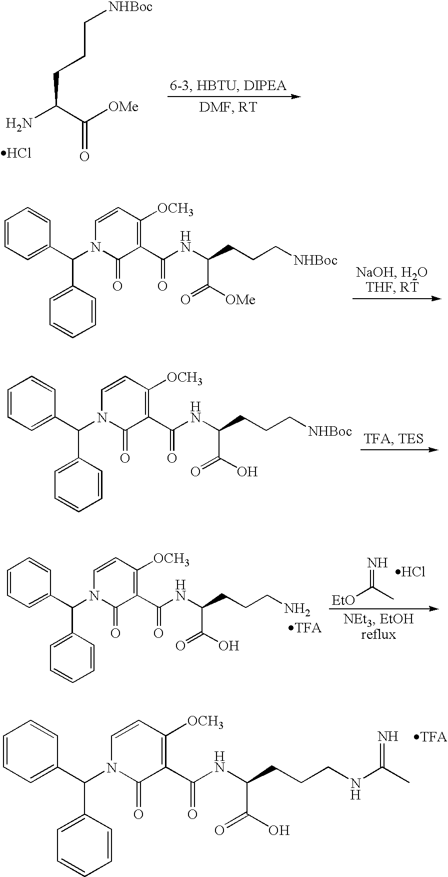

### I. 4-Methoxypyridin-2(1H)-one

A solution of 4-methoxypyridine-N-oxide (1.50 g) in acetic anhydride (45 mL) was heated to reflux for 6.5 hours then was cooled and concentrated under reduced pressure. The residue was taken up in methanol (15 mL) and water (15 mL) and was stirred overnight at room temperature. The resulting mixture was concentrated under reduced pressure and the residue was purified by silica gel chromatography, eluting with 5% methanol/dichloromethane increasing to 10% methanol/dichloromethane to give 4-methoxypyridin-2-ol (734 mg) as a pale yellow-green solid.

### II. 4-Methoxy-2-oxo-1,2-dihydropyridine-3-carboxylic acid dilithium salt

To a solution of 4-methoxypyridin-2(1H)-one (632 mg) in THF (40 mL) cooled to −78° C. under nitrogen, butyllithium (2.1 M in hexanes, 7.5 mL) was added dropwise. The resulting mixture was warmed to room temperature, was stirred for 50 minutes and then was cooled to −78° C. To the resulting mixture, powdered dry ice was added and the resulting mixture was allowed to warm to room temperature and was stirred overnight. The resulting suspension was filtered and the solid was washed with ether and dried under vacuum to give 2-hydroxy-4-methoxynicotinic acid dilithium salt (1.61 g) as a yellow solid. This material was used without purification.

### III. Methyl 4-methoxy-2-oxo-1,2-dihydropyridine-3-carboxylate

To as suspension of 4-methoxy-2-oxo-1,2-dihydropyridine-3-carboxylic acid dilithium salt (1.61 g) in dichloromethane (25 mL) and THF (25 mL), SOCl₂ (5.5 mL) was added and the resulting mixture was heated to 55° C. for 1 hour. To the resulting mixture, anhydrous methanol (20 mL) was added and heating was continued overnight. The resulting mixture was cooled to room temperature, concentrated under reduced pressure and the residue was purified by automated silica gel column chromatography (Biotage®) eluting with a methanol/dichloromethane gradient to give the title compound (0.329 g).

### IV. Methyl 1-(diphenylmethyl)-4-methoxy-2-oxo-1,2-dihydropyridine-3-carboxylate

To a solution of methyl 4-methoxy-2-oxo-1,2-dihydropyridine-3-carboxylate (0.329 g) in DMF (15 mL) at room temperature, sodium hydride (60% dispersion in mineral oil, 239 mg) was added. The resulting mixture was stirred at room temperature for 25 minutes, diphenylmethyl bromide (962 mg) was added and stirring was continued overnight. The resulting mixture was quenched with water and diluted with ethyl acetate. The organic layer was washed with water (3 times) and brine, dried over Na₂SO₄, filtered and concentrated under reduced pressure. The residue was purified by automated silica gel column chromatography (Biotage®) eluting with 50% ethyl acetate/hexanes increasing to 100% ethyl acetate to give the title compound (430 mg) as a pale yellow foam.

### V. 1-(Diphenylmethyl)-4-methoxy-2-oxo-1,2-dihydropyridine-3-carboxylic acid

To a solution of methyl 1-(diphenylmethyl)-4-methoxy-2-oxo-1,2-dihydropyridine-3-carboxylate (430 mg) in THF (2 mL) and MeOH (2 mL), aqueous NaOH (2.0 M, 0.75 mL) was added. The resulting mixture was heated to 40° C. for 4 h, then was diluted with ether and water. The aqueous layer was washed with ether and then acidified with HCl (2M) and extracted with ethy acetate. The ethyl acetate layer was washed with water and brine, dried over Na₂SO₄, filtered and concentrated under reduced pressure to give the title compound (76 mg).

### VI. Methyl (2S)-5-[(tert-butoxycarbonyl)amino]-2-({[1-(diphenylmethyl)-4-methoxy-2-oxo-1,2-dihydropyridin-3-yl]carbonyl}amino)pentanoate

A solution of methyl (2S)-2-amino-5-[(tert-butoxycarbonyl)amino]pentanoate hydrochloride (65 mg), 1-(diphenylmethyl)-4-methoxy-2-oxo-1,2-dihydropyridine-3-carboxylic acid (76 mg), diisopropylethylamine (0.14 mL) and HBTU (122 mg) in DMF(2 mL) was stirred at room temperature overnight. The resulting mixture was diluted with ethyl acetate, washed with water (3 times) and with brine. The organic layer was dried over MgSO₄, filtered and concentrated under reduced pressure. The residue was purified by automated silica gel column chromatography (Biotage™) eluting with 40% ethyl acetate/hexanes increasing to 100% ethyl acetate and finally 10: methanol/dichloromethane to give the title compound (95 mg).

### VII. (2S)-5-[(tert-Butoxycarbonyl)amino]-2-({[1-(diphenylmethyl)-4-methoxy-2-oxo-1,2-dihydropyridin-3-yl]carbonyl}amino)pentanoic acid

To a solution of methyl (2S)-5-[(tert-butoxycarbonyl)amino]-2-({[1-(diphenylmethyl)-4-methoxy-2-oxo-1,2-dihydropyridin-3-yl]carbonyl}amino)pentanoate (95 mg) in THF (2 mL) and MeOH (2 mL), aqueous NaOH (2.0 M, 0.51 mL) was added. The resulting mixture was stirred at room temperature for 5 h, then was diluted with ether and water. The aqueous layer was washed with ether and then acidified with HCl (2M) and extracted with ethy acetate. The ethyl acetate layer was washed with water and brine, dried over Na₂SO₄, filtered and concentrated under reduced pressure to give the title compound (61 mg).

### VII. (2S)-5-Amino-2-({[1-(diphenylmethyl)-4-methoxy-2-oxo-1,2-dihydropyridin-3-yl]carbonyl}amino)pentanoic acid•TFA

To a solution of (2S)-5-[(tert-butoxycarbonyl)amino]-2-({[1-(diphenylmethyl)-4-methoxy-2-oxo-1,2-dihydropyridin-3-yl]carbonyl}amino)pentanoic acid (61 mg) in TFA (2 mL), triethylsilane (0.2 mL) was added. The resulting mixture was stirred at room temperature for 1.5 hour and the reaction mixture was concentrated under reduced pressure to give a the title compound (0.6 g).

### VIII. ((2S)-2-([{1-(Diphenylmethyl)-4-methoxy-2-oxo-1,2-dihydropyridin-3-yl]carbonyl}amino)-5-(ethanimidoylamino)pentanoic acid•TFA

To a solution of (2S)-5-amino-2-({[1-(diphenylmethyl)-4-methoxy-2-oxo-1,2-dihydropyridin-3-yl]carbonyl}amino)pentanoic acid•TFA (0.6 g) in ethanol (2 mL) at room temperature, ethyl acetimidate hydrochloride (27 mg) and NEt₃ (0.076 mL) were added sequentially. The resulting suspension was stirred overnight and the resulting mixture was concentrated under reduced pressure. The residue was dissolved in a acetonitrile:water:TFA mixture (1: 1:0.001, 3 mL) and the resulting solution was lyophilized to give the title compound (54.3 mg) as a white solid.

### Example 33

### Compound 103

### Synthesis of (2S)-2-({[5-(diphenylmethyl)thiophen-2-yl]carbonyl}amino)-5-(ethanimidoylamino)pentanoic acid•TFA

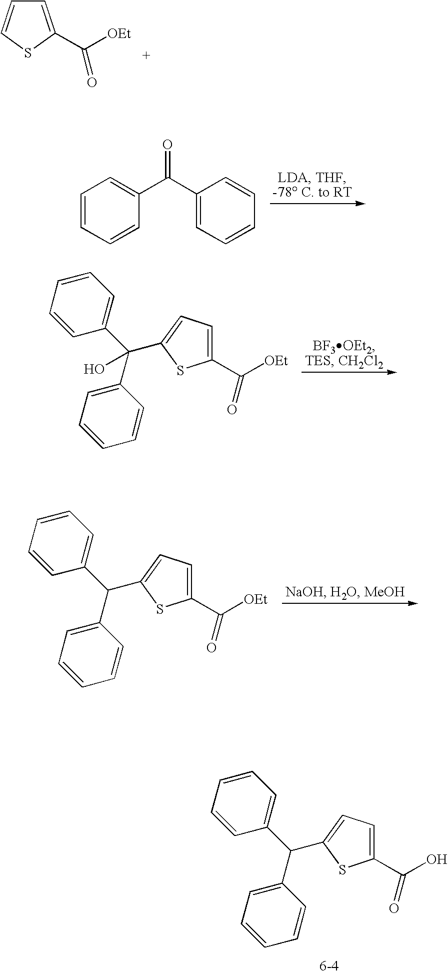

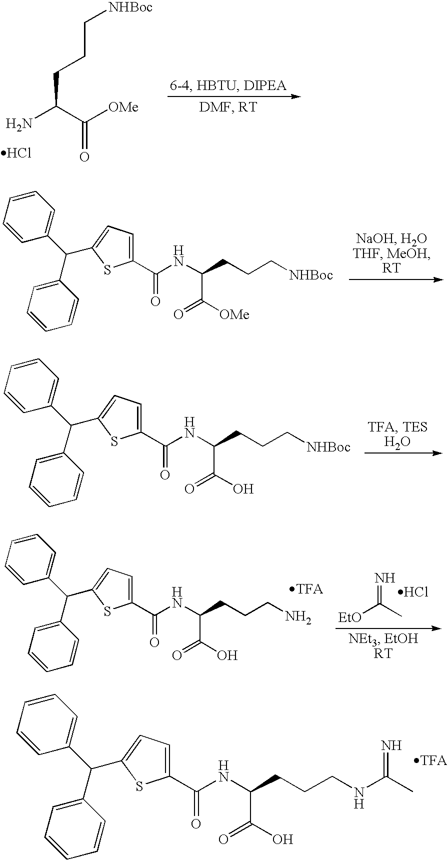

### I. Ethyl 5-[hydroxy(diphenyl)methyl]thiophene-2-carboxylate

To a solution of lithium diisopropylamide (LDA, 1.8 M in THF/heptane/ethyl benzene, 3.0 mL) in THF (10 mL) cooled to −78° C. under a dry nitrogen atmosphere, a solution of ethyl thiophene-2-carboxylate (0.73 mL) and benzophenone (1.0 g) in THF (15 mL) was added dropwise by cannula. The resulting mixture was allowed to warm to room temperature and was stirred for two hours. The resulting mixture was quenched with HCl (2 M) and extracted twice with ethyl acetate. The combined organic layers were dried over Na₂SO₄, filtered and concentrated and the residue was purified by automated silica gel column chromatography (Biotage®) eluting with an ethyl acetate/hexanes gradient to give the title compound (1.20 g) as a colorless oil.

### II. Ethyl 5-(diphenylmethyl)thiophene-2-carboxylate

To a solution of ethyl 5-[hydroxy(diphenyl)methyl]thiophene-2-carboxylate (1.20 g) in dichloromethane (15 mL), excess BF₃—OEt₂ and Et₃SiH were added. The reaction was monitored by TLC and additional BF₃—OEt₂ and Et₃SiH were added as needed. Once the starting material had been completely consumed according to TLC, the reaction mixture was diluted with ether and saturated aqueous NaHCO₃. The organic layer washed with water and brine, dried over Na₂SO₄, filtered and concentrated under reduced pressure. The residue was purified by automated silica gel column chromatography (Biotage®) eluting with an ethyl acetate/hexanes gradient to give the title compound (0.96 g) as a colorless oil.

### III. 5-(Diphenylmethyl)thiophene-2-carboxylic acid

To a solution of ethyl 5-(diphenylmethyl)thiophene-2-carboxylate (960 mg) in methanol (15 mL), aqueous NaOH (6.0 M, 5 mL) was added. The resulting mixture was stirred at room temperature overnight, then was diluted with water and ether. The aqueous layer was acidified with HCl (2M), the resulting suspension was filtered and the solid was dried under vacuum to give the title compound (0.88 g) as an off-white solid.

### IV. Methyl (2S)-5-[(tert-butoxycarbonyl)amino]-2-({[5-(diphenylmethyl)thiophen-2-yl]carbonyl}amino)pentanoate

A solution of methyl (2S)-2-amino-5-[(tert-butoxycarbonyl)amino]pentanoate hydrochloride (1.0 g), 5-(diphenylmethyl)thiophene-2-carboxylic acid (0.88 g), diisopropylethylamine (1.5 mL) and HBTU (1.7 g) in DMF(15 mL) was stirred at room temperature overnight. The resulting mixture was diluted with ethyl acetate, washed with water (3 times) and with brine. The organic layer was dried over MgSO₄, filtered and concentrated under reduced pressure. The residue was purified by automated silica gel column chromatography (Biotage®) eluting with an ethyl acetate/hexanes gradient to give the title compound (1.2 g) as a pale yellow oil.

V. (2S)-5-[(tert-Butoxycarbonyl)amino]-2-([{5-(diphenylmethyl)thiophen-2-yl]carbonyl}amino)pentanoic acid.

To a solution of methyl (2S)-5-[(tert-butoxycarbonyl)amino]-2-({[5-(diphenylmethyl)thiophen-2-yl]carbonyl}amino)pentanoate (1.2 g) in THF (10 mL) and MeOH (1 mL), aqueous NaOH (2.0 M, 5 mL) was added. The resulting mixture was stirred at room temperature for 4 hours, then was diluted with ether and water. The aqueous layer was washed with ether and then acidified with HCl (2M) and extracted with ethy acetate. The ethyl acetate layer was washed with brine, dried over Na₂SO₄, filtered and concentrated under reduced pressure to give the title compound (1.2 g) as a sticky white solid.

### VI. (2S)-5-Amino-2-({[5-(diphenylmethyl)thiophen-2-yl]carbonyl}amino)pentanoic acid•TFA

To a solution of (2S)-5-[(tert-butoxycarbonyl)amino]-2-({[5-(diphenylmethyl)thiophen-2-yl]carbonyl}amino)pentanoic acid (1.2 g) in TFA (10 mL), triethylsilane (1 mL) and water (1 mL) were added. The resulting mixture was stirred at room temperature for 2 hour and the reaction mixture was diluted with water and lyophilized to give a the title compound (1.25 g) as a sticky white solid.

### VII. (2S)-2-({[5-(Diphenylmethyl)thiophen-2-yl]carbonyl}amino)-5-(ethanimidoylamino)pentanoic acid•TFA

To a solution of (2S)-5-amino-2-({[5-(diphenylmethyl)thiophen-2-yl]carbonyl}amino)pentanoic acid•TFA (6-5, 0.45 g) in ethanol (25 mL) at room temperature, ethyl acetimidate hydrochloride (200 mg) and NEt₃ (2 mL) were added sequentially. The resulting suspension was stirred at reflux overnight and the resulting mixture was concentrated under reduced pressure. The residue was purified by reverse phase HPLC, eluting with eluting with acetonitrile/water/TFA mixture and the fractions containing the desired product were combined and lyophilized to give the title compound (250 mg) as a white solid

The following compounds were synthesized by modifications of the general procedure described in Example 33.

Compound 118: Step VII was performed using (2S)-5-amino-2-({[5-(diphenylmethyl)thiophen-2-yl]carbonyl}amino)pentanoic acid•TFA (6-5, 0.2 g), ethanol (3 mL), ethyl butyrimidate hydrochloride (60 mg) and NEt₃ (0.12 mL) and the reaction mixture was stirred at room temperature overnight to give (2S)-5-(butanimidoylamino)-2-({[5-(diphenylmethyl)thiophen-2-yl]carbonyl}amino)pentanoic acid•TFA (118, 108 mg) as an off-white solid.

Compound 119. Ethyl picolinimidate was prepared by heating 2-cyanopyridine (0.50 g) in anhydrous ethanol (20 mL) in the presence of a trace amount of NaBH₄ to 75° C. overnight. The crude mixture was diluted with water, extracted with ethyl acetate and the organic layer was washed with brine, dried over Na₂SO₄ and concentrated under reduced pressure to give ethyl picolinimidate (0.62 g) as a yellow oil. Step VII was performed using (2S)-5-amino-2-({[5-(diphenylmethyl)thiophen-2-yl]carbonyl}amino)pentanoic acid•TFA (6-5, 0.20 g), ethanol (3 mL), ethyl picolinimidate hydrochloride (50 mg) and NEt₃ (0.13 mL) and the reaction mixture was stirred at room temperature overnight to give (2S)-2-({[5-(diphenylmethyl)thiophen-2-yl]carbonyl}amino)-5-{[imino(pyridin-2-yl)methyl]amino}pentanoic acid•TFA (119, 85 mg) as an off-white solid.

Compound 120: HCl gas was bubbled through a solution of benzonitrile (1.0 mL) in anhydrous ethanol for 45 minutes. The reaction mixture was stirred an additiona 10 minutes and then was concentrated under reduced pressure. The residue was taken up in acetonitrile, swirled to mix well and then the suspension was filtered. The solid was collected and dried under vacuum to give ethyl benzimidate hydrochloride (1.65 g) as an off-white solid. Step VII was performed using (2S)-5-amino-2-({[5-(diphenylmethyl)thiophen-2-yl]carbonyl}amino)pentanoic acid•TFA (6-5, 0.14 g), ethanol (3 mL), ethyl benzimidate hydrochloride (50 mg) and NEt₃ (0.08 mL) and the reaction mixture was stirred at room temperature overnight to give (2S)-2-({[5-(diphenylmethyl)thiophen-2-yl]carbonyl}amino)-5-{[imino(phenyl)methyl]amino}pentanoic acid•TFA (120, 73.8 mg) as an off-white solid.

Compound 121: Step VII was performed using (2S)-5-amino-2-({[5-(diphenylmethyl)thiophen-2-yl]carbonyl}amino)pentanoic acid•TFA (6-5, 0.17 g), ethanol (3 mL), ethyl cyclopropanecarbimidate hydrochloride (36 mg) and NEt₃ (0.1 mL) and the reaction mixture was stirred at room temperature overnight to give (2S)-5-{[cyclopropyl(imino)methyl]amino}-2-({[5-(diphenylmethyl)thiophen-2-yl]carbonyl}amino)pentanoic acid•TFA (121, 84.2 mg) as an off-white solid.

Compound 123: Step VII was performed using (2S)-5-amino-2-({[5-(diphenylmethyl)thiophen-2-yl]carbonyl}amino)pentanoic acid•TFA (6-5, 0.20 g), ethanol (3 mL), ethyl 3-ethoxy-3-iminopropanoate hydrochloride (70 mg) and NEt₃ (0.1 mL) and the reaction mixture was stirred at room temperature overnight to give (2S)-2-({[5-(diphenylmethyl)thiophen-2-yl]carbonyl}amino)-5-[(3-ethoxy-3-oxopropanimidoyl)amino]pentanoic acid•TFA (123, 80 mg) as an off-white solid.

Compound 132: HCl gas was bubbled through a solution of isobutyronitrile (0.90 g) in anhydrous ethanol (10 mL) for 2 hours minutes. The reaction mixture was then stirred overnight and then was concentrated under reduced pressure. The residue was taken up in acetonitrile, swirled to mix well and then the acetonitrile was decanted. The solid was dried under vacuum to give ethyl isobutyrimidate hydrochloride (1.71 g) as a white solid. Step VII was performed using (2S)-5-amino-2-({[5-(diphenylmethyl)thiophen-2-yl]carbonyl}amino)pentanoic acid•TFA (6-5, 0.25 g), ethanol (3 mL), ethyl isobutyrimidate hydrochloride (70 mg) and NEt₃ (0.14 mL) and the reaction mixture was stirred at room temperature overnight to give (2S)-2-({[5-(diphenylmethyl)thiophen-2-yl]carbonyl}amino)-5-[(2-methylpropanimidoyl)amino]pentanoic acid•TFA (132, 85 mg) as an off-white solid.

Compound 133: Step VII was performed using (2S)-5-amino-2-({[5-(diphenylmethyl)thiophen-2-yl]carbonyl}amino)pentanoic acid•TFA (6-5, 0.21 g), ethanol (3 mL), ethyl formimidate hydrochloride (40 mg) and NEt₃ (0.08 mL) and the reaction mixture was stirred at room temperature overnight to give (2S)-2-({[5-(diphenylmethyl)thiophen-2-yl]carbonyl}amino)-5-[(iminomethyl)amino]pentanoic acid•TFA (133, 89 mg) as an off-white solid.

Compound 136: Step VII was performed using (2S)-5-amino-2-({[5-(diphenylmethyl)thiophen-2-yl]carbonyl}amino)pentanoic acid•TFA (6-5, 0.215 g), methanol (3 mL), (E)-ethyl N-hydroxyacetimidate (200 mg) and NEt₃ (0.5 mL) and the reaction mixture was stirred at reflux for 1.5 hour then at room temperature for three days to give (2S)-2-({[5-(diphenylmethyl)thiophen-2-yl]carbonyl}amino)-5-{[1-(hydroxyamino)ethylidene]amino}pentanoic acid•TFA (136, 14 mg) as a white powder.

### Example 34

### Compound 124

### Synthesis of (2S)-2-[({5-[bis(4-methylphenyl)methyl]thiophen-2-yl}carbonyl)amino]-5-(ethanimidoylamino)pentanoic acid•TFA

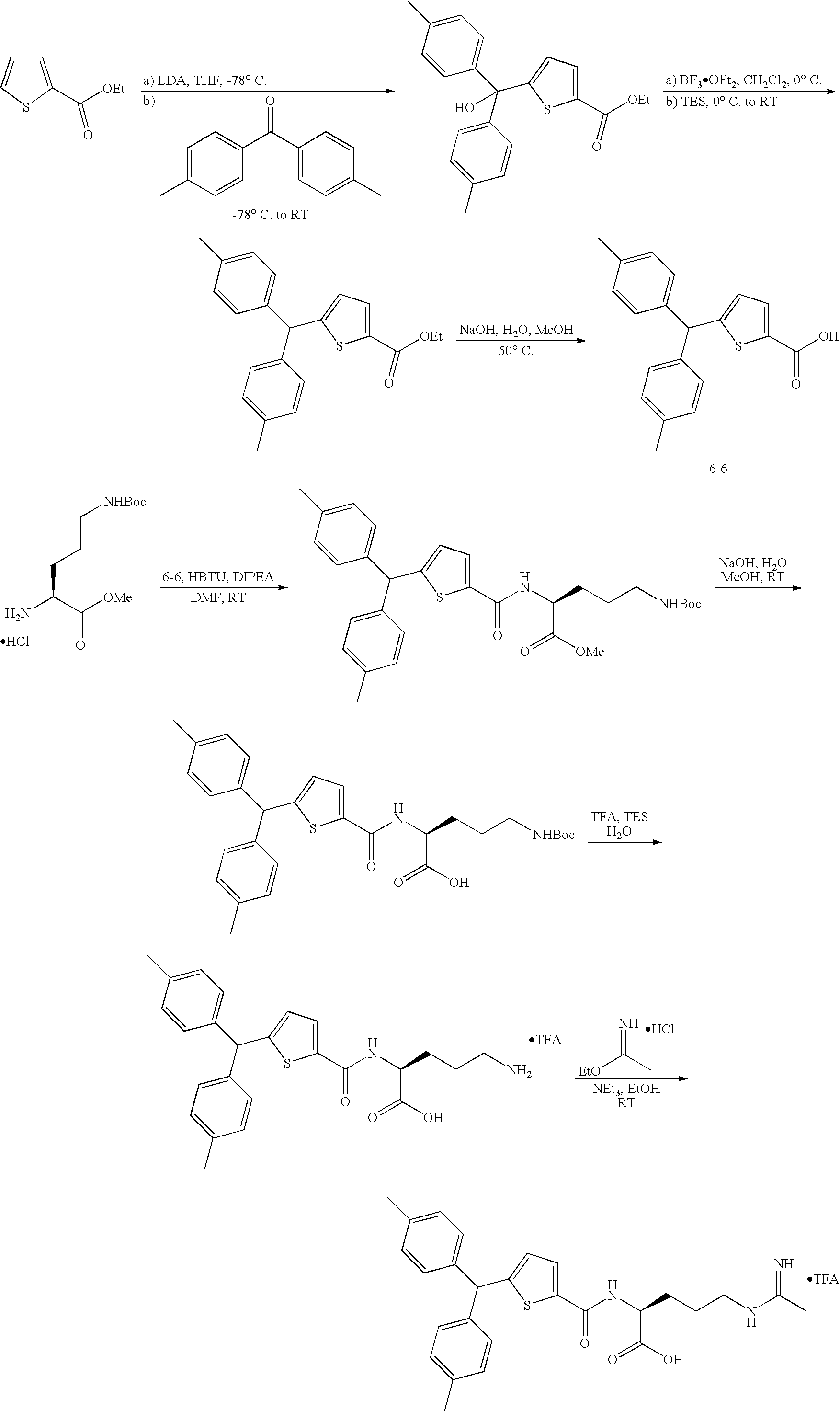

### I. Ethyl 5-{hydroxy[bis(4-methylphenyl)]methyl}thiophene-2-carboxylate

To a solution of ethyl thiophene-2-carboxylate (1.0 g) in THF (30 mL) cooled to −78° C. under a dry nitrogen atmosphere, LDA (1.8 M in THF/heptane/ethyl benzene, 4.1 mL) was added. The resulting mixture was stirred at −78° C. for 30 minutes, 4-4′-dimethylbenzophenone (1.29 g) was added and the mixture was allowed to warm to room temperature and was stirred overnight. The resulting mixture was quenched with HCl (2 M) and extracted with ethyl acetate. The organic layer was washed with water and brine, dried over MgSO₄, filtered and concentrated and the residue was purified by silica gel column chromatography eluting with an ethyl acetate/hexanes gradient to give ethyl 5-(hydroxydi-p-tolylmethyl)thiophene-2-carboxylate (1.63 g) as a light brown oil.

### II. Ethyl 5-[bis(4-methylphenyl)methyl]thiophene-2-carboxylate

To a ethyl 5-{hydroxy[bis(4-methylphenyl)]methyl}thiophene-2-carboxylate (1.63 g) in dichloromethane (10 mL) cooled to ° C., BF₃—OEt₂ (1.89 g) was added and the resulting mixture was stirred at 0° C. for 20 minutes. Triethylsilane (1.1 mL) was added, the mixture was allowed to warm to room temperature and was stirred for 2 hours. The reaction mixture was diluted with ethyl acetate and water and the organic layer was washed with brine, dried over MgSO₄, filtered and concentrated under reduced pressure to give the title compound (01.49 g) as a light brown oil. This material was used without purification.

### III. 5-[Bis(4-methylphenyl)methyl]thiophene-2-carboxylic acid

To a solution of ethyl 5-[bis(4-methylphenyl)methyl]thiophene-2-carboxylate (1.49 g) in methanol (25 mL), aqueous NaOH (2.0 M, 6 mL) was added. The resulting mixture was stirred at 50° C. overnight, then was diluted with water and ether. The aqueous layer was extracted with ether three times, then acidified with HCl (2M). The resulting suspension was extracted with ethyl acetate and the ethyl acetate layer was dried over MgSO₄, filtered and concentrated under reduced pressure to give the title compound (1.19 g) as a brown solid.

### IV. Methyl (2S)-2-[({5-[bis(4-methylphenyl)methyl]thiophen-2-yl}amino]-5-[(tert-butoxycarbonyl)amino]pentanoate

A solution of methyl (2S)-2-amino-5-[(tert-butoxycarbonyl)amino]pentanoate hydrochloride (1.25 g), 5-[Bis(4-methylphenyl)methyl]thiophene-2-carboxylic acid (1.19 g), diisopropylethylamine (2.0 mL) and HBTU (2.1 g) in DMF(15 mL) was stirred at room temperature overnight. The resulting mixture was diluted with ethyl acetate, washed with water, aqueous HCl (0.5 M) and brine. The organic layer was dried over MgSO₄, filtered and concentrated under reduced pressure and the residue was purified by silica gel column chromatography eluting with an ethyl acetate/hexanes gradient to give the title compound (1.76 g) as a pale pink solid.

### V. (2S)-2-[({5-[Bis(4-methylphenyl)methyl]thiophen-2-yl}carbonyl)amino]-5-[(tert-butoxycarbonyl)amino]pentanoic acid

To a solution of methyl (2S)-2-[({5-[bis(4-methylphenyl)methyl]thiophen-2-yl}carbonyl)amino]-5-[(tert-butoxycarbonyl)amino]pentanoate (1.0 g) in MeOH (25 mL), aqueous NaOH (2.0 M, 6 mL) was added. The resulting mixture was stirred at room temperature overnight, then was diluted with water and extracted with ether (3 times). The aqueous layer was acidified with HCl (2M) and extracted with ethy acetate. The ethyl acetate layer was dried over MgSO₄, filtered and concentrated under reduced pressure to give the title compound (0.90 g) as a light brown solid.

### VI. (2S)-5-Amino-2-[({5-[bis(4-methylphenyl)methyl]thiophen-2-yl}carbonyl)amino]pentanoic acid•TFA

To a solution of (2S)-2-[({5-[bis(4-methylphenyl)methyl]thiophen-2-yl}carbonyl)amino]-5-[(tert-butoxycarbonyl)amino]pentanoic acid (0.90 g) in TFA (8 mL), triethylsilane (1.1 mL) and water (1 mL) were added. The resulting mixture was stirred at room temperature for 2 hour and the reaction mixture was diluted with water and lyophilized to give a the title compound (1.04 g) as a light brown solid.

### VII. (2S)-2-[({5-[Bis(4-methylphenyl)methyl]thiophen-2-yl}carbonyl)amino]-5-(ethanimidoylamino)pentanoic acid•TFA

To a solution of (2S)-5-amino-2-[({5-[bis(4-methylphenyl)methyl]thiophen-2-yl}carbonyl)amino]pentanoic acid•TFA (0.20 g) in ethanol (4 mL) at room temperature, ethyl acetimidate hydrochloride (46 mg) and NEt₃ (0.11 mL) were added sequentially. The resulting mixture was stirred at room temperature overnight and was concentrated under reduced pressure. The residue was purified by reverse phase HPLC, eluting with eluting with acetonitrile/water/TFA mixture and the fractions containing the desired product were combined and lyophilized to give the title compound (45 mg) as an off-white solid.

The following compounds were synthesized by modifications of the general procedure described in Example 34.

Compound 125: Step I was performed using ethyl thiophene-2-carboxylate (1.0 g), THF (30 mL), LDA (1.8 M in THF/heptane/ethyl benzene, 4.1 mL) and 4-4′-dichlorobenzophenone (1.54 g) to give ethyl 5-[bis(4-chlorophenyl)(hydroxy)methyl]thiophene-2-carboxylate (2.20 g) as a light yellow oil. Step II was performed using ethyl 5-[bis(4-chlorophenyl)(hydroxy)methyl]thiophene-2-carboxylate (2.20 g), dichloromethane (10 mL), BF₃—OEt₂ (2.1 mL) and triethylsilane (1.3 mL) to give ethyl 5-[bis(4-chlorophenyl)methyl]thiophene-2-carboxylate (2.04 g) as a light brown oil. Step III was performed using ethyl 5-[bis(4-chlorophenyl)methyl]thiophene-2-carboxylate (2.04 g), methanol (30 mL) and aqueous NaOH (2.0 M, 8 mL) to give 5-[bis(4-chlorophenyl)methyl]thiophene-2-carboxylic acid (1.76 g) as a light brown solid. Step IV was performed using methyl (2S)-2-amino-5-[(tert-butoxycarbonyl)amino]pentanoate hydrochloride (0.93 g), 5-[bis(4-chlorophenyl)methyl]thiophene-2-carboxylic acid (1.0 g), diisopropylethylamine (1.5 mL), HBTU (1.57 g) and DMF(15 mL) to give methyl (2S)-2-[({5-[bis(4-chlorophenyl)methyl]thiophen-2-yl}carbonyl)amino]-5-[(tert-butoxycarbonyl)amino]pentanoate (1.31 g) as a light yellow solid. Step V was performed using methyl (2S)-2-[({5-[bis(4-chlorophenyl)methyl]thiophen-2-yl}carbonyl)amino]-5-[(tert-butoxycarbonyl)amino]pentanoate (1.0 g) in MeOH (25 mL), aqueous NaOH (2.0 M, 6 mL) to give (2S)-2-[({5-[bis(4-chlorophenyl)methyl]thiophen-2-yl}carbonyl)amino]-5-[(tert-butoxycarbonyl)amino]pentanoic acid (0.85 g) as an off-white solid. Step VI was performed using (2S)-2-[({5-[bis(4-chlorophenyl)methyl]thiophen-2-yl}carbonyl)amino]-5-[(tert-butoxycarbonyl)amino]pentanoic acid (0.85 g), TFA (8 mL), triethylsilane (0.95 mL) and water (1 mL) to give (2S)-5-amino-2-[({5-[bis(4-chlorophenyl)methyl]thiophen-2-yl}carbonyl)amino]pentanoic acid•TFA (0.93 g) as an off-white solid. Step VII was performed using (2S)-5-amino-2-[({5-[bis(4-chlorophenyl)methyl]thiophen-2-yl}carbonyl)amino]pentanoic acid•TFA (0.22 g), ethanol (4 mL), acetimidate hydrochloride (47 mg) and NEt₃ (0.11 mL) to give (2S)-2-[({5-[bis(4-chlorophenyl)methyl]thiophen-2-yl}carbonyl)amino]-5-(ethanimidoylamino)pentanoic acid•TFA (125, 38 mg) as a white solid.

Compound 126: Step I was performed using ethyl thiophene-2-carboxylate (0.64 g), THF (20 mL), LDA (1.8 M in THF/heptane/ethyl benzene, 2.6 mL) and 2,2′-dichlorobenzophenone (1.0 g) to give ethyl 5-[bis(2-chlorophenyl)(hydroxy)methyl]thiophene-2-carboxylate (1.28 g) as a light yellow oil. Step II was performed using ethyl 5-[bis(2-chlorophenyl)(hydroxy)methyl]thiophene-2-carboxylate (1.28 g), dichloromethane (10 mL), BF₃—OEt₂ (1.2 mL) and triethylsilane (0.76 mL) to give ethyl 5-[bis(2-chlorophenyl)methyl]thiophene-2-carboxylate (1.12 g) as a light brown oil. Step III was performed using ethyl 5-[bis(2-chlorophenyl)methyl]thiophene-2-carboxylate (1.12 g), methanol (25 mL), aqueous NaOH (2.0 M, 6 mL) to give 5-[bis(2-chlorophenyl)methyl]thiophene-2-carboxylic acid (0.95 g) as a brown solid. Step IV was performed using methyl (2S)-2-amino-5-[(tert-butoxycarbonyl)amino]pentanoate hydrochloride (0.89 g), 5-[bis(2-chlorophenyl)methyl]thiophene-2-carboxylic acid (0.95 g), diisopropylethylamine (1.4 mL), HBTU (1.49 g) and DMF(15 mL) to give methyl (2S)-2-[({5-[bis(2-chlorophenyl)methyl]thiophen-2-yl}carbonyl)amino]-5-[(tert-butoxycarbonyl)amino]pentanoate (1.77 g) as a light brown solid. Step V was performed using methyl (2S)-2-[({5-[bis(2-chlorophenyl)methyl]thiophen-2-yl}carbonyl)amino]-5-[(tert-butoxycarbonyl)amino]pentanoate (1.0 g) in MeOH (25 mL), aqueous NaOH (2.0 M, 6 mL) to give (2S)-2-[({5-[bis(2-chlorophenyl)methyl]thiophen-2-yl}carbonyl)amino]-5-[(tert-butoxycarbonyl)amino]pentanoic acid (0.88 g) as an light brown solid. Step VI was performed using (2S)-2-[(5-{bis(2-chlorophenyl)methyl thiophen-2-yl}carbonyl)amino]-5-[(tert-butoxycarbonyl)amino]pentanoic acid (0.88 g), TFA (8 mL), triethylsilane (1 mL) and water (1 mL) to give (2S)-5-amino-2-[({5-[bis(2-chlorophenyl)methyl]thiophen-2-yl}carbonyl)amino]pentanoic acid•TFA (0.89 g) as an off-white solid. Step VII was performed using (2S)-5-amino-2-[({5-[bis(2-chlorophenyl)methyl]thiophen-2-yl}carbonyl)amino]pentanoic acid•TFA (0.22 g), ethanol (4 mL), acetimidate hydrochloride (47 mg) and NEt₃ (0.11 mL) to give (2S)-2-[({5-[bis(2-chlorophenyl)methyl]thiophen-2-yl}carbonyl)amino]-5-(ethanimidoylamino)pentanoic acid•TFA (126, 55 mg) as a white solid.

Compound 131: Step I was performed using ethyl thiophene-2-carboxylate (0.65 g), THF (20 mL), LDA (1.8 M in THF/heptane/ethyl benzene, 2.7 mL) and 3,3′-dichlorobenzophenone (1.0 g) to give ethyl 5-[bis(3-chlorophenyl)(hydroxy)methyl]thiophene-2-carboxylate (1.35 g) as a light yellow oil. Step II was performed using ethyl 5-[bis(3-chlorophenyl)(hydroxy)methyl]thiophene-2-carboxylate (1.35 g), dichloromethane (10 mL), BF₃—OEt₂ (1.3 mL) and triethylsilane (0.8 mL) to give ethyl 5-[bis(3-chlorophenyl)methyl]thiophene-2-carboxylate (1.20 g) as a light brown oil. Step III was performed using ethyl 5-[bis(3-chlorophenyl)methyl]thiophene-2-carboxylate (1.20 g), methanol (25 mL), aqueous NaOH (2.0 M, 6 mL) to give 5-[bis(3-chlorophenyl)methyl]thiophene-2-carboxylic acid (1.15 g) as a brown solid. Step IV was performed using methyl (2S)-2-amino-5-[(tert-butoxycarbonyl)amino]pentanoate hydrochloride (1.07 g), 5-[bis(3-chlorophenyl)methyl]thiophene-2-carboxylic acid (1.15 g), diisopropylethylamine (1.7 mL), HBTU (1.81 g) and DMF(15 mL) to give methyl (2S)-2-[({5-[bis(3-chlorophenyl)methyl]thiophen-2-yl}carbonyl)amino]-5-[(tert-butoxycarbonyl)amino]pentanoate (1.21 g) as a light yellow solid. Step V was performed using methyl (2S)-2-[({5-[bis(3-chlorophenyl)methyl]thiophen-2-yl}carbonyl)amino]-5-[(tert-butoxycarbonyl)amino]pentanoate (1.21 g) in MeOH (20 mL), aqueous NaOH (2.0 M, 6 mL) to give (2S)-2-[({5-[bis(3-chlorophenyl)methyl]thiophen-2-yl}carbonyl)amino]-5-[(tert-butoxycarbonyl)amino]pentanoic acid (11.15 g) as an light yellow solid. Step VI was performed using (2S)-2-[({5-[bis(3-chlorophenyl)methyl]thiophen-2-yl}carbonyl)amino]-5-[(tert-butoxycarbonyl)amino]pentanoic acid (1.15 g), TFA (10 mL), triethylsilane (1.3 mL) and water (1 mL) to give (2S)-5-amino-2-[({5-[bis(3-chlorophenyl)methyl]thiophen-2-yl}carbonyl)amino]pentanoic acid•TFA (1.17 g) as a light yellow solid. Step VII was performed using (2S)-5-amino-2-[({5-[bis(3-chlorophenyl)methyl]thiophen-2-yl}carbonyl)amino]pentanoic acid•TFA (0.22 g), ethanol (4 mL), acetimidate hydrochloride (47 mg) and NEt₃ (0.11 mL) to give (2S)-2-[({5-[bis(3-chlorophenyl)methyl]thiophen-2-yl}carbonyl)amino]-5-(ethanimidoylamino)pentanoic acid•TFA (131, 50 mg) as a white solid.

Compound 129: Step I was performed using ethyl thiophene-2-carboxylate (1.0 g), THF (30 mL), LDA (1.8 M in THF/heptane/ethyl benzene, 4.1 mL) and 3,3′-difluorobenzophenone (1.34 g) to give ethyl 5-[bis(3-fluorophenyl)(hydroxy)methyl]thiophene-2-carboxylate (1.73 g) as a light yellow oil. Step II was performed using ethyl 5-[bis(3-fluorophenyl)(hydroxy)methyl]thiophene-2-carboxylate (1.73 g), dichloromethane (12 mL), BF₃—OEt₂ (1.8 mL) and triethylsilane (1.1 mL) to give ethyl 5-[bis(3-fluorophenyl)methyl]thiophene-2-carboxylate (1.60 g) as a light yellow oil. Step III was performed using ethyl 5-[bis(3-fluorophenyl)methyl]thiophene-2-carboxylate (1.60 g), methanol (20 mL), aqueous NaOH (2.0 M, 6 mL) to give 5-[bis(3-fluorophenyl)methyl]thiophene-2-carboxylic acid (1.32 g) as a reddish oil. Step IV was performed using methyl (2S)-2-amino-5-[(tert-butoxycarbonyl)amino]pentanoate hydrochloride (0.62 g), 5-[bis(3-fluorophenyl)methyl]thiophene-2-carboxylic acid (0.60 g), diisopropylethylamine (1.0 mL), HBTU (1.04 g) and DMF(12 mL) to give methyl (2S)-2-[({5-[bis(3-fluorophenyl)methyl]thiophen-2-yl}carbonyl)amino]-5-[(tert-butoxycarbonyl)amino]pentanoate (0.89 g) as a light brown solid. Step V was performed using methyl (2S)-2-[({5-[bis(3-fluorophenyl)methyl]thiophen-2-yl}carbonyl)amino]-5-[(tert-butoxycarbonyl)amino]pentanoate (0.89 g) in MeOH (20 mL), aqueous NaOH (2.0 M, 5 mL) to give (2S)-2-[({5-[bis(3-fluorophenyl)methyl]thiophen-2-yl}carbonyl)amino]-5-[(tert-butoxycarbonyl)amino]pentanoic acid (0.83 g) as an off-white solid. Step VI was performed using (2S)-2-[({5-[bis(3-fluorophenyl)methyl]thiophen-2-yl}carbonyl)amino]-5-[(tert-butoxycarbonyl)amino]pentanoic acid (0.83 g), TFA (8 mL), triethylsilane (1.0 mL) and water (1 mL) to give (2S)-5-amino-2-[({5-[bis(3-fluorophenyl)methyl]thiophen-2-yl}carbonyl)amino]pentanoic acid•TFA (0.99 g) as a light brown solid. Step VII was performed using (2S)-5-amino-2-[({5-[bis(3-fluorophenyl)methyl]thiophen-2-yl}carbonyl)amino]pentanoic acid•TFA (0.22 g), ethanol (4 mL), acetimidate hydrochloride (50 mg) and NEt₃ (0.12 mL) to give (2S)-2-[({5-[bis(3-fluorophenyl)methyl]thiophen-2-yl}carbonyl)amino]-5-(ethanimidoylamino)pentanoic acid•TFA (129, 36 mg) as a white solid.

Compound 128: Step I was performed using ethyl thiophene-2-carboxylate (1.0 g), THF (30 mL), LDA (1.8 M in THF/heptane/ethyl benzene, 4.1 mL) and 4,4′-difluorobenzophenone (1.34 g) to give ethyl 5-[bis(4-fluorophenyl)(hydroxy)methyl]thiophene-2-carboxylate (2.19 g) as a light orange oil. Step II was performed using ethyl 5-[bis(4-fluorophenyl)(hydroxy)methyl]thiophene-2-carboxylate (2.19 g), dichloromethane (12 mL), BF₃—OEt₂ (2.2 mL) and triethylsilane (1.4 mL) to give ethyl 5-[bis(4-fluorophenyl)methyl]thiophene-2-carboxylate (1.99 g) as a light brown oil. Step III was performed using ethyl 5-[bis(4-fluorophenyl)methyl]thiophene-2-carboxylate (1.99 g), methanol (25 mL), aqueous NaOH (2.0 M, 8 mL) to give 5-[bis(4-fluorophenyl)methyl]thiophene-2-carboxylic acid (1.78 g) as a light purple solid. Step IV was performed using methyl (2S)-2-amino-5-[(tert-butoxycarbonyl)amino]pentanoate hydrochloride (0.62 g), 5-[bis(4-fluorophenyl)methyl]thiophene-2-carboxylic acid (0.60 g), diisopropylethylamine (1.0 mL), HBTU (1.04 g) and DMF(12 mL) to give methyl (2S)-2-[({5-[bis(4-fluorophenyl)methyl]thiophen-2-yl}carbonyl)amino]-5-[(tert-butoxycarbonyl)amino]pentanoate (0.89 g) as a light brown solid. Step V was performed using methyl (2S)-2-[({5-[bis(4-fluorophenyl)methyl]thiophen-2-yl}carbonyl)amino]-5-[(tert-butoxycarbonyl)amino]pentanoate (0.89 g) in MeOH (20 mL), aqueous NaOH (2.0 M, 5 mL) to give (2S)-2-[({5-[bis(4-fluorophenyl)methyl]thiophen-2-yl}carbonyl)amino]-5-[(tert-butoxycarbonyl)amino]pentanoic acid (0.84 g) as an off-white solid. Step VI was performed using (2S)-2-[({5-[bis(4-fluorophenyl)methyl]thiophen-2-yl}carbonyl)amino]-5-[(tert-butoxycarbonyl)amino]pentanoic acid (0.84 g), TFA (8 mL), triethylsilane (1.0 mL) and water (1 mL) to give (2S)-5-amino-2-[({5-[bis(4-fluorophenyl)methyl]thiophen-2-yl}carbonyl)amino]pentanoic acid•TFA (0.91 g) as an off-white solid. Step VII was performed using (2S)-5-amino-2-[({5-[bis(4-fluorophenyl)methyl]thiophen-2-yl}carbonyl)amino]pentanoic acid•TFA (0.22 g), ethanol (4 mL), acetimidate hydrochloride (50 mg) and NEt₃ (0.12 mL) to give (2S)-2-[({5-[bis(4-fluorophenyl)methyl]thiophen-2-yl}carbonyl)amino]-5-(ethanimidoylamino)pentanoic acid•TFA (128, 41 mg) as a white solid.

Compound 130: Step I was performed using ethyl thiophene-2-carboxylate (1.0 g), THF (30 mL), LDA (1.8 M in THF/heptane/ethyl benzene, 4.1 mL) and 3,3′-bis(trifluoromethyl)benzophenone (1.97 g) to give ethyl 5-(hydroxy{bis[3-(trifluoromethyl)phenyl]}methyl)thiophene-2-carboxylate (1.92 g) as a yellow oil. Step II was performed using ethyl 5-(hydroxy {bis[3-(trifluoromethyl)phenyl]} methyl)thiophene-2-carboxylate (1.92 g), dichloromethane (12 mL), BF₃—OEt₂ (1.5 mL) and triethylsilane (1.0 mL) to give ethyl 5-{bis[3-(trifluoromethyl)phenyl]methyl}thiophene-2-carboxylate (1.78 g) as a light yellow oil. Step III was performed using ethyl 5-{bis[3-(trifluoromethyl)phenyl]methyl}thiophene-2-carboxylate (1.78 g), methanol (20 mL), aqueous NaOH (2.0 M, 6 mL) to give 5-{bis[3-(tritluoromethyl)phenyl]methyl}thiophene-2-carboxylic acid (1.30 g) as a reddish oil. Step IV was performed using methyl (2S)-2-amino-5-[(tert-butoxycarbonyl)amino]pentanoate hydrochloride (1.02 g), 5-{bis[3-(trifluoromethyl)phenyl]methyl}thiophene-2-carboxylic acid (1.30 g), diisopropylethylamine (1.60 mL), HBTU (1.72 g) and DMF(15 mL) to give methyl (2S)-2-{[(5-{bis[3-(trifluoromethyl)phenyl]methyl}thiophen-2-yl)carbonyl]amino}-5-[(tert-butoxycarbonyl)amino]pentanoate (1.41 g) as a brown solid. Step V was performed using methyl (2S)-2-{[(5-{bis[3-(trifluoromethyl)phenyl]methyl}thiophen-2-yl)carbonyl]amino}-5-[(tert-butoxycarbonyl)amino]pentanoate (1.0 g) in MeOH (20 mL), aqueous NaOH (2.0 M, 5 mL) to give (2S)-2-{[(5-{bis[3-(trifluoromethyl)phenyl]methyl}thiophen-2-yl)carbonyl]amino}-5-[(tert-butoxycarbonyl)amino]pentanoic acid (0.88 g) as a light yellow solid. Step VI was performed using (2S)-2-{[(5-{bis[3-(trifluoromethyl)phenyl]methyl}thiophen-2-yl)carbonyl]amino}-5-[(tert-butoxycarbonyl)amino]pentanoic acid (0.88 g), TFA (8 mL), triethylsilane (0.9 mL) and water (1 mL) to give (2S)-5-amino-2-{[(5-{bis[3-(trifluoromethyl)phenyl]methyl}thiophen-2-yl)carbonyl]amino}pentanoic acid•TFA (1.17 g) as a light brown solid. Step VII was performed using (2S)-5-amino-2-{[(5-{bis[3-(trifluoromethyl)phenyl]methyl}thiophen-2-yl)carbonyl]amino}pentanoic acid•TFA (0.30 g), ethanol (5 mL), acetimidate hydrochloride (59 mg) and NEt₃ (0.14 mL) to give (2S)-2-{[(5-{bis[3-(trifluoromethyl)phenyl]methyl}thiophen-2-yl)carbonyl]amino}-5-(ethanimidoylamino)pentanoic acid•TFA (130, 20 mg) as a white solid.

Compound 127: Step I was performed using ethyl thiophene-2-carboxylate (0.82 g), THF (20 mL), LDA (1.8 M in THF/heptane/ethyl benzene, 3.3 mL) and di-(2-thienyl)ketone (1.0 g) to give ethyl 5-[hydroxy(dithiophen-2-yl)methyl]thiophene-2-carboxylate (0.58 g) as a light brown solid. Step II was performed using ethyl 5-[hydroxy(dithiophen-2-yl)methyl]thiophene-2-carboxylate (0.58 g), dichloromethane (12 mL), BF₃—OEt₂ (0.63 mL) and triethylsilane (0.40 mL) to give ethyl 5-(dithiophen-2-ylmethyl)thiophene-2-carboxylate (0.53 g) as a purple oil. Step III was performed using ethyl 5-(dithiophen-2-ylmethyl)thiophene-2-carboxylate (0.53 g), methanol (20 mL), aqueous NaOH (2.0 M, 5 mL) to give 5-(dithiophen-2-ylmethyl)thiophene-2-carboxylic acid (0.26 g) as a brown solid. Step IV was performed using methyl (2S)-2-amino-5-[(tert-butoxycarbonyl)amino]pentanoate hydrochloride (0.29 g), give 5-(dithiophen-2-ylmethyl)thiophene-2-carboxylic acid (0.26 g), diisopropylethylamine (0.44 mL), HBTU (0.49 g) and DMF(10 mL) to give methyl (2S)-5-[(tert-butoxycarbonyl)amino]-2-({[5-(dithiophen-2-ylmethyl)thiophen-2-yl]carbonyl}amino)pentanoate (0.34 g) as a light orange oil. Step V was performed using methyl (2S)-5-[(tert-butoxycarbonyl)amino]-2-({[5-(dithiophen-2-ylmethyl)thiophen-2-yl]carbonyl}amino)pentanoate (0.34 g) in MeOH (12 mL), aqueous NaOH (2.0 M, 3 mL) to give (2S)-5-[(tert-butoxycarbonyl)amino]-2-({[5-(dithiophen-2-ylmethyl)thiophen-2-yl]carbonyl}amino)pentanoic acid (0.30 g) as a brown solid. Step VI was performed using (2S)-5-[(tert-butoxycarbonyl)amino]-2-({[5-(dithiophen-2-ylmethyl)thiophen-2-yl]carbonyl}amino)pentanoic acid (0.30 g), TFA (4 mL), triethylsilane (0.4 mL) and water (1 mL) to give (2S)-5-amino-2-({[5-(dithiophen-2-ylmethyl)thiophen-2-yl]carbonyl}amino)pentanoic acid•TFA (0.31 g) as a dark brown solid. Step VII was performed using (2S)-5-amino-2-({[5-(dithiophen-2-ylmethyl)thiophen-2-yl]carbonyl}amino)pentanoic acid•TFA (0.31 g), ethanol (5 mL), acetimidate hydrochloride (74 mg) and NEt₃ (0.18 mL) to give (2S)-2-({[5-(dithiophen-2-ylmethyl)thiophen-2-yl]carbonyl}amino)-5-(ethanimidoylamino)pentanoic acid•TFA (127, 47 mg) as a brown solid.

### Example 35

### Compound 98

### Synthesis of (2S)-5-carbamimidamido-2-[({5-[hydroxy(diphenyl)methyl]thiophen-2-yl}carbonyl)amino]pentanoic acid•TFA

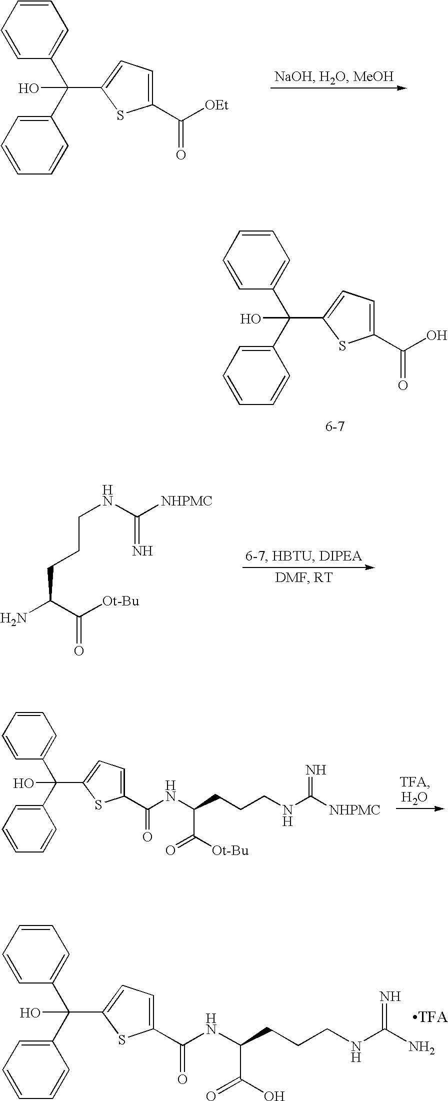

### I. 5-[Hydroxy(diphenyl)methyl]thiophene-2-carboxylic acid

To a solution of ethyl 5-[hydroxy(diphenyl)methyl]thiophene-2-carboxylate (100 mg) in methanol (2 mL), aqueous NaOH (2.0 M, 0.2 mL) was added. The resulting mixture was stirred at room temperature overnight, then was diluted with water and ether and the mixture was acidified with HCl (2M). The organic layer was washed with brine, dried over Na₂SO₄, filtered and concentrated under reduced pressure to give the title compound (0.08 g).

### II. tert-Butyl (2S)-2-[({5-[hydroxy(diphenyl)methyl]thiophen-2-yl}carbonyl)amino]-5-{[(2,2,5,7,8-pentamethyl-3,4-dihydro-2H-chromen-6-yl)sulfonyl]carbamimidamido}pentanoate

A solution of tert-butyl (2S)-2-amino-5-{[(2,2,5,7,8-pentamethyl-3,4-dihydro-2H-chromen-6-yl)sulfonyl]carbamimidamido}pentanoate (154 mg), 5-[hydroxy(diphenyl)methyl]thiophene-2-carboxylic acid (80 mg), HBTU (118 mg) and DIPEA (0.14 mL) in anhydrous DMF was stirred at room temperature overnight, then was diluted with water and EtOAc. The organic layer was washed with brine, dried over Na₂SO₄, filtered and concentrated under reduced pressure. The residue was purified by automated silica gel column chromatography (Biotage™) eluting with an ethyl acetate/hexanes gradient to give the title compound (110 mg).

### III. (2S)-5-Carbamimidamido-2-[({5-[hydroxy(diphenyl)methyl]thiophen-2-yl}carbonyl)amino]pentanoic acid•TFA

To a solution of tert-butyl (2S)-2-[({5-[hydroxy(diphenyl)methyl]thiophen-2-yl}carbonyl)amino]-5-{[(2,2,5,7,8-pentamethyl-3,4-dihydro-2H-chromen-6-yl)sulfonyl]carbamimidamido}pentanoate (110 mg) in TFA (2 mL), water (0.5 mL) was added. The resulting mixture was stirred at room temperature for 3 hours and the reaction mixture was concentrated under reduced pressure. The residue was purified by reverse phase HPLC, eluting with eluting with acetonitrile/water/TFA mixture and the fractions containing the desired product were combined and lyophilized to give the title compound (20 mg) as a yellow powder.

The following compounds were synthesized by modifications of the general procedure described in Example 35.

Compound 97: Step II was performed using tert-butyl (2S)-2-amino-5-{[(2,2,5,7,8-pentamethyl-3,4-dihydro-2H-chromen-6-yl(sulfonyl]carbamimidamido}pentanoate (202 mg), 5-(diphenylmethyl)thiophene-2-carboxylic acid (6-4, 100 mg), HBTU (193 mg), DIPEA (0.18 mL, 0.42 mmol) and DMF (10 mL) to give tert-butyl (2S)-2-({[5-(diphenylmethyl)thiophen-2-yl]carbonyl}amino)-5-{[(2,2,5,7,8-pentamethyl-3,4-dihydro-2H-chromen-6-yl)sulfonyl]carbamimidamido}pentanoate as a clear oil. Step III was performed using the material from step II, TFA (2 mL) and water (0.1 mL) and triethylsilane (0.1 mL) was also added to the reaction mixture. Purification by reverse phase HPLC gave (2S)-5-carbamimidamido-2-({[5-(diphenylmethyl)thiophen-2-yl]carbonyl}amino)pentanoic acid•TFA (145 mg) as an off-white powder.

### Example 36

### Compound 112

### Synthesis of (2S)-2-({[3-(diphenylmethyl)phenyl]carbonyl}amino)-5-(ethanimidoylamino)pentanoic acid•TFA

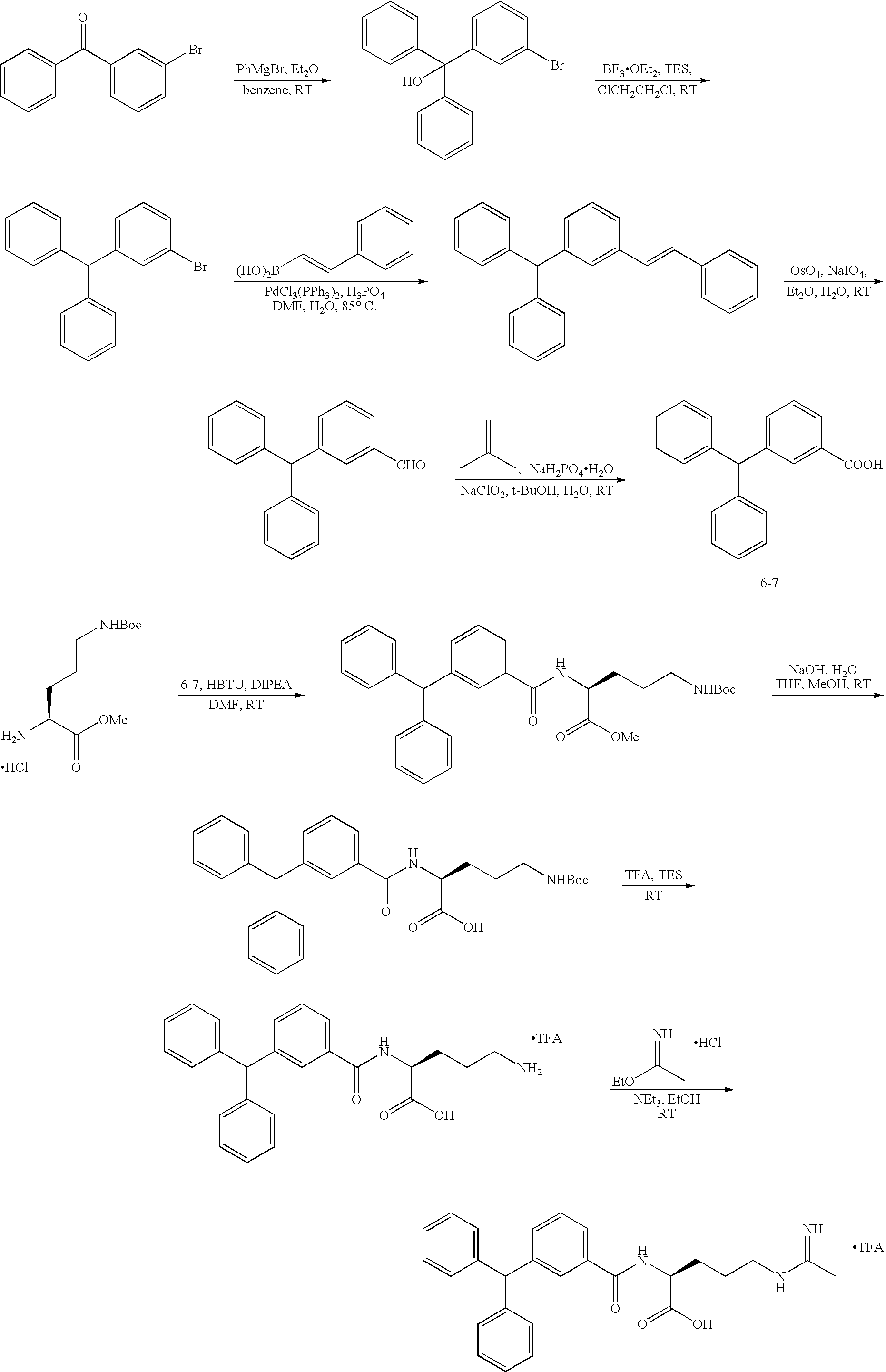

### I. (3-Bromophenyl)(diphenyl)methanol

To a solution of 3-bromobenzophenone (0.80 g) in anhydrous benzene (12 mL) at room temperature, phenylmagnesium bromide (3.0 M in ether, 1.6 mL) was added. The resulting mixture was stirred at room temperature overnight, HCl (2N) was added and the mixture was extracted with ethyl acetate. The organic layer was washed with water and brine, dried over Na₂SO₄, filtered and concentrated under reduced pressure. The residue was purified by automated silica gel column chromatography (Biotage®) eluting with 5% ethyl acetate/hexanes increasing to 10% ethyl acetate/hexanes to give the title compound (0.92 g) as a colorless oil.

### II. 1-Bromo-3-(diphenylmethyl)benzene

To a solution of (3-bromophenyl)(diphenyl)methanol (0.92 g) in dichloroethane (15 mL), Et₃SiH (0.52 mL) and BF₃—OEt₂ (1.03 mL) and were added sequentially. The reaction was stirred at room temperature for 15 minutes and the reaction mixture was diluted with ether and water. The organic layer washed with water and brine, dried over Na₂SO₄, filtered and concentrated under reduced pressure. The residue was purified by silica gel column chromatography, eluting with a hexanes to give the title compound (0.82 g).

### II. 1-(Diphenylmethyl)-3-[(E)-2-phenylethenyl]benzene

A solution of 1-bromo-3-(diphenylmethyl)benzene (0.82 g), (E)-styrylboronic acid (413 mg), PdCl₂(PPh₃)₂ (71 mg) and H₃PO₄ (1.68 g) in DMF(15 mL) and water (3 mL) under a nitrogen atmosphere was stirred at 85° C. overnight. The resulting mixture was diluted with ethyl acetate and washed with water (3 times) and brine. The organic layer was dried over Na₂SO₄, filtered and concentrated under reduced pressure. The residue was purified by silica gel column chromatography, eluting with hexanes to give the title compound (594 mg).

### IV. 3-(Diphenylmethyl)benzaldehyde

To a solution of 1-(diphenylmethyl)-3-[(E)-2-phenylethenyl]benzene (191 mg) in ether (5 mL) and water (1 mL), OSO₄ (0.16 M, 0.17 mL) and NaIO₄ (316 mg) were added. The resulting mixture was stirred at room temperature for 3 days, then was diluted with water and ethyl acetate. The organic layer was washed with water and brine, dried over Na₂SO₄, filtered and concentrated under reduced pressure. The residue was purified by silica gel column chromatography, eluting with 5% ethyl acetate/hexanes to give the title compound (107 mg).

### V. 3-(Diphenylmethyl)benzoic acid

Isobutylene gas was bubbled through a solution of 3-(diphenylmethyl)benzaldehyde (107 mg) in t-butanol (8 mL) for 10 seconds and a solution of NaH₂PO₄.H₂O (490 mg) and NaClO₂ (440 mg) in water (2 mL) was added. The resulting mixture was stirred at room temperature overnight and the reaction mixture was diluted with water. The mixture was extracted with a hexanes/ether mixture and the organic layer was washed with aqueous NaOH (2M, 3 times). The aqueous layers were combined, acidified with HCl (2M) and extracted with ethyl acetate. The ethyl acetate layer was washed with water and brine, dried over Na₂SO₄, filtered and concentrated under reduced pressure to give the title compound (118 mg) as a pale green solid.

### VI. Methyl (2S)-5-[(tert-butoxycarbonyl)amino]-2-({[3-(diphenylmethyl)phenyl]carbonyl}amino)pentanoate

A solution of methyl (2S)-2-amino-5-[(tert-butoxycarbonyl)amino]pentanoate hydrochloride (114 mg), 3-(diphenylmethyl)benzoic acid (6-7, 118 mg), diisopropylethylamine (0.26 mL) and HBTU (218 mg) in DMF(4 mL) was stirred at room temperature overnight. The resulting mixture was diluted with ethyl acetate, washed with water (3 times) and with brine. The organic layer was dried over MgSO₄, filtered and concentrated under reduced pressure. The residue was purified by automated silica gel column chromatography (Biotage®) eluting with 30% ethyl acetate/hexanes increasing to 70% ethyl acetate/hexanes to give the title compound (152 mg) as a white foam.

### VII. (2S)-5-[(tert-Butoxycarbonyl)amino]-2-({[3-(diphenylmethyl)phenyl]carbonyl}amino)pentanoic acid

To a solution of methyl (2S)-5-[(tert-butoxycarbonyl)amino]-2-({[3-(diphenylmethyl)phenyl]carbonyl}amino)pentanoate (152 mg) in THF (2 mL) and MeOH (2 mL), aqueous NaOH (2.0 M, 0.88 mL) was added. The resulting mixture was stirred at room temperature for 3 h, then was diluted with ether and water. The aqueous layer was washed with ether and then acidified with HCl (2M) and extracted with ethy acetate. The ethyl acetate layer was washed with water and brine, dried over Na₂SO₄, filtered and concentrated under reduced pressure to give the title compound (130 mg) as a white solid.

### VIII. (2S)-5-Amino-2-({[3-(diphenylmethyl)phenyl]carbonyl}amino)pentanoic acid•TFA

To a solution of (2S)-5-[(tert-butoxycarbonyl)amino]-2-({[3-(diphenylmethyl)phenyl]carbonyl}amino)pentanoic acid (130 mg) in TFA (2 mL), triethylsilane (0.2 mL) was added. The resulting mixture was stirred at room temperature for 2 hours and the reaction mixture was concentrated under reduced pressure to give the title compound. This material used without purification.

### IX. (2S)-2-({[3-(Diphenylmethyl)phenyl]carbonyl}amino)-5-(ethanimidoylamino)pentanoic acid•TFA

To a solution of (2S)-5-amino-2-({[3-(diphenylmethyl)phenyl]carbonyl}amino)pentanoic acid•TFA(crude material from previous reaction) in ethanol (2 mL) at room temperature, ethyl acetimidate hydrochloride (64 mg) and triethylamine (0.18 mL) were added sequentially. The resulting mixture was stirred overnight and was then concentrated under reduced pressure. The residue was purified by reverse phase HPLC, eluting with acetonitrile/water/TFA mixture and the fractions containing the desired product were lyophilized to give the title compound (88.7 mg) as a white solid.

The following compounds were synthesized by modifications of the general procedure described in Example 36.

Compound 108: Step I was performed using 4-bromobenzophenone (4.00 g) in anhydrous benzene (25 mL) and phenylmagnesium bromide (3.0 M in ether, 8.2 mL) to give (4-bromophenyl)(diphenyl)methanol (3.69 g) as a clear oil. Step II was performed using (4-bromophenyl)(diphenyl)methanol (1.94 g) in dichloroethane (30 mL) and Et₃SiH (1.1 mL) and BF₃ OEt₂ (2.2 mL) to give 1-bromo-4-(diphenylmethyl)benzene as a white solid. Step III was performed using 1-bromo-4-(diphenylmethyl)benzene (1.25 g), (E)-styrylboronic acid (0.63 g), PdCl₂(PPh₃)₂ (110 mg), H₃PO₄ (2.55 g), DMF(20 mL) and water (5 mL) to give 1-(diphenylmethyl)-4-[(E)-2-phenylethenyl]benzene (1.01 g) as an off-white solid. Step IV was performed using 1-(diphenylmethyl)-4-[(E)-2-phenylethenyl]benzene (1.01 g), ether (25 mL), water (8 mL), OSO₄ (0.16 M, 0.56 mL) and NaIO₄ (1.37 g) to give 4-(diphenylmethyl)benzaldehyde (0.80 g) as a light brown oil. Step V was performed using 4-(diphenylmethyl)benzaldehyde (0.80 g), butanol (18 mL), NaH₂PO₄.H₂O (3.66 g), NaClO₂ (3.32 g) and water (8 mL) to give 4-(diphenylmethyl)benzoic acid (0.52 g) as a yellow-green solid. Step VI was performed using methyl (2S)-2-amino-5-[(tert-butoxycarbonyl)amino]pentanoate hydrochloride (221 mg), 4-(diphenylmethyl)benzoic acid (225 mg), diisopropylethylamine (0.49 mL), HBTU (414 mg) and DMF(4 mL) to give methyl (2S)-5-[(tert-butoxycarbonyl)amino]-2-({[4-(diphenylmethyl)phenyl]carbonyl}amino)pentanoate (316 mg) as a pale yellow solid. Step VII was performed using methyl (2S)-5-[(tert-butoxycarbonyl)amino]-2-({[4-(diphenylmethyl)phenyl]carbonyl}amino)pentanoate (316 mg), THF (4 mL), MeOH (4 mL) and aqueous NaOH (2.0 M, 1.8 mL) to give (2S)-5-[(tert-butoxycarbonyl)amino]-2-({[4-(diphenylmethyl)phenyl]carbonyl}amino)pentanoic acid (289 mg) as a white foam. Step VIII was performed using (2S)-5-[(tert-butoxycarbonyl)amino]-2-({[4-(diphenylmethyl)phenyl]carbonyl}amino)pentanoic acid (289 mg), TFA (3 mL) and triethylsilane (0.3 mL) to give (2S)-5-amino-2-({[4-(diphenylmethyl)phenyl]carbonyl}amino)pentanoic acid•TFA (354 mg) as a white solid. Step IX was performed using (2S)-5-amino-2-({[4-(diphenylmethyl)phenyl]carbonyl}amino)pentanoic acid•TFA (354 mg), ethanol (6 mL), ethyl acetimidate hydrochloride (141 mg) and triethylamine (0.40 mL) to give (2S)-2-({[4-(diphenylmethyl)phenyl]carbonyl}amino)-5-(ethanimidoylamino)pentanoic acid•TFA (108, 194 mg) as a white solid.

### Example 37

### Compound III

### Synthesis of (2S)-5-carbamimidamido-2-({[4-(diphenylmethyl)phenyl]carbonyl}amino)-5-oxopentanoic acid•TFA

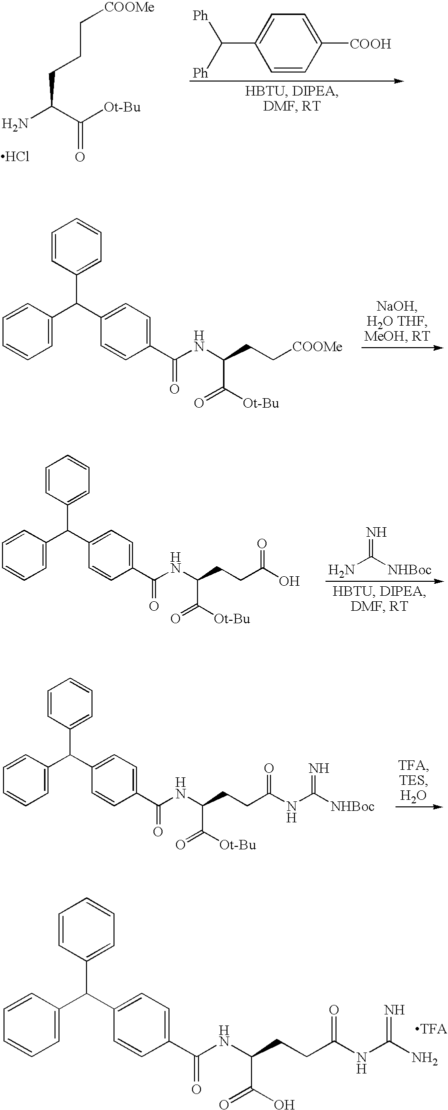

### I. 1-tert-Butyl 5-methyl (2S)-2-({[4-(diphenylmethyl)phenyl]carbonyl}amino)pentanedioate

A solution of 1-tert-butyl 5-methyl (2S)-2-aminopentanedioate hydrochloride (107 mg), 4-(diphenylmethyl)benzoic acid (from Step IV, compound 108, 122 mg), diisopropylethylamine (0.26 mL) and HBTU (223 mg) in DMF (4 mL) was stirred at room temperature overnight. The resulting mixture was diluted with ethyl acetate, washed with water (3 times) and with brine. The organic layer was dried over MgSO₄, filtered and concentrated under reduced pressure. The residue was purified by silica gel column chromatography eluting with 25% ethyl acetate/hexanes increasing to 33% ethyl acetate/hexanes to give the title compound (182 mg) as a colorless oil.

### II. (4S)-5-tert-Butoxy-4-({[4-(diphenylmethyl)phenyl]carbonyl}amino)-5-oxopentanoic acid

To a solution of 1-tert-butyl 5-methyl (2S)-2-({[4-(diphenylmethyl)phenyl]carbonyl}amino)pentanedioate (182 mg) in THF (2 mL) and MeOH (2 mL), aqueous NaOH (2.0 M, 0.37 mL) was added. The resulting mixture was stirred at room temperature for 3 h, then was diluted with ether and water. The aqueous layer was washed with ether and then acidified with HCl (2M) and extracted with ethy acetate. The ethyl acetate layer was washed with water and brine, dried over Na₂SO₄, filtered and concentrated under reduced pressure to give the title compound (155 mg).

### III. tert-Butyl (2S)-5-[(tert-butoxycarbonyl)carbamimidamido]-2-({[4-(diphenylmethyl)phenyl]carbonyl}amino)-5-oxopentanoate

A solution of (4S)-5-tert-butoxy-4-({[4-(diphenylmethyl)phenyl]carbonyl}amino)-5-oxopentanoic acid (155 mg), (tert-butoxycarbonyl)guanidine (57 mg), diisopropylethylamine (0.21 mL) and HBTU (179 mg) in DMF (3 mL) was stirred at room temperature overnight. The resulting mixture was diluted with ethyl acetate, washed with water (3 times) and with brine. The organic layer was dried over MgSO₄, filtered and concentrated under reduced pressure. The residue was purified by silica gel column chromatography eluting with 40% ethyl acetate/hexanes increasing to 100% ethyl acetate to give the title compound (140 mg) as a colorless oil.

### IV. (2S)-5-Carbamimidamido-2-({[4-(diphenylmethyl)phenyl]carbonyl}amino)-5-oxopentanoic acid•TFA

To a solution of tert-butyl (2S)-5-[(tert-butoxycarbonyl)carbamimidamido]-2-({[4-(diphenylmethyl)phenyl]carbonyl}amino)-5-oxopentanoate (140 mg) in TFA (2 mL), triethylsilane (0.2 mL) and water (0.2 mL) were added. The resulting mixture was stirred at room temperature for 3.5 hours, the reaction mixture was diluted with methyl tert-butyl ether and was washed twice with water. The organic layer was concentrated under reduced pressure and the residue was taken up in acetonitrile/water and lyophilized to give the title compound (194 mg) as a white solid.

The following compounds were synthesized by modifications of the general procedure described in Example 36.

Compound 99: Step I was conducted using 1-tert-butyl 5-methyl (2S)-2-aminopentanedioate hydrochloride (147 mg), 5-(diphenylmethyl)thiophene-2-carboxylic acid (6-4, 170 mg), diisopropylethylamine (0.3 mL), HBTU (264 mg) and DMF(5 mL) to give 1-tert-butyl 5-methyl (2S)-2-({[5-(diphenylmethyl)thiophen-2-yl]carbonyl}amino)pentanedioate (220 mg). Step II was conducted using 1-tert-butyl 5-methyl (2S)-2-({[5-(diphenylmethyl)thiophen-2-yl]carbonyl}amino)pentanedioate (220 mg) in methanol (5 mL) and aqueous NaOH (6.0 M, 1 mL). The crude material was purified by automated silica gel chromatography (Biotage™) eluting with 5% MeOH/ethyl acetate to give (4S)-5-tert-butoxy-4-({[5-(diphenylmethyl)thiophen-2-yl]carbonyl}amino)-5-oxopentanoic acid (100 mg). Step III was conducted using (4S)-5-tert-butoxy-4-({[5-(diphenylmethyl)thiophen-2-yl]carbonyl}amino)-5-oxopentanoic acid (100 mg), (tert-butoxycarbonyl)guanidine (66 mg), diisopropylethylamine (0.1 mL), HBTU (150 mg) and DMF (3 mL) to give tert-butyl (2S)-5-[(tert-butoxycarbonyl)carbamimidamido]-2-({[5-(diphenylmethyl)thiophen-2-yl]carbonyl}amino)-5-oxopentanoate (120 mg). Step IV was conducted using tert-butyl (2S)-5-[(tert-butoxycarbonyl)carbamimidamido]-2-({[5-(diphenylmethyl)thiophen-2-yl]carbonyl}amino)-5-oxopentanoate (120 mg), TFA (2 mL), triethylsilane (0.1 mL) and water (0.1 mL to give (2S)-5-carbamimidamido-2-({[5-(diphenylmethyl)thiophen-2-yl]carbonyl}amino)-5-oxopentanoic acid•TFA (99, 75 mg) as a white solid.

### Example 38

### Compound 134

### Synthesis of (2S)-({[5-(diphenylmethyl)thiophen-2-yl]carbonyl}amino)[3-(ethanimidoylamino)phenyl]ethanoic acid•TFA

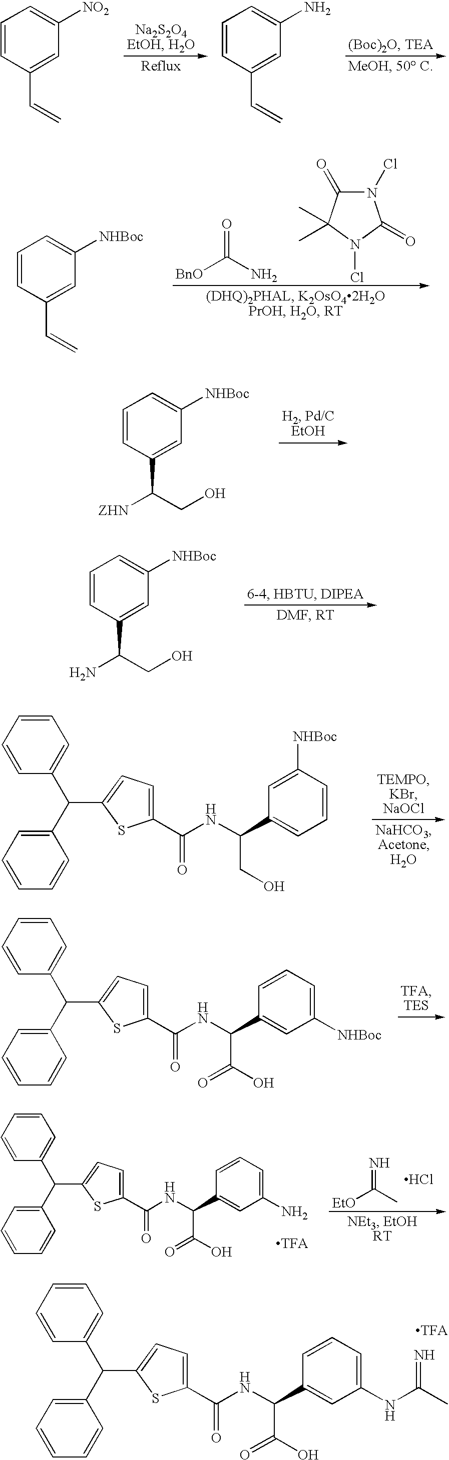

### I. 3-Vinylaniline

To a solution of 1-nitro-3-vinylbenzene (2.52 g) in ethanol (80 mL) and water (40 mL) heated to reflux, Na₂S₂O₄ (11.8 g) was added in small portions. The resulting mixture was heated to reflux for 1.5 h and additional Na₂S₂O₄ (5.8 g) was added in a single portion. Heating was continued until TLC showed no starting material remaining. The resulting mixture was diluted with water, and extracted twice with ether. The aqueous layer was basified with K₂CO₃ and extracted with ether. The ether layers were combined, washed with water and brine, dried over Na₂SO₄ and filtered. The filtrate was concentrated under reduced pressure to give the title compound (293 mg).

### II. tert-Butyl (3-ethenylphenyl)carbamate

To a solution of 3-vinylaniline (293 mg) and di-tert-butyl dicarbonate (805 mg) in methanol (15 mL), triethylamine (1.8 mL) was added. The resulting mixture was heated to 50° C. overnight. An additional portion of di-tert-butyl dicarbonate (290 mg) was added and the resulting mixture was heated a second night. The mixture was diluted with water and extracted with ethyl acetate and the organic layer was washed with water and brine, dried over Na₂SO₄ and filtered. The filtrate was concentrated under reduced pressure and the residue was purified by silica gel chromatography, eluting with 10% ethyl acetate/hexanes to give the title compound (485 mg) as a colorless oil.

### III. Benzyl [(1S)-1-{3-[(tert-butoxycarbonyl)amino]phenyl}-2-hydroxyethyl]carbamate

To a suspension of benzylcarbamate (1.03 g) in n-propanol (20 mL), a solution of NaOH (263 mg) in water (20 mL) was added followed by 1,3-dichloro-5,5-dimethylhydantoin (656 mg). The resulting mixture was stirred for 5 minutes to give a homogeneous solution. To this mixture, solutions of (DHQ)₂PHAL (90 mg) in n-propanol (5 mL) and tert-butyl (3-ethenylphenyl)carbamate (485 mg) in n-propanol (20 mL) were added sequentially followed by K₂OSO₄.2H₂O (34 mg). The resulting mixture was stirred at room temperature overnight and was diluted with water and ethyl acetate. The organic layer was washed with water and brine, dried over Na₂SO₄, filtered and concentrated under reduced pressure. The residue was purified by automated silica gel column chromatography (Biotage®) eluting with 10% ethyl acetate/hexanes increasing to 50% ethyl acetate/hexanes and finally 100% ethyl acetate to give the title compound (399 mg) as a white foam

### IV. tert-Butyl {3-[(1S)-1-amino-2-hydroxyethyl]phenyl}carbamate

To a solution of benzyl [(1S)—1-{3-[(tert-butoxycarbonyl)amino]phenyl}-2-hydroxyethyl]carbamate (399 mg) in ethanol (10 mL), palladium on carbon (10% Pd, 80 mg) was added. The atmosphere was replaced with hydrogen from a balloon and the resulting mixture was stirred for 1 hour. The resulting mixture was filtered through Celite® and the filtrate was concentrated under reduced pressure to give the title compound (234 mg).

### V. tert-Butyl {3-[(1S)-1-({[5-(diphenylmethyl)thiophen-2-yl]carbonyl}amino)-2-hydroxyethyl]phenyl} carbamate

A solution of 5-(diphenylmethyl)thiophene-2-carboxylic acid (6-4, 223 mg), (S)-tert-butyl {3-[(1S)-1-amino-2-hydroxyethyl]phenyl} carbamate (234 mg), diisopropylethylamine (0.50 mL) and HBTU (494 mg) in DMF(5 mL) was stirred at room temperature overnight. The resulting mixture was diluted with ethyl acetate, washed with water (3 times) and with brine. The organic layer was dried over MgSO₄, filtered and concentrated under reduced pressure. The residue was purified by silica gel column chromatography eluting with 30% ethyl acetate/hexanes increasing to 75% ethyl acetate/hexanes to give the title compound (393 mg).

### VI. (2S)-{3-[(tert-Butoxycarbonyl)amino]phenyl} ({[5-(diphenylmethyl)thiophen-2-yl]carbonyl}amino)ethanoic acid

To a solution of tert-butyl {3-[(1S)-1-({[5-(diphenylmethyl)thiophen-2-yl]carbonyl}amino)-2-hydroxyethyl]phenyl}carbamate (202 mg) in acetone (8 mL), saturated aqueous NaHCO₃ (4 mL) was added. To the resulting suspension, potassium bromide (8 mg) was added and the mixture was cooled to 0° C. To the cold mixture, TEMPO (78 mg) was added followed by Clorox bleach (0.7 mL). The resulting mixture was stirred at 0° C. for 1.5 h, then acetone (4 mL), TEMPO (41 mg) and Clorox bleach (0.4 mL) were added. The reaction mixture was allowed to warm to room temperature and was stirred 4 hours. The mixture was diluted with water and extracted twice with ether. The aqueous layer was acidified with HCl (2 M) and extracted with ethyl acetate. The ethyl acetate layer was washed with water amd brine, dried over Na₂SO₄, filtered and concentrated under reduced pressure to give the title compound (87 mg) as a pale yellow foam.

### VII. 2S)-(3-Aminophenyl)({[5-(diphenylmethyl)thiophen-2-yl]carbonyl}amino)ethanoic acid•TFA

To a solution of (2S)-{3-[(tert-butoxycarbonyl)amino]phenyl}({[5-(diphenylmethyl)thiophen-2-yl]carbonyl}amino)ethanoic acid (87 mg) in TFA (2 mL), triethylsilane (0.2 mL) was added. The resulting mixture was stirred at room temperature for 1.5 hours and the reaction mixture was concentrated under reduced pressure. The residue was suspended in ether and concentrated to give the title compound. This material used without purification.

### VIII. (2S)-({[5-(Diphenylmethyl)thiophen-2-yl]carbonyl}amino)[3-(ethanimidoylamino)phenyl]ethanoic acid•TFA

To a solution of (2S)-(3-aminophenyl)({[5-(diphenylmethyl)thiophen-2-yl]carbonyl}amino)ethanoic acid•TFA (crude material from previous reaction) in ethanol (2 mL) at room temperature, ethyl acetimidate hydrochloride (40 mg) and triethylamine (0.11 mL) were added sequentially. The resulting mixture was stirred at room temperature for 3 days then was heated to 55° C. overnight. Additional ethyl acetimidate hydrochloride (60 mg) and triethylamine (0.15 mL) were added and heating was continued overnight. The mixture was then concentrated under reduced pressure and the residue was purified by reverse phase HPLC, eluting with acetonitrile/water/TFA mixture. Fractions containing the desired product were lyophilized to give the title compound (8 mg) as a white solid.

The following compound were synthesized by modifications of the general procedure described in Example 38.

Compound 135: Step II was conducted using 4-vinylaniline (250 mg), di-tert-butyl dicarbonate (596 mg), methanol (10 mL) and triethylamine (0.88 mL) to give tert-butyl (4-ethenylphenyl)carbamate (419 mg) as a white solid. Step III was conducted using benzylcarbamate (267 mg) in n-propanol (5 mL), a solution of NaOH (70 mg) in water (4.2 mL), 1,3-dichloro-5,5-dimethylhydantoin (168 mg), (DHQ)₂PHAL (22 mg) in n-propanol (5 mL), tert-butyl (4-ethenylphenyl)carbamate (125 mg) in n-propanol (5 mL) and K₂OSO₄ (10 mg) to give benzyl [(1S)-1-{4-[(tert-butoxycarbonyl)amino]phenyl}-2-hydroxyethyl]carbamate (56 mg) as a white foam. Step IV was conducted using benzyl [(1S)-1-{4-[(tert-butoxycarbonyl)amino]phenyl}-2-hydroxyethyl]carbamate (56 mg), ethanol (3 mL) and palladium on carbon (10% Pd, 11 mg) to give tert-butyl {4-[(1S)-1-amino-2-hydroxyethyl]phenyl}carbamate (40 mg). Step V was conducted using 5-(diphenylmethyl)thiophene-2-carboxylic acid (6-4, 40 mg), tert-butyl {4-[(1S)-1-amino-2-hydroxyethyl]phenyl}carbamate (34 mg), diisopropylethylamine (0.007 mL), HBTU (69 mg) and DMF(1 mL) to give tert-butyl {4-[(1S)-1-({[5-(diphenylmethyl)thiophen-2-yl]carbonyl}amino)-2-hydroxyethyl]phenyl}carbamate (50 mg) as a white solid. Step VI was conducted using tert-butyl {4-[(1S)-1-({[5-(diphenylmethyl)thiophen-2-yl]carbonyl}amino)-2-hydroxyethyl]phenyl}carbamate (50 mg), acetone (2 mL), saturated aqueous NaHCO₃ (1 mL), potassium bromide (4 mg), TEMPO (18 mg) and Clorox bleach (0.15 mL) to give (2S)-{4-[(tert-butoxycarbonyl)amino]phenyl}({[5-(diphenylmethyl)thiophen-2-yl]carbonyl}amino)ethanoic acid (30 mg) as an orange oil. Step VII was conducted using (2S)-{4-[(tert-butoxycarbonyl)amino]phenyl}({[5-(diphenylmethyl)thiophen-2-yl]carbonyl}amino)ethanoic acid (30 mg), TFA (1 mL) and triethylsilane (0.1 mL) to give (2S)-(4-aminophenyl)({[5-(diphenylmethyl)thiophen-2-yl]carbonyl}amino)ethanoic acid•TFA. This material used without purification. Step VIII was conducted using (2S)-(4-aminophenyl)({[5-(diphenylmethyl)thiophen-2-yl]carbonyl}amino)ethanoic acid•TFA(crude material from previous reaction), ethanol (2 mL), ethyl acetimidate hydrochloride (14 mg) and triethylamine (0.04 mL) to give (2S)-({[5-(diphenylmethyl)thiophen-2-yl]carbonyl}amino)[4-(ethanimidoylamino)phenyl]ethanoic acid (135 1.6 mg) as a white solid.

### Example 39

### Compound 105

### Synthesis of (2S)-2-({[1-(diphenylmethyl)-2-oxo-1,2-dihydropyridin-3-yl]carbonyl}amino)-3-(1H-indol-3-yl)propanoic acid

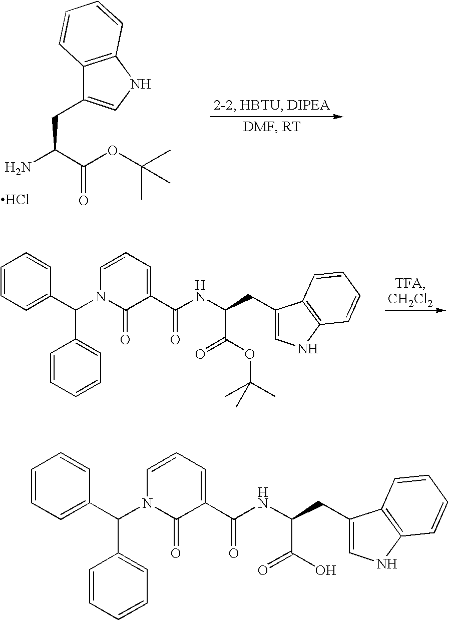

### I. tert-Butyl (2S)-2-({[1-(diphenylmethyl)-2-oxo-1,2-dihydropyridin-3-yl]carbonyl}amino)-3-(1H-indol-3-yl)propanoate

A solution tert-butyl (2S)-2-amino-3-(1H-indol-3-yl)propanoate (0.39 g), 1-(diphenylmethyl)-2-oxo-1,2-dihydropyridine-3-carboxylic acid (2-2, 0.40 g), diisopropylethylamine (0.3 mL) and HBTU (0.71 g) in DMF(7 mL) was stirred at room temperature for 3 days. The resulting mixture was diluted with ethyl acetate, washed with water, aqueous HCl (0.5 M) and brine. The organic layer was dried over MgSO₄, filtered and concentrated under reduced pressure and the residue was purified by silica gel column chromatography eluting with an ethyl acetate/hexanes gradient to give the title compound (0.72 g) as an off-white foam.

### II. (2S)-2-({[1-(Diphenylmethyl)-2-oxo-1,2-dihydropyridin-3-yl]carbonyl}amino)-3-(1H-indol-3-yl)propanoic acid

A solution of tert-butyl (2S)-2-({[1-(diphenylmethyl)-2-oxo-1,2-dihydropyridin-3-yl]carbonyl}amino)-3-(1H-indol-3-yl)propanoate (0.1 g) in dichloromethane (2 mL) and TFA (2 mL) was stirred at room temperature for 3 hours and the reaction mixture was concentrated under reduced pressure. The residue was purified by reverse phase HPLC, eluting with acetonitrile/water/TFA mixture and the fractions containing the desired product were combined and lyophilized to give the title compound (30 mg) as a white solid.

### Example 40

### Compound 87 Synthesis of (2S)-6-carbamimidamido-2-({[1-(diphenylmethyl)-2-oxo-1,2-dihydropyridin-3-yl]carbonyl}amino)hexanoic acid•TFA

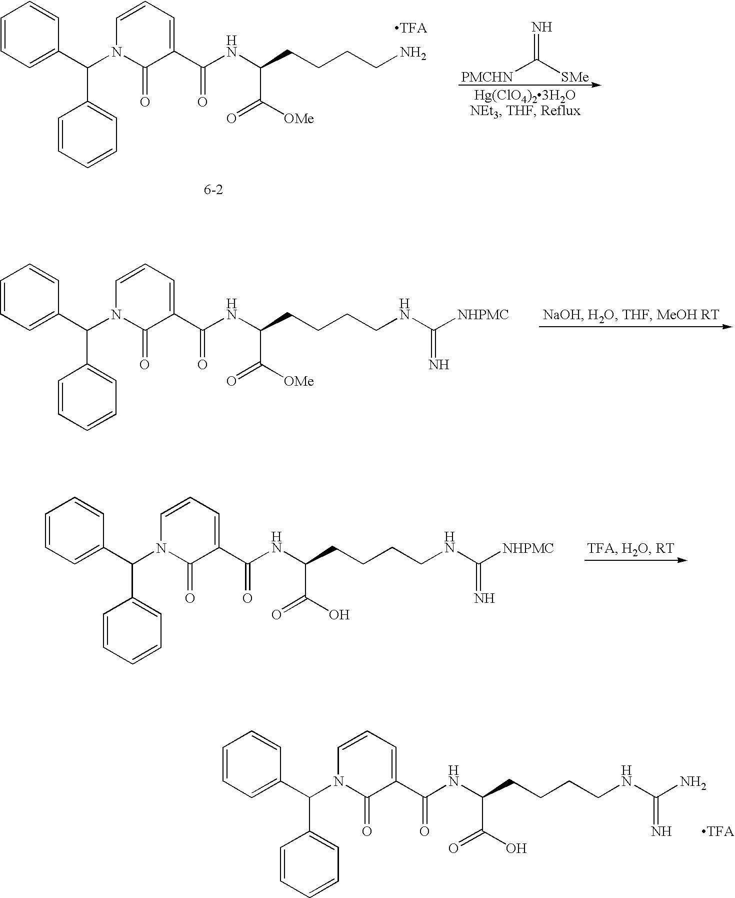

### I. Methyl (2S)-2-({[1-(diphenylmethyl)-2-oxo-1,2-dihydropyridin-3-yl]carbonyl}amino)-6-{[(2,2,5,7,8-pentamethyl-3,4-dihydro-2H-chromen-6-yl)sulfonyl]carbamimidamido}hexanoate

A solution of methyl (2S)-6-amino-2-({[1-(diphenylmethyl)-2-oxo-1,2-dihydropyridin-3-yl]carbonyl}amino)hexanoate•TFA (350 mg), PMC-S-methylisothiourea (311 mg), mercuric perchlorate trihydrate (372 mg) and triethylamine (0.35 mL) in THF (7 mL) was refluxed for 3 h. Additional PMC-S-methylisothiourea (210 mg) was added and the mixture was refluxed overnight. The resulting mixture was concentrated and the residue was taken up in ethyl acetate and filtered. The filtrate was washed with water, saturated aqueous NaHCO₃ and brine, dried over Na₂SO₄ and filtered. The filtrate was concentrated and the residue was purified by silica gel chromatography, eluting with 60% ethyl acetate/hexanes then 100% ethyl acetate and finally 10% methanol/dichloromethane to give the title compound (94 mg) as a colorless oil.

### II. (2S)-2-({[1-(Diphenylmethyl)-2-oxo-1,2-dihydropyridin-3-yl]carbonyl}amino)-6-{[(2,2,5,7,8-pentamethyl-3,4-dihydro-2H-chromen-6-yl)sulfonyl]carbamimidamido} hexanoic acid

To a solution of methyl (2S)-2-({[1-(diphenylmethyl)-2-oxo-1,2-dihydropyridin-3-yl]carbonyl}amino)-6-{[(2,2,5,7,8-pentamethyl-3,4-dihydro-2H-chromen-6-yl)sulfonyl]carbamimidamido}hexanoate (94 mg) in THF (1.5 mL) and MeOH (1.5 mL), aqueous NaOH (2.0 M, 0.37 mL) was added. The resulting mixture was stirred at room temperature for 4 h, then was diluted with ether and water. The aqueous layer was washed with ether and then acidified with HCl (2M) and extracted with ethy acetate. The ethyl acetate layer was washed with water and brine, dried over Na₂SO₄, filtered and concentrated under reduced pressure to give the title compound (80 mg) as a colorless oil.

### III. (2S)-6-Carbamimidamido-2-({[1-(diphenylmethyl)-2-oxo-1,2-dihydropyridin-3-yl]carbonyl}amino)hexanoic acid•TFA

To a solution of (2S)-2-({[1-(diphenylmethyl)-2-oxo-1,2-dihydropyridin-3-yl]carbonyl}amino)-6-{[(2,2,5,7,8-pentamethyl-3,4-dihydro-2H-chromen-6-yl)sulfonyl]carbamimidamido}hexanoic acid (80 mg) in TFA (2 mL), water (0.2 mL) was added. The resulting mixture was stirred at room temperature for 3.25 hours and the reaction mixture was diluted with methyl tert-butylether (20 mL) and the resulting suspension was centrifuged and the supernatant was decanted. The solid was taken up in acetonitrile and water and lyophilized. The resulting white solid was purified by reverse phase HPLC, eluting with acetonitrile/water/TFA mixture and the fractions containing the desired product were combined and lyophilized to give the title compound (30.9 mg) as a white solid.

### Example 41

### Compound 91

### Synthesis of (2S)-5-(acetylamino)-2-({[1-(diphenylmethyl)-2-oxo-1,2-dihydropyridin-3-yl]carbonyl}amino)pentanoic acid

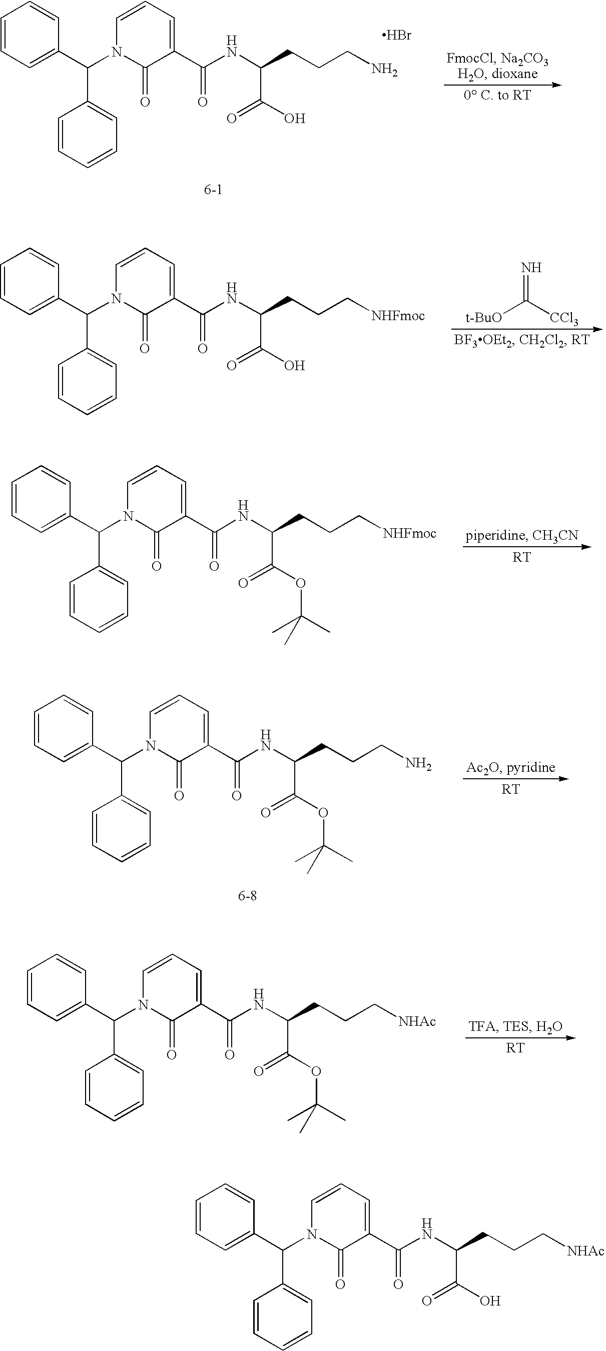

### I. (2S)-2-({[1-(Diphenylmethyl)-2-oxo-1,2-dihydropyridin-3-yl]carbonyl}amino)-5-{[(9H-fluoren-9-ylmethoxy)carbonyl]amino}pentanoic acid

To a mixture of (2S)-5-amino-2-({[1-(diphenylmethyl)-2-oxo-1,2-dihydropyridin-3-yl]carbonyl}amino)pentanoic acid (6-1, 2.12 g) and Na₂CO₃ (2.18 g) in water (20 mL) cooled to 0° C., a solution of (9H-fluoren-9-yl)methyl chloroformate (1.21 g) in dioxane (40 mL) was added slowly. The resulting mixture was allowed to warm to room temperature and was stirred for 4 hours. The resulting mixture was diluted with water and ether and the aqueous layer was acidified with HCl (2N) and then extracted with ethyl acetate. The ethyl acetate layer was washed twice with water and with brine, dried over Na₂SO₄, filtered and concentrated under reduced pressure to give the title compound (2.25 g) as a pale orange solid.

### II. tert-Butyl (2S)-2-({[1-(diphenylmethyl)-2-oxo-1,2-dihydropyridin-3-yl]carbonyl}amino)-5-{[(9H-fluoren-9-ylmethoxy)carbonyl]amino}pentanoate

To a solution of (2S)-2-({[1-(diphenylmethyl)-2-oxo-1,2-dihydropyridin-3-yl]carbonyl}amino)-5-{[(9H-fluoren-9-ylmethoxy)carbonyl]amino}pentanoic acid (2.25 g) in dichloromethane (20 mL), tert-butyl 2,2,2-trichloroacetimidate (0.94 mL) and BF₃.OEt₂ (0.12 mL) were added sequentially. The resulting mixture was stirred at room temperature for 3 days and was diluted with water and ethyl acetate. The organic layer was washed with water and brine, dried over Na₂SO₄, filtered and concentrated. The residue was purified by automated silica gel chromatography (Biotage®), eluting with 60% ethyl acetate/hexanes then 100% ethyl acetate and finally 10% methanol/dichloromethane to give the title (1.08 g).

### III. tert-Butyl (2S)-5-amino-2-({[1-(diphenylmethyl)-2-oxo-1,2-dihydropyridin-3-yl]carbonyl}amino)pentanoate

To a solution of tert-butyl (2S)-2-({[1-(diphenylmethyl)-2-oxo-1,2-dihydropyridin-3-yl]carbonyl}amino)-5-{[(9H-fluoren-9-ylmethoxy)carbonyl]amino}pentanoate (150 mg) in dichloromethane (2 mL), piperidine (0.4 mL) was added. The resulting mixture was stirred at room temperature for 1 hour and was concentrated. The residue was taken up in dichloromethane and was concentrated to give the title compound. This material was used directly without purification.

### IV. tert-Butyl (2S)-5-(acetylamino)-2-({[1-(diphenylmethyl)-2-oxo-1,2-dihydropyridin-3-yl]carbonyl}amino)pentanoate

To a solution of tert-butyl (2S)-5-amino-2-({[1-(diphenylmethyl)-2-oxo-1,2-dihydropyridin-3-yl]carbonyl}amino)pentanoate (crude material from step III) in pyridine (2 mL), acetic anhydride (0.031 mL) was added. The mixture was stirred at room temperature for 2 hours, then was diluted with ethyl acetate and washed sequentially with water, 5% aqueous CuSO₄ (3 times), water (3 times) and brine. The organic layer was dried over Na₂SO₄, filtered and concentrated under reduced pressure and the residue was purified by silica gel chromatography, eluting with ethyl acetate to give the title compound (91 mg).

### V. (2S)-5-(Acetylamino)-2-({[1-(diphenylmethyl)-2-oxo-1,2-dihydropyridin-3-yl]carbonyl}amino)pentanoic acid

To a solution of tert-butyl (2S)-5-(acetylamino)-2-({[1-(diphenylmethyl)-2-oxo-1,2-dihydropyridin-3-yl]carbonyl}amino)pentanoate (91 mg) in TFA (2 mL), triethylsilane (0.2 mL) and water (0.2 mL) were added. The resulting mixture was stirred at room temperature for 2 hours and the reaction mixture was diluted with water and ether and the ether layer was extracted with aqueous NaOH (2N) and the combined aqueous layers were acidified with HCl (2N). The resulting suspension was extracted with ethyl acetate and the ethyl acetate layer was washed with water and brine, dried over Na₂SO₄, filtered and concentrated under reduced pressure. The residue was taken up in acetonitrile and water and the solution was lyophilized to give the title compound (71 mg) as a white solid.

### Example 42

### Compound 95

### Synthesis of (2S)-5-(diethylamino)-2-({[1-(diphenylmethyl)-2-oxo-1,2-dihydropyridin-3-yl]carbonyl}amino)pentanoic acid•TFA

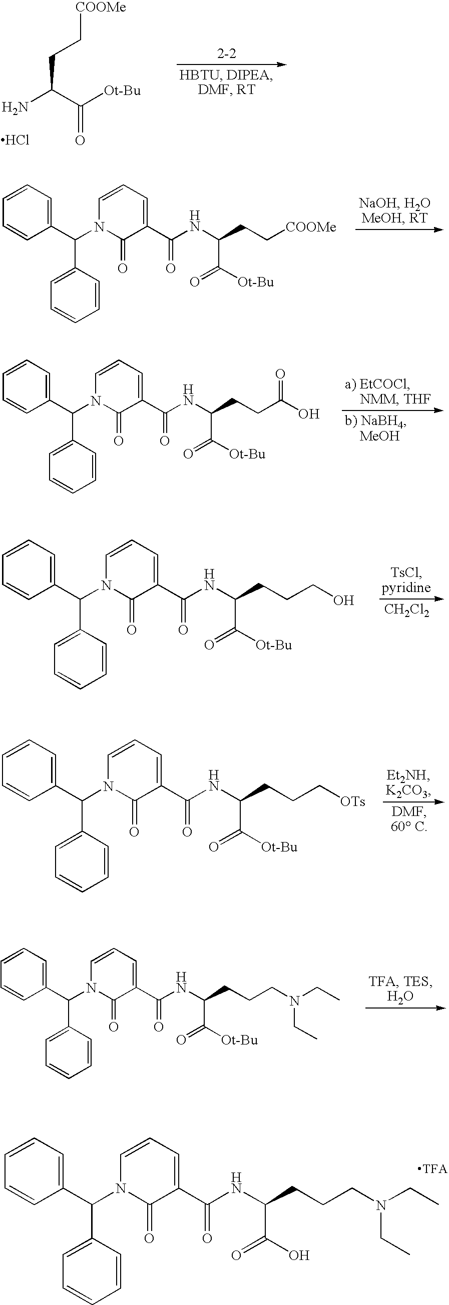

### I. 1-tert-Butyl 5-methyl (2S)-2-({[1-(diphenylmethyl)-2-oxo-1,2-dihydropyridin-3-yl]carbonyl}amino)pentanedioate

A solution of 1-tert-butyl 5-methyl (2S)-2-aminopentanedioate hydrochloride (415 mg), 1-(diphenylmethyl)-2-oxo-1,2-dihydropyridine-3-carboxylic acid (2-2, 549 mg), diisopropylethylamine (0.87 mL) and HBTU (933 mg) in DMF(10 mL) was stirred at room temperature overnight. The resulting mixture was diluted with ethyl acetate, washed with water (3 times) and with brine. The organic layer was dried over MgSO₄, filtered and concentrated under reduced pressure. The residue was purified by automated silica gel column chromatography (Biotage™), eluting with 10% ethyl acetate/hexanes increasing to 80% ethyl acetate/hexanes to give the title compound (870 mg) as a colorless oil.

### II. (4S)-5-tert-Butoxy-4-({[1-(diphenylmethyl)-2-oxo-1,2-dihydropyridin-3-yl]carbonyl}amino)-5-oxopentanoic acid

To a solution of 1-tert-butyl 5-methyl (2S)-2-({[1-(diphenylmethyl)-2-oxo-1,2-dihydropyridin-3-yl]carbonyl}amino)pentanedioate (360 mg) in MeOH (40 mL), aqueous NaOH (6.0 M, 3 mL) was added. The resulting mixture was stirred at room temperature for 3 h, then was diluted with ether and water. The aqueous layer was washed with ether and then acidified with HCl (2M) and extracted with ethy acetate. The ethyl acetate layer was washed with water and brine, dried over Na₂SO₄, filtered and concentrated under reduced pressure. The residue was purified by automated silica gel column chromatography (Biotage®), eluting with 100% ethyl acetate increasing to 10% methanol/ethyl acetate to give the title compound (240 mg).

### III. tert-Butyl (2S)-2-({[1-(diphenylmethyl)-2-oxo-1,2-dihydropyridin-3-yl]carbonyl}amino)-5-hydroxypentanoate

To a solution of (4S)-5-tert-butoxy-4-({[1-(diphenylmethyl)-2-oxo-1,2-dihydropyridin-3-yl]carbonyl}amino)-5-oxopentanoic acid (240 mg) in THF (4 mL) cooled to −10° C., N-methylmorpholine (0.08 mL) and ethyl chloroformate (0.07 mL) were added. The reaction was stirred for 15 minutes and methanol (10 mL) and NaBH₄ (57 mg) were added. The reaction mixture was stirred for 20 minutes, then was acidified with HCl (2N) and extracted with ethyl acetate. The organic layer was washed with water and brine, dried over Na₂SO₄, filtered and concentrated under reduced pressure. The residue was purified by automated silica gel column chromatography (Biotage®), eluting with 20% ethyl acetate/hexanes increasing to 100% ethyl acetate/hexanes to give the title compound (190 mg).

### IV. tert-Butyl (2S)-2-({[1-(diphenylmethyl)-2-oxo-1,2-dihydropyridin-3-yl]carbonyl}amino)-5-{[(4-methylphenyl)sulfonyl]oxy} pentanoate

To a solution of tert-butyl (2S)-2-({[1-(diphenylmethyl)-2-oxo-1,2-dihydropyridin-3-yl]carbonyl}amino)-5-hydroxypentanoate (190 mg) in dichloromethane (10 mL), tosylchloride (228 mg) and pyridine (0.1 mL) were added. The resulting mixture was stirred at room temperature for 2 hours and was concentrated. The residue was purified by automated silica gel column chromatography (Biotage®), eluting with hexanes increasing to 60% ethyl acetate/hexanes to give the title compound (55 mg).

### V. tert-Butyl (2S)-5-(diethylamino)-2-({[1-(diphenylmethyl)-2-oxo-1,2-dihydropyridin-3-yl]carbonyl}amino)pentanoate

To a solution of tert-butyl (2S)-2-({[1-(diphenylmethyl)-2-oxo-1,2-dihydropyridin-3-yl]carbonyl}amino)-5-{[(4-methylphenyl)sulfonyl]oxy}pentanoate (43 mg) in DMF (3 mL), diethylamine (0.02 mL) and K₂CO₃ (29 mg) were added. The resulting mixture was heated to 60° C. for 6 hours and was concentrated. The residue was purified by automated silica gel column chromatography (Biotage®), eluting with ethyl acetate increasing to 15% methanol/ethyl acetate to give the title compound (29 mg).

### VI. (2S)-5-(Diethylamino)-2-({[1-(diphenylmethyl)-2-oxo-1,2-dihydropyridin-3-yl]carbonyl}amino)pentanoic acid•TFA

To a solution of tert-butyl (2S)-5-(diethylamino)-2-({[1-(diphenylmethyl)-2-oxo-1,2-dihydropyridin-3-yl]carbonyl}amino)pentanoate (29 mg) in TFA (2 mL), triethylsilane (0.1 mL) and water (0.1 mL) were added. The resulting mixture was stirred at room temperature for 3 hours and the reaction mixture was concentrated. The residue was was purified by reverse phase HPLC, eluting with acetonitrile/water/TFA mixture and the fractions containing the desired product were combined and lyophilized to give the title compound (15 mg) as a white solid.

### Example 43

### Compound 93

### Synthesis of (2S)-5-({bis[(1-methylethyl)amino]methylidene}amino)-2-({[1-(diphenylmethyl)-2-oxo-1,2-dihydropyridin-3-yl]carbonyl}amino)pentanoic acid•TFA

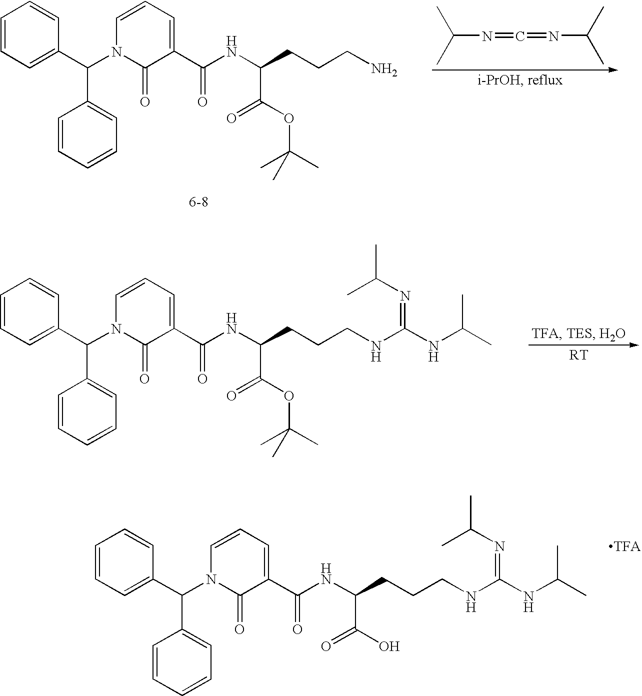

### I. tert-Butyl (2S)-5-({bis[(1-methylethyl)amino]methylidene}amino)-2-({[1-(diphenylmethyl)-2-oxo-1,2-dihydropyridin-3-yl]carbonyl}amino)pentanoate

To a solution of (S)-tert-butyl 5-amino-2-(1-benzhydryl-2-oxo-1,2-dihydropyridine-3-carboxamido)pentanoate (6-8, 122 mg theoretical) in isopropanol (5 mL), diisopropylcarbodiimide (0.060 mL) was added. The resulting mixture was heated to reflux overnight and then was concentrated under reduced pressure. The residue was purified by reverse phase HPLC, eluting with acetonitrile/water/TFA mixture and the fractions containing the desired product were combined and lyophilized to give the title compound (50 mg) as a white solid.

### II. (2S)-5-({Bis[(1-methylethyl)amino]methylidene}amino)-2-({[1-(diphenylmethyl)-2-oxo-1,2-dihydropyridin-3-yl]carbonyl}amino)pentanoic acid•TFA

To a solution of tert-butyl (2S)-5-({bis[(1-methylethyl)amino]methylidene}amino)-2-({[1-(diphenylmethyl)-2-oxo-1,2-dihydropyridin-3-yl]carbonyl}amino)pentanoate (50 mg) in TFA (2 mL), triethylsilane (0.2 mL) and water (0.2 mL) were added. The resulting mixture was stirred at room temperature for 3 hours and the reaction mixture was concentrated. The residue was purified by reverse phase HPLC, eluting with acetonitrile/water/TFA mixture and the fractions containing the desired product were combined and lyophilized to give the title compound (23 mg) as a white solid.

### Example 44

### Compound 92

### Synthesis of (2S)-2-({[1-(diphenylmethyl)-2-oxo-1,2-dihydropyridin-3-yl]carbonyl}amino)-5-(hydroxycarbamimidamido)pentanoic acid

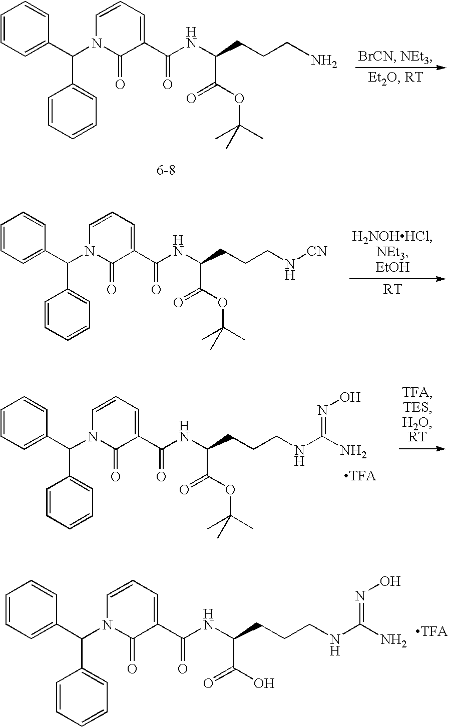

### I. tert-Butyl (2S)-5-(cyanoamino)-2-({[1-(diphenylmethyl)-2-oxo-1,2-dihydropyridin-3-yl]carbonyl}amino)pentanoate

To a solution of (tert-butyl (2S)-5-amino-2-({[1-(diphenylmethyl)-2-oxo-1,2-dihydropyridin-3-yl]carbonyl}amino)pentanoate (6-8, 219 mg theoretical) in ether (3 mL), cyanogen bromide (54 mg) and triethylamine (0.084 mL) were added. The resulting mixture was stirred at room temperature for 45 minutes and dichloromethane was added. Stirring was continued for 2 hours then the reaction mixture was concentrated under reduced pressure. The residue was purified by silica gel chromatography, eluting with 60% ethyl acetate/hexanes to give the title compound (114 mg) as a white foam.

### II. tert-Butyl (2S)-2-({[1-(diphenylmethyl)-2-oxo-1,2-dihydropyridin-3-yl]carbonyl}amino)-5-(hydroxycarbamimidamido)pentanoate•TFA

To a solution of tert-butyl (2S)-5-(cyanoamino)-2-({[1-(diphenylmethyl)-2-oxo-1,2-dihydropyridin-3-yl]carbonyl}amino)pentanoate (70 mg) in ethanol (2 mL), hydroxylamine hydrochloride (18 mg) and triethylamine (0.050 mL) were added. The resulting mixture was stirred at room temperature for 1.5 hours and then was diluted with ethyl acetate and washed with water, brine, dried over Na₂SO₄, filtered and concentrated under reduced pressure. The residue was purified by reverse phase HPLC, eluting with acetonitrile/water/TFA mixture and the fractions containing the desired product were combined and lyophilized to give the title compound (50 mg).

### III. (2S)-2-({[1-(Diphenylmethyl)-2-oxo-1,2-dihydropyridin-3-yl]carbonyl}amino)-5-(hydroxycarbamimidamido)pentanoic acid•TFA

To a solution of tert-butyl (2S)-2-({[1-(diphenylmethyl)-2-oxo-1,2-dihydropyridin-3-yl]carbonyl}amino)-5-(hydroxycarbamimidamido)pentanoate•TFA (50 mg) in TFA (2 mL), triethylsilane (0.2 mL) and water (0.2 mL) were added. The resulting mixture was stirred at room temperature for 2 hours and the reaction mixture was hours and the reaction mixture was diluted with methyl tert-butylether (20 mL) and was washed with water (3 times) and brine. The organic layer was concentrated then the residue was dissolved in acetonitrile and water and lyophilized to give the title compound (29 mg) as a white solid.

### Example 45

### Compound 89

### Synthesis of (3S)-6-carbamimidamido-3-({[1-(diphenylmethyl)-2-oxo-1,2-dihydropyridin-3-yl]carbonyl}amino)hexanoic acid•TFA

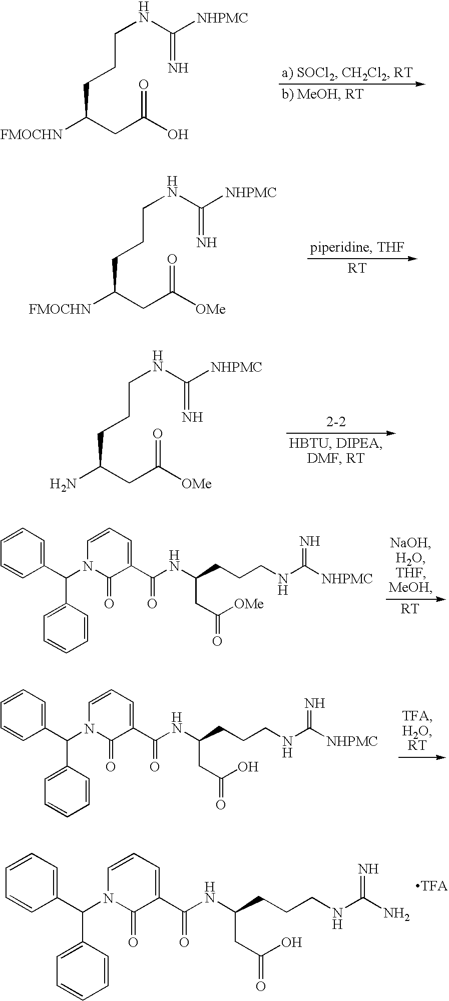

### I. Methyl (3S)-3-{[(9H-fluoren-9-ylmethoxy)carbonyl]amino}-6-{[(2,2,5,7,8-pentamethyl-3,4-dihydro-2H-chromen-6-yl)sulfonyl]carbamimidamido}hexanoate

To a solution of (3S)-3-{[(9H-fluoren-9-ylmethoxy)carbonyl]amino}-6-{[(2,2,5,7,8-pentamethyl-3,4-dihydro-2H-chromen-6-yl)sulfonyl]carbamimidamido}hexanoic acid (50 mg) in dichlormethane (1 mL) at room temperature, thionyl chloride (0.03 mL) was added. The resulting mixture was stirred for 2 hours and methanol (1 mL) was added. The reaction was stirred an additional 15 minutes and then concentrated under reduced pressure. The residue was taken up in hexanes and concentrate (3 times) to give the title compound (50 mg) as an off-white solid. This reaction was repeated using (S)-3-(((9H-fluoren-9-yl)methoxy)carbonylamino)-6-(3-(2,2,5,7,8-pentamethylchroman-6-ylsulfonyl)guanidino)hexanoic acid (0.23 g), dichlormethane (3.4 mL), thionyl chloride (0.12 mL) and methanol (1 mL) to give the title compound (0.23 g) as an off-white solid.

### II. Methyl (3S)-3-amino-6-{[(2,2,5,7,8-pentamethyl-3,4-dihydro-2H-chromen-6-yl)sulfonyl]carbamimidamido}hexanoate

To a solution of methyl (3S)-3-{[(9H-fluoren-9-ylmethoxy)carbonyl]amino}-6-{[(2,2,5,7,8-pentamethyl-3,4-dihydro-2H-chromen-6-yl)sulfonyl]carbamimidamido}hexanoate (0.28 g) in THF (2 mL) at room temperature, piperidine (0.2 mL) was added and the resulting mixture was stirred for 2 hours and concentrated under reduced pressure. The residue was purified by automated silica gel column chromatography (Biotage®), eluting with 30% ethyl acetate/hexanes increasing to 100% ethyl acetate, then 10% methanol/ethyl acetate and finally 30% methanol/ethyl acetate to give the title compound (0.10 g) as a white solid.

### III. Methyl (3S)-3-({[1-(diphenylmethyl)-2-oxo-1,2-dihydropyridin-3-yl]carbonyl}amino)-6-{[(2,2,5,7,8-pentamethyl-3,4-dihydro-2H-chromen-6-yl)sulfonyl]carbamimidamido}hexanoate

A solution of methyl (3S)-3-amino-6-{[(2,2,5,7,8-pentamethyl-3,4-dihydro-2H-chromen-6-yl)sulfonyl]carbamimidamido}hexanoate (0.10 g), 1-(diphenylmethyl)-2-oxo-1,2-dihydropyridine-3-carboxylic acid (2-2, 72 mg), diisopropylethylamine (0.05 mL) and HBTU (0.13 g) in DMF(1.2 mL) was stirred at room temperature for three days. The resulting mixture was diluted with ethyl acetate, washed with water (3 times) and with brine. The organic layer was dried over MgSO₄, filtered and concentrated under reduced pressure. The residue was purified by automated silica gel column chromatography (Biotage™), eluting with 30% ethyl acetate/hexanes increasing to 100% ethyl acetate to give the title compound (0.14 g) as a white foam.

### IV. (3S)-3-({[1-(Diphenylmethyl)-2-oxo-1,2-dihydropyridin-3-yl]carbonyl}amino)-6-{[(2,2,5,7,8-pentamethyl-3,4-dihydro-2H-chromen-6-yl)sulfonyl]carbamimidamido}hexanoic acid

To a solution of methyl (3S)-3-({[1-(diphenylmethyl)-2-oxo-1,2-dihydropyridin-3-yl]carbonyl}amino)-6-{[(2,2,5,7,8-pentamethyl-3,4-dihydro-2H-chromen-6-yl)sulfonyl]carbamimidamido}hexanoate (0.14 g) in THF (3 mL) and MeOH (0.5 mL), aqueous NaOH (2.0 M, 1.5 mL) was added. The resulting mixture was stirred at room temperature for 3 h, then was diluted with ether and water. The aqueous layer was washed with ether and then acidified with HCl (2M) and extracted with ethy acetate. The ethyl acetate layer was washed with water and brine, dried over MgSO₄, filtered and concentrated under reduced pressure to give the title compound (0.12 g) as a light yellow oil.

### V. (3S)-6-Carbamimidamido-3-([{1-(diphenylmethyl)-2-oxo-1,2-dihydropyridin-3-yl]carbonyl}amino)hexanoic acid•TFA

To a solution of (3S)-3-({[1-(diphenylmethyl)-2-oxo-1,2-dihydropyridin-3-yl]carbonyl}amino)-6-{[(2,2,5,7,8-pentamethyl-3,4-dihydro-2H-chromen-6-yl)sulfonyl]carbamimidamido}hexanoic acid (0.12 g) in TFA (3 mL), water (0.3 mL) was added. The resulting mixture was stirred at room temperature for 4 hours, the reaction mixture was diluted with methyl tert-butylether (30 mL), the resulting suspension was centrifuged and the supernatant was decanted. The solid was again taken up in methyl tert-butylether (25 mL) and the resulting suspension was agitated for several minutes and then centrifuged, decanting the supernatant. The resulting solid material was taken up in acetonitrile and water and lyophilized to give the title compound (64 mg) as a white solid.

### Example 46

### Compound 102

### Synthesis of (2S)-2-({[5-(diphenylmethyl)furan-2-yl]carbonyl}amino)-5-(ethanimidoylamino)pentanoic acid•TFA

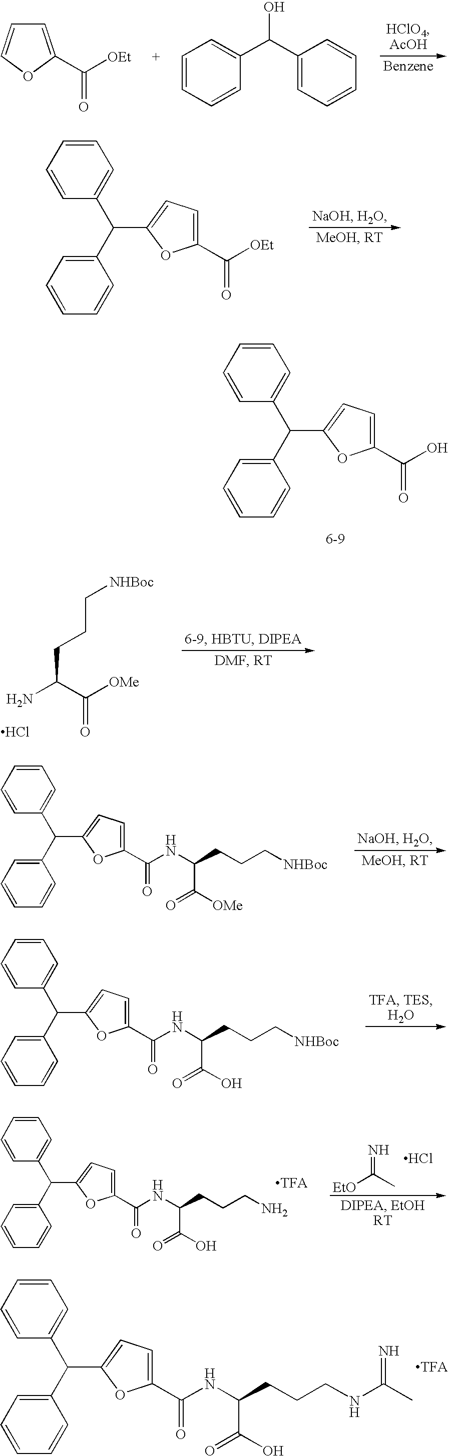

### I. Ethyl 5-(diphenylmethyl)furan-2-carboxylate

To a solution of ethyl furan-2-carboxylate (500 mg) and diphenylmethanol (657 mg) in benzene (10 mL), acetic acid (1.8 mL) and perchloric acid (0.5 mL were added. The mixture was heated to 100° C. for 1 hour, then was allowed to cool to room temperature and was diluted with ether and saturated aqueous NaHCO₃. The organic layer was washed with brine, dried over Na₂SO₄, filtered and concentrated under reduced pressure. The residue was purified by automated silica gel column chromatography (Biotage®), eluting with hexanes increasing to 10% ethyl acetate/hexanes to give the title compound (550 mg).

### II. 5-(Diphenylmethyl)furan-2-carboxylic acid

To a solution of ethyl 5-(diphenylmethyl)furan-2-carboxylate (550 mg) in methanol (20 mL), aqueous NaOH (6.0 M, 2 mL) was added. The resulting mixture was stirred at room temperature for 7 hours, then was diluted with water and extracted with ether. The aqueous layer was acidified with HCl (1 M) and was extracted with ethyl acetate. The ethyl acetate layer was washed with brine, dried over Na₂SO₄, filtered and concentrated under reduced pressure to give the title compound as a yellow solid.

### III. Methyl (2S)-5-[(tert-butoxycarbonyl)amino]-2-({[5-(diphenylmethyl)furan-2-yl]carbonyl}amino)pentanoate

A solution of methyl (2S)-2-amino-5-[(tert-butoxycarbonyl)amino]pentanoate hydrochloride (310 mg), 5-(Diphenylmethyl)furan-2-carboxylic acid (305 mg), diisopropylethylamine (0.6 mL) and HBTU (625 mg) in DMF(10 mL) was stirred at room temperature overnight. The resulting mixture was diluted with ethyl acetate, washed with water (3 times) and with brine. The organic layer was dried over MgSO₄, filtered and concentrated under reduced pressure to give the title compound (450 mg).

### IV. (2S)-5-[(tert-Butoxycarbonyl)amino]-2-({[5-(diphenylmethyl)furan-2-yl]carbonyl}amino)pentanoic acid

To a solution of methyl (2S)-5-[(tert-butoxycarbonyl)amino]-2-({[5-(diphenylmethyl)furan-2-yl]carbonyl}amino)pentanoate (550 mg) in MeOH (10 mL), aqueous NaOH (6.0 M, 5 mL) was added. The resulting mixture was stirred at room temperature overnight, then was acidified with HCl (1 M) and extracted with ethy acetate. The ethyl acetate layer was washed with brine, dried over Na₂SO₄, filtered and concentrated under reduced pressure to give the title compound (500 mg).

### V. (2S)-5-Amino-2-({[5-(diphenylmethyl)furan-2-yl]carbonyl}amino)pentanoic acid•TFA

To a solution of (2S)-5-[(tert-butoxycarbonyl)amino]-2-({[5-(diphenylmethyl)furan-2-yl]carbonyl}amino)pentanoic acid (500 mg) in TFA (5 mL), triethylsilane (0.1 mL) and water (0.1 mL) were added. The resulting mixture was stirred at room temperature for 1 hour and the reaction mixture was concentrated under reduced pressure. The residue was taken up in ethanol and concentrated under reduced pressure to give the title compound. This material was used without purification.

### VI. (2S)-2-({[5-(Diphenylmethyl)furan-2-yl]carbonyl}amino)-5-(ethanimidoylamino)pentanoic acid•TFA

To a solution of (2S)-5-amino-2-({[5-(diphenylmethyl)furan-2-yl]carbonyl}amino)pentanoic acid•TFA (crude material from previous reaction) in ethanol (20 mL) at room temperature, ethyl acetimidate hydrochloride (625 mg) and disopropylethylamine (1.7 mL) were added sequentially. The resulting suspension was stirred at room temperature overnight and the resulting mixture was filtered through Celite® and concentrated under reduced pressure. The residue was purified by reverse phase HPLC, eluting with eluting with acetonitrile/water/TFA mixture and the fractions containing the desired product were combined and lyophilized to give the title compound (270 mg) as a white solid

### Example 47

### Inhibition of human [¹²⁵I]-C3a binding to human C3a receptor

Binding of human [¹²⁵I]-C3a to human C3a receptor (C3aR) was done using cell membranes from HEK293 cells stably expressing recombinant C3aR, in a homogeneous scintillation proximity assay (SPA). The C3aR cell membranes were pre-coupled over-night at 4° C. to WGA-PVT SPA beads (Amersham) at a ratio of 10 μg cell membrane to 0.5 mg beads/assay. Assay was performed in 96-well microtiter Optiplates (Packard) by mixing coupled beads and 0.1 nM [¹²⁵I]-C3a (2200 Ci/mmol, Perkin Elmer Life Sciences), in a total volume of 10 μl binding buffer (20 mM HEPES, pH 7.4, 125 mM NaCl, 5 mM KCl, 1 mM CaCl₂, 1 mM MgCl₂, 0.25% BSA, 0.2% CHAPS). Test compounds were diluted in DMSO and were tested for their inhibitory potential in the assay (final concentration≦1% DMSO). Incubation was done for 3 hours at room temperature followed by reading in a TopCount scintillation microplate reader. Non-specific binding was determined by adding 1 μM unlabeled C3a (Calbiochem) to the assay mixture. Calculation of the IC₅₀ was done as above using a nonlinear least square fitting program, with Graphpad Prism. The IC₅₀ values for exemplary compounds are provided in Tables 1-6.

TABLE 1

| | | | | Binding |
|---|---|---|---|---|
| Compound | Structure | Name | Mass Spectra | IC₅₀ |
| Number | (Neutral form) | (Neutral Form) | (m/z) | (μM) |
| 1 | 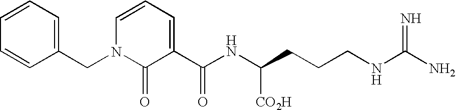 | (2S)-2-{[(1-benzyl-2-oxo-1,2-dihydropyridin-3-yl)carbonyl]amino}-5-carbamimidamidopentanoicacid | Measured (M + H)⁺ = 386.19Calculated (M + H)⁺ = 386.18 | B |
| 2 | 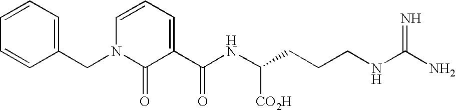 | (2R)-2-{[(1-benzyl-2-oxo-1,2-dihydropyridin-3-yl)carbonyl]amino}-5-carbamimidamidopentanoicacid | Measured (M + H)⁺ = 386.17Calculated (M + H)⁺ = 386.18 | C |
| 3 | 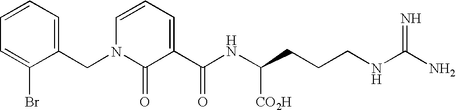 | (2S)-2-({[1-(2-bromobenzyl)-2-oxo-1,2-dihydropyridin-3-yl]carbonyl}amino)-5-carbamimidamidopentanoic acid | Measured (M − H)⁻ = 462.11Calculated (M − H)⁻ = 462.08 | A |
| 4 | 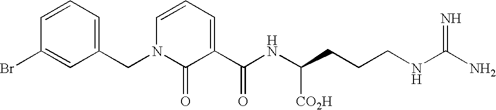 | (2S)-2-({[1-(3-bromobenzyl)-2-oxo-1,2-dihydropyridin-3-yl]carbonyl}amino)-5-carbamimidamidopentanoic acid | Measured (M − H)⁻ = 462.12Calculated (M − H)⁻ = 462.08 | A |
| 5 | 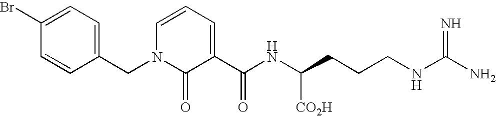 | (2S)-2-({[1-(4-bromobenzyl)-2-oxo-1,2-dihydropyridin-3-yl]carbonyl}amino)-5-carbamimidamidopentanoic acid | Measured (M − H)⁻ = 462.12Calculated (M − H)⁻ = 462.08 | A |
| 6 | 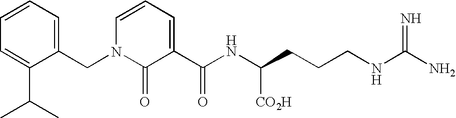 | (2S)-5-carbamimidamido-2-({[1-(2-isopropylbenzyl)-2-oxo-1,2-dihydropyridin-3-yl]carbonyl}amino)pentanoic acid | Measured (M + H)⁺ = 428.23Calculated (M + H)⁺ = 428.23 | B |
| 7 | 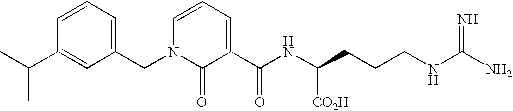 | (2S)-5-carbamimidamido-2-({[1-(3-isopropylbenzyl)-2-oxo-1,2-dihydropyridin-3-yl]carbonyl}amino)pentanoic acid | Measured (M + H)⁺ = 428.24Calculated (M + H)⁺ = 428.23 | B |
| 8 | 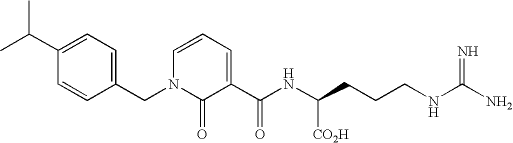 | (2S)-5-carbamimidamido-2-({[1-(4-isopropylbenzyl)-2-oxo-1,2-dihydropyridin-3-yl]carbonyl}amino)pentanoic acid | Measured (M + H)⁺ = 428.23Calculated (M + H)⁺ = 428.23 | A |
| 9 | 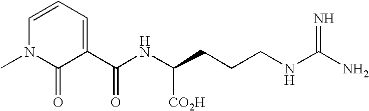 | (2S)-5-carbamimidamido-2-{[(1-methyl-2-oxo-1,2-dihydropyridin-3-yl)carbonyl]amino}pentanoic acid | Measured (M + H)⁺ = 310.20Calculated (M + H)⁺ = 310.15 | C |
| 10 | 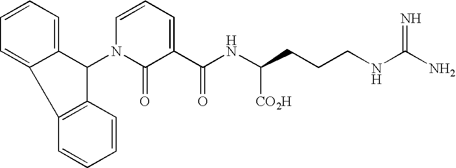 | (2S)-5-carbamimidamido-2-({[1-(9H-fluoren-9-yl)-2-oxo-1,2-dihydropyridin-3-yl]carbonyl}amino)pentanoic acid | Measured (M + H)⁺ = 460.17Calculated (M + H)⁺ = 460.20 | B |
| 11 | 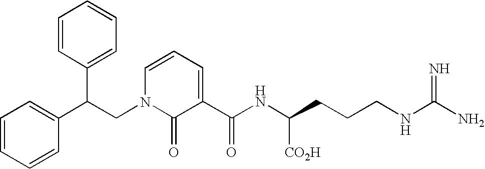 | (2S)-5-carbamimidamido-2-({[1-(2,2-diphenylethyl)-2-oxo-1,2-dihydropyridin-3-yl]carbonyl}amino)pentanoic acid | Measured (M + H)⁺ = 476.16Calculated (M + H)⁺ = 476.23 | A |
| 12 | 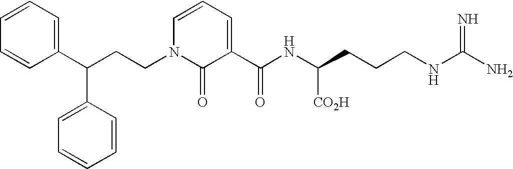 | (2S)-5-carbamimidamido-2-({[1-(3,3-diphenylpropyl)-2-oxo-1,2-dihydropyridin-3-yl]carbonyl}amino)pentanoic acid | Measured (M − H)⁻ = 488.28Calculated (M − H)⁻ = 488.23 | A |
| 13 | 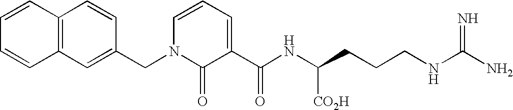 | (2S)-5-carbamimidamido-2-({[1-(2-naphthylmethyl)-2-oxo-1,2-dihydropyridin-3-yl]carbonyl}amino)pentanoic acid | Measured (M − H)⁻ = 434.21Calculated (M − H)⁻ = 434.18 | A |
| 14 | 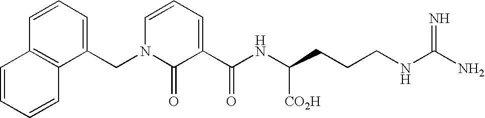 | (2S)-5-carbamimidamido-2-({[1-(1-naphthylmethyl)-2-oxo-1,2-dihydropyridin-3-yl]carbonyl}amino)pentanoic acid | Measured (M − H)⁻ = 434.26Calculated (M − H)⁻ = 434.18 | A |
| 15 | 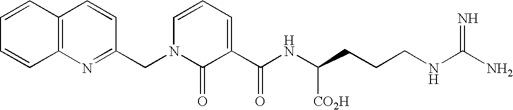 | (2S)-5-carbamimidamido-2-({[2-oxo-1-(quinolin-2-ylmethyl)-1,2-dihydropyridin-3-yl]carbonyl}amino)pentanoic acid | Measured (M − H)⁻ = 435.22Calculated (M − H)⁻ = 435.18 | B |
| 16 | 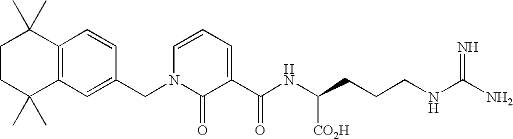 | (2S)-5-carbamimidamido-2-[({2-oxo-1-[(5,5,8,8-tetramethyl-5,6,7,8-tetrahydronaphthalen-2-yl)methyl]-1,2-dihydropyridin-3-yl}carbonyl)amino]pentanoic acid | Measured (M + H)⁺ = 496.30Calculated (M + H)⁺ = 496.29 | C |
| 17 | 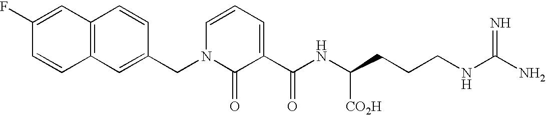 | (2S)-5-carbamimidamido-2-[({1-[(6-fluoro-2-naphthyl)methyl]-2-oxo-1,2-dihydropyridin-3-yl}carbonyl)amino]pentanoic acid | Measured (M − H)⁻ = 452.21Calculated (M − H)⁻ = 452.17 | A |
| 18 | 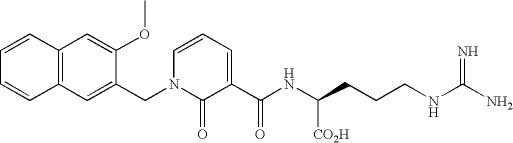 | (2S)-5-carbamimidamido-2-[({1-[(3-methoxy-2-naphthyl)methyl]-2-oxo-1,2-dihydropyridin-3-yl}carbonyl)amino]pentanoic acid | Measured (M − H)⁻ = 464.26Calculated (M − H)⁻ = 464.19 | B |
| 19 | 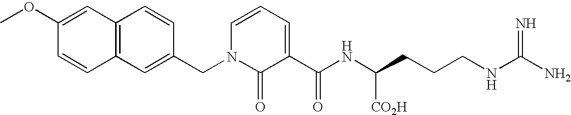 | (2S)-5-carbamimidamido-2-[({1-[(6-methoxy-2-naphthyl)methyl]-2-oxo-1,2-dihydropyridin-3-yl}carbonyl)amino]pentanoic acid | Measured (M − H)⁻ = 464.23Calculated (M − H)⁻ = 464.19 | B |
| 20 | 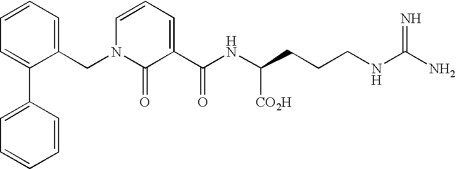 | (2S)-2-({[1-(biphenyl-2-ylmethyl)-2-oxo-1,2-dihydropyridin-3-yl]carbonyl}amino)-5-carbamimidamidopentanoic acid | Measured (M + H)⁺ = 462.22Calculated (M + H)⁺ = 462.21 | A |
| 21 | 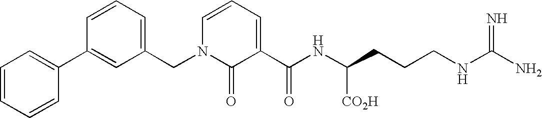 | (2S)-2-({[1-(biphenyl-3-ylmethyl)-2-oxo-1,2-dihydropyridin-3-yl]carbonyl}amino)-5-carbamimidamidopentanoic acid | Measured (M + H)⁺ = 462.21Calculated (M + H)⁺ = 462.21 | B |
| 22 | 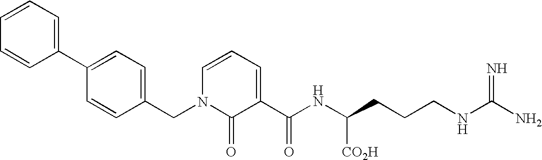 | (2S)-2-({[1-(biphenyl-4-ylmethyl)-2-oxo-1,2-dihydropyridin-3-yl]carbonyl}amino)-5-carbamimidamidopentanoic acid | Measured (M + H)⁺ = 462.24Calculated (M + H)⁺ = 462.21 | A |
| 23 | 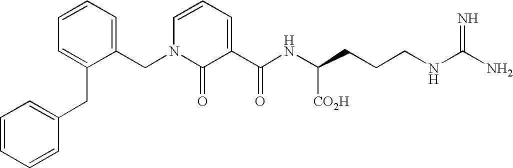 | (2S)-2-({[1-(2-benzylbenzyl)-2-oxo-1,2-dihydropyridin-3-yl]carbonyl}amino)-5-carbamimidamidopentanoic acid | Measured (M + H)⁺ = 476.26Calculated (M + H)⁺ = 476.23 | A |
| 24 | 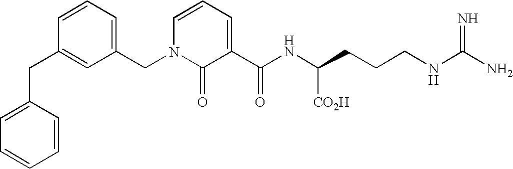 | (2S)-2-({[1-(3-benzylbenzyl)-2-oxo-1,2-dihydropyridin-3-yl]carbonyl}amino)-5-carbamimidamidopentanoic acid | Measured (M + H)⁺ = 476.26Calculated (M + H)⁺ = 476.23 | B |
| 25 | 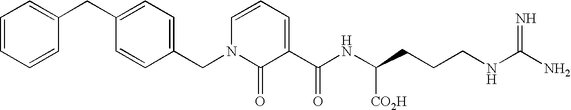 | (2S)-2-({[1-(4-benzylbenzyl)-2-oxo-1,2-dihydropyridin-3-yl]carbonyl}amino)-5-carbamimidamidopentanoic acid | Measured (M + H)⁺ = 476.25Calculated (M + H)⁺ = 476.23 | B |
| 26 | 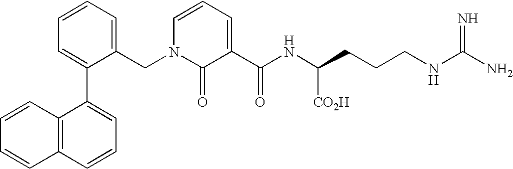 | (2S)-5-carbamimidamido-2-[({1-[2-(1-naphthyl)benzyl]-2-oxo-1,2-dihydropyridin-3-yl}carbonyl)amino]pentanoic acid | Measured (M − H)⁻ = 510.25Calculated (M − H)⁻ = 510.21 | A |
| 27 | 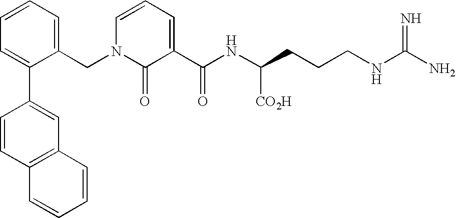 | (2S)-5-carbamimidamido-2-[({1-[2-(2-naphthyl)benzyl]-2-oxo-1,2-dihydropyridin-3-yl}carbonyl)amino]pentanoic acid | Measured (M − H)⁻ = 510.26Calculated (M − H)⁻ = 510.21 | A |
| 28 | 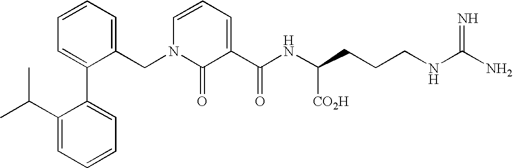 | (2S)-5-carbamimidamido-2-[({1-[(2′-isopropylbiphenyl-2-yl)methyl]-2-oxo-1,2-dihydropyridin-3-yl}carbonyl)amino]pentanoic acid | Measured (M + H)⁺ = 504.30Calculated (M + H)⁺ = 504.26 | A |
| 29 | 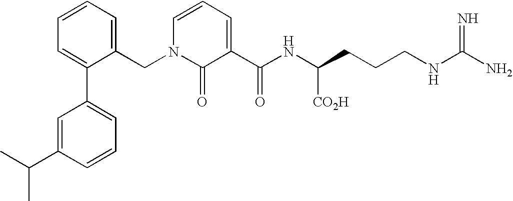 | (2S)-5-carbamimidamido-2-[({1-[(3′-isopropylbiphenyl-2-yl)methyl]-2-oxo-1,2-dihydropyridin-3-yl}carbonyl)amino]pentanoic acid | Measured (M + H)⁺ = 504.28Calculated (M + H)⁺ = 504.26 | A |
| 30 | 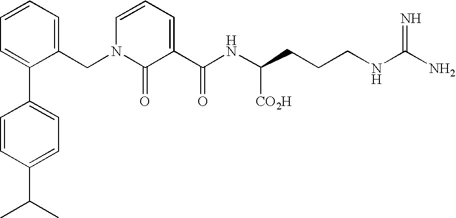 | (2S)-5-carbamimidamido-2-[({1-[(4′-isopropylbiphenyl-2-yl)methyl]-2-oxo-1,2-dihydropyridin-3-yl}carbonyl)amino]pentanoic acid | Measured (M + H)⁺ = 504.29Calculated (M + H)⁺ = 504.26 | B |
| 31 | 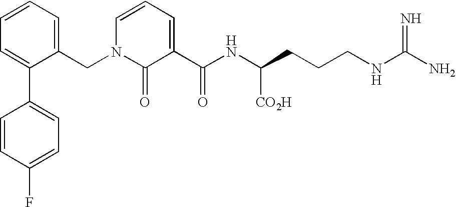 | (2S)-5-carbamimidamido-2-[({1-[(4′-fluorobiphenyl-2-yl)methyl]-2-oxo-1,2-dihydropyridin-3-yl}carbonyl)amino]pentanoic acid | Measured (M + H)⁺ = 480.20Calculated (M + H)⁺ = 480.20 | A |
| 32 | 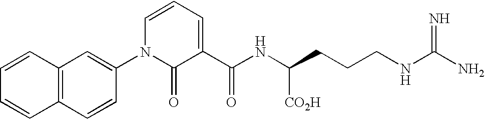 | (2S)-5-carbamimidamido-2-({[1-(2-naphthyl)-2-oxo-1,2-dihydropyridin-3-yl]carbonyl}amino)pentanoicacid | Measured (M − H)⁻ = 420.22Calculated (M − H)⁻ = 420.17 | B |
| 33 | 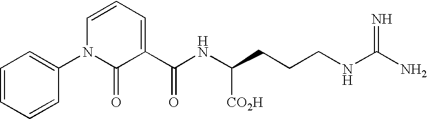 | (2S)-5-carbamimidamido-2-{[(2-oxo-1-phenyl-1,2-dihydropyridin-3-yl)carbonyl]amino}pentanoic acid | Measured (M + H)⁺ = 372.15Calculated (M + H)⁺ = 372.17 | C |
| 34 | 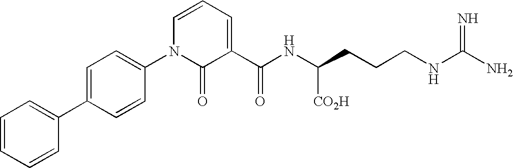 | (2S)-2-{[(1-biphenyl-4-yl-2-oxo-1,2-dihydropyridin-3-yl)carbonyl]amino}-5-carbamimidamidopentanoicacid | Measured (M + H)⁺ = 448.19Calculated (M + H)⁺ = 448.20 | C |
| 35 | 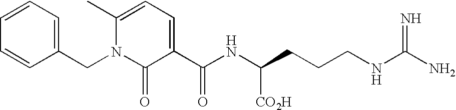 | (2S)-2-{[(1-benzyl-6-methyl-2-oxo-1,2-dihydropyridin-3-yl)carbonyl]amino}-5-carbamimidamidopentanoic acid | Measured (M + H)⁺ = 400.22Calculated (M + H)⁺ = 400.20 | B |
| 36 | 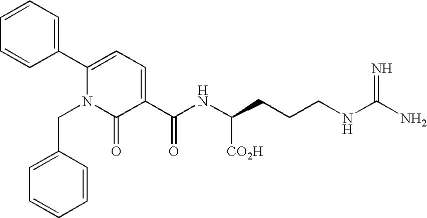 | (2S)-2-{[(1-benzyl-2-oxo-6-phenyl-1,2-dihydropyridin-3-yl)carbonyl]amino}-5-carbamimidamidopentanoic acid | Measured (M − H)⁻ = 460.23Calculated (M − H)⁻ = 460.20 | C |
| 37 | 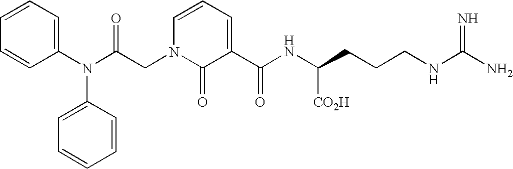 | (2S)-5-carbamimidamido-2-[({1-[2-(diphenylamino)-2-oxoethyl]-2-oxo-1,2-dihydropyridin-3-yl}carbonyl)amino]pentanoic acid | Measured (M − H)⁻ = 503.22Calculated (M − H)⁻ = 503.20 | B |
| 38 | 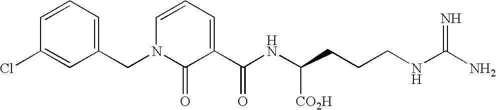 | (2S)-5-carbamimidamido-2-({[1-(3-chlorobenzyl)-2-oxo-1,2-dihydropyridin-3-yl]carbonyl}amino)pentanoic acid | Measured (M + H)⁺ = 420.13Calculated (M + H)⁺ = 420.14 | A |
| 39 | 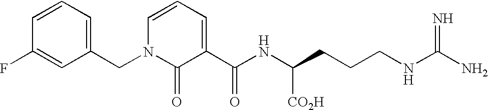 | (2S)-5-carbamimidamido-2-({[1-(3-fluorobenzyl)-2-oxo-1,2-dihydropyridin-3-yl]carbonyl}amino)pentanoic acid | Measured (M + H)⁺ = 404.17Calculated (M + H)⁺ = 404.17 | A |

TABLE 2

| Com- | | | | Binding |
|---|---|---|---|---|
| pound | Structure | Name | Mass Spectra | IC₅₀ |
| Number | (Neutral form) | (Neutral Form) | (m/z) | (μM) |
| 40 | 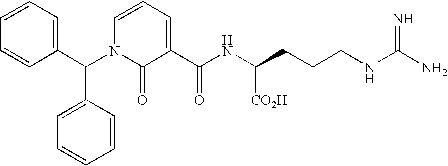 | (2S)-5-carbamimidamido-2-({[1-(diphenylmethyl)-2-oxo-1,2-dihydropyridin-3-yl]carbonyl}amino)pentanoic acid | Measured(M + H)⁺ = 462.22Calculated(M + H)⁺ = 462.21 | A |
| 41 | 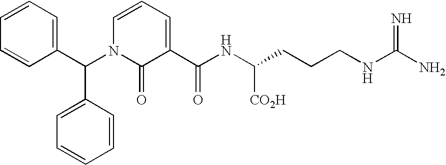 | (2R)-5-carbamimidamido-2-({[1-(diphenylmethyl)-2-oxo-1,2-dihydropyridin-3-yl]carbonyl}amino)pentanoic acid | Measured(M + H)⁺ = 462.21Calculated(M + H)⁺ = 462.21 | A |
| 42 | 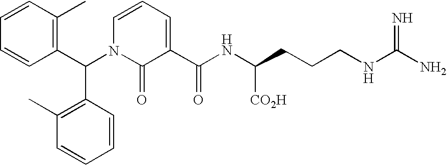 | (2S)-2-[({1-[bis(2-methylphenyl)methyl]-2-oxo-1,2-dihydropyridin-3-yl}carbonyl)amino]-5-carbamimidamidopentanoic acid | Measured(M + H)⁺ = 490.26Calculated(M + H)⁺ = 490.25 | A |
| 43 | 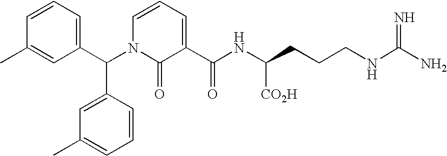 | (2S)-2-[({1-[bis(3-methylphenyl)methyl]-2-oxo-1,2-dihydropyridin-3-yl}carbonyl)amino]-5-carbamimidamidopentanoic acid | Measured(M + H)⁺ = 490.24Calculated(M + H)⁺ = 490.25 | A |
| 44 | 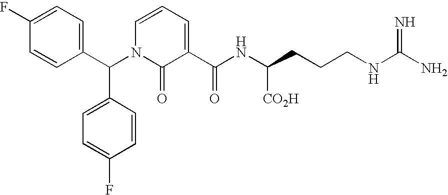 | (2S)-2-[({1-[bis(4-fluorophenyl)methyl]-2-oxo-1,2-dihydropyridin-3-yl}carbonyl)amino]-5-carbamimidamidopentanoic acid | Measured(M − H)⁻ = 496.23Calculated(M − H)⁻ = 496.18 | A |
| 45 | 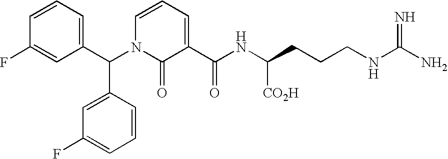 | (2S)-2-[({1-[bis(3-fluorophenyl)methyl]-2-oxo-1,2-dihydropyridin-3-yl}carbonyl)amino]-5-carbamimidamidopentanoic acid | Measured(M − H)⁻ = 496.22Calculated(M − H)⁻ = 496.18 | A |
| 46 | 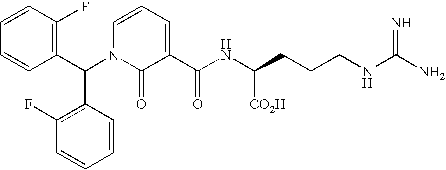 | (2S)-2-[({1-[bis(2-fluorophenyl)methyl]-2-oxo-1,2-dihydropyridin-3-yl}carbonyl)amino]-5-carbamimidamidopentanoic acid | Measured(M − H)⁻ = 496.22Calculated(M − H)⁻ = 496.18 | A |
| 47 | 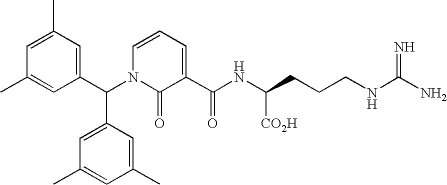 | (2S)-2-[({1-[bis(3,5-dimethylphenyl)methyl]-2-oxo-1,2-dihydropyridin-3-yl}carbonyl)amino]-5-carbamimidamidopentanoic acid | Measured(M − H)⁻ = 516.31Calculated(M − H)⁻ = 516.26 | A |
| 48 | 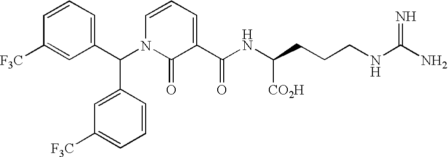 | (2S)-2-{[(1-{bis[3-(trifluoromethyl)phenyl]methyl}-2-oxo-1,2-dihydropyridin-3-yl)carbonyl]amino}-5-carbamimidamidopentanoic acid | Measured(M + H)⁺ = 598.2 Calculated(M + H)⁺ = 598.2 | A |
| 49 | 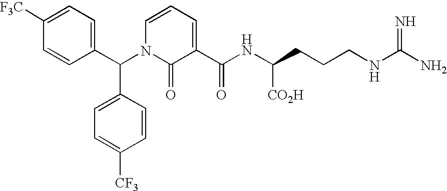 | (2S)-2-{[(1-{bis[4-(trifluoromethyl)phenyl]methyl}-2-oxo-1,2-dihydropyridin-3-yl)carbonyl]amino}-5-carbamimidamidopentanoic acid | Measured(M + H)⁺ = 598.2 Calculated(M + H)⁺ = 598.2 | B |
| 50 | 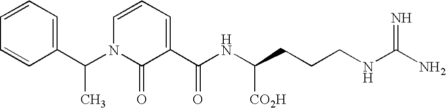 | (2S)-5-carbamimidamido-2-({[2-oxo-1-(1-phenylethyl)-1,2-dihydropyridin-3-yl]carbonyl}amino)pentanoic acid | Measured(M − H)⁻ = 398.23Calculated(M − H)⁻ = 398.18 | A |

TABLE 3

| Com- | | | Mass | Binding |
|---|---|---|---|---|
| pound | Structure | Name | Spectra | IC₅₀ |
| Number | (Neutral form) | (Neutral Form) | (m/z) | (μM) |
| 51 | 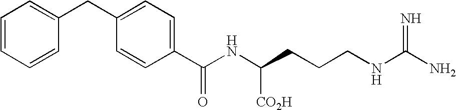 | (2S)-2-[(4-benzylbenzoyl)amino]-5-carbamimidamidopentanoic acid | Measured(M + H)⁺ =369.22Calculated(M + H)⁺ =369.19 | B |
| 52 | 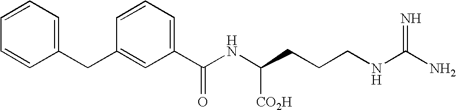 | (2S)-2-[(3-benzylbenzoyl)amino]-5-carbamimidamidopentanoic acid | Measured(M + H)⁺ =369.28Calculated(M + H)⁺ =369.19 | B |
| 53 | 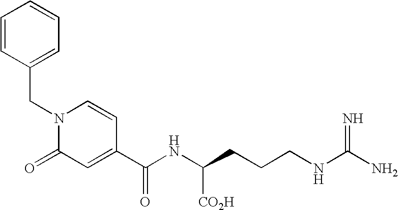 | (2S)-2-{[(1-benzyl-2-oxo-1,2-dihydropyridin-4-yl)carbonyl]amino}-5-carbamimidamidopentanoic acid | Measured(M + H)⁺ =386.18Calculated(M + H)⁺ =386.18 | B |
| 54 | 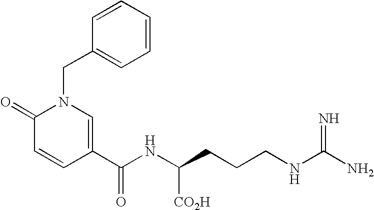 | (2S)-2-{[(1-benzyl-6-oxo-1,6-dihydropyridin-3-yl)carbonyl]amino}-5-carbamimidamidopentanoic acid | Measured(M + H)⁺ =386.18Calculated(M + H)⁺ =386.18 | B |
| 55 | 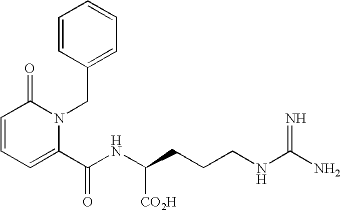 | (2S)-2-{[(1-benzyl-6-oxo-1,6-dihydropyridin-2-yl)carbonyl]amino}-5-carbamimidamidopentanoic acid | Measured(M − H)⁻ =384.23Calculated(M − H)⁻ =384.17 | B |
| 56 | 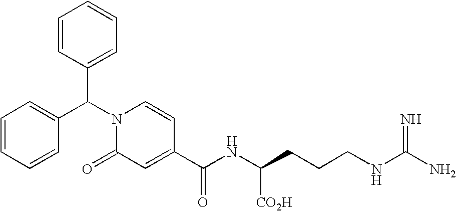 | (2S)-5-carbamimidamido-2-({[1-(diphenylmethyl)-2-oxo-1,2-dihydropyridin-4-yl]carbonyl}amino)pentanoic acid | Measured(M − H)⁻ =460.25Calculated(M − H)⁻ =460.20 | B |
| 57 | 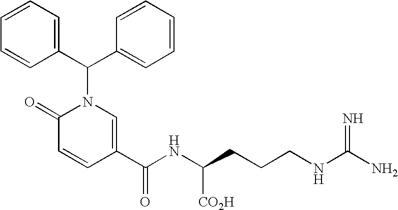 | (2S)-5-carbamimidamido-2-({[1-(diphenylmethyl)-6-oxo-1,6-dihydropyridin-3-yl]carbonyl}amino)pentanoic acid | Measured(M − H)⁻ =460.23Calculated(M − H)⁻ =460.20 | C |
| 58 | 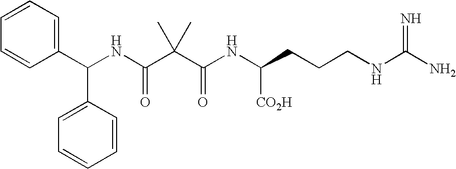 | (2S)-5-carbamimidamido-2-({3-[(diphenylmethyl)amino]-2,2-dimethyl-3-oxopropanoyl}amino)pentanoic acid | Measured(M − H)⁻ =452.26Calculated(M − H)⁻ =452.23 | A |

TABLE 4

| Com- | | | | Binding |
|---|---|---|---|---|
| pound | Structure | Name | Mass Spectra | IC₅₀ |
| Number | (Neutral form) | (Neutral Form) | (m/z) | (μM) |
| 60 | 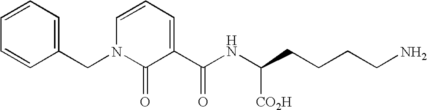 | (2S)-6-amino-2-{[(1-benzyl-2-oxo-1,2-dihydropyridin-3-yl)carbonyl]amino}hexanoic acid | Measured(M + H)⁺ =358.20Calculated(M + H)⁺ =358.18 | C |
| 61 | 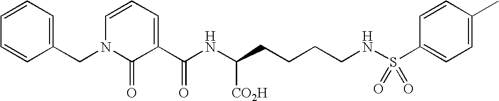 | (2S)-2-{[(1-benzyl-2-oxo-1,2-dihydropyridin-3-yl)carbonyl]amino}-6-{[(4-methylphenyl)sulfonyl]amino}hexanoic acid | Measured(M + H)⁺ =512.23Calculated(M + H)⁺ =512.19 | C |
| 62 | 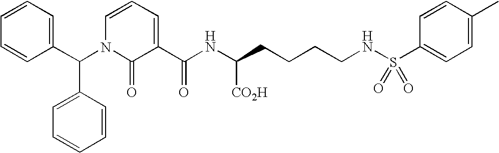 | (2S)-2-({[1-(diphenylmethyl)-2-oxo-1,2-dihydropyridin-3-yl]carbonyl}amino)-6-{[(4-methylphenyl)sulfonyl]amino}hexanoic acid | Measured(M + H)⁺ =588.19Calculated(M + H)⁺ =588.22 | B |
| 63 | 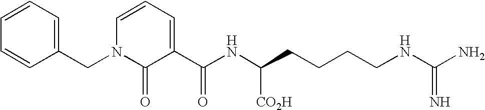 | (2S)-2-{[(1-benzyl-2-oxo-1,2-dihydropyridin-3-yl)carbonyl]amino}-6-carbamimidamido-hexanoic acid | Measured(M + H)⁺ =400.29Calculated(M + H)⁺ =400.20 | C |
| 64 | 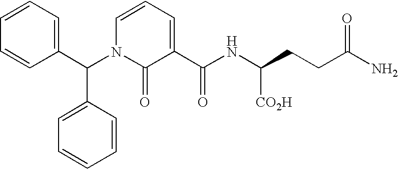 | (2S)-5-amino-2-({[1-(diphenylmethyl)-2-oxo-1,2-dihydropyridin-3-yl]carbonyl}amino)-5-oxopentanoic acid | Measured(M + H)⁺ =434.16Calculated(M + H)⁺ =434.17 | C |
| 65 | 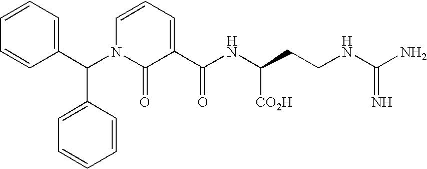 | (2S)-4-carbamimidamido-2-({[1-(diphenylmethyl)-2-oxo-1,2-dihydropyridin-3-yl]carbonyl}amino)butanoic acid | Measured(M + H)⁺ =448.20Calculated(M + H)⁺ =448.20 | B |
| 66 | 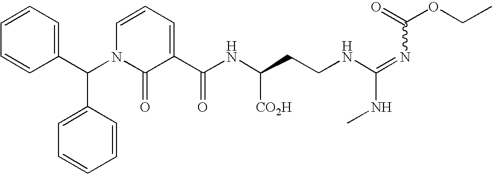 | (2S)-2-({[1-(diphenylmethyl)-2-oxo-1,2-dihydropyridin-3-yl]carbonyl}amino)-4-{[{(ethoxycarbonyl)amino}(methylamino)methylene]amino}butanoic acid | Measured(M − H)⁻ =532.22Calculated(M − H)⁻ =532.22 | C |
| 67 | 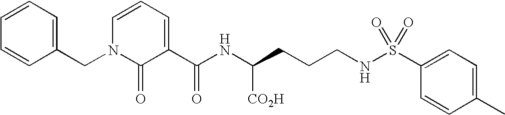 | (2S)-2-{[(1-benzyl-2-oxo-1,2-dihydropyridin-3-yl)carbonyl]amino}-5-{[(4-methylphenyl)sulfonyl]amino}pentanoic acid | Measured(M + H)⁺ =498.20Calculated(M + H)⁺ =498.17 | C |
| 68 | 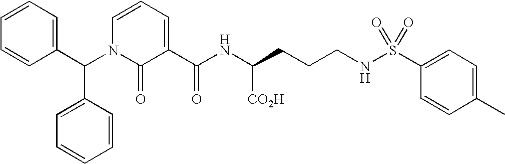 | (2S)-2-({[1-(diphenylmethyl)-2-oxo-1,2-dihydropyridin-3-yl]carbonyl}amino)-5-{[(4-methylphenyl)sulfonyl]amino}pentanoic acid | Measured(M − H)⁻ =572.24Calculated(M − H)⁻ =572.19 | C |
| 69 | 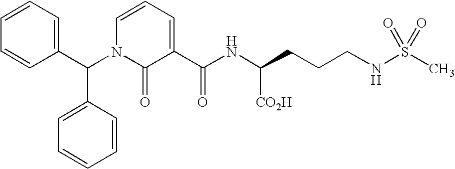 | (2S)-2-({[1-(diphenylmethyl)-2-oxo-1,2-dihydropyridin-3-yl]carbonyl}amino)-5-[(methylsulfonyl)amino]pentanoic acid | Measured(M − H)⁻ =496.13Calculated(M − H)⁻ =496.15 | B |
| 70 | 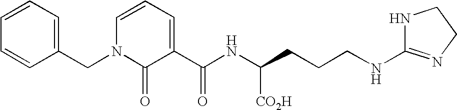 | (2S)-2-{[(1-benzyl-2-oxo-1,2-dihydropyridin-3-yl)carbonyl]amino}-5-(4,5-dihydro-1H-imidazol-2-ylamino)pentanoic acid | Measured(M + H)⁺ =412.19Calculated(M + H)⁺ =412.20 | C |
| 71 | 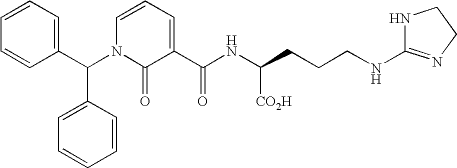 | (2S)-5-(4,5-dihydro-1H-imidazol-2-ylamino)-2-({[1-(diphenylmethyl)-2-oxo-1,2-dihydropyridin-3-yl]carbonyl}amino)pentanoic acid | Measured(M − H)⁻ =486.24Calculated(M − H)⁻ =486.21 | A |
| 72 | 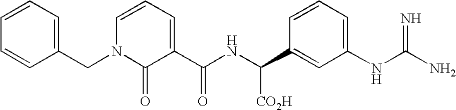 | (2S)-{[(1-benzyl-2-oxo-1,2-dihydropyridin-3-yl)carbonyl]amino}(3-carbamimidamidophenyl)acetic acid | Measured(M + H)⁺ =420.21Calculated(M + H)⁺ =420.17 | B |
| 73 | 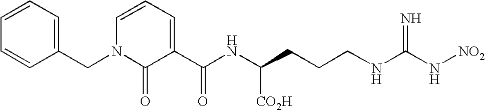 | (2S)-2-{[(1-benzyl-2-oxo-1,2-dihydropyridin-3-yl)carbonyl]amino}-5-(nitrocarbamimidamido)pentanoic acid | Measured(M + H)⁺ =431.20Calculated(M + H)⁺ =431.17 | C |
| 74 | 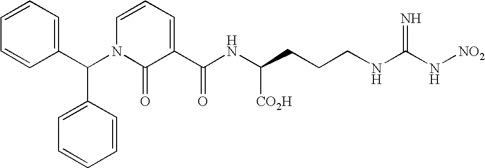 | (2S)-2-({[1-(diphenylmethyl)-2-oxo-1,2-dihydropyridin-3-yl]carbonyl}amino)-5-(nitrocarbamimidamido)pentanoic acid | Measured(M + H)⁺ =507.21Calculated(M + H)⁺ =507.20 | B |
| 75 | 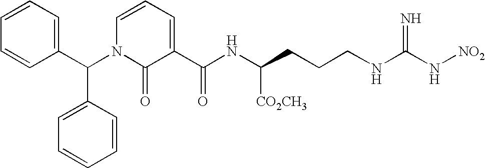 | methyl (2S)-2-({[1-(diphenylmethyl)-2-oxo-1,2-dihydropyridin-3-yl]carbonyl}amino)-5-(nitrocarbamimidamido)pentanoate | Measured(M + H)⁺ =521.21Calculated(M + H)⁺ =521.21 | C |
| 76 | 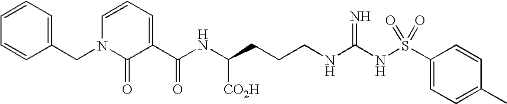 | (2S)-2-{[(1-benzyl-2-oxo-1,2-dihydropyridin-3-yl)carbonyl]amino}-5-{[(4-methylphenyl)sulfonyl]carbamimidamido}pentanoic acid | Measured(M + H)⁺ =540.16Calculated(M + H)⁺ =540.19 | C |
| 77 | 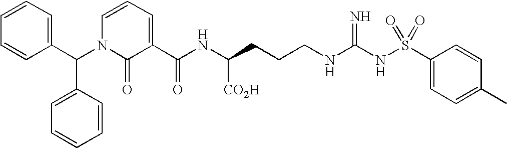 | (2S)-2-({[1-(diphenylmethyl)-2-oxo-1,2-dihydropyridin-3-yl]carbonyl}amino)-5-{[(4-methylphenyl)sulfonyl]carbamimidamido}pentanoic acid | Measured(M + H)⁺ =616.20Calculated(M + H)⁺ =616.22 | B |
| 78 | 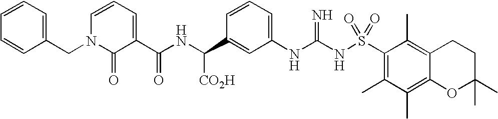 | (2S)-{[(1-benzyl-2-oxo-1,2-dihydropyridin-3-yl)carbonyl]amino}(3-{[(2,2,5,7,8-pentamethyl-3,4-dihydro-2H-chromen-6-yl)sulfonyl]carbamimidamido}phenyl)acetic acid | Measured(M + H)⁺ =686.33Calculated(M + H)⁺ =686.26 | C |
| 79 | 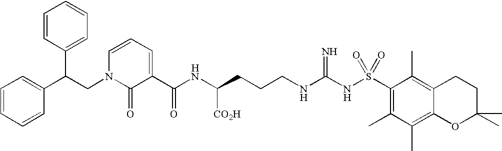 | (2S)-2-({[1-(2,2-diphenylethyl)-2-oxo-1,2-dihydropyridin-3-yl]carbonyl}amino)-5-{[(2,2,5,7,8-pentamethyl-3,4-dihydro-2H-chromen-6-yl)sulfonyl]carbamimidamido}pentanoic acid | Measured(M + H)⁺ =742.22Calculated(M + H)⁺ =742.33 | B |
| 80 | 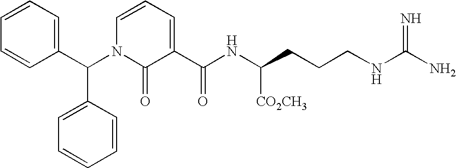 | methyl (2S)-5-carbamimidamido-2-({[1-(diphenylmethyl)-2-oxo-1,2-dihydropyridin-3-yl]carbonyl}amino)pentanoate | Measured(M + 2NA—H)⁻ =520.29Calculated(M + 2Na—H)⁻ =520.19 | A |
| 81 | 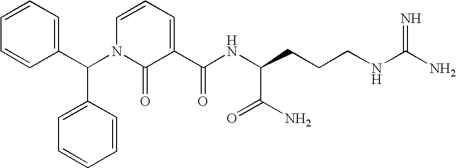 | N-[(1S)-4-carbamimidamido-1-carbamoylbutyl]-1-(diphenylmethyl)-2-oxo-1,2-dihydropyridine-3-carboxamide | Measured(M + H)⁺ =461.2Calculated(M + H)⁺ =461.2 | C |
| 82 | 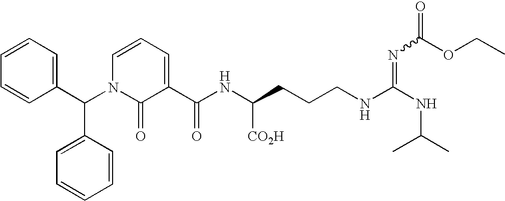 | (2S)-2-({[1-(diphenylmethyl)-2-oxo-1,2-dihydropyridin-3-yl]carbonyl}amino)-5-{[{(ethoxycarbonyl)amino}(isopropylamino)methylene]amino}pentanoic acid | Measured(M − H)⁻ =574.25Calculated(M − H)⁻ =574.27 | B |
| 83 | 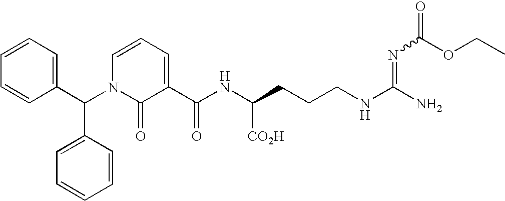 | (2S)-2-({[1-(diphenylmethyl)-2-oxo-1,2-dihydropyridin-3-yl]carbonyl}amino)-5-[(ethoxycarbonyl)carbamimidamido]pentanoic acid | Measured(M − H)⁻ =532.14Calculated(M − H)⁻ =532.22 | C |

TABLE 5

| Com- | | | | Binding |
|---|---|---|---|---|
| pound | Structure | Name | Mass Spectra | IC₅₀ |
| Number | (Neutral form) | (Neutral form) | (m/z) | (μM) |
| 84 | 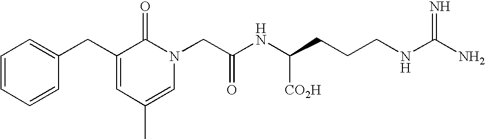 | (2S)-2-{[(3-benzyl-5-methyl-2-oxopyridin-1(2H)-yl)acetyl]amino}-5-carbamimidamidopentanoic acid | Measured(M + H)⁺ =414.34Calculated(M + H)⁺ =414.21 | C |
| 85 | 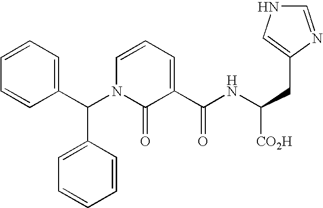 | (2S)-2-({[1-(diphenylmethyl)-2-oxo-1,2-dihydropyridin-3-yl]carbonyl}amino)-3-(1H-imidazol-4-yl)propanoic acid | Measured(M + H)⁺ =443.18Calculated(M + H)⁺ =443.17 | B |

TABLE 6

| Com- | | | | Binding |
|---|---|---|---|---|
| pound | Structure | Name | Mass Spectra | IC₅₀ |
| Number | (Neutral form) | (Neutral form) | (m/z) | (μM) |
| 86 | 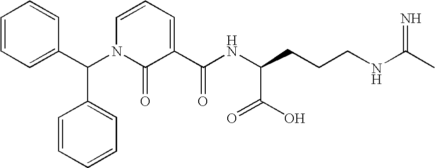 | (2S)-2-({[1-(Diphenylmethyl)-2-oxo-1,2-dihydropyridin-3-yl]carbonyl}amino)-5-(ethanimidoylamino)pentanoic acid | Measured(M + H)⁺ =461.21Calculated(M + H)⁺ =461.22 | A |
| 87 | 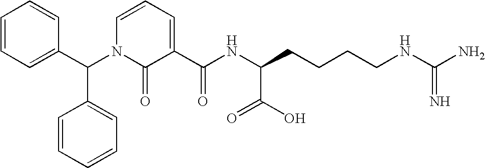 | (2S)-6-Carbamimidamido-2-({[1-(diphenylmethyl)-2-oxo-1,2-dihydropyridin-3-yl]carbonyl}amino)hexanoic acid | Measured(M + H)⁺ =476.24Calculated(M + H)⁺ =476.23 | A |
| 88 | 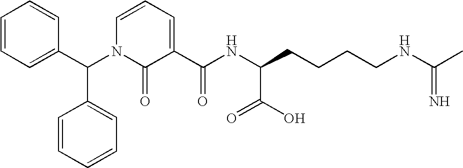 | (2S)-2-({[1-(Diphenylmethyl)-2-oxo-1,2-dihydropyridin-3-yl]carbonyl}amino)-6-(ethanimidoylamino)hexanoic acid | Measured(M + H)⁺ =475.24Calculated(M + H)⁺ =475.23 | B |
| 89 | 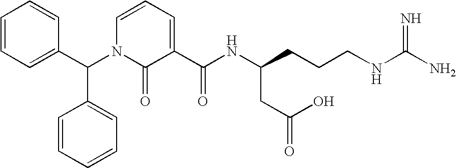 | (3S)-6-Carbamimidamido-3-({[1-(diphenylmethyl)-2-oxo-1,2-dihydropyridin-3-yl]carbonyl}amino)hexanoic acid | Measured(M + H)⁺ =476.24Calculated(M + H)⁺ =476.23 | A |
| 90 | 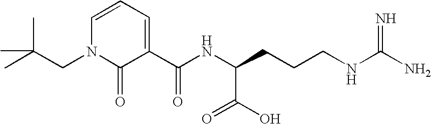 | (2S)-5-Carbamimidamido-2-({[1-(2,2-dimethylpropyl)-2-oxo-1,2-dihydropyridin-3-yl]carbonyl}amino)pentanoic acid | Measured(M − H)⁻ =364.19Calculated(M − H)⁻ =364.20 | A |
| 91 | 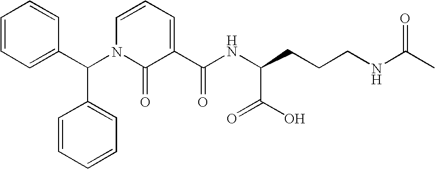 | (2S)-5-(Acetylamino)-2-({[1-(diphenylmethyl)-2-oxo-1,2-dihydropyridin-3-yl]carbonyl}amino)pentanoic acid | Measured(M − H)⁻ =460.18Calculated(M − H)⁻ =460.19 | B |
| 92 | 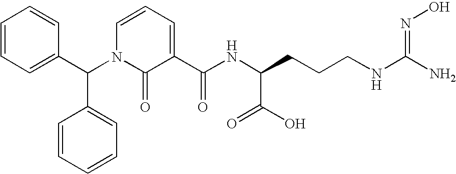 | (2S)-2-({[1-(Diphenylmethyl)-2-oxo-1,2-dihydropyridin-3-yl]carbonyl}amino)-5-(hydroxycarb-amimidamido)pentanoicacid | Measured(M + H)⁺ =478.21Calculated(M + H)⁺ =478.21 | A |
| 93 | 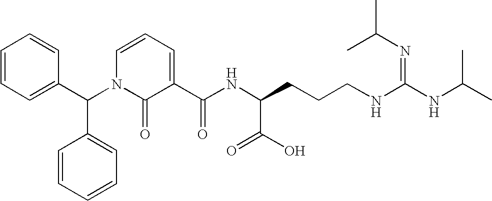 | (2S)-5-({Bis[(1-methylethyl)amino]methylidene}amino)-2-({[1-(diphenylmethyl)-2-oxo-1,2-dihydropyridin-3-yl]carbonyl}amino)pentanoic acid | Measured(M + H)⁺ =546.32Calculated(M + H)⁺ =546.31 | B |
| 94 | 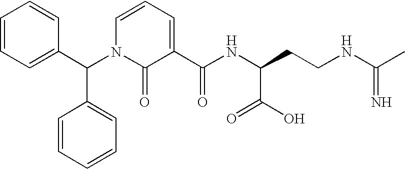 | (2S)-2-({[1-(Diphenylmethyl)-2-oxo-1,2-dihydropyridin-3-yl]carbonyl}amino)-4-(ethanimidoylamino)butanoic acid | Measured(M + H)⁺ =447.20Calculated(M + H)⁺ =447.20 | B |
| 95 | 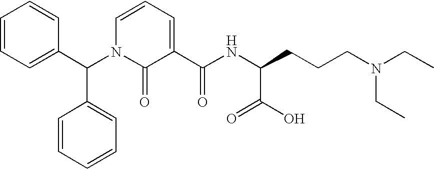 | (2S)-5-(Diethylamino)-2-({[1-(diphenylmethyl)-2-oxo-1,2-dihydropyridin-3-yl]carbonyl}amino)pentanoic acid | Measured(M + H)⁺ =476.26Calculated(M + H)⁺ =476.25 | A |
| 96 | 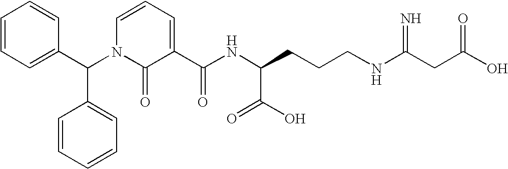 | (2S)-5-[(2-Carboxyethanimidoyl)amino]-2-({[1-(diphenylmethyl)-2-oxo-1,2-dihydropyridin-3-yl]carbonyl}amino)pentanoic acid | Measured(M + H)⁺ =505.21Calculated(M + H)⁺ =505.21 | A |
| 97 | 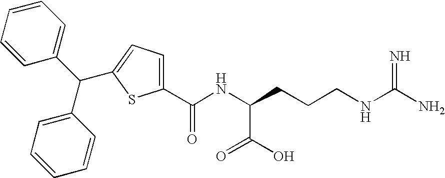 | (2S)-5-Carbamimidamido-2-({[5-(diphenylmethyl)thiophen-2-yl]carbonyl}amino)pentanoic acid | Measured(M + H)⁺ =451.18Calculated(M + H)⁺ =451.18 | A |
| 98 | 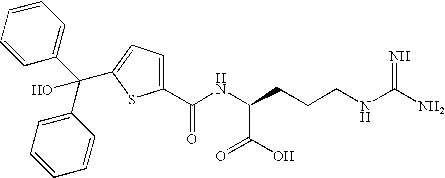 | (2S)-5-Carbamimidamido-2-[({5-[hydroxy(diphenyl)methyl]thiophen-2-yl}carbonyl)amino]pentanoic acid | Measured(M + H)⁺ =467.17Calculated(M + H)⁺ =467.17 | A |
| 99 | 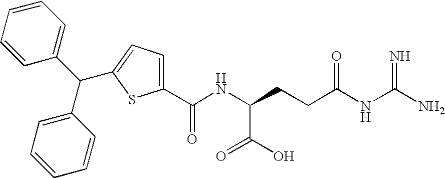 | (2S)-5-Carbamimidamido-2-({[5-(diphenylmethyl)thiophen-2-yl]carbonyl}amino)-5-oxopentanoicacid | Measured(M + H)⁺ =465.16Calculated(M + H)⁺ =465.16 | A |
| 100 | 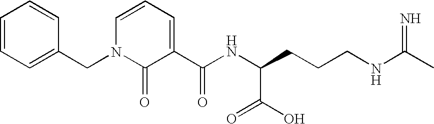 | (2S)-2-{[(1-Benzyl-2-oxo-1,2-dihydropyridin-3-yl)carbonyl]amino}-5-(ethanimidoylamino)pentanoic acid | Measured(M − H)⁻ =383.18Calculated(M − H)⁻ =383.17 | C |
| 101 | 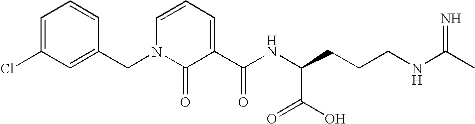 | (2S)-2-({[1-(3-Chlorobenzyl)-2-oxo-1,2-dihydropyridin-3-yl]carbonyl}amino)-5-(ethanimidoylamino)pentanoic acid | Measured(M + H)⁺ =419.13Calculated(M + H)⁺ =419.15 | B |
| 102 | 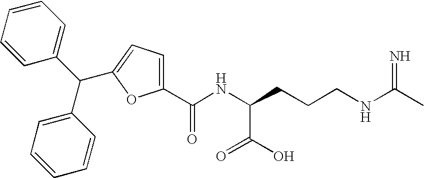 | (2S)-2-({[5-(Diphenylmethyl)furan-2-yl]carbonyl}amino)-5-(ethanimidoylamino)pentanoic acid | Measured(M + H)⁺ =434.21Calculated(M + H)⁺ =434.21 | B |
| 103 | 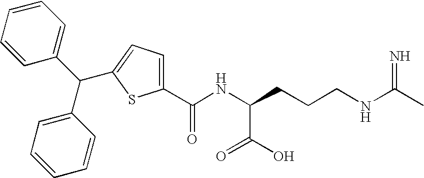 | (2S)-2-({[5-(Diphenylmethyl)thiophen-2-yl]carbonyl}amino)-5-(ethanimidoylamino)pentanoic acid | Measured(M + H)⁺ =450.17Calculated(M + H)⁺ =450.18 | B |
| 104 | 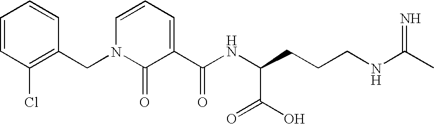 | (2S)-2-({[1-(2-Chlorobenzyl)-2-oxo-1,2-dihydropyridin-3-yl]carbonyl}amino)-5-(ethanimidoylamino)pentanoic acid | Measured(M + H)⁺ =419.15Calculated(M + H)⁺ =419.15 | C |
| 105 | 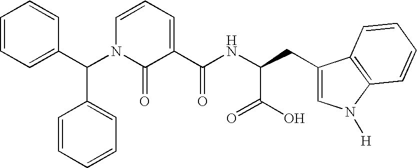 | (2S)-2-({[1-(Diphenylmethyl)-2-oxo-1,2-dihydropyridin-3-yl]carbonyl}amino)-3-(1H-indol-3-yl)propanoicacid | Measured(M − H)⁻ =490.17Calculated(M − H)⁻ =490.18 | B |
| 106 | 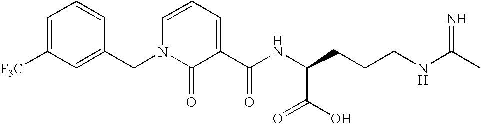 | (2S)-5-(Ethanimidoylamino)-2-[({2-oxo-1-[3-(trifluoromethyl)benzyl]-1,2-dihydropyridin-3-yl}carbonyl)amino]pentanoic acid | Measured(M + H)⁺ =453.2Calculated(M + H)⁺ =453.17 | B |
| 107 | 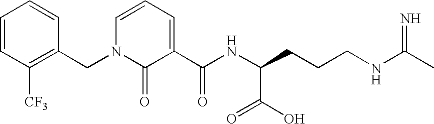 | (2S)-5-(Ethanimidoylamino)-2-[({2-oxo-1-[2-(trifluoromethyl)benzyl]-1,2-dihydropyridin-3-yl}carbonyl)amino]pentanoic acid | Measured(M + H)⁺ =453.2Calculated(M + H)⁺ =453.17 | C |
| 108 | 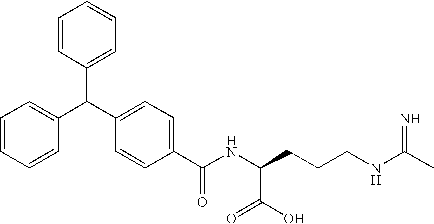 | (2S)-2-({[4-(Diphenylmethyl)phenyl]carbonyl}amino)-5-(ethanimidoylamino)pentanoic acid | Measured(M + H)⁺ =444.2Calculated(M + H)⁺ =444.23 | C |
| 109 | 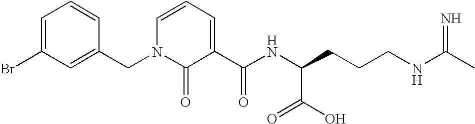 | (2S)-2-({[1-(3-Bromobenzyl)-2-oxo-1,2-dihydropyridin-3-yl]carbonyl}amino)-5-(ethanimidoylamino)pentanoic acid | Measured(M + H)⁺ =463.15Calculated(M + H)⁺ =463.10 | B |
| 110 | 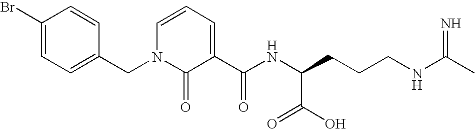 | (2S)-2-({[1-(4-Bromobenzyl)-2-oxo-1,2-dihydropyridin-3-yl]carbonyl}amino)-5-(ethanimidoylamino)pentanoic acid | Measured(M + H)⁺ =463.17Calculated(M + H)⁺ =463.10 | B |
| 111 | 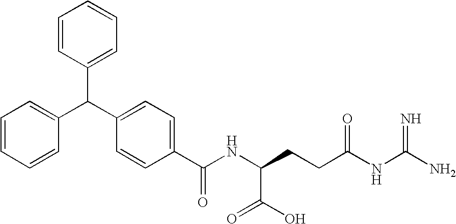 | (2S)-5-Carbamimidamido-2-({[4-(diphenylmethyl)phenyl]carbonyl}amino)-5-oxopentanoic acid | Measured(M + H)⁺ =459.21Calculated(M + H)⁺ =459.20 | A |
| 112 | 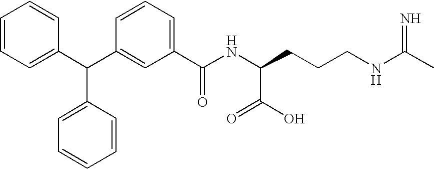 | (2S)-2-({[3-(Diphenylmethyl)phenyl]carbonyl}amino)-5-(ethanimidoylamino)pentanoic acid | Measured(M − H)⁻ =442.21Calculated(M − H)⁻ =442.21 | B |
| 113 | 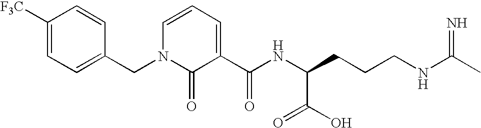 | (2S)-5-(Ethanimidoylamino)-2-[({2-oxo-1-[4-(trifluoromethyl)benzyl]-1,2-dihydropyridin-3-yl}carbonyl)amino]pentanoic acid | Measured(M − H)⁻ =451.16Calculated(M − H)⁻ =451.16 | C |
| 114 | 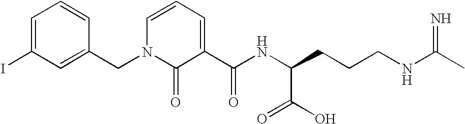 | (2S)-5-(Ethanimidoylamino)-2-({[1-(3-iodobenzyl)-2-oxo-1,2-dihydropyridin-3-yl]carbonyl}amino)pentanoic acid | Measured(M − H)⁻ =509.06Calculated(M − H)⁻ =509.07 | B |
| 115 | 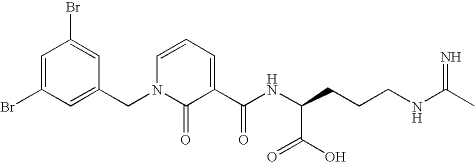 | (2S)-2-({[1-(3,5-Dibromobenzyl)-2-oxo-1,2-dihydropyridin-3-yl]carbonyl}amino)-5-(ethanimidoylamino)pentanoic acid | Measured(M − H)⁻ =538.99Calculated(M − H)⁻ =538.99 | A |
| 116 | 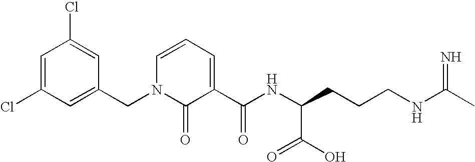 | (2S)-2-({[1-(3,5-Dichlorobenzyl)-2-oxo-1,2-dihydropyridin-3-yl]carbonyl}amino)-5-(ethanimidoylamino)pentanoic acid | Measured(M − H)⁻ =451.09Calculated(M − H)⁻ =451.09 | A |
| 117 | 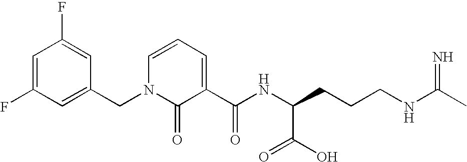 | (2S)-2-({[1-(3,5-Difluorobenzyl)-2-oxo-1,2-dihydropyridin-3-yl]carbonyl}amino)-5-(ethanimidoylamino)pentanoic acid | Measured(M − H)⁻ =419.15Calculated(M − H)⁻ =419.15 | B |
| 118 | 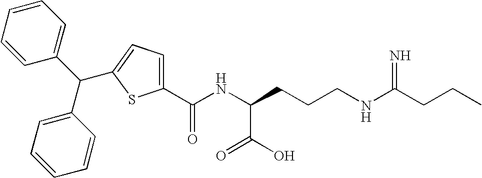 | (2S)-5-(Butanimidoylamino)-2-({[5-(diphenylmethyl)thiophen-2-yl]carbonyl}amino)pentanoic acid | Measured(M − H)⁻ =476.21Calculated(M − H)⁻ =476.20 | C |
| 119 | 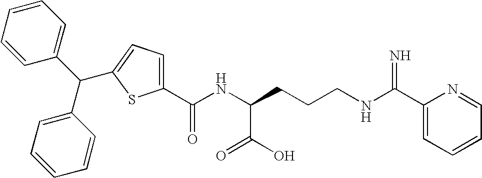 | (2S)-2-({[5-(Diphenylmethyl)thiophen-2-yl]carbonyl}amino)-5-{[imino(pyridin-2-yl)methyl]amino}pentanoic acid | Measured(M + H)⁺ =513.19Calculated(M + H)⁺ =513.20 | C |
| 120 | 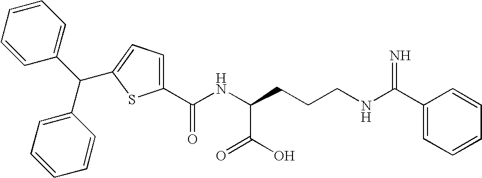 | (2S)-2-({[5-(Diphenylmethyl)thiophen-2-yl]carbonyl}amino)-5-{[imino(phenyl)methyl]amino}pentanoic acid | Measured(M − H)⁻ =510.2Calculated(M − H)⁻ =510.19 | C |
| 121 | 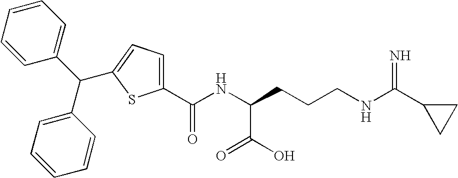 | (2S)-5-{[Cyclopropyl(imino)methyl]amino}-2-({[5-(diphenylmethyl)thiophen-2-yl]carbonyl}amino)pentanoic acid | Measured(M + H)⁺ =476.22Calculated(M + H)⁺ =476.20 | C |
| 122 | 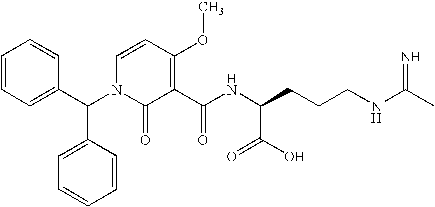 | (2S)-2-({[1-(Diphenylmethyl)-4-methoxy-2-oxo-1,2-dihydropyridin-3-yl]carbonyl}amino)-5-(ethanimidoylamino)pentanoic acid | Measured(M − H)⁻ =489.21Calculated(M − H)⁻ =489.21 | C |
| 123 | 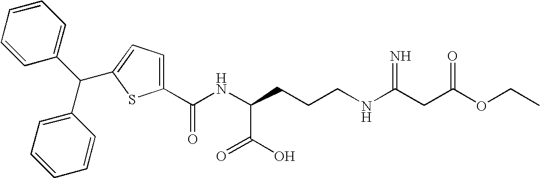 | (2S)-2-({[5-(Diphenylmethyl)thiophen-2-yl]carbonyl}amino)-5-[(3-ethoxy-3-oxopropanimidoyl)amino]pentanoic acid | Measured(M − H)⁻ =520.19Calculated(M − H)⁻ =520.19 | C |
| 124 | 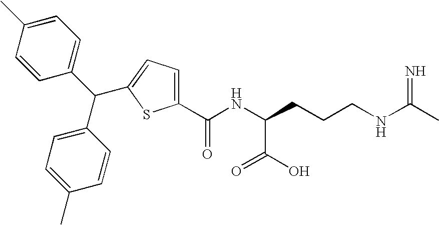 | (2S)-2-[({5-[Bis(4-methylphenyl)methyl]thiophen-2-yl}carbonyl)amino]-5-(ethanimidoylamino)pentanoic acid | Measured(M + H)⁺ =478.21Calculated(M + H)⁺ =478.22 | C |
| 125 | 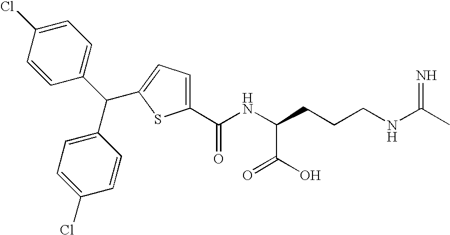 | (2S)-2-[({5-[Bis(4-chlorophenyl)methyl]thiophen-2-yl}carbonyl)amino]-5-(ethanimidoylamino)pentanoic acid | Measured(M − H)⁻ =516.2Calculated(M − H)⁻ =516.09 | B |
| 126 | 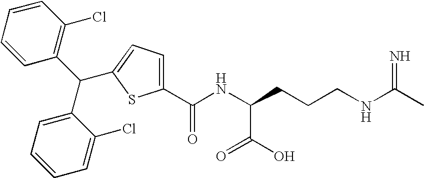 | (2S)-2-[({5-[Bis(2-chlorophenyl)methyl]thiophen-2-yl}carbonyl)amino]-5-(ethanimidoylamino)pentanoic acid | Measured(M − H)⁻ =516.2Calculated(M − H)⁻ =516.09 | C |
| 127 | 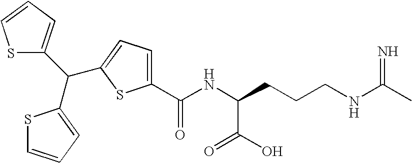 | (2S)-2-({[5-[(Dithiophen-2-ylmethyl)thiophen-2-yl]carbonyl}amino)-5-(ethanimidoylamino)pentanoic acid | Measured(M − H)⁻ =460.1Calculated(M − H)⁻ =460.08 | B |
| 128 | 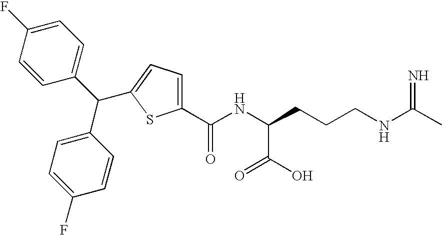 | (2S)-2-[({5-[Bis(4-fluorophenyl)methyl]thiophen-2-yl}carbonyl)amino]-5-(ethanimidoylamino)pentanoic acid | Measured(M + H)⁺ =486.17Calculated(M + H)⁺ =486.17 | B |
| 129 | 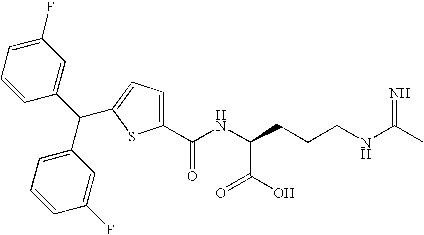 | (2S)-2-[({5-[Bis(3-fluorophenyl)methyl]thiophen-2-yl}carbonyl)amino]-5-(ethanimidoylamino)pentanoic acid | Measured(M + H)⁺ =486.17Calculated(M + H)⁺ =486.17 | B |
| 130 | 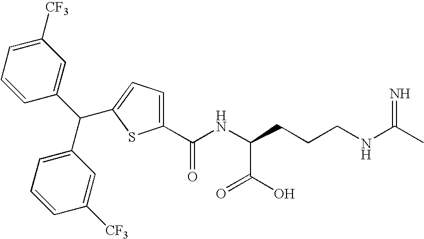 | (2S)-2-{[(5-{Bis[3-(trifluoromethyl)phenyl]methyl}thiophen-2-yl)carbonyl]amino}-5-(ethanimidoylamino)pentanoic acid | Measured(M − H)⁻ =584.15Calculated(M − H)⁻ =584.14 | C |
| 131 | 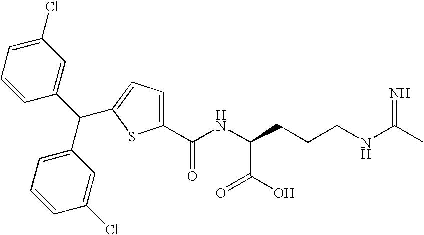 | (2S)-2-[({5-[Bis(3-chlorophenyl)methyl]thiophen-2-yl}carbonyl)amino]-5-(ethanimidoylamino)pentanoic acid | Measured(M − H)⁻ =516.10Calculated(M − H)⁻ =516.09 | A |
| 132 | 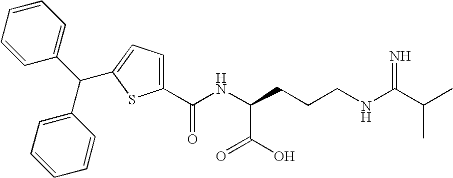 | (2S)-2-({[5-(Diphenylmethyl)thiophen-2-yl]carbonyl}amino)-5-[(2-methylpropanimidoyl)amino]pentanoic acid | Measured(M − H)⁻ =476.20Calculated(M − H)⁻ =476.20 | C |
| 133 | 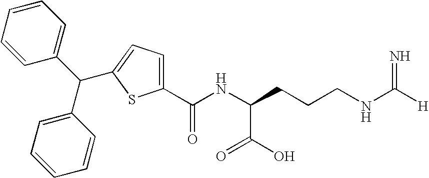 | (2S)-2-({[5-(Diphenylmethyl)thiophen-2-yl]carbonyl}amino)-5-[(iminomethyl)amino]pentanoic acid | Measured(M + H)⁺ =436.19Calculated(M + H)⁺ =436.17 | B |
| 134 | 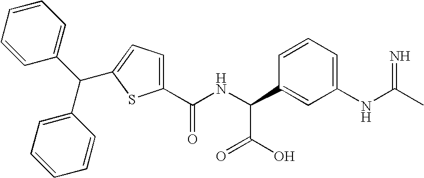 | (2S)-({[5-(Diphenylmethyl)thiophen-2-yl]carbonyl}amino)[3-(ethanimidoylamino)phenyl]ethanoicacid | Measured(M + H)⁺ =484.17Calculated(M + H)⁺ =484.17 | C |
| 135 | 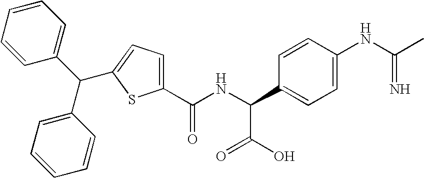 | (2S)-({[5-(Diphenylmethyl)thiophen-2-yl]carbonyl}amino)[4-(ethanimidoylamino)phenyl]ethanoicacid | Measured(M + H)⁺ =484.17Calculated(M + H)⁺ =484.17 | C |
| 136 | 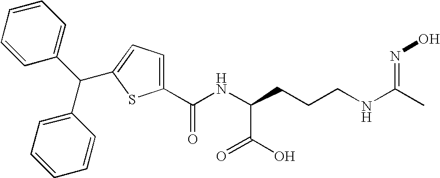 | (2S)-2-({[5-(Diphenylmethyl)thiophen-2-yl]carbonyl}amino)-5-{[(1Z)-1-(hydroxyamino)ethylidene]amino}pentanoic acid | Measured(M + H)⁺ =466.16Calculated(M + H)⁺ =466.18 | B |

In Tables 1-6, the IC₅₀ for the compounds are represented as follows: A < 0.05 μM; B = 0.05-0.5 μM; C = 0.5-20 μM.

The embodiments described above are intended to be merely exemplary, and those skilled in the art will recognize, or will be able to ascertain using no more than routine experimentation, numerous equivalents of specific compounds, materials, and procedures. All such equivalents are considered to be within the scope of the claimed subject matter and are encompassed by the appended claims.

What is claimed is:

1. A compound of Formula I: 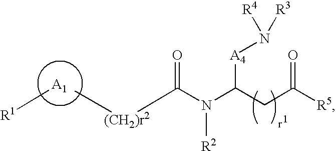
or pharmaceutically acceptable derivatives thereof,
wherein A₁ is arylene, heteroarylene or heterocyclylene;
R¹ is aryl, aralkyl, alkylaryl, cycloalkyl, cycloalkylalkyl, heterocyclyl, heterocyclylalkyl, heteroaryl or heteroarylalkyl;
R² is hydrogen, alkyl, alkenyl, alkynyl, cycloalkyl, aryl, heteroaryl or heterocyclyl;
R⁵ is OR or NR^{5a}R^{5b};
R^{5a} and R^{5b} are selected as follows:
i) R^{5a} and R^{5b} are each independently hydrogen, alkyl, alkenyl, alkynyl, cycloalkyl, aryl, heteroaryl or heterocyclyl; or
ii) R^{5a} and R^{5b} together with the nitrogen atom on which they are substituted form a 3-7 membered heterocyclic or heteroaryl ring;
A₄ is alkylene, alkenylene, alkynylene, alk(en)(yn)ylene, cycloalkylene, arylene, aralkylene, alkylarylene, heteroarylene or heterocyclylene;
R³ and R⁴ are selected as follow:
i) R³ is hydrogen, alkyl, alkenyl, alkynyl, cycloalkyl, aryl, heteroaryl or heterocyclyl; and R⁴ is R³, —C(═R⁶)NR⁷R⁸, —C(═NR)R, —C(O)R⁹, —S(O)R⁹, —S(O)₂NHR^{9a} —C(O)NHR^{9a} or CH₂)ₓOH;
ii) R³ and R⁴ together form ═CRNR^{5a}R^{5b}; or
iii) R³ is absent, hydrogen or lower alkyl and R⁴ forms a 5-7 membered heteroaromatic or heterocyclic ring with along with A₄ and the nitrogen atom on which it is substituted;
each R is independently hydrogen, hydroxy, alkoxy, alkyl, alkenyl, alkynyl, aryl, alkylaryl, heteroaryl, heterocyclyl, cycloalkyl or aralkyl;
R⁶ is NR^{6x} or O;
R^{6x} is hydrogen, hydroxy, alkyl, alkenyl, alkynyl, aryl, alkoxy, C(O)R⁹ or S(O)ₙR⁹;
R⁷ and R⁸ are selected as follows:
i) R⁷ is hydrogen, alkyl, alkenyl, alkynyl, cycloalkyl, aryl, heteroaryl or heterocyclyl; and R⁸ is selected from R⁷, nitro, C(O)R⁹ and S(O)ₙR⁹; or
ii) R⁷ and R⁸ together with the nitrogen atom on which they are substituted form a 3-7 membered heterocyclic or heteroaryl ring;
R⁹ is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy or aralkoxy;
R^{9a} is hydrogen, alkyl, alkenyl, alkynyl, cycloalkyl, aryl, heteroaryl or heterocyclyl;
r¹ is 0-3; r² is 0-3; n is 0-2 and x is 1-6,
where R, R¹—R⁹, A₁, R^{5a}, R^{5b}, R^{6x} and R^{9a} are optionally substituted with 1, 2, 3 or 4 substituents, each independently selected from Q¹, where Q¹ is halo, pseudohalo, hydroxy, oxo, thioxo, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q¹ groups, which substitute atoms in a 1, 2 or 1,3 arrangement, together form alkylene, alkylenoxy (i.e., —O—(CH₂)_{y}—), alkylenthioxy (i.e., —S—(CH₂)_{y}—), alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹, groups, which substitute the same atom, together form an alkylene; and
each Q¹ is independently unsubstituted or substituted with one, two or three substituents, each independently selected from Q²;
each Q² is independently halo, pseudohalo, hydroxy, oxo, thioxo, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, —P(R⁵⁰⁾₂, —P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylene, alkyleneoxy (i.e., —O—(CH₂)_{y}—), alkylenethioxy (i.e., —S—(CH₂)_{y}—), alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form an alkylene;
R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;
R⁵¹, R⁵² and R⁵³ are each independently hydrogen, alkyl, aryl, aralkyl, heteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and
R⁶³ is alkoxy, aralkoxy, alkyl, heteroaryl, heterocyclyl, aryl or —NR⁷⁰R⁷¹.

2. The compound of claim 1, wherein the compound has formula II: 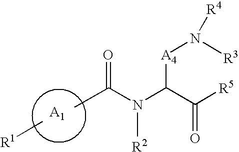
or a pharmaceutically acceptable derivative thereof.

3. The compound of claim 1, or a pharmaceutically acceptable derivative thereof, wherein A₁ is arylele or heterocyclylene.

4. The compound of claim 1, or a pharmaceutically acceptable derivative thereof, wherein A₁ is 1,2-dihydropyridine, optionally substituted with an oxo group.

5. The compound of claim 1, or a pharmaceutically acceptable derivative thereof, wherein A₁ is phenyl or 2-oxo-1,2-dihydropyridinyl.

6. The compound of claim 1, wherein the compound has formula III: 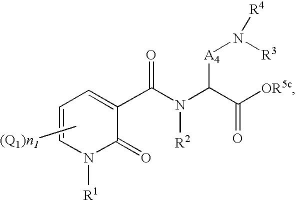
or a pharmaceutically acceptable derivative thereof, wherein
R^{5c} is hydrogen or lower alkyl; and n, is 0 to 3.

7. The compound of claim 1, or a pharmaceutically acceptable derivative thereof, wherein R¹ is aryl, aralkyl, alkylaryl, cycloalkyl, cycloalkylalkyl, heterocyclylalkyl or heteroarylalkyl.

8. The compound of claim 1, or a pharmaceutically acceptable derivative thereof, wherein R¹ is benzyl, phenyl, 2,2-diphenylethyl, 3,3-diphenylpropyl, naphthylmethyl, diphenylmethyl, naphthyl or dithiophen-2-ylmethyl.

9. The compound of claim 1, or a pharmaceutically acceptable derivative thereof, wherein R¹ is optionally substituted with one, two, three or four groups, selected from alkyl, halo, haloalkyl, aryl, aralkyl, alkylaryl, haloaryl, alkoxy, haloaryl and haloalkylaryl.

10. The compound of claim 1, or a pharmaceutically acceptable derivative thereof, wherein R¹ has formula: 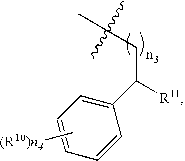
where n₃ is 0 to 3;
n4 is 0 to 5;
R¹¹ is hydrogen, alkyl, aryl, alkylaryl, haloaryl or haloalkylaryl; and
R¹⁰ is hydrogen, alkyl, halo, haloalkyl, aryl, aralkyl, alkylaryl, haloaryl, alkoxy, haloaryl or haloalkylaryl.

11. The compound of claim 10, or a pharmaceutically acceptable derivative thereof, wherein R¹¹ is hydrogen, methyl, phenyl, o-tolyl, m-tolyl, p-tolyl, 4-fluorophenyl, 3-fluorophenyl, 2-fluorophenyl, 3,5-dimethylphenyl, 3-trifluoromethylphenyl or 4-trifluoromethylphenyl.

12. The compound of claim 10, or a pharmaceutically acceptable derivative thereof, wherein R¹⁰ is hydrogen, methyl, fluoro, bromo, isopropyl, phenyl, benzyl, naphthyl, isopropylphenyl, fluorophenyl or methoxy.

13. The compound of claim 10, or a pharmaceutically acceptable derivative thereof, wherein n₃ is 0, 1, 2 or 3.

14. The compound of claim 10, or a pharmaceutically acceptable derivative thereof, wherein n₃ is 1 or 2.

15. The compound of claim 10, wherein R¹ has formula: 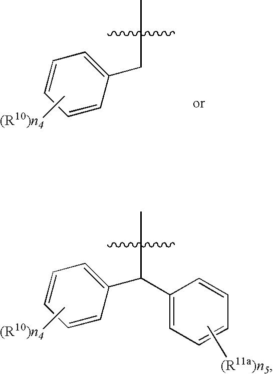
or a pharmaceutically acceptable derivative thereof, wherein R¹⁰ is hydrogen, methyl, fluoro, chloro, bromo, iodo, isopropyl, trifluoromethyl, phenyl, benzyl, naphthyl, isopropylphenyl, fluorophenyl or methoxy; R^{11a} is hydrogen, methyl, fluoro or trifluoromethyl and n₅ is 1, 2 or 3.

16. The compound of claim 10, or a pharmaceutically acceptable derivative thereof, wherein R¹ has formula:

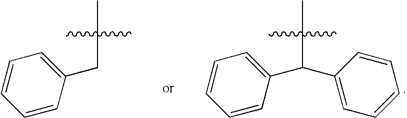

17. The compound of claim 1, or a pharmaceutically acceptable derivative thereof, wherein R² is hydrogen or lower alkyl.

18. The compound of claim 1, or a pharmaceutically acceptable derivative thereof, wherein R² is hydrogen.

19. The compound of claim 1, or a pharmaceutically acceptable derivative thereof, wherein R³ is hydrogen, lower alkyl or aryl.

20. The compound of claim 1, or a pharmaceutically acceptable derivative thereof, wherein R³ is hydrogen, methyl, ethyl, or phenyl.

21. The compound of claim 1, or a pharmaceutically acceptable derivative thereof, wherein R³ is hydrogen.

22. The compound of claim 1, or a pharmaceutically acceptable derivative thereof, wherein R⁵ is hydroxy or alkoxy.

23. The compound of claim 1, or a pharmaceutically acceptable derivative thereof, wherein R⁵ is hydroxy or methoxy.

24. The compound of claim 1, or a pharmaceutically acceptable derivative thereof, wherein R⁴ is alkyl, —C(═NR)R, C(═R⁶)NR⁷R⁸, C(O)R⁹ or S(O)ₙR⁹.

25. The compound of claim 1, or a pharmaceutically acceptable derivative thereof, wherein R⁴ is aryloxycarbonyl, alkylarylsulfonyl, heterocyclylsulfonyl, alkylsulfonyl, haloalkylsulfonyl, heterocyclyl, heteroaryl or C(═R⁶)NR⁷R⁸.

26. The compound of claim 1, or a pharmaceutically acceptable derivative thereof, wherein R⁴ is hydrogen, ethyl, benzyloxycarbonyl, p-tolylsulfonyl, methylsulfonyl, trifluoromethylsulfonyl, 4,5-dihydro-1H-imidazol-2-yl, pyrimidin-2-yl or C(═R⁶)NR⁷R⁸.

27. The compound of claim 1, or a pharmaceutically acceptable derivative thereof, wherein R⁴ is —C(═R⁶)NR⁷R⁸ or —C(═R⁶)NR⁷R⁸, wherein
R⁶ is NR^{6x} or O;
R^{6x} is hydrogen, hydroxy, alkyl, —C(O)R⁹ or —S(O)ₙR⁹;
R⁷ is hydrogen or alkyl; and
R⁸ is hydrogen, alkyl, nitro, C(O)R⁹ or S(O)ₙR⁹; and
each R is independently selected from hydrogen, hydroxy, alkyl, carboxyalkyl, cycloalkyl, alkoxycarbonyl, aryl and heteroaryl.

28. The compound of claim 24, or a pharmaceutically acceptable derivative thereof, wherein R⁶ is NR^{6x} or O;
R^{6x} is hydrogen, methyl or ethoxycarbonyl;
R⁷ is hydrogen; and
R⁸ is hydrogen, nitro, ethoxycarbonyl or p-tolylsulfonyl.

29. The compound of claim 1, or a pharmaceutically acceptable derivative thereof, wherein R⁴ has formula: 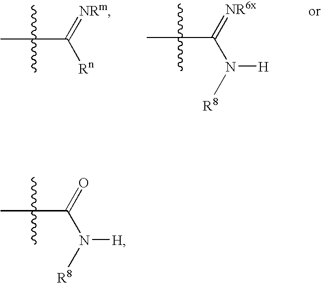
where R^{6x} is hydrogen, hydroxy, methyl or ethoxycarbonyl; R⁸ is hydrogen, nitro, ethoxycarbonyl or p-tolylsulfonyl; R^{m} is hydrogen, hydroxy or alkyl; and Rⁿ is hydrogen, alkyl, cycloalkyl, aryl, alkoxycarbonylalyl or carboxyalkyl.

30. The compound of claim 1, or a pharmaceutically acceptable derivative thereof, wherein R⁴ has formula:

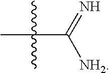

31. The compound of claim 1, or a pharmaceutically acceptable derivative thereof, wherein A₄ is alkylene or arylene.

32. The compound of claim 1, or a pharmaceutically acceptable derivative thereof, wherein A₄ is —(CH₂)n₂— or phenylene and n2 is 1-5.

33. The compound of claim 10, or a pharmaceutically acceptable derivative thereof, wherein the compound has formula: 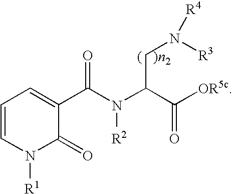
where R⁴ is —C(═NH)NH₂; n₂ is 3; R²R³ and R⁵ are each hydrogen and R¹ is selected from

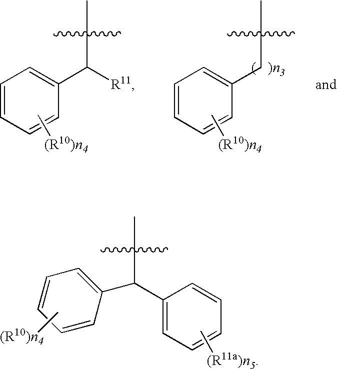

34. The compound of claim 1, wherein the compound has formula: 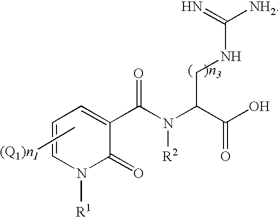
or a pharmaceutically acceptable derivative thereof.

35. The compound of claim 10, wherein the compound has formula: 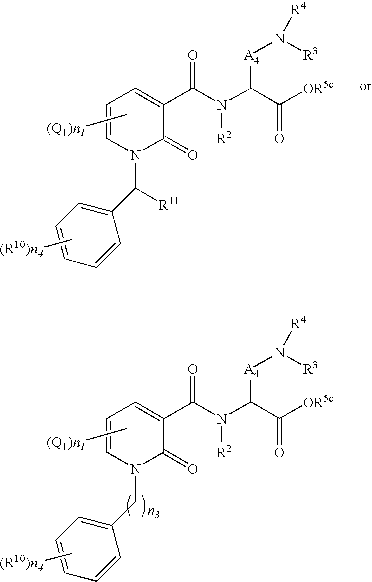
or a pharmaceutically acceptable derivative thereof.

36. The compound of claim 10, wherein the compound has formula: 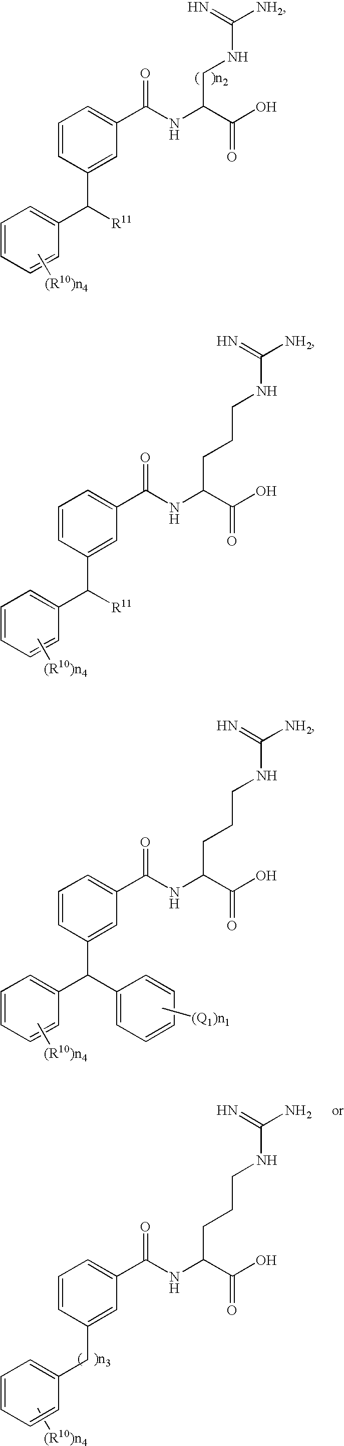 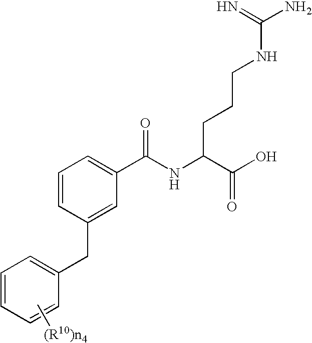
or a pharmaceutically acceptable derivative thereof.

37. The compound of claim 10, wherein the compound has formula: 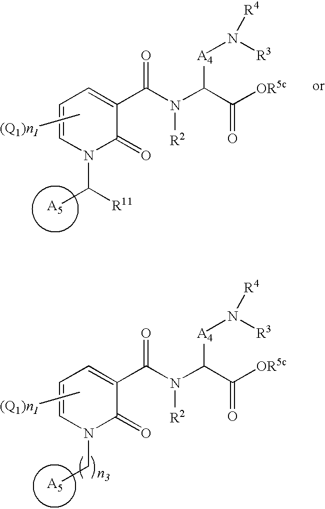
or a pharmaceutically acceptable derivative thereof, where A₅ is 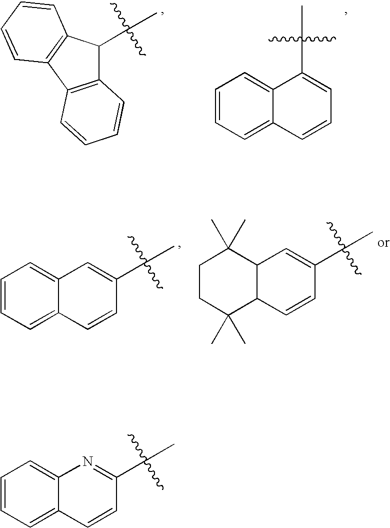
and where A₅ is optionally substituted with one, two or three groups selected from halo, alkyl, and alkoxy.

38. The compound of claim 1, wherein the compound has formula: 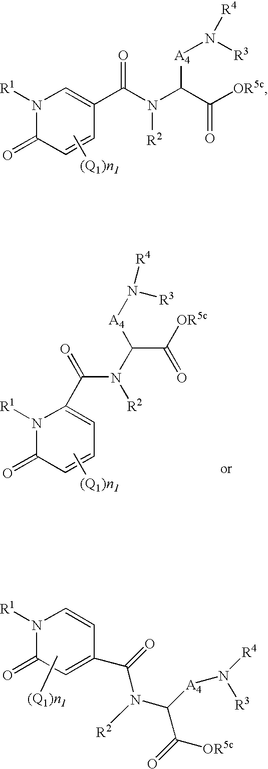
or a pharmaceutically acceptable derivative thereof.

39. The compound of claim 1, wherein 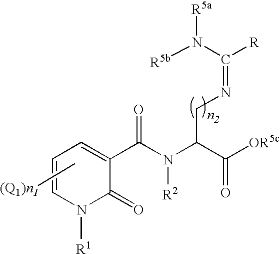
or a pharmaceutically acceptable derivative thereof.

40. The compound of claim 1, wherein 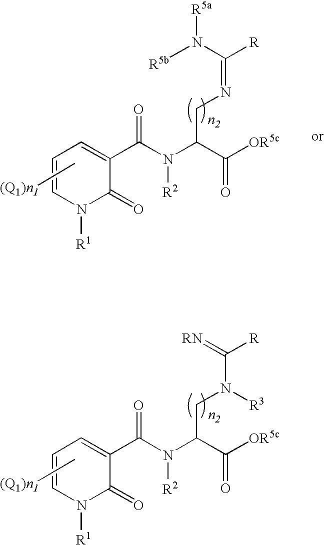
or a pharmaceutically acceptable derivative thereof.

41. The compound of claim 40, wherein 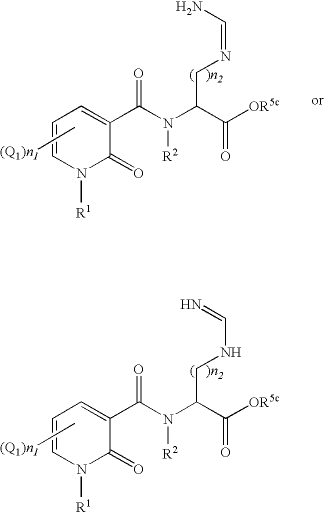
or a pharmaceutically acceptable derivative thereof.

42. The compound of claim 41, wherein 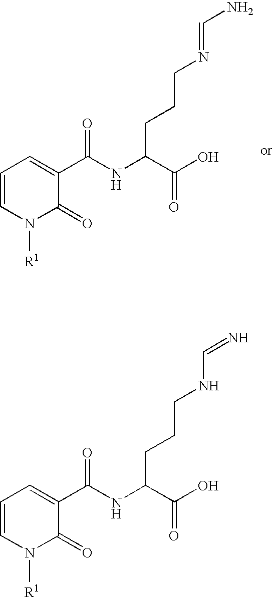
or a pharmaceutically acceptable derivative thereof.

43. The compound of claim 1, wherein the compound has formula: 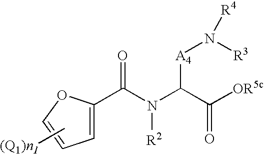
or a pharmaceutically acceptable derivative thereof.

44. The compound of claim 43, wherein the compound has formula: 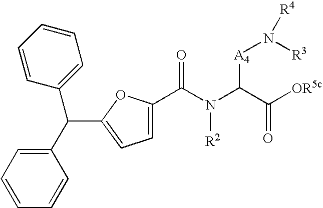
or a pharmaceutically acceptable derivative thereof.

45. The compound of claim 1, wherein the compound has formula: 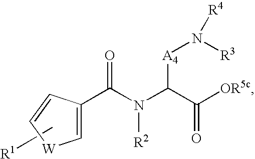
or a pharmaceutically acceptable derivative thereof, wherein
W is O or S;
R is aralkyl;
A₄ is alkylene;
R¹ is alkyl, aralkyl or heteroaralkyl, optionally substituted with one or two alkyl or halo;
R² is hydrogen or alkyl
R^{5c} is hydrogen or alkyl;
R^{5a} and R^{5b} are selected as follows:
R³ is hydrogen or alkyl;
R⁴ is alkyl, —C(═R⁶)NR⁷R⁸ or —C(═NR^{m})Rⁿ;
R^{m} is hydrogen or hydroxy;
Rⁿ is hydrogen, alkyl, aryl, heteroaryl, cycloalkyl, alkoxycarbonylalkyl or hydroxy;
R⁶ is NR^{6x};
R^{6x} is hydrogen, OH or alkyl;
R⁷ is hydrogen or alkyl; and
R⁸ is hydrogen or alkyl.

46. The compound of claim 45 having formula: 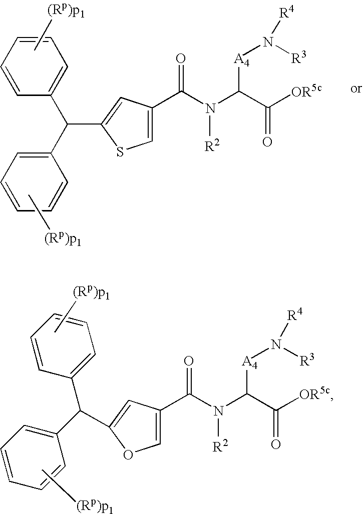
or a pharmaceutically acceptable derivative thereof, where each R^{p} is independently hydrogen, halo or alkyl; and p₁ is 1 or 2.

47. A compound of formula: 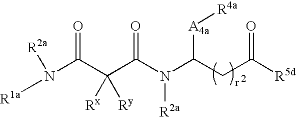
or a pharmaceutically acceptable derivative thereof, wherein
R^{1a} is aryl, aralkyl, alkylaryl, cycloalkyl, cycloalkylalkyl, heterocyclyl, heterocyclylalkyl, heteroaryl or heteroarylalkyl;
R^{2a} and R^{3a} are each independently hydrogen, alkyl, alkenyl, alkynyl, cycloalkyl, aryl, heteroaryl or heterocyclyl;
R^{5d} is OR^{a} or NR^{5e}R^{5f};
R^{a} is hydrogen, alkyl, alkenyl, alkynyl, aryl, alkylaryl, heteroaryl, heterocyclyl, cycloalkyl or aralkyl;
R^{5e} and R^{5f} are selected as follows:
i) R^{5e} and R^{5f} are each independently hydrogen, alkyl, alkenyl, alkynyl, cycloalkyl, aryl, heteroaryl or heterocyclyl; or
ii) R^{5e} and R^{5f} together with the nitrogen atom on which they are substituted form a 3-7 membered heterocyclic or heteroaryl ring;
A₄ is alkylene, alkenylene, alkynylene, alk(en)(yn)ylene, cycloalkylene, arylene, aralkylene, alkylarylene, heteroarylene or heterocyclylene;
R^{4a} is 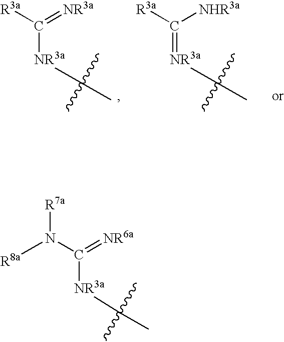
R^{6a} is hydrogen, alkyl, alkenyl, alkynyl, aryl, C(O)R^{9a} or S(O)ₚR^{9a};
R^{7a} and R^{8a} are selected as follows:
i) R^{7a} is hydrogen, alkyl, alkenyl, alkynyl, cycloalkyl, aryl, heteroaryl or heterocyclyl; and R^{8a} is selected from R^{7a}, nitro, C(O)R^{9a} and S(O)ₚR^{9a}; or
ii) R^{7a} and R^{8a} together with the nitrogen atom on which they are substituted form a 3-7 membered heterocyclic or heteroaryl ring;
R^{9a} is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy or aralkoxy;
R^{x} and R^{y} are selected as follows:
i) R^{x} and R^{y} are each independently hydrogen, alkyl, alkenyl, alkynyl, cycloalkyl, aryl, heteroaryl or heterocyclyl; or
ii) R^{x} and R^{y} together with the carbon on which they are substituted form a 3-7 membered ring;
r² is 0-3 and p is 0-2,
where R^{1a}, R^{2a}, R^{3a}, R^{6a}, R^{7a}, R^{8a}, R^{5d}, R^{x}, R^{y}, A₄ₐ, R^{5e} and R^{5f} are optionally substituted with 1, 2, 3 or 4 substituents, each independently selected from Q¹, where Q¹ is halo, pseudohalo, hydroxy, oxo, thioxo, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —NR⁵¹R⁵²R⁵³P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q¹ groups, which substitute atoms in a 1, 2 or 1,3 arrangement, together form alkylene, alkyleneoxy (i.e., —O—(CH₂)_{y}—), alkylenethioxy (i.e., —S—(CH₂)_{y}—), alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form an alkylene; and
each Q¹ is independently unsubstituted or substituted with one, two or three substituents, each independently selected from Q²;
each Q² is independently halo, pseudohalo, hydroxy, oxo, thioxo, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N+R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylene, alkyleneoxy (i.e., —O—(CH₂)_{y}—), alkylenethioxy (i.e., —S—(CH₂)_{y}—), alkylenedioxy (i.e., —O—(CH₂)_{y}—O—), thioalkylenoxy (i.e. —S—(CH₂)_{y}—O—) or alkylenedithioxy (i.e., —S—(CH₂)_{y}—S—) where y is 1 or 2; or two Q² groups, which substitute the same atom, together form an alkylene;
R⁵⁰ is hydroxy, alkoxy, aralkoxy, alkyl, heteroaryl, heterocyclyl, aryl or —NR⁷⁰R⁷¹, where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;
R⁵¹, R⁵² and R⁵³ are each independently hydrogen, alkyl, aryl, aralkyl, heteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and
R⁶³ is alkoxy, aralkoxy, alkyl, heteroaryl, heterocyclyl, aryl or —NR⁷⁰R⁷¹.

48. The compound of claim 47, wherein R^{x} and R^{y} are selected as follows:
i) R^{x} and R^{y} are each independently alkyl, alkenyl, alkynyl, cycloalkyl, aryl, heteroaryl or heterocyclyl; or
ii) R^{x} and R^{y} together with the carbon on which they are substituted form a 3-7 membered ring.

49. The compound of claim 47, wherein the compound has formula: 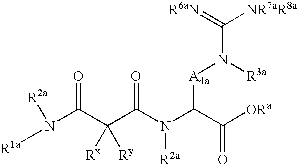
or a pharmaceutically acceptable derivative thereof.

50. The compound of claim 49, wherein the compound has formula: 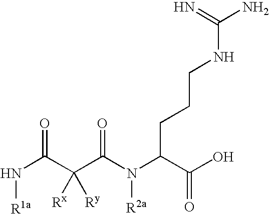
or a pharmaceutically acceptable derivative thereof.

51. The compound of claim 50, wherein the compound has formula: 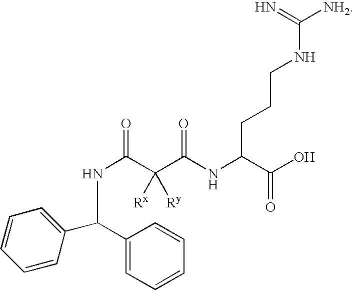
or a pharmaceutically acceptable derivative thereof, where R^{x} and R^{y} are both methyl.

52. A compound of formula: 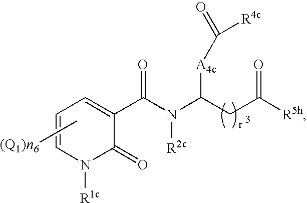
or a pharmaceutically acceptable derivative thereof, wherein
R^{1c} is aralkyl;
R^{2c} is hydrogen, alkyl, alkenyl, alkynyl, cycloalkyl, aryl, heteroaryl or heterocyclyl;
R^{5h} is OR^{c} or NR⁵ⁱR^{5j};
R^{c} is hydrogen, alkyl, alkenyl, alkynyl, aryl, alkylaryl, heteroaryl, heterocyclyl, cycloalkyl or aralkyl;
R⁵ⁱ and R^{5j} are selected as follows:
i) R⁵ⁱ and R^{5j} are each independently hydrogen, alkyl, alkenyl, alkynyl, cycloalkyl, aryl, heteroaryl or heterocyclyl; or
ii) R⁵ⁱ and R^{5j} together with the nitrogen atom on which they are substituted form a 3-7 membered heterocyclic or heteroaryl ring;
A_{4c} is alkylene, alkenylene, alkynylene, alk(en)(yn)ylene, cycloalkylene, arylene, aralkelene, alkylarylene, heteroarylene or heterocyclylene;
R^{4c} is R^{5h} 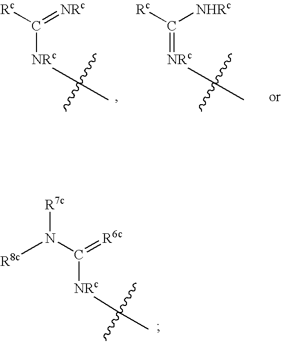
R^{6c} is NR^{6y} or O;
R^{6y} is hydrogen, alkyl, alkenyl, alkynyl, aryl, C(O)R^{9c} or S(O)ₚR^{9c};
R^{7c} and R^{8c} are selected as follows:
i) R^{7c} is hydrogen, alkyl, alkenyl, alkynyl, cycloalkyl, aryl, heteroaryl or heterocyclyl; and R^{8c} is selected from R^{7c}, nitro, C(O)R^{9c} and S(O)ₚR^{9c}; or
ii) R^{7c} and R^{8c} together with the nitrogen atom on which they are substituted form a 3-7 membered heterocyclic or heteroaryl ring;
R^{9c} is hydrogen, hydroxy, alkyl, haloalkyl, alkenyl, alkynyl, aryl, alkylaryl, heterocyclyl, cycloalkyl, aralkyl, alkoxy, alkenyloxy, alkynyloxy, aryloxy, alkylaryloxy, heterocyclyloxy, cycloalkyloxy or aralkoxy;
r³ is 0-3; p is 0-2 and n₆ is 0-3, and
where R^{1c}, R^{2c}, R^{4c}, R^{5h}, R^{c}, R⁵ⁱ, R^{5j} and A_{4c} are optionally substituted with 1, 2, 3 or 4 substituents, each independently selected from Q¹, where Q¹ is halo, pseudohalo, hydroxy, oxo, thioxo, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q¹ groups, which substitute atoms in a 1, 2 or 1,3 arrangement, together form alkylene, alkyleneoxy (i.e., —O—(CH₂)_{y}—), alkylenethioxy (i.e., —S—(CH₂)_{y}—), alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q¹ groups, which substitute the same atom, together form an alkylene; and
each Q¹ is independently unsubstituted or substituted with one, two or three substituents, each independently selected from Q²;
each Q² is independently halo, pseudohalo, hydroxy, oxo, thioxo, nitrile, nitro, formyl, mercapto, hydroxycarbonyl, hydroxycarbonylalkyl, alkyl, haloalkyl, polyhaloalkyl, aminoalkyl, diaminoalkyl, alkenyl containing 1 to 2 double bonds, alkynyl containing 1 to 2 triple bonds, cycloalkyl, cycloalkylalkyl, heterocyclyl, heterocyclylalkyl, aryl, heteroaryl, aralkyl, aralkenyl, aralkynyl, heteroarylalkyl, trialkylsilyl, dialkylarylsilyl, alkyldiarylsilyl, triarylsilyl, alkylidene, arylalkylidene, alkylcarbonyl, arylcarbonyl, heteroarylcarbonyl, alkoxycarbonyl, alkoxycarbonylalkyl, aryloxycarbonyl, aryloxycarbonylalkyl, aralkoxycarbonyl, aralkoxycarbonylalkyl, arylcarbonylalkyl, aminocarbonyl, alkylaminocarbonyl, dialkylaminocarbonyl, arylaminocarbonyl, diarylaminocarbonyl, arylalkylaminocarbonyl, alkoxy, aryloxy, heteroaryloxy, heteroaralkoxy, heterocyclyloxy, cycloalkoxy, perfluoroalkoxy, alkenyloxy, alkynyloxy, aralkoxy, alkylcarbonyloxy, arylcarbonyloxy, aralkylcarbonyloxy, alkoxycarbonyloxy, aryloxycarbonyloxy, aralkoxycarbonyloxy, aminocarbonyloxy, alkylaminocarbonyloxy, dialkylaminocarbonyloxy, alkylarylaminocarbonyloxy, diarylaminocarbonyloxy, guanidino, isothioureido, ureido, N-alkylureido, N-arylureido, N′-alkylureido, N′,N′-dialkylureido, N′-alkyl-N′-arylureido, N′,N′-diarylureido, N′-arylureido, N,N′-dialkylureido, N-alkyl-N′-arylureido, N-aryl-N′-alkylureido, N,N′-diarylureido, N,N′,N′-trialkylureido, N,N′-dialkyl-N′-arylureido, N-alkyl-N′,N′-diarylureido, N-aryl-N′,N′-dialkylureido, N,N′-diaryl-N′-alkylureido, N,N′,N′-triarylureido, amidino, alkylamidino, arylamidino, aminothiocarbonyl, alkylaminothiocarbonyl, arylaminothiocarbonyl, amino, aminoalkyl, alkylaminoalkyl, dialkylaminoalkyl, arylaminoalkyl, diarylaminoalkyl, alkylarylaminoalkyl, alkylamino, dialkylamino, haloalkylamino, arylamino, diarylamino, alkylarylamino, alkylcarbonylamino, alkoxycarbonylamino, aralkoxycarbonylamino, arylcarbonylamino, arylcarbonylaminoalkyl, aryloxycarbonylaminoalkyl, aryloxyarylcarbonylamino, aryloxycarbonylamino, alkylsulfonylamino, arylsulfonylamino, heteroarylsulfonylamino, heterocyclylsulfonylamino, heteroarylthio, azido, —N⁺R⁵¹R⁵²R⁵³, P(R⁵⁰)₂, P(═O)(R⁵⁰)₂, OP(═O)(R⁵⁰)₂, —NR⁶⁰C(═O)R⁶³, dialkylphosphonyl, alkylarylphosphonyl, diarylphosphonyl, hydroxyphosphonyl, alkylthio, arylthio, perfluoroalkylthio, hydroxycarbonylalkylthio, thiocyano, isothiocyano, alkylsulfinyloxy, alkylsulfonyloxy, arylsulfinyloxy, arylsulfonyloxy, hydroxysulfonyloxy, alkoxysulfonyloxy, aminosulfonyloxy, alkylaminosulfonyloxy, dialkylaminosulfonyloxy, arylaminosulfonyloxy, diarylaminosulfonyloxy, alkylarylaminosulfonyloxy, alkylsulfinyl, alkylsulfonyl, arylsulfinyl, arylsulfonyl, hydroxysulfonyl, alkoxysulfonyl, aminosulfonyl, alkylaminosulfonyl, dialkylaminosulfonyl, arylaminosulfonyl, diarylaminosulfonyl or alkylarylaminosulfonyl; or two Q² groups, which substitute atoms in a 1, 2 or 1,3 arrangement, together form alkylene, alkyleneoxy (i.e., —O—(CH₂)_{y}—), alkylenethioxy (i.e., —S—(CH₂)_{y}—), alkylenedioxy (i.e., —O—(CH₂)_{y}—O—), thioalkylenoxy (i.e., —S—(CH₂)_{y}—O—) or alkylenedithioxy (i.e., —S—(CH₂)_{y}—S—) where y is 1 or 2; or two Q² groups, which substitute the same atom, together form an alkylene;
R⁵⁰ is hydroxy, alkoxy, aralkoxy, alkyl, heteroaryl, heterocyclyl, aryl or —NR⁷⁰R⁷¹,
where R⁷⁰ and R⁷¹ are each independently hydrogen, alkyl, aralkyl, aryl, heteroaryl, heteroaralkyl or heterocyclyl, or R⁷⁰ and R⁷¹ together form alkylene, azaalkylene, oxaalkylene or thiaalkylene;
R⁵¹, R⁵² and R⁵³ are each independently hydrogen, alkyl, aryl, aralkyl, heteroaryl, heteroaralkyl, heterocyclyl or heterocyclylalkyl;
R⁶⁰ is hydrogen, alkyl, aryl, aralkyl, heteroaryl, heteroaralkyl, heterocyclyl or heterocyclylalkyl; and
R⁶³ is alkoxy, aralkoxy, alkyl, heteroaryl, heterocyclyl, aryl or —NR⁷⁰R⁷¹.

53. The compound of claim 52, wherein the compound has formula: 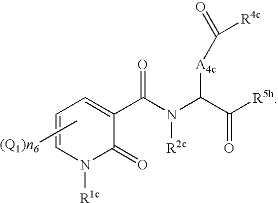
or a pharmaceutically acceptable derivative thereof.

54. The compound of claim 52, wherein the compound has formula: 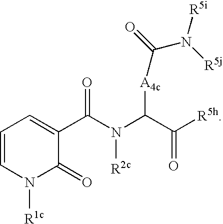
or a pharmaceutically acceptable derivative thereof.

55. The compound of claim 52, wherein the compound has formula: 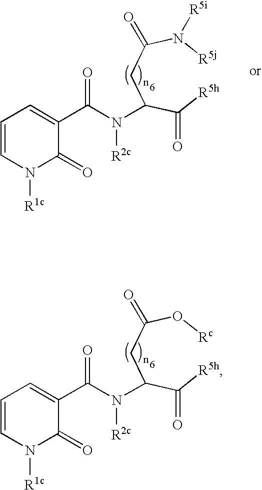
where n₆ is 1-5, or a pharmaceutically acceptable derivative thereof.

56. The compound of claim 52, wherein the compound has formula: 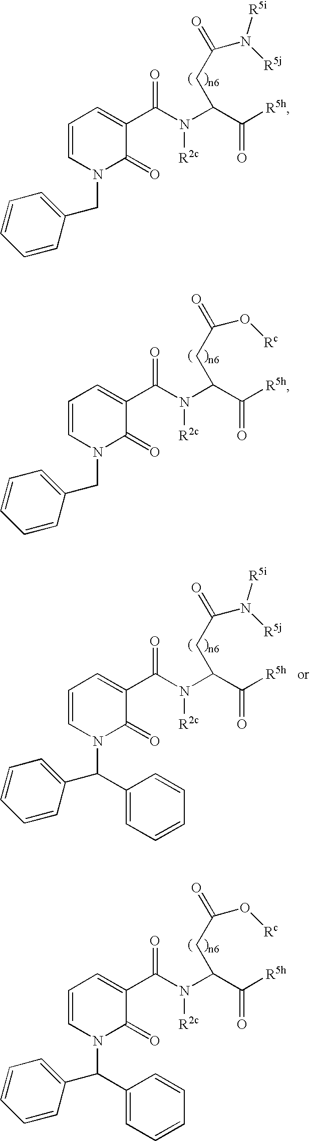 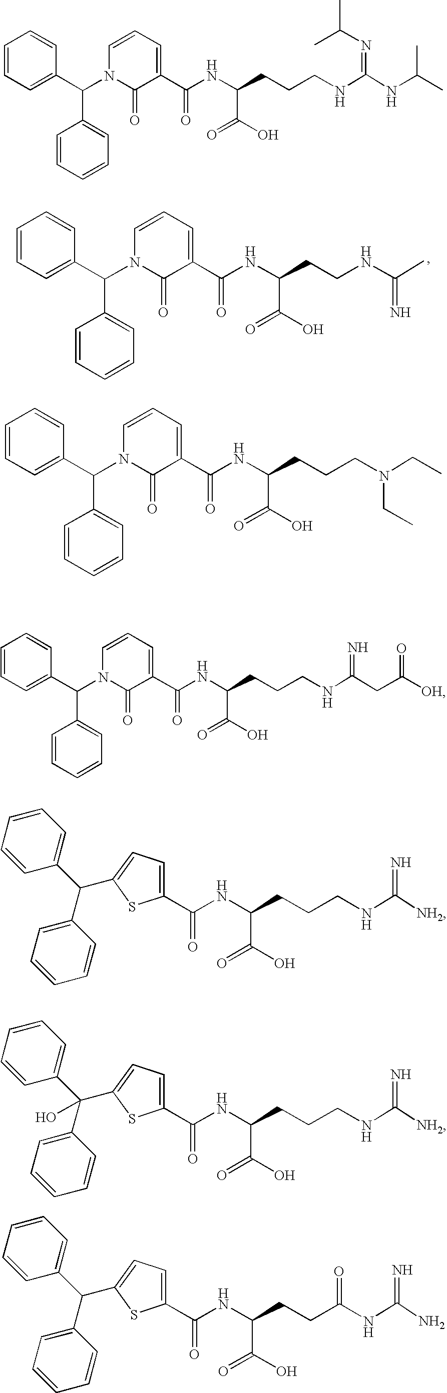
or a pharmaceutically acceptable derivative thereof.

57. The compound of claim 1, selected from: 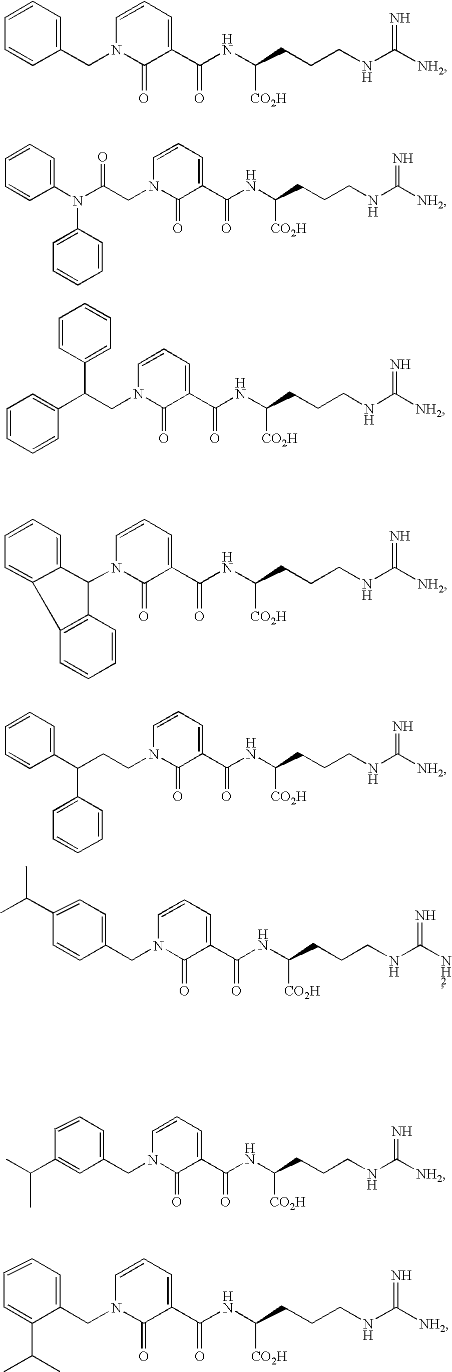 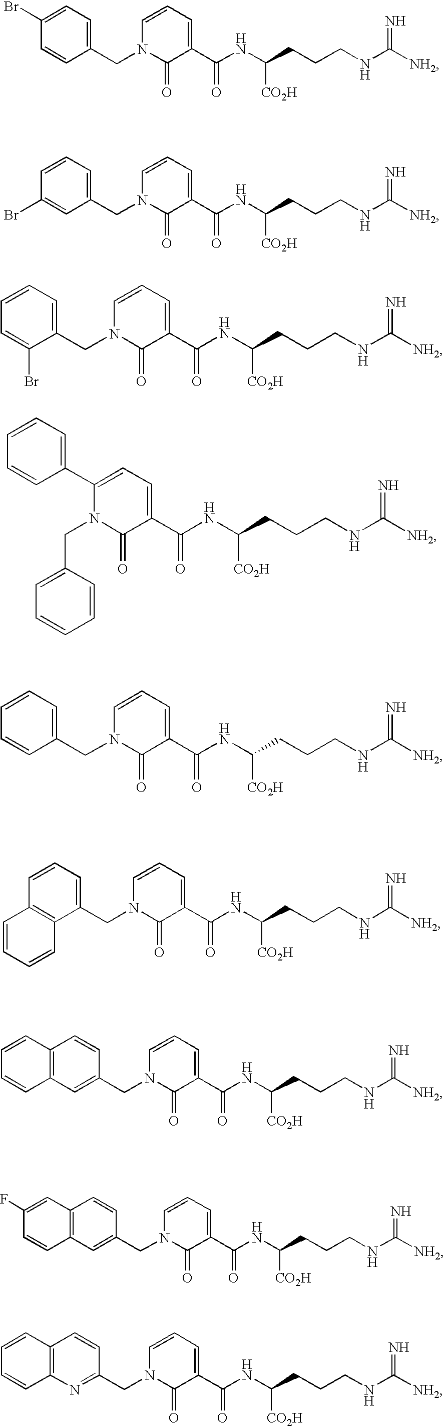 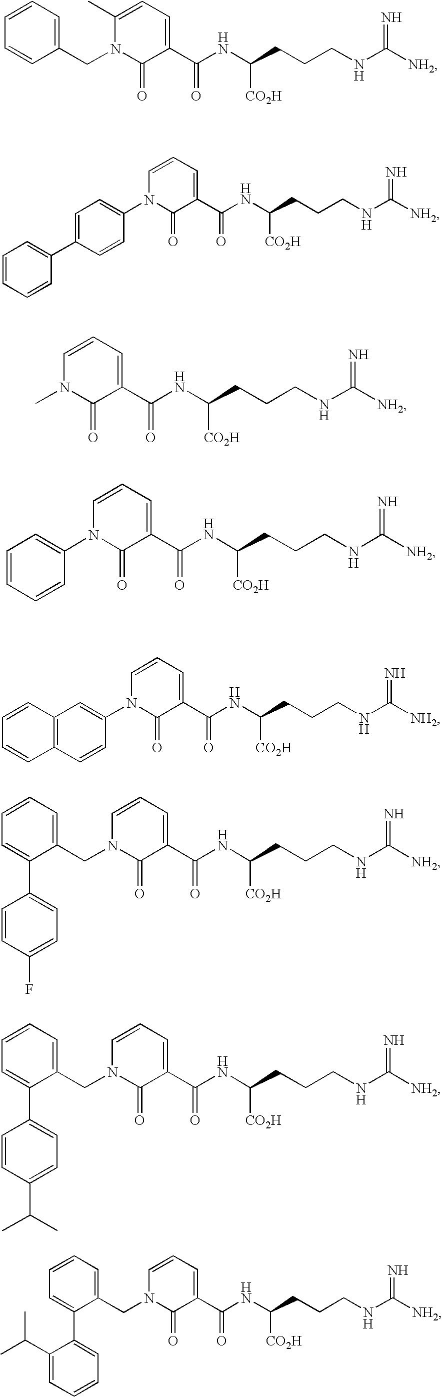 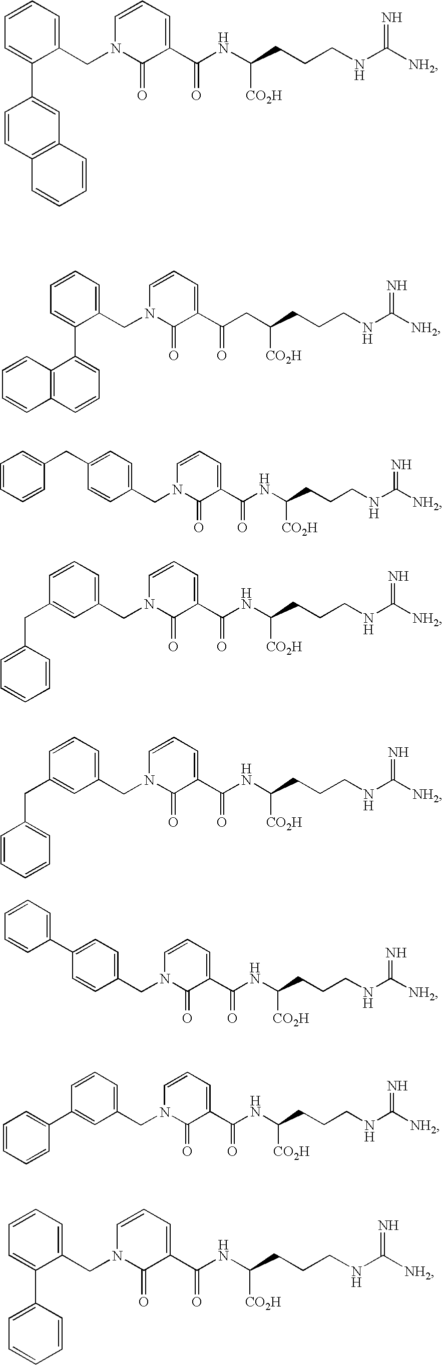 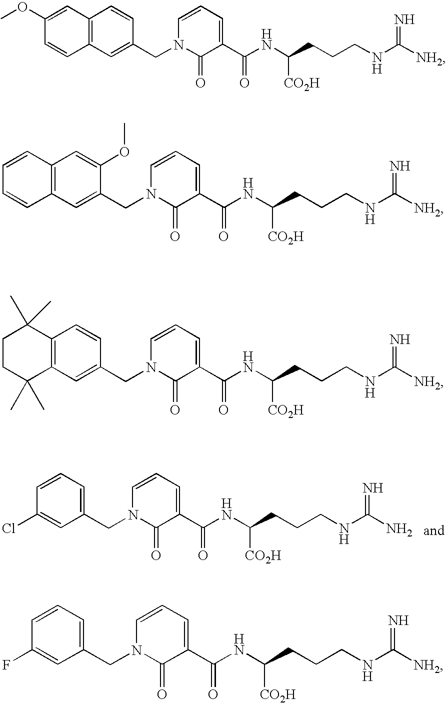
and pharmaceutically acceptable derivatives thereof.

58. The compound of claim 1, selected from: 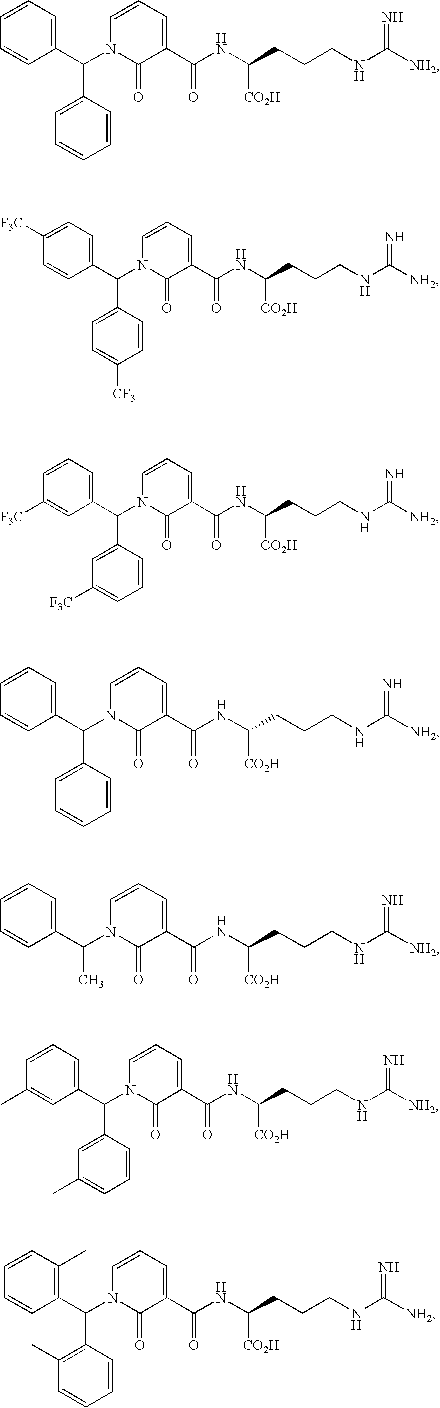 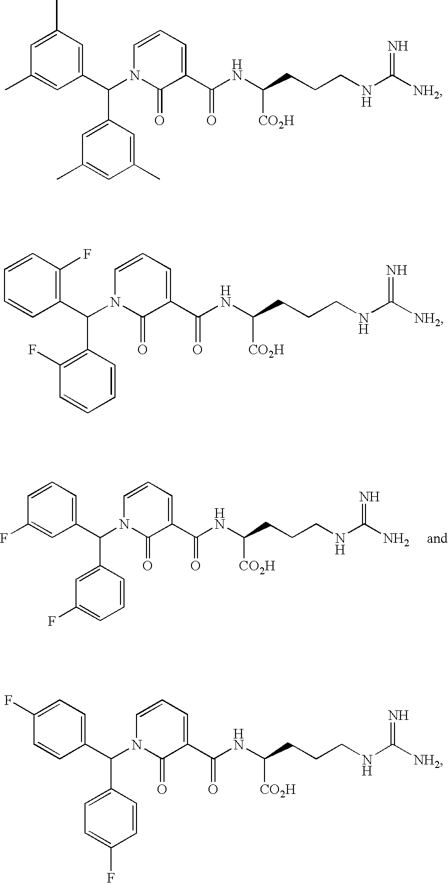
and pharmaceutically acceptable derivatives thereof.

59. The compound of claim 1, selected from: 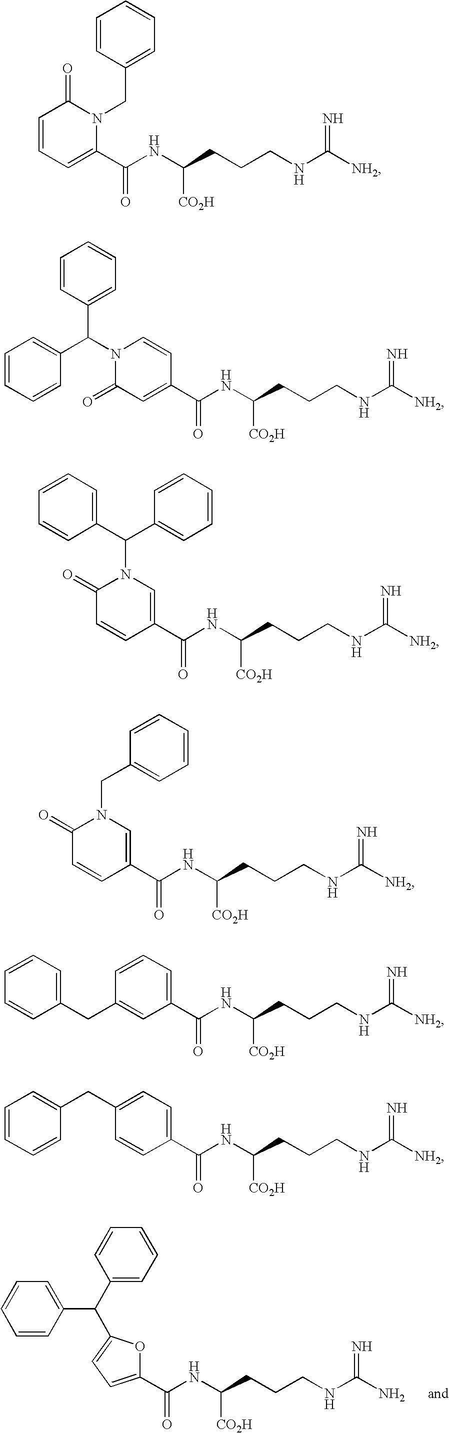 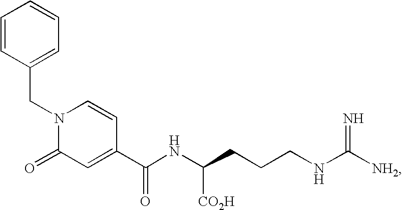
and pharmaceutically acceptable derivatives thereof.

60. The compound of claim 1, selected from: 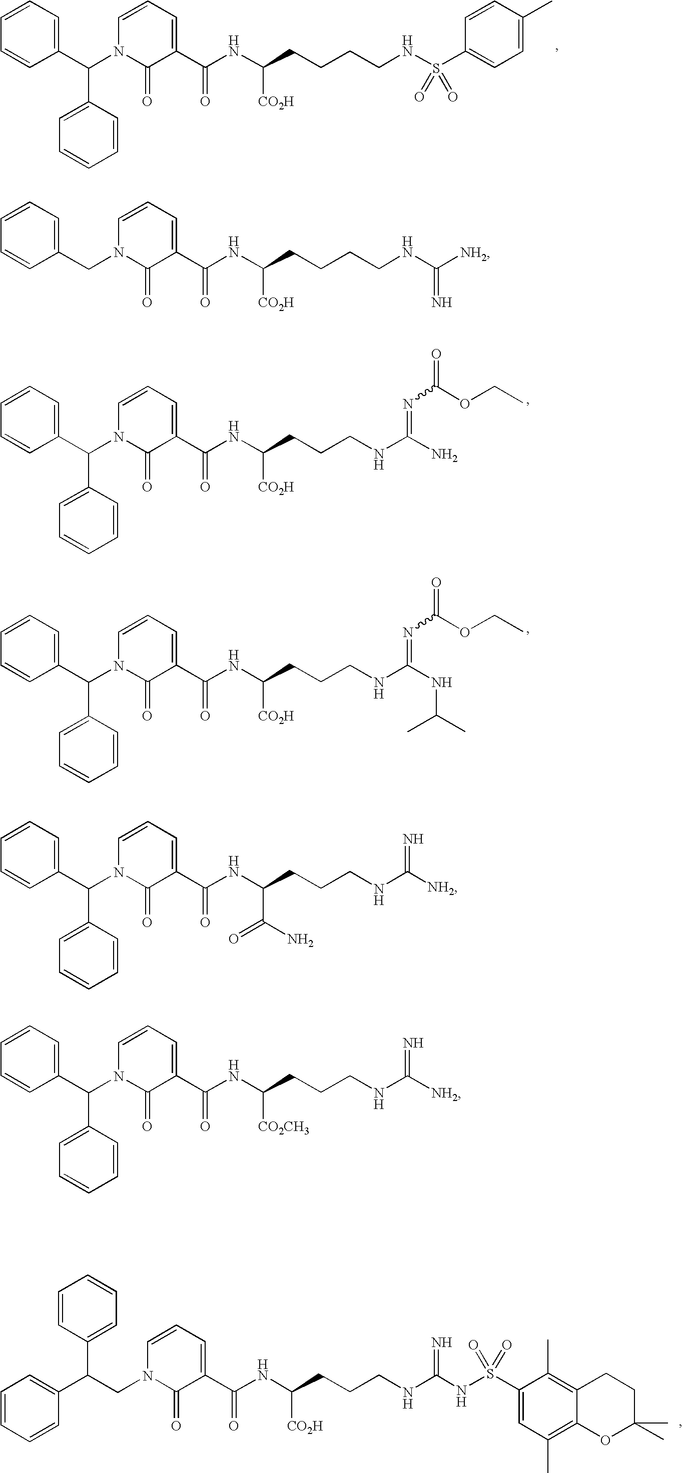 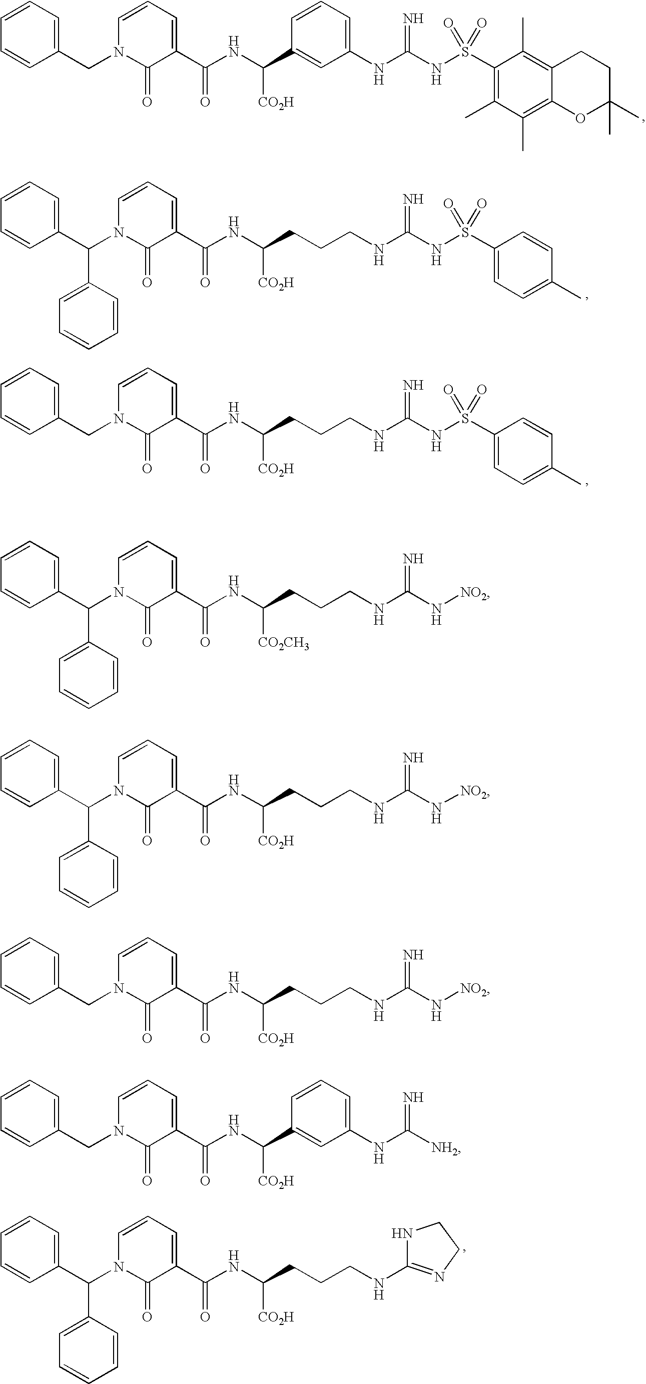 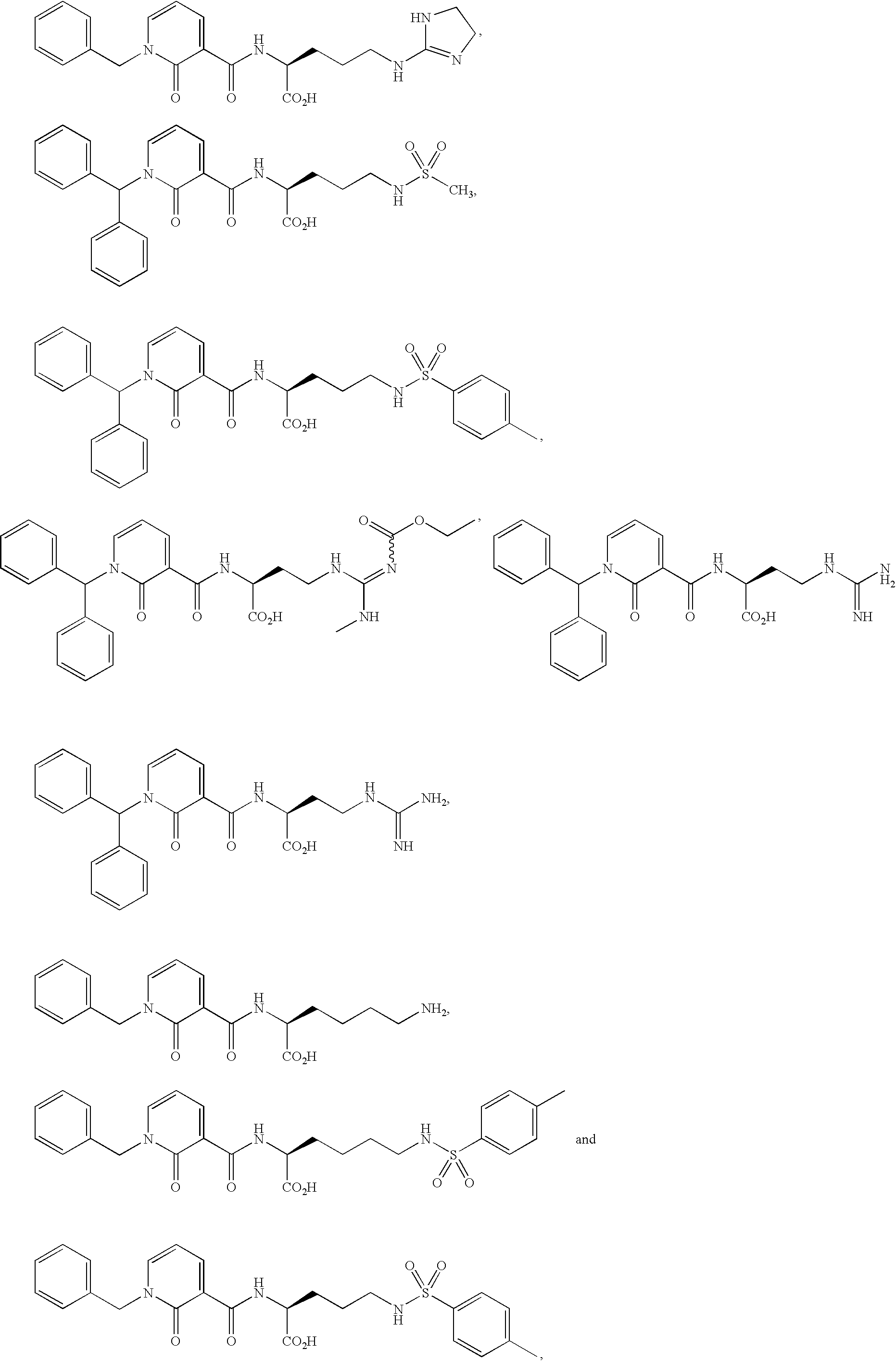
and pharmaceutically acceptable derivatives thereof.

61. The compound of claim 1, selected from: 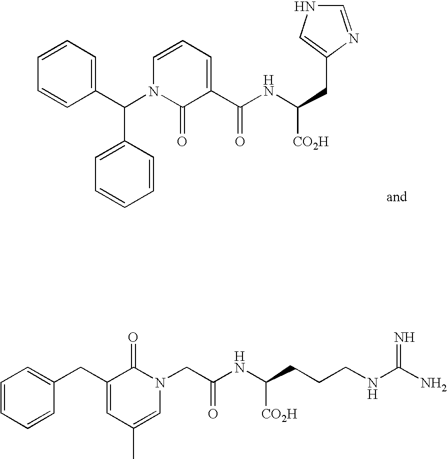
and pharmaceutically acceptable derivatives thereof.

62. The compound of claim 47, wherein the compound is: 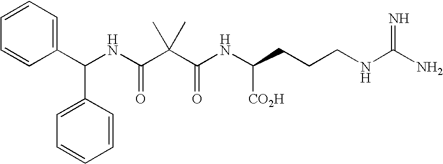
or a pharmaceutically acceptable derivative thereof.

63. The compound of claim 52, selected from: 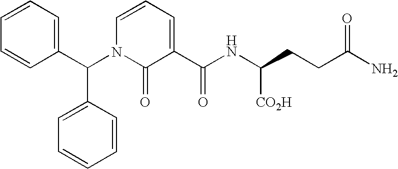
or a pharmaceutically acceptable derivative thereof.

64. The compound of claim 1, selected from 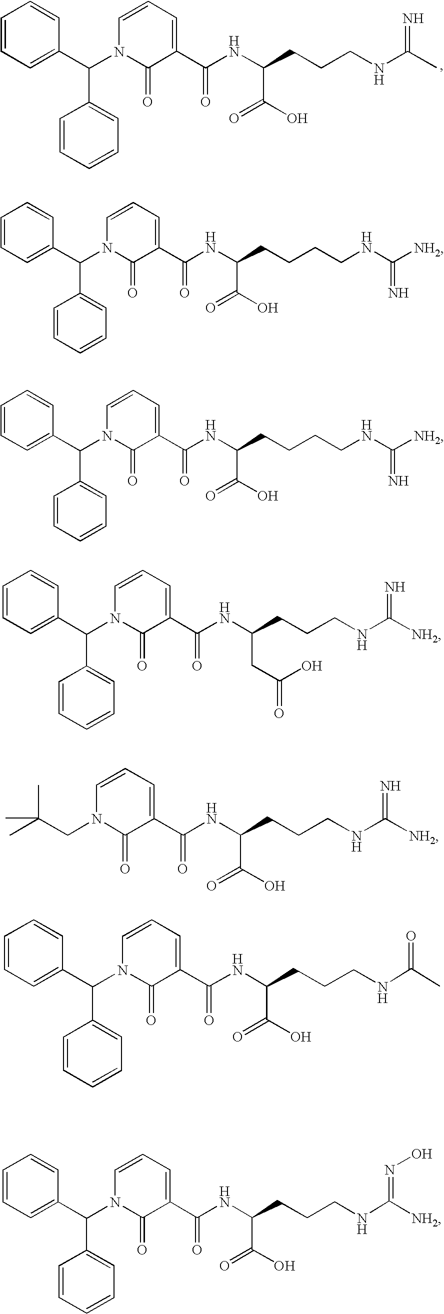 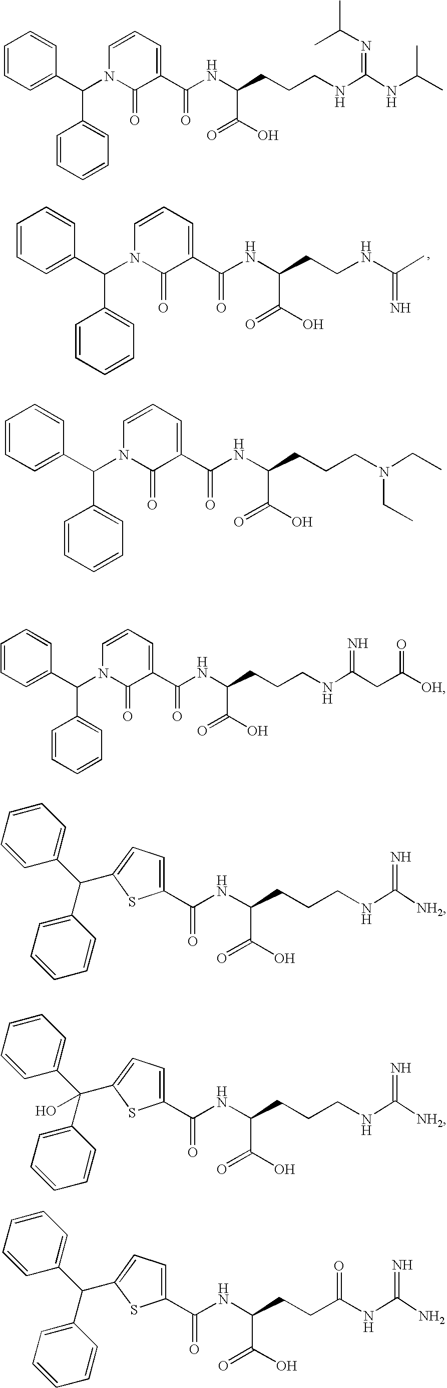 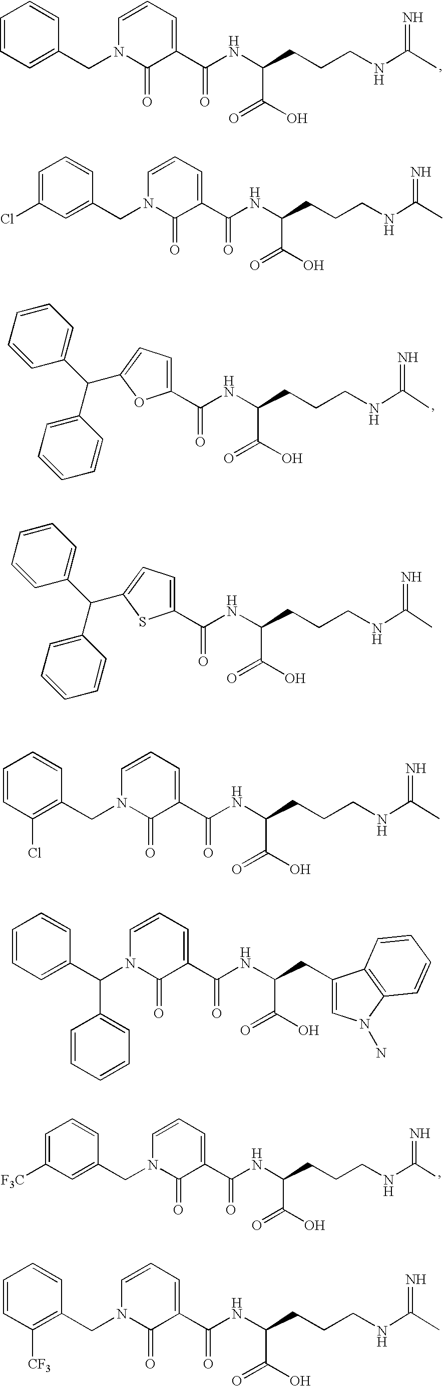 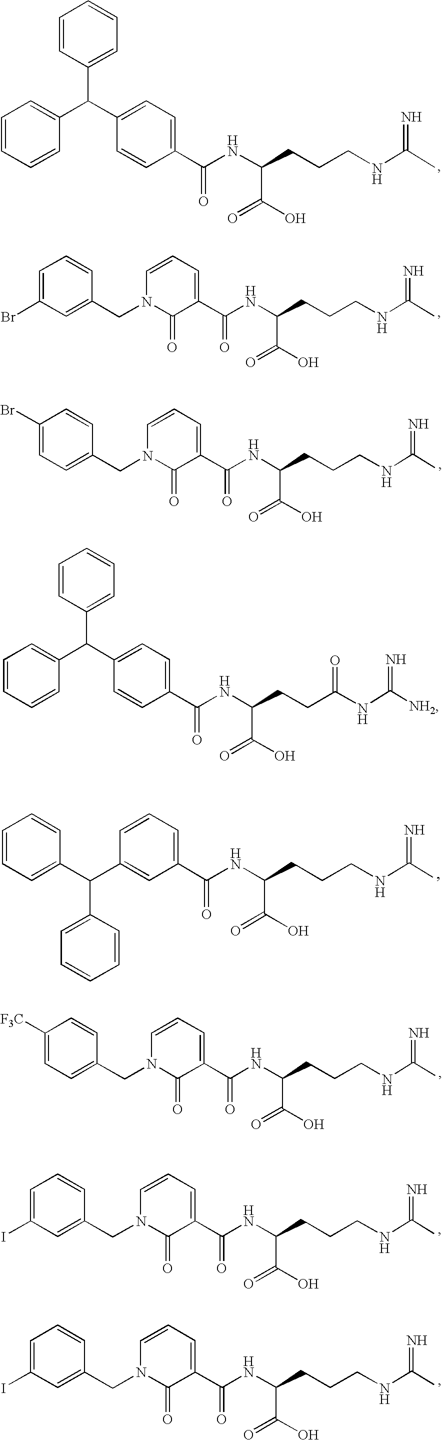 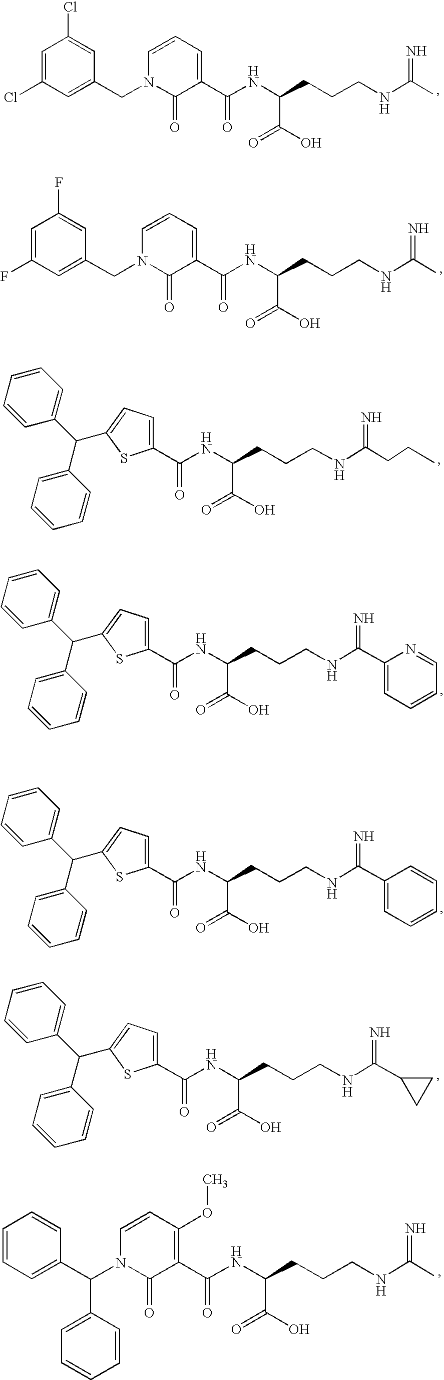 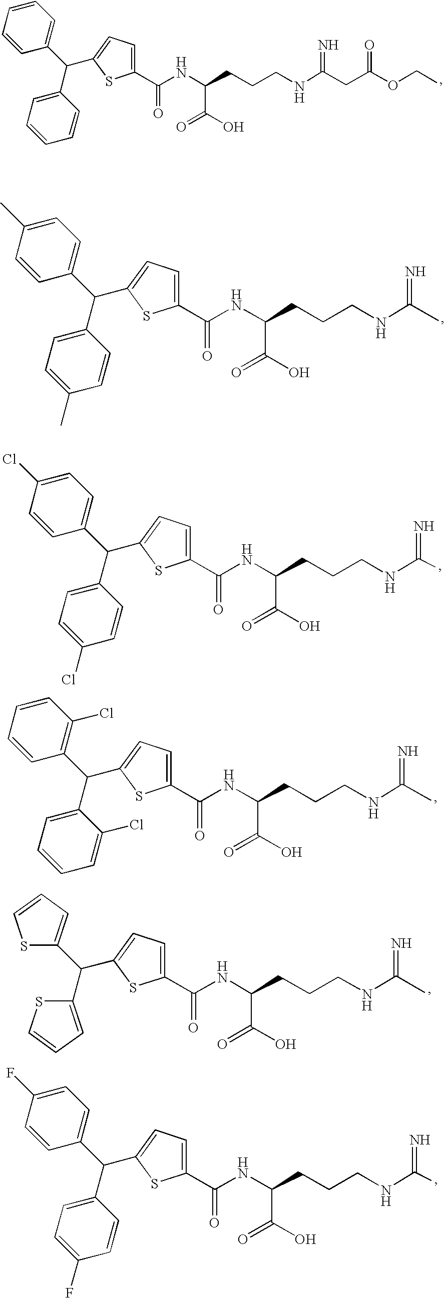 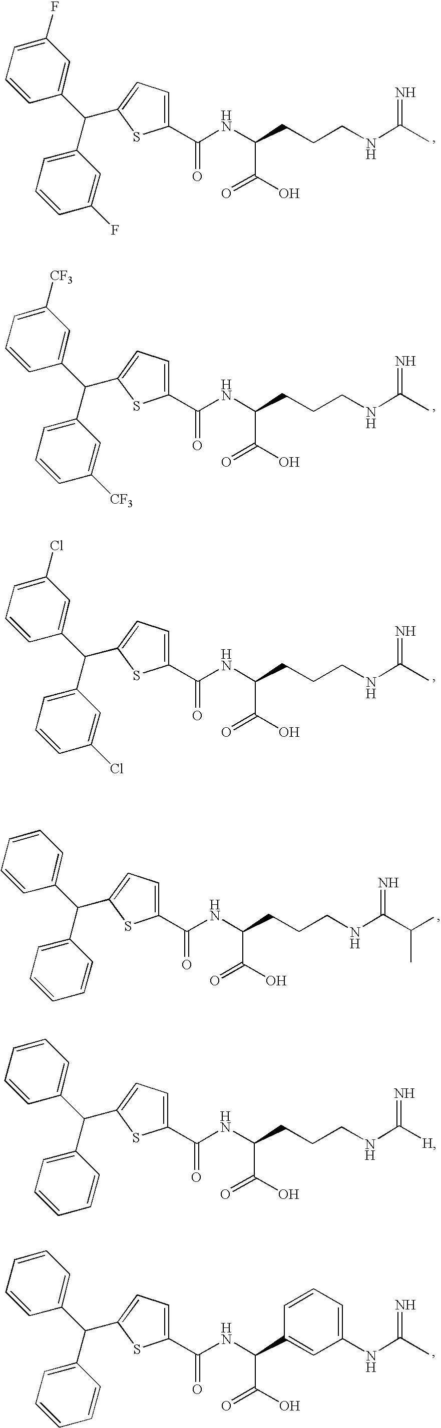 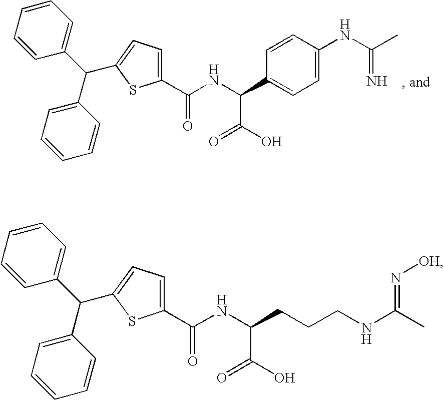
or a pharmaceutically acceptable derivative thereof.

65. A pharmaceutical composition comprising a compound of claim 1 or a pharmaceutically acceptable derivative thereof, and a pharmaceutically acceptable carrier.

66. A method for modulating C3a receptor activity comprising contacting the C3a receptor with the compound of claim 1 or a pharmaceutically acceptable derivative thereof.

67. A method for treating, preventing or ameliorating a disease associated with C3a receptor modulation comprising administering the compound of claim 1 or a pharmaceutically acceptable derivative thereof.

68. The method of claim 67, wherein the disease is acute and chronic inflammatory disease, atherosclerosis, chronic polyarthritis, systemic vasculitis, multiple sclerosis, Alzheimer's Disease, CNS inflammatory disease, Crohn's Disease, food allergy, non-bronchial allergy, osteoarthritis, osteoporosis, thyroid disease, coronary heart disease, renal disease, rheumatological disease, neurological diseases, dermatological disease, biocompatibility/shock disease, atheroma, bowel inflammation, thyroiditis, infertility, suseptibility to pyogenic infections, glomerulonephritis, suseptibility to neisserial infections, recurrent subcutaneous swelling, mucosal oedema, and recurrent episodes of thrombosis/haemolysis.

69. A pharmaceutical composition comprising a compound of claim 47 or a pharmaceutically acceptable derivative thereof, and a pharmaceutically acceptable carrier.

70. A method for treating, preventing or ameliorating a disease associated with C3a receptor modulation comprising administering the compound of claim 47 or a pharmaceutically acceptable derivative thereof.

71. A pharmaceutical composition comprising a compound of claim 52 or a pharmaceutically acceptable derivative thereof, and a pharmaceutically acceptable carrier.

72. A method for treating, preventing or ameliorating a disease associated with C3a receptor modulation comprising administering the compound of claim 52 or a pharmaceutically acceptable derivative thereof.
